(19) United States
(12) Patent Application Publication
(10) Pub. No.: US 2025/0169356 A1
(43) Pub. Date: May 22, 2025

(54) HETEROCYCLIC COMPOUND, AND COMPOSITION FOR FORMING ORGANIC LIGHT-EMITTING ELEMENT AND ORGANIC MATERIAL LAYER COMPRISING SAME

(71) Applicant: LT MATERIALS CO., LTD., Yongin-si, Gyeonggi-do (KR)

(72) Inventors: Gi-Back LEE, Yongin-si (KR); Jun-Tae MO, Yongin-si (KR); Dong-Jun KIM, Yongin-si (KR)

(73) Assignee: LT MATERIALS CO., LTD., Yongin-si, Gyeonggi-do (KR)

(21) Appl. No.: 18/837,705

(22) PCT Filed: Feb. 9, 2023
(86) PCT No.: PCT/KR2023/001941

§ 371 Date: Aug. 12, 2024

(30) Foreign Application Priority Data
Feb. 14, 2022 (KR) 10-2022-0018650

(51) Int. Cl. H10K 85/60; C07C 211/54; C07D 307/91; C07D 333/76; C07D 405/04; C09K 11/02; H10K 50/11

(52) U.S. Cl. CPC H10K 85/636; C07C 211/54; C07D 307/91; C07D 333/76; C07D 405/04; C09K 11/02; H10K 85/633; H10K 85/657; H10K 85/6572; H10K 50/11; H10K 85/615; H10K 85/622; H10K 85/626; H10K 85/654; H10K 85/6574; H10K 85/6576

(57) ABSTRACT

The present specification relates to a heterocyclic compound of Chemical Formula 1, an organic light emitting device including the same and a composition for forming an organic material layer. When used in an organic light emitting device, the heterocyclic compound can lower the driving voltage of the device, improve the light emitting efficiency, and improve the service life characteristics of the device.

### TECHNICAL FIELD

The present specification relates to a heterocyclic compound, an organic light emitting device including the same and a composition for forming an organic material layer.

### CROSS-REFERENCE TO RELATED APPLICATIONS

This application claims priority to and the benefits of Korean Patent Application No. 10-2022-0018650 filed in the Korean Intellectual Property Office on Feb. 14, 2022, the entire contents of which are incorporated herein by reference.

### BACKGROUND ART

An electroluminescence device is a kind of self-emitting type display device, and has an advantage in that the viewing angle is wide, the contrast is excellent, and the response speed is fast.

An organic light emitting device has a structure in which an organic thin film is disposed between two electrodes. When a voltage is applied to an organic light emitting device having the structure, electrons and holes injected from the two electrodes combine with each other in an organic thin film to make a pair, and then, emit light while being extinguished. The organic thin film may be composed of a single layer or multiple layers, if necessary.

A material for the organic thin film may have a light emitting function, if necessary. For example, as the material for the organic thin film, it is also possible to use a compound, which may itself constitute a light emitting layer alone, or it is also possible to use a compound, which may serve as a host or a dopant of a host-dopant-based light emitting layer. In addition, as a material for the organic thin film, it is also possible to use a compound, which may play a role such as a hole injection, hole transport, electron blocking, hole blocking, electron transport or electron injection.

In order to improve the performance, service life, or efficiency of the organic light emitting device, there is a continuous need for developing a material for an organic thin film.

### CITATION LIST

(Patent Document 1) U.S. Pat. No. 4,356,429

### BRIEF DESCRIPTION

### Technical Problem

The present specification has been made in an effort to provide a heterocyclic compound, an organic light emitting device including the same and a composition for forming an organic material layer.

### Technical Solution

In an exemplary embodiment of the present specification, provided is a heterocyclic compound of the following Chemical Formula 1.

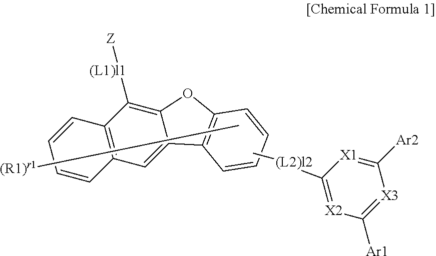

In Chemical Formula 1,
L1 and L2 are each independently a direct bond; a phenylene group; or a naphthylene group,
l1 and l2 are each independently an integer from 0 to 3, and when l1 and l2 are each 2 or higher, substituents in the parenthesis are the same as or different from each other,
R1 is hydrogen; or deuterium,
r1 is an integer from 0 to 8, and when r1 is 2 or higher, R1's are the same as or different from each other,
Z is —NR′R″; a substituted or unsubstituted alkyl group having 1 to 60 carbon atoms; a substituted or unsubstituted cycloalkyl group having 3 to 60 carbon atoms; a substituted or unsubstituted heterocycloalkyl group having 3 to 60 carbon atoms; an aryl group having 6 to 15 carbon atoms, which is unsubstituted or substituted with deuterium or an aryl group; a triphenylene group which is unsubstituted or substituted with deuterium or an aryl group; an aryl group having 20 to 60 carbon atoms, which is unsubstituted or substituted with deuterium or an aryl group; a substituted or unsubstituted dibenzofuran group; a substituted or unsubstituted dibenzothiophene group; or a substituted or unsubstituted benzocarbazole group,
X1 to X3 are each independently N or CR, and at least one thereof is N,
Ar1 and Ar2 are each independently hydrogen; deuterium; a halogen group; a cyano group; a substituted or unsubstituted alkyl group having 1 to 60 carbon atoms; a substituted or unsubstituted cycloalkyl group having 3 to 60 carbon atoms; a substituted or unsubstituted heterocycloalkyl group having 2 to 60 carbon atoms; an aryl group having 6 to 60 carbon atoms, which is unsubstituted or substituted with deuterium or an aryl group; or a substituted or unsubstituted heteroaryl group having 2 to 60 carbon atoms,
R, R′ and R″ are each independently hydrogen; deuterium; a halogen group; a cyano group; a substituted or unsubstituted alkyl group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; or a substituted or unsubstituted heteroaryl group having 2 to 60 carbon atoms, or
R is bonded to Ar1 or Ar2 to form a substituted or unsubstituted hetero ring having 2 to 60 carbon atoms, and
when Z is an aryl group having 6 to 15 carbon atoms, which is unsubstituted or substituted with an aryl group, Ar1 and Ar2 are each independently hydrogen; deuterium; a halogen group; a cyano group; a substituted or unsubstituted alkyl group having 1 to 60 carbon atoms; a substituted or unsubstituted cycloalkyl group having 3 to 60 carbon atoms; a substituted or unsubstituted heterocycloalkyl group having 2 to 60 carbon atoms; an aryl group having 6 to 60 carbon atoms, which is unsubstituted or substituted with deuterium or an aryl group; a substituted or unsubstituted dibenzofuran group; or a substituted or unsubstituted dibenzothiophene group.

In another exemplary embodiment of the present specification, provided is an organic light emitting device including: a first electrode; a second electrode; and an organic material layer having one or more layers provided between the first electrode and the second electrode, in which one or more layers of the organic material layer include one or more of the heterocyclic compounds.

In still another exemplary embodiment of the present specification, provided is a composition for forming an organic material layer, including the heterocyclic compound.

### Advantageous Effects

When used in an organic light emitting device, the heterocyclic compound described in the present specification can lower the driving voltage of the device, improve the light emitting efficiency, and improve the service life characteristics of the device. Specifically, the heterocyclic compound of the present invention is characterized by having two substituents at a specific position of naphthobenzofuran, and one of the two substituents includes an azine group, and thus, can lower the driving voltage of the device and improve the light emitting efficiency by regulating the band gap and T1 (energy level of the triplet state) values through the excellent electron withdrawing characteristics of the azine functional group to regulate the electron transfer ability and hole blocking ability. Further, by bonding the other substituent of the two substituents to the specific position to strengthen hole characteristics and simultaneously increase the planarity and glass transition temperature of the azine derivative, the heterocyclic compound of the present invention has a feature capable of improving the service life characteristics of the device by improving the thermal stability of the compound.

### DESCRIPTION OF DRAWINGS

FIGS. 1 to 4 are views each exemplarily illustrating a stacking structure of an organic light emitting device according to an exemplary embodiment of the present specification.

### EXPLANATION OF REFERENCE NUMERALS AND SYMBOLS

100: Substrate
200: Positive electrode
300: Organic material layer
301: Hole injection layer
302: Hole transport layer
303: Electron blocking layer
304: Light emitting layer
305: Hole blocking layer
306: Electron transport layer
307: Electron injection layer
400: Negative electrode

### DETAILED DESCRIPTION

Hereinafter, the present specification will be described in more detail.

When one part “includes” one constituent element in the present specification, unless otherwise specifically described, this does not mean that another constituent element is excluded, but means that another constituent element may be further included.

In the present specification,

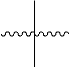

of a chemical formula means a position to which a constituent element is bonded.

The term “substitution” means that a hydrogen atom bonded to a carbon atom of a compound is changed into another substituent, and a position to be substituted is not limited as long as the position is a position at which the hydrogen atom is substituted, that is, a position at which the substituent may be substituted, and when two or more are substituted, the two or more substituents may be the same as or different from each other.

In the present specification, “substituted or unsubstituted” means being unsubstituted or substituted with one or more substituents selected from the group consisting of deuterium; a halogen group; —CN; a C1 to C60 alkyl group; a C2 to C60 alkenyl group; a C2 to C60 alkynyl group; a C1 to C60 haloalkyl group; a C1 to C60 alkoxy group; a C6 to C60 aryloxy group; a C1 to C60 alkylthioxy group; a C6 to C60 arylthioxy group; a C1 to C60 alkylsulfoxy group; a C6 to C60 arylsulfoxy group; a C3 to C60 cycloalkyl group; a C2 to C60 heterocycloalkyl group; a C6 to C60 aryl group; a C2 to C60 heteroaryl group; —SiRR′R″; —P(═O)RR′; and —NRR′, or a substituent to which two or more substituents selected among the exemplified substituents are linked, and R, R′ and R″ are each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group.

In the present specification, “when a substituent is not indicated in the structure of a chemical formula or compound” means that a hydrogen atom is bonded to a carbon atom. However, since deuterium (²H) is an isotope of hydrogen, some hydrogen atoms may be deuterium.

In an exemplary embodiment of the present application, “when a substituent is not indicated in the structure of a chemical formula or compound” may mean that all the positions that may be reached by the substituent are hydrogen or deuterium. That is, deuterium is an isotope of hydrogen, and some hydrogen atoms may be deuterium which is an isotope, and in this case, the content of deuterium may be 0% to 100%.

In an exemplary embodiment of the present application, in “the case where a substituent is not indicated in the structure of a chemical formula or compound”, when the content of deuterium is 0%, the content of hydrogen is 100%, and all the substituents do not explicitly exclude deuterium, hydrogen and deuterium may be mixed and used in the compound.

In an exemplary embodiment of the present application, deuterium is one of the isotopes of hydrogen, is an element that has a deuteron composed of one proton and one neutron as a nucleus, and may be represented by hydrogen-2, and the element symbol may also be expressed as D or 2H.

In an exemplary embodiment of the present application, the isotope means an atom with the same atomic number (Z), but different mass numbers (A), and the isotope may also be interpreted as an element which has the same number of protons, but different number of neutrons.

In an exemplary embodiment of the present application, when the total number of substituents of a basic compound is defined as T1 and the number of specific substituents among the substituents is defined as T2, the content T % of the specific substituent may be defined as T2/T1×100=T %.

That is, in an example, the deuterium content of 20% in a phenyl group represented by

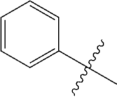 [SMILES: *c1ccccc1]

may be represented by 20% when the total number of substituents that the phenyl group can have is 5 (T1 in the formula) and the number of deuteriums among the substituents is 1 (T2 in the formula). That is, a deuterium content of 20% in the phenyl group may be represented by the following structural formula.

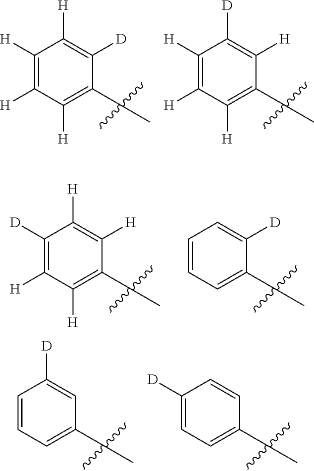

Further, in an exemplary embodiment of the present application, “a phenyl group having a deuterium content of 0%” may mean a phenyl group that does not include a deuterium atom, that is, has five hydrogen atoms.

In the present specification, the halogen may be fluorine, chlorine, bromine or iodine.

In the present specification, an alkyl group includes a straight-chain or branched-chain having 1 to 60 carbon atoms, and may be additionally substituted with another substituent. The number of carbon atoms of the alkyl group may be 1 to 60, specifically 1 to 40, and more specifically 1 to 20. Specific examples thereof include a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group, and the like, but are not limited thereto.

In the present specification, an alkenyl group includes a straight-chain or branched-chain having 2 to 60 carbon atoms, and may be additionally substituted with another substituent. The number of carbon atoms of the alkenyl group may be 2 to 60, specifically 2 to 40, and more specifically 2 to 20. Specific examples thereof include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl) vinyl-1-yl group, a 2,2-bis(diphenyl-1-yl) vinyl-1-yl group, a stilbenyl group, a styrenyl group, and the like, but are not limited thereto.

In the present specification, an alkynyl group includes a straight-chain or branched-chain having 2 to 60 carbon atoms, and may be additionally substituted with another substituent. The number of carbon atoms of the alkynyl group may be 2 to 60, specifically 2 to 40, and more specifically 2 to 20.

In the present specification, a haloalkyl group means an alkyl group substituted with a halogen group, and specific examples thereof include —CF₃, —CF₂CF₃, and the like, but are not limited thereto.

In the present specification, an alkoxy group is represented by —O(R101), and the above-described examples of the alkyl group may be applied to R101.

In the present specification, an aryloxy group is represented by —O(R102), and the above-described examples of the aryl group may be applied to R102.

In the present specification, an alkylthioxy group is represented by —S(R103), and the above-described examples of the alkyl group may be applied to R103.

In the present specification, an arylthioxy group is represented by —S(R104), and the above-described examples of the aryl group may be applied to R104.

In the present specification, an alkylsulfoxy group is represented by —S(═O) 2 (R105), and the above-described examples of the alkyl group may be applied to R105.

In the present specification, an arylsulfoxy group is represented by —S(═O) 2 (R106), and the above-described examples of the aryl group may be applied to R106.

In the present specification, a cycloalkyl group includes a monocycle or polycycle having 3 to 60 carbon atoms, and may be additionally substituted with another substituent. Here, the polycycle means a group in which a cycloalkyl group is directly linked to or fused with another cyclic group. Here, another cyclic group may also be a cycloalkyl group, but may also be another kind of cyclic group, for example, a heterocycloalkyl group, an aryl group, a heteroaryl group, and the like. The number of carbon atoms of the cycloalkyl group may be 3 to 60, specifically 3 to 40, and more specifically 5 to 20. Specific examples thereof include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group, and the like, but are not limited thereto.

In the present specification, a heterocycloalkyl group includes O, S, Se, N, or Si as a heteroatom, includes a monocycle or polycycle having 2 to 60 carbon atoms, and may be additionally substituted with another substituent. Here, the polycycle means a group in which a heterocycloalkyl group is directly linked to or fused with another cyclic group. Here, another cyclic group may also be a heterocycloalkyl group, but may also be another kind of cyclic group, for example, a cycloalkyl group, an aryl group, a heteroaryl group, and the like. The number of carbon atoms of the heterocycloalkyl group may be 2 to 60, specifically 2 to 40, and more specifically 3 to 20.

In the present specification, an aryl group includes a monocycle or polycycle having 6 to 60 carbon atoms, and may be additionally substituted with another substituent. Here, the polycycle means a group in which an aryl group is directly linked to or fused with another cyclic group. Here, another cyclic group may also be an aryl group, but may also be another kind of cyclic group, for example, a cycloalkyl group, a heterocycloalkyl group, a heteroaryl group, and the like. The aryl group includes a spiro group. The number of carbon atoms of the aryl group may be 6 to 60, specifically 6 to 40, and more specifically 6 to 25. Specific examples of the aryl group include a phenyl group, a biphenyl group, a terphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused cyclic group thereof, and the like, but are not limited thereto.

In the present specification, the terphenyl group may be selected from the following structures.

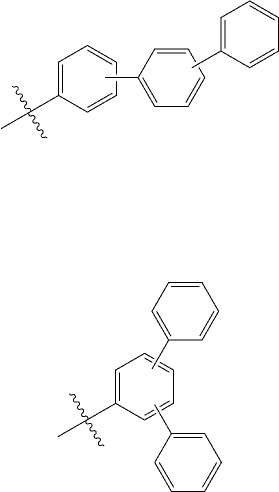

In the present specification, the fluorenyl group may be substituted, and adjacent substituents may be bonded to each other to form a ring.

When the fluorenyl group is substituted, the substituent may be the following structures, but is not limited thereto.

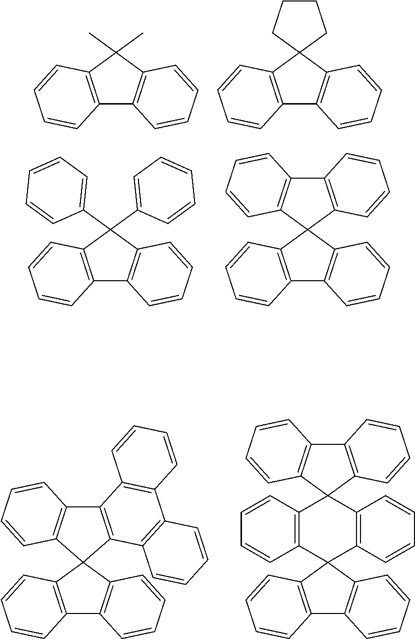

In the present specification, a heteroaryl group includes S, O, Se, N, or Si as a heteroatom, includes a monocycle or polycycle having 2 to 60 carbon atoms, and may be additionally substituted with another substituent. Here, the polycycle means a group in which a heteroaryl group is directly linked to or fused with another cyclic group. Here, another cyclic group may also be a heteroaryl group, but may also be another kind of cyclic group, for example, a cycloalkyl group, a heterocycloalkyl group, an aryl group, and the like. The number of carbon atoms of the heteroaryl group may be 2 to 60, specifically 2 to 40, and more specifically 3 to 25. Specific examples of the heteroaryl group include a pyridine group, a pyrrole group, a pyrimidine group, a pyridazine group, a furan group, a thiophene group, an imidazole group, a pyrazole group, an oxazole group, an isoxazole group, a thiazole group, an isothiazole group, a triazole group, a furazan group, an oxadiazole group, a thiadiazole group, a dithiazole group, a tetrazolyl group, a pyran group, a thiopyran group, a diazine group, an oxazine group, a thiazine group, a dioxin group, a triazine group, a tetrazine group, a quinoline group, an isoquinoline group, a quinazoline group, an isoquinazoline group, a quinozoline group, a naphthyridine group, an acridine group, a phenanthridine group, an imidazopyridine group, a diazanaphthalene group, a triazaindene group, an indole group, an indolizine group, a benzothiazole group, a benzoxazole group, a benzimidazole group, a benzothiophene group, a benzofuran group, a dibenzothiophene group, a dibenzofuran group, a carbazole group, a benzocarbazole group, a dibenzocarbazole group, a phenazine group, a dibenzosilole group, spirobi (dibenzosilole) group, a dihydrophenazine group, a phenoxazine group, a thienyl group, an indolo[2,3-a]carbazole group, an indolo[2,3-b]carbazole group, an indoline group, a 10,11-dihydro-dibenzo[b,f]azepine group, a 9,10-dihydroacridine group, a phenanthrazine group, a phenothiathiazine group, a phthalazine group, a phenanthroline group, a naphthobenzofuran group, a naphthobenzothiophene group, a benzo[c][1,2,5]thiadiazole group, a 2,3-dihydrobenzo[b]thiophene group, a 2,3-dihydrobenzofuran group, a 5,10-dihydrodibenzo[b,e][1,4]azasiline group, a pyrazolo[1,5-c]quinazoline group, a pyrido[1,2-b]indazole group, a pyrido[1,2-a]imidazo[1,2-e]indoline group, a 5,11-dihydroindeno[1,2-b]carbazole group, and the like, but are not limited thereto.

In the present specification, a benzocarbazole group may be any one of the following structures.

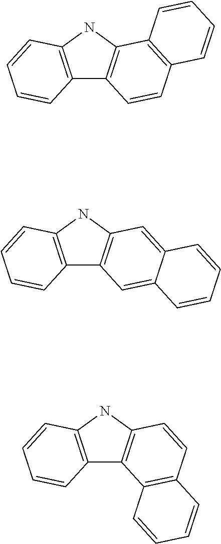 [SMILES: c1ccc2c(c1)ccc1c3ccccc3[nH]c21 ; c1ccc2cc3c(cc2c1)[nH]c1ccccc13 ; c1ccc2c(c1)ccc1[nH]c3ccccc3c12]

In the present specification, a dibenzocarbazole group may be any one of the following structures.

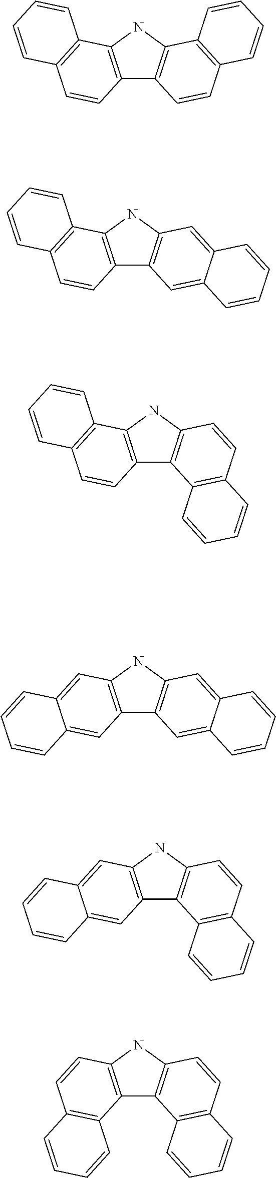 [SMILES: c1ccc2c(c1)ccc1c3ccc4ccccc4c3[nH]c21 ; c1ccc2cc3c(cc2c1)[nH]c1c2ccccc2ccc31 ; c1ccc2c(c1)ccc1c2[nH]c2ccc3ccccc3c21 ; c1ccc2cc3c(cc2c1)[nH]c1cc2ccccc2cc13 ; c1ccc2cc3c(cc2c1)[nH]c1ccc2ccccc2c13 ; c1ccc2c(c1)ccc1[nH]c3ccc4ccccc4c3c12]

In the present specification, when the substituent is a carbazole group, a benzocarbazole group, or a dibenzocarbazole group, it means being bonded to the nitrogen or carbon of the carbazole group, the benzocarbazole group, or the dibenzocarbazole group.

In the present specification, when a carbazole group, a benzocarbazole group, or a dibenzocarbazole group is substituted, an additional substituent may be substituted at the nitrogen or carbon of the carbazole group, the benzocarbazole group, or the dibenzocarbazole group.

In the present specification, a naphthobenzofuran group may be any one of the following structures.

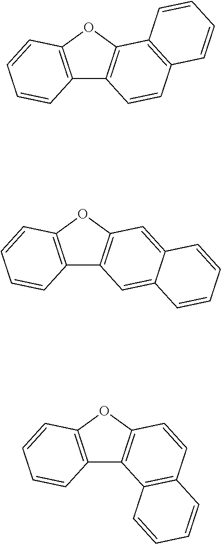 [SMILES: c1ccc2c(c1)ccc1c3ccccc3oc21 ; c1ccc2cc3c(cc2c1)oc1ccccc13 ; c1ccc2c(c1)ccc1oc3ccccc3c12]

In the present specification, a naphthobenzothiophene group may be any one of the following structures.

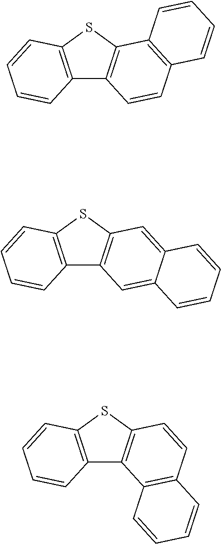 [SMILES: c1ccc2c(c1)ccc1c3ccccc3sc21 ; c1ccc2cc3c(cc2c1)sc1ccccc13 ; c1ccc2c(c1)ccc1sc3ccccc3c12]

In the present specification, the azine group is not limited as long as the azine group includes the following structure, and in the following structures, an additional ring may be fused. The additional ring to be fused may be, for example, an aliphatic hydrocarbon ring, an aliphatic hetero ring, an aromatic hydrocarbon ring, an aromatic hetero ring, and the like, but is not limited thereto.

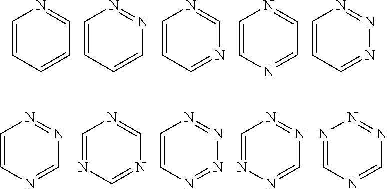

In the present specification, a silyl group includes Si and is a substituent to which the Si atom is directly linked as a radical, and is represented by —Si(R107)(R108)(R109), and R107 to R109 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group.

Specific examples of the silyl group include the following structures, but are not limited thereto.

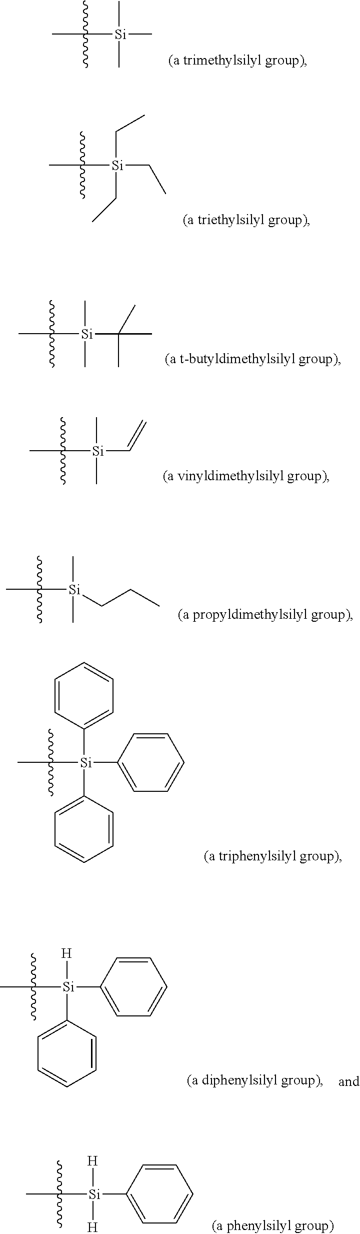

In the present specification, a phosphine oxide group is represented by —P(—O)(R110)(R111), and R110 and R111 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. Specifically, the phosphine oxide group may be substituted with an alkyl group or an aryl group, and the above-described example may be applied to the alkyl group and the aryl group. Examples of the phosphine oxide group include a dimethylphosphine oxide group, a diphenylphosphine oxide group, dinaphthylphosphine oxide group, and the like, but are not limited thereto.

In the present specification, an amine group is represented by —N(R112)(R113), and R112 and R113 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. The amine group may be selected from the group consisting of —NH₂; a monoalkylamine group; a monoarylamine group; a monoheteroarylamine group; a dialkylamine group; a diarylamine group; a diheteroarylamine group; an alkylarylamine group; an alkylheteroarylamine group; and an arylheteroarylamine group, and the number of carbon atoms thereof is not particularly limited, but is preferably 1 to 30. Specific examples of the amine group include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group, and the like, but are not limited thereto.

In the present specification, the “adjacent” group may mean a substituent substituted with an atom directly linked to an atom in which the corresponding substituent is substituted, a substituent disposed to be sterically closest to the corresponding substituent, or another substituent substituted with an atom in which the corresponding substituent is substituted. For example, two substituents substituted at the ortho position in a benzene ring and two substituents substituted at the same carbon in an aliphatic ring may be interpreted as groups which are “adjacent” to each other.

Hydrocarbon rings and hetero rings that adjacent groups may form include an aliphatic hydrocarbon ring, an aromatic hydrocarbon ring, an aliphatic hetero ring and an aromatic hetero ring, and structures exemplified by the above-described cycloalkyl group, aryl group, heterocycloalkyl group and heteroaryl group may be applied to the rings, except for those that are not monovalent groups.

An exemplary embodiment of the present specification provides the heterocyclic compound represented by Chemical Formula 1.

In an exemplary embodiment of the present specification, Chemical Formula 1 may be represented by any one of the following Chemical Formulae 1-1 to 1-4.

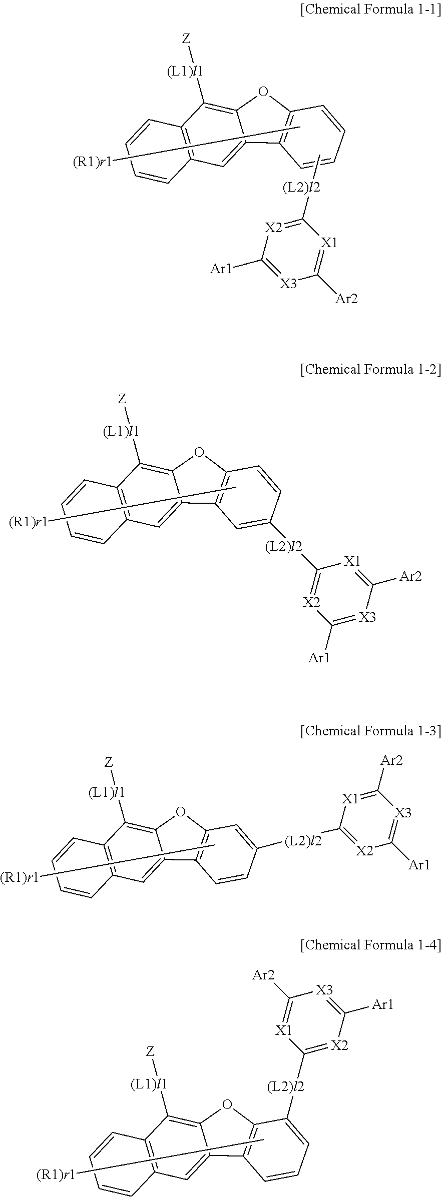

In Chemical Formulae 1-1 to 1-4, the definition of each substituent is the same as that in Chemical Formula 1.

In an exemplary embodiment of the present specification, L1 and L2 are each independently a direct bond; a phenylene group; or a naphthylene group.

In an exemplary embodiment of the present specification, l1 and l2 are each independently an integer from 0 to 3, and when l1 and l2 are each 2 or higher, substituents in the parenthesis are the same as or different from each other.

In an exemplary embodiment of the present specification, when l1 or l2 is 0, this means a direct bond.

In an exemplary embodiment of the present specification, R1 is hydrogen; or deuterium.

In an exemplary embodiment of the present specification, r1 is an integer from 0 to 8, and when r1 is 2 or higher, two or more R1's are the same as or different from each other.

That is, the heterocyclic compound according to an exemplary embodiment of the present specification includes only two substituents, except for hydrogen or deuterium.

In an exemplary embodiment of the present specification, when r1 is 0, this means that all positions where R1 may be substituted are hydrogen.

In an exemplary embodiment of the present specification, Z is —NR′R″; a substituted or unsubstituted alkyl group having 1 to 60 carbon atoms; a substituted or unsubstituted cycloalkyl group having 3 to 60 carbon atoms; a substituted or unsubstituted heterocycloalkyl group having 3 to 60 carbon atoms; an aryl group having 6 to 15 carbon atoms, which is unsubstituted or substituted with deuterium or an aryl group; a triphenylene group which is unsubstituted or substituted with deuterium or an aryl group; an aryl group having 20 to 60 carbon atoms, which is unsubstituted or substituted with deuterium or an aryl group; a substituted or unsubstituted dibenzofuran group; a substituted or unsubstituted dibenzothiophene group; or a substituted or unsubstituted benzocarbazole group.

In an exemplary embodiment of the present specification, Z is —NR′R″; a substituted or unsubstituted alkyl group having 1 to 20 carbon atoms; a substituted or unsubstituted cycloalkyl group having 3 to 30 carbon atoms; a substituted or unsubstituted heterocycloalkyl group having 3 to 30 carbon atoms; an aryl group having 6 to 15 carbon atoms, which is unsubstituted or substituted with deuterium or an aryl group; a triphenylene group which is unsubstituted or substituted with deuterium or an aryl group; an aryl group having 20 to 30 carbon atoms, which is unsubstituted or substituted with deuterium or an aryl group; a substituted or unsubstituted dibenzofuran group; a substituted or unsubstituted dibenzothiophene group; or a substituted or unsubstituted benzocarbazole group.

In an exemplary embodiment of the present specification, Z is —NR′R″; an aryl group having 6 to 15 carbon atoms, which is unsubstituted or substituted with deuterium or an aryl group; a triphenylene group which is unsubstituted or substituted with deuterium or an aryl group; an aryl group having 20 to 60 carbon atoms, which is unsubstituted or substituted with deuterium or an aryl group; or a substituted or unsubstituted benzocarbazole group.

In an exemplary embodiment of the present specification, Z is —NR′R″; a phenyl group which is unsubstituted or substituted with deuterium or an aryl group; a biphenyl group which is unsubstituted or substituted with deuterium or an aryl group; a naphthyl group which is unsubstituted or substituted with deuterium or an aryl group; a phenanthrene group which is unsubstituted or substituted with deuterium or an aryl group; a triphenylene group which is unsubstituted or substituted with deuterium or an aryl group; or a substituted or unsubstituted benzocarbazole group.

In an exemplary embodiment of the present specification, Z is —NR′R″; a phenyl group which is unsubstituted or substituted with deuterium; a biphenyl group; a naphthyl group; a phenanthrene group; a triphenylene group; or a benzocarbazole group.

In an exemplary embodiment of the present specification, R′ and R″ are each independently hydrogen; deuterium; a halogen group; a cyano group; a substituted or unsubstituted alkyl group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; or a substituted or unsubstituted heteroaryl group having 2 to 60 carbon atoms.

In an exemplary embodiment of the present specification, R′ and R″ are each independently hydrogen; deuterium; a halogen group; a cyano group; a substituted or unsubstituted alkyl group having 1 to 20 carbon atoms; a substituted or unsubstituted cycloalkyl group having 3 to 30 carbon atoms; a substituted or unsubstituted heterocycloalkyl group having 2 to 30 carbon atoms; a substituted or unsubstituted aryl group having 6 to 30 carbon atoms; or a substituted or unsubstituted heteroaryl group having 2 to 30 carbon atoms.

In an exemplary embodiment of the present specification, R′ and R″ are each independently hydrogen; deuterium; a halogen group; a cyano group; a substituted or unsubstituted aryl group having 6 to 30 carbon atoms; or a substituted or unsubstituted heteroaryl group having 2 to 30 carbon atoms.

In an exemplary embodiment of the present specification, R′ and are R″ each independently a substituted or unsubstituted aryl group having 6 to 30 carbon atoms; or a substituted or unsubstituted heteroaryl group having 2 to 30 carbon atoms.

In an exemplary embodiment of the present specification, R′ and R″ may be each independently a substituted or unsubstituted aryl group having 6 to 30 carbon atoms.

In an exemplary embodiment of the present specification, R′ and R″ may be each independently a substituted or unsubstituted phenyl group; a substituted or unsubstituted biphenyl group; a substituted or unsubstituted naphthyl group; or a substituted or unsubstituted fluorenyl group.

In an exemplary embodiment of the present specification, R′ and R″ may be each independently an aryl group having 6 to 30 carbon atoms, which is unsubstituted or substituted with one or more substituents of deuterium and an alkyl group.

In an exemplary embodiment of the present specification, R′ and R″ may be each independently a phenyl group which is unsubstituted or substituted with deuterium; a biphenyl group which is unsubstituted or substituted with deuterium; a naphthyl group which is unsubstituted or substituted with deuterium; or a fluorenyl group which is unsubstituted or substituted with one or more substituents of deuterium and an alkyl group.

In an exemplary embodiment of the present specification, R′ and R″ are each independently a phenyl group which is unsubstituted or substituted with deuterium; a biphenyl group which is unsubstituted or substituted with deuterium; a naphthyl group which is unsubstituted or substituted with deuterium; or a dimethylfluorenyl group which is unsubstituted or substituted with deuterium.

In an exemplary embodiment of the present specification, X1 to X3 are each independently N or CR, at least one thereof is N, and R is hydrogen; deuterium; a halogen group; a cyano group; a substituted or unsubstituted alkyl group having 1 to 60 carbon atoms; a substituted or unsubstituted aryl group having 6 to 60 carbon atoms; or a substituted or unsubstituted heteroaryl group having 2 to 60 carbon atoms, or may be bonded to Ar1 or Ar2 to form a substituted or unsubstituted hetero ring having 2 to 60 carbon atoms.

In an exemplary embodiment of the present specification, R is hydrogen; deuterium; a halogen group; a cyano group; a substituted or unsubstituted alkyl group having 1 to 20 carbon atoms; a substituted or unsubstituted aryl group having 6 to 30 carbon atoms; or a substituted or unsubstituted heteroaryl group having 2 to 30 carbon atoms, or may be bonded to Ar1 or Ar2 to form a substituted or unsubstituted hetero ring having 2 to 30 carbon atoms.

In an exemplary embodiment of the present specification, R is hydrogen; or deuterium, or may be bonded to Ar1 or Ar2 to form a hetero ring having 2 to 30 carbon atoms.

In an exemplary embodiment of the present specification, R is hydrogen; or deuterium, or may be bonded to Ar1 or Ar2 to form a benzofuran ring or a benzothiophene ring.

In an exemplary embodiment of the present specification, R may be hydrogen; or deuterium.

In an exemplary embodiment of the present specification, R may be bonded to Ar1 or Ar2 to form a substituted or unsubstituted benzofuran ring or a substituted or unsubstituted benzothiophene ring.

In an exemplary embodiment of the present specification, R may be bonded to Ar1 or Ar2 to form a benzofuran ring or a benzothiophene ring.

In an exemplary embodiment of the present specification, X1 to X3 are each independently N or CR, and at least two thereof are N.

In an exemplary embodiment of the present specification, X1 and X2 are N, and X3 may be CR.

In an exemplary embodiment of the present specification, X1 and X2 are N, X3 is CR, and R may be bonded to Ar1 or Ar2 to form a substituted or unsubstituted hetero ring having 2 to 60 carbon atoms.

In an exemplary embodiment of the present specification, X1 and X3 are N, and X2 may be CR.

In an exemplary embodiment of the present specification, X1 and X3 are N, X2 is CR, and R may be bonded to Ar1 to form a substituted or unsubstituted hetero ring having 2 to 60 carbon atoms.

In an exemplary embodiment of the present specification, X1 to X3 may be N.

In an exemplary embodiment of the present specification,

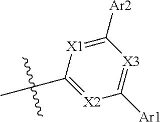

may be represented by any one of the following structures.

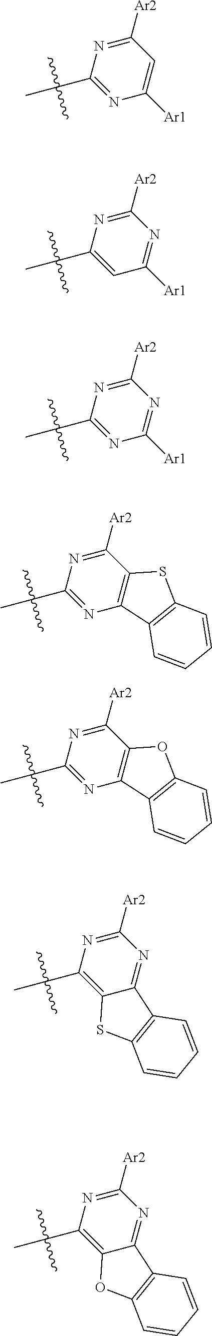

In an exemplary embodiment of the present specification, Ar1 and Ar2 are each independently hydrogen; deuterium; a halogen group; a cyano group; a substituted or unsubstituted alkyl group having 1 to 60 carbon atoms; a substituted or unsubstituted cycloalkyl group having 3 to 60 carbon atoms; a substituted or unsubstituted heterocycloalkyl group having 2 to 60 carbon atoms; an aryl group having 6 to 60 carbon atoms, which is unsubstituted or substituted with deuterium or an aryl group; or a substituted or unsubstituted heteroaryl group having 2 to 60 carbon atoms.

In an exemplary embodiment of the present specification, Ar1 and Ar2 are each independently hydrogen; deuterium; a halogen group; a cyano group; a substituted or unsubstituted alkyl group having 1 to 20 carbon atoms; a substituted or unsubstituted cycloalkyl group having 3 to 30 carbon atoms; a substituted or unsubstituted heterocycloalkyl group having 2 to 30 carbon atoms; an aryl group having 6 to 30 carbon atoms, which is unsubstituted or substituted with deuterium or an aryl group; or a substituted or unsubstituted heteroaryl group having 2 to 30 carbon atoms.

In an exemplary embodiment of the present specification, Ar1 and Ar2 are each independently hydrogen; deuterium; a halogen group; a cyano group; an aryl group having 6 to 30 carbon atoms, which is unsubstituted or substituted with deuterium or an aryl group; or a substituted or unsubstituted heteroaryl group having 2 to 30 carbon atoms.

In an exemplary embodiment of the present specification, Ar1 and Ar2 are each independently an aryl group having 6 to 30 carbon atoms, which is unsubstituted or substituted with deuterium or an aryl group; or a substituted or unsubstituted heteroaryl group having 2 to 30 carbon atoms.

In an exemplary embodiment of the present specification, Ar1 and Ar2 are each independently a phenyl group which is unsubstituted or substituted with deuterium or an aryl group; a biphenyl group which is unsubstituted or substituted with deuterium or an aryl group; a naphthyl group which is unsubstituted or substituted with deuterium or an aryl group; a phenanthrene group which is unsubstituted or substituted with deuterium or an aryl group; a triphenylene group which is unsubstituted or substituted with deuterium or an aryl group; a substituted or unsubstituted dibenzofuran group; or a substituted or unsubstituted dibenzothiophene group.

In an exemplary embodiment of the present specification, Ar1 and Ar2 are each independently a phenyl group which is unsubstituted or substituted with deuterium or an aryl group having 6 to 20 carbon atoms; a biphenyl group which is unsubstituted or substituted with deuterium or an aryl group having 6 to 20 carbon atoms; a naphthyl group which is unsubstituted or substituted with deuterium or an aryl group having 6 to 20 carbon atoms; a phenanthrene group which is unsubstituted or substituted with deuterium or an aryl group having 6 to 20 carbon atoms; a triphenylene group which is unsubstituted or substituted with deuterium or an aryl group having 6 to 20 carbon atoms; a substituted or unsubstituted dibenzofuran group; or a substituted or unsubstituted dibenzothiophene group.

In an exemplary embodiment of the present specification, Ar1 and Ar2 are each independently a phenyl group which is unsubstituted or substituted with deuterium or an aryl group having 6 to 20 carbon atoms; a biphenyl group; a naphthyl group which is unsubstituted or substituted with deuterium or an aryl group having 6 to 20 carbon atoms; a phenanthrene group; a triphenylene group; a dibenzofuran group; or a dibenzothiophene group.

In an exemplary embodiment of the present specification, Z does not include an aryl group having 6 to 15 carbon atoms, which is substituted with a substituent except for an aryl group, a triphenylene group which is substituted with a substituent except for an aryl group, an aryl group having 20 to 60 carbon atoms, which is substituted with a substituent except for an aryl group, or a heteroaryl group except for a dibenzofuran group, a dibenzothiophene group and a benzocarbazole group.

For example, Z does not include an aryl group having 6 to 15 carbon atoms, which is substituted with a halogen group, a triphenylene group which is substituted with a halogen group, an aryl group having 20 to 60 carbon atoms, which is substituted with a halogen group, or a carbazole group, a triazine group, and the like.

In an exemplary embodiment of the present specification, Ar1 and Ar2 do not include an aryl group having 6 to 60 carbon atoms, which is substituted with a substituent except for an aryl group.

For example, Ar1 and Ar2 do not include an aryl group having 6 to 60 carbon atoms, which is substituted with a heteroaryl group.

In an exemplary embodiment of the present specification, Ar1 and Ar2 do not include the case where

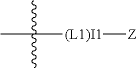

is

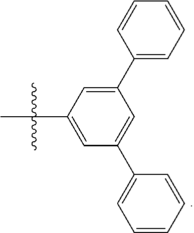 [SMILES: *c1cc(-c2ccccc2)cc(-c2ccccc2)c1]

In an exemplary embodiment of the present specification, when Z is an aryl group having 6 to 15 carbon atoms, which is unsubstituted or substituted with an aryl group, Ar1 and Ar2 are hydrogen; deuterium; a halogen group; a cyano group; a substituted or unsubstituted alkyl group having 1 to 60 carbon atoms; a substituted or unsubstituted cycloalkyl group having 3 to 60 carbon atoms; a substituted or unsubstituted heterocycloalkyl group having 2 to 60 carbon atoms; an aryl group having 6 to 60 carbon atoms, which is unsubstituted or substituted with deuterium or an aryl group; a substituted or unsubstituted dibenzofuran group; or a substituted or unsubstituted dibenzothiophene group.

That is, when Z is an aryl group having 6 to 15 carbon atoms, which is unsubstituted or substituted with an aryl group, Ar1 and Ar2 do not include naphthobenzothiophene. For example, when Z is a phenyl group, Ar1 and Ar2 cannot include naphthobenzothiophene.

In an exemplary embodiment of the present specification, Chemical Formula 1 may be represented by any one of the following Chemical Formulae 1-1-1 to 1-1-3.

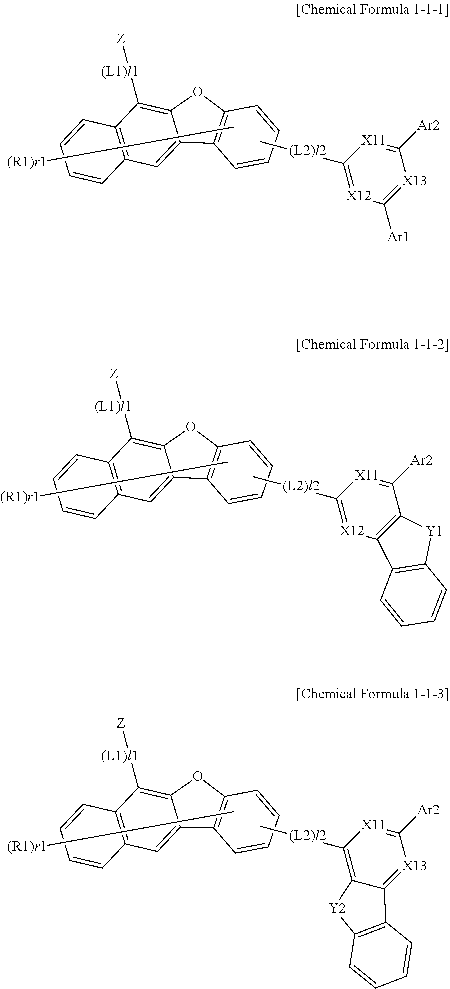

In Chemical Formulae 1-1-1 to 1-1-3,
X11 to X13 are each independently N or CH, and at least one thereof is N,
Y1 and Y2 are each independently O; or S, and
the definitions of the other substituents are the same as those in Chemical Formula 1.

In an exemplary embodiment of the present specification, the deuterium content of Chemical Formula 1 may be 0% to 100%.

In an exemplary embodiment of the present specification, the deuterium content of Chemical Formula 1 may be 0% or 5% to 100%.

In an exemplary embodiment of the present specification, the deuterium content of Chemical Formula 1 may be 0% or 10% to 100%.

In an exemplary embodiment of the present specification, the deuterium content of Chemical Formula 1 may be 08 or 20% to 100%.

In an exemplary embodiment of the present specification, the deuterium content of Chemical Formula 1 may be 0% or 50% to 100%.

In an exemplary embodiment of the present specification, Chemical Formula 1 may be represented by any one of the following compounds.

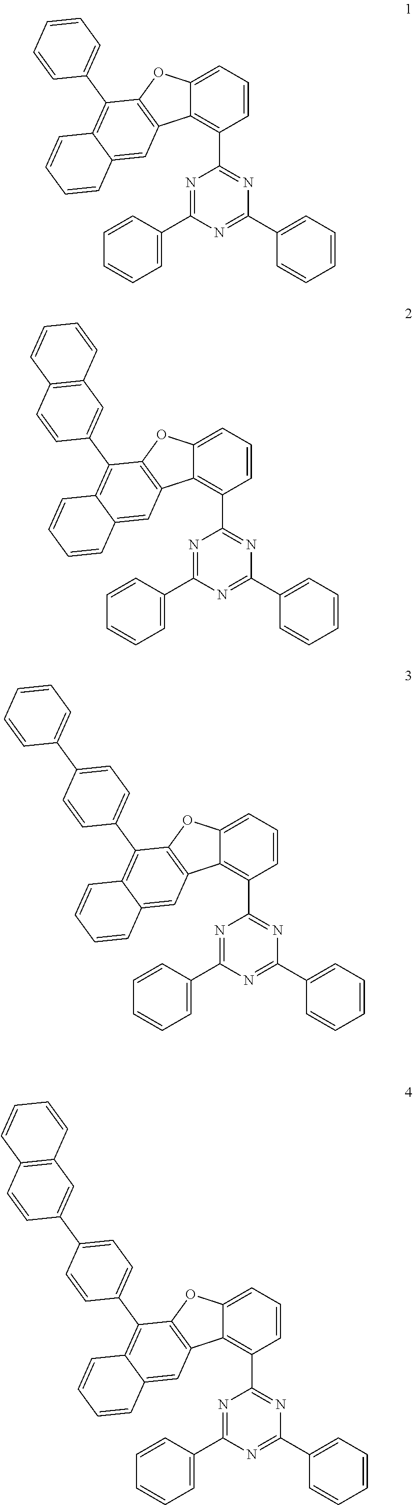 [SMILES: c1ccc(-c2nc(-c3ccccc3)nc(-c3cccc4oc5c(-c6ccccc6)c6ccccc6cc5c34)n2)cc1 ; c1ccc(-c2nc(-c3ccccc3)nc(-c3cccc4oc5c(-c6ccc7ccccc7c6)c6ccccc6cc5c34)n2)cc1 ; c1ccc(-c2ccc(-c3c4ccccc4cc4c3oc3cccc(-c5nc(-c6ccccc6)nc(-c6ccccc6)n5)c34)cc2)cc1 ; c1ccc(-c2nc(-c3ccccc3)nc(-c3cccc4oc5c(-c6ccc(-c7ccc8ccccc8c7)cc6)c6ccccc6cc5c34)n2)cc1]

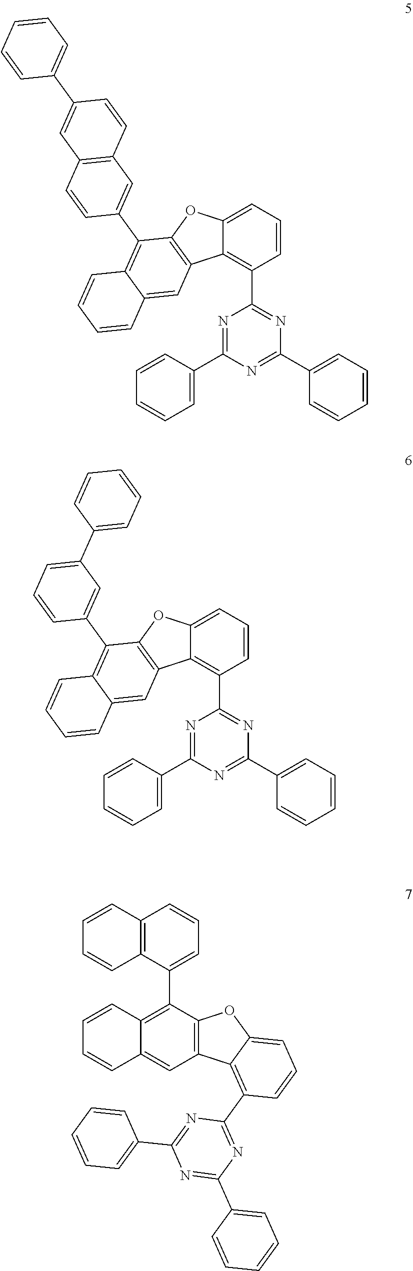 [SMILES: c1ccc(-c2ccc3cc(-c4c5ccccc5cc5c4oc4cccc(-c6nc(-c7ccccc7)nc(-c7ccccc7)n6)c45)ccc3c2)cc1 ; c1ccc(-c2cccc(-c3c4ccccc4cc4c3oc3cccc(-c5nc(-c6ccccc6)nc(-c6ccccc6)n5)c34)c2)cc1 ; c1ccc(-c2nc(-c3ccccc3)nc(-c3cccc4oc5c(-c6cccc7ccccc67)c6ccccc6cc5c34)n2)cc1]

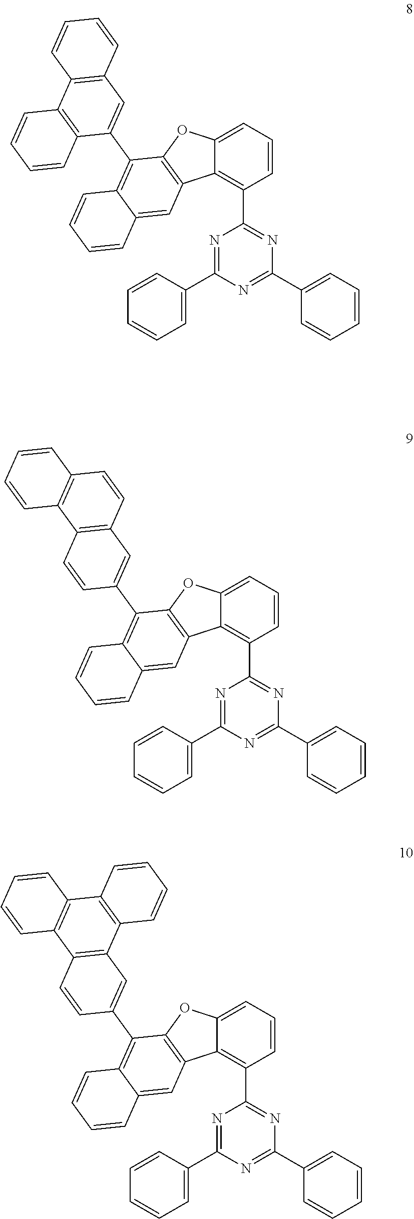 [SMILES: c1ccc(-c2nc(-c3ccccc3)nc(-c3cccc4oc5c(-c6cc7ccccc7c7ccccc67)c6ccccc6cc5c34)n2)cc1 ; c1ccc(-c2nc(-c3ccccc3)nc(-c3cccc4oc5c(-c6ccc7c(ccc8ccccc87)c6)c6ccccc6cc5c34)n2)cc1 ; c1ccc(-c2nc(-c3ccccc3)nc(-c3cccc4oc5c(-c6ccc7c8ccccc8c8ccccc8c7c6)c6ccccc6cc5c34)n2)cc1]

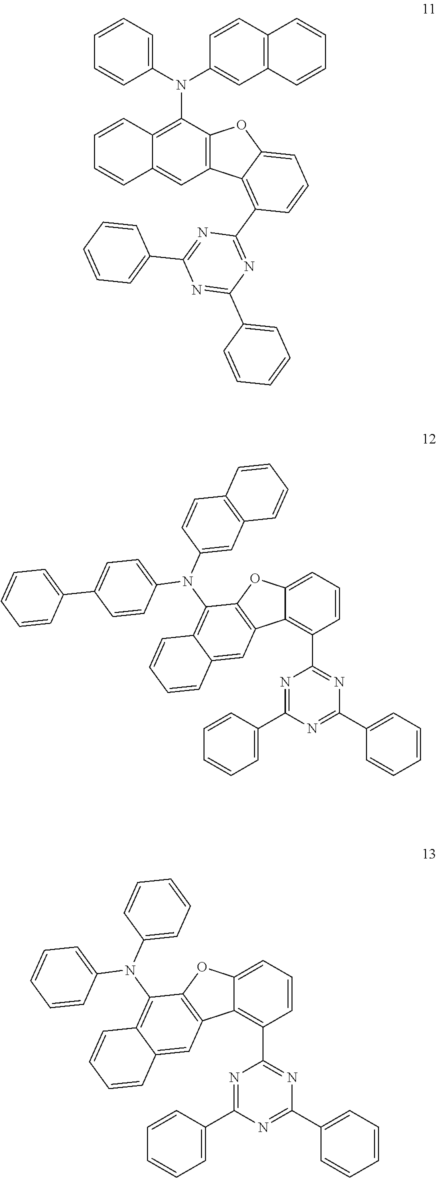 [SMILES: c1ccc(-c2nc(-c3ccccc3)nc(-c3cccc4oc5c(N(c6ccccc6)c6ccc7ccccc7c6)c6ccccc6cc5c34)n2)cc1 ; c1ccc(-c2ccc(N(c3ccc4ccccc4c3)c3c4ccccc4cc4c3oc3cccc(-c5nc(-c6ccccc6)nc(-c6ccccc6)n5)c34)cc2)cc1 ; c1ccc(-c2nc(-c3ccccc3)nc(-c3cccc4oc5c(N(c6ccccc6)c6ccccc6)c6ccccc6cc5c34)n2)cc1]

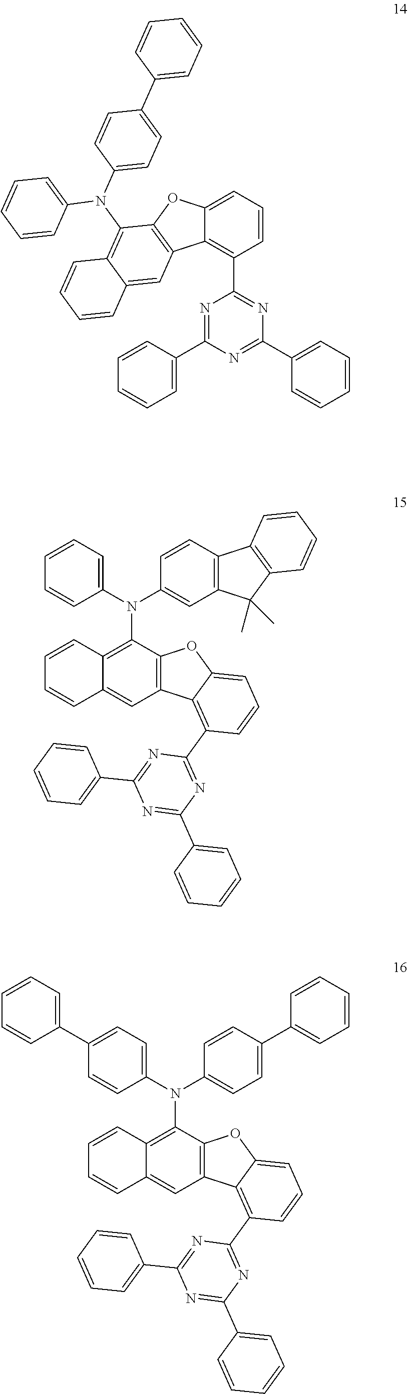 [SMILES: c1ccc(-c2ccc(N(c3ccccc3)c3c4ccccc4cc4c3oc3cccc(-c5nc(-c6ccccc6)nc(-c6ccccc6)n5)c34)cc2)cc1 ; CC1(C)c2ccccc2-c2ccc(N(c3ccccc3)c3c4ccccc4cc4c3oc3cccc(-c5nc(-c6ccccc6)nc(-c6ccccc6)n5)c34)cc21 ; c1ccc(-c2ccc(N(c3ccc(-c4ccccc4)cc3)c3c4ccccc4cc4c3oc3cccc(-c5nc(-c6ccccc6)nc(-c6ccccc6)n5)c34)cc2)cc1]

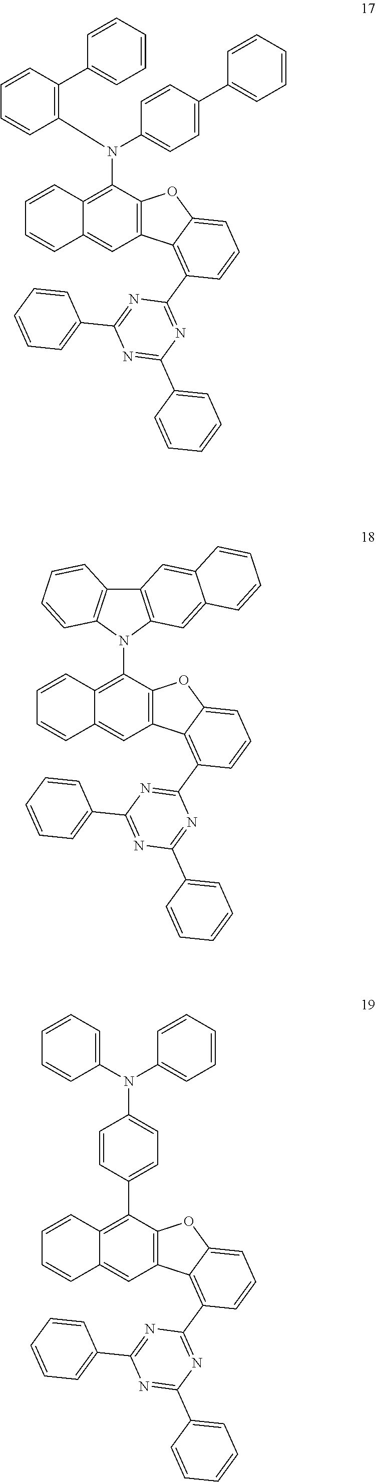 [SMILES: c1ccc(-c2ccc(N(c3ccccc3-c3ccccc3)c3c4ccccc4cc4c3oc3cccc(-c5nc(-c6ccccc6)nc(-c6ccccc6)n5)c34)cc2)cc1 ; c1ccc(-c2nc(-c3ccccc3)nc(-c3cccc4oc5c(-n6c7ccccc7c7cc8ccccc8cc76)c6ccccc6cc5c34)n2)cc1 ; c1ccc(-c2nc(-c3ccccc3)nc(-c3cccc4oc5c(-c6ccc(N(c7ccccc7)c7ccccc7)cc6)c6ccccc6cc5c34)n2)cc1]

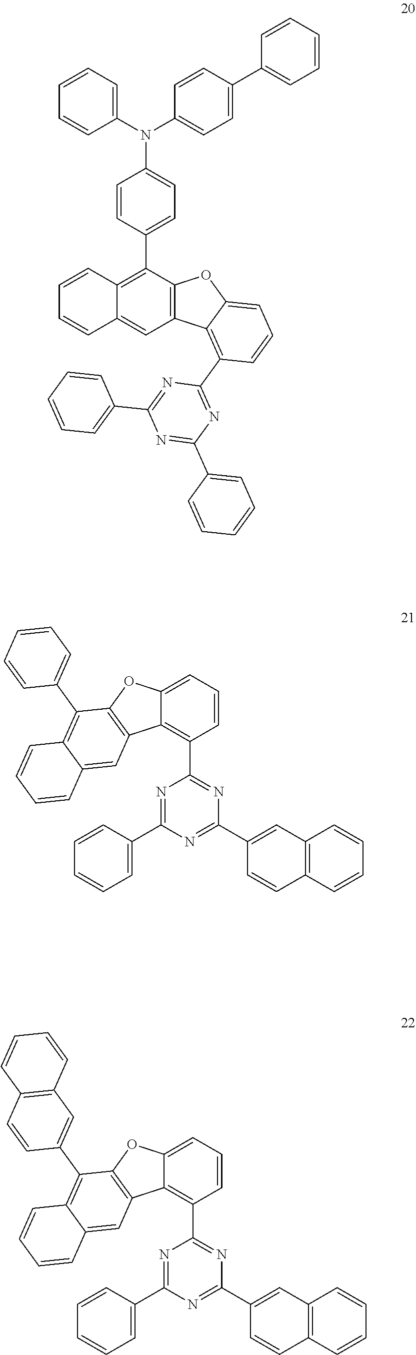 [SMILES: c1ccc(-c2ccc(N(c3ccccc3)c3ccc(-c4c5ccccc5cc5c4oc4cccc(-c6nc(-c7ccccc7)nc(-c7ccccc7)n6)c45)cc3)cc2)cc1 ; c1ccc(-c2nc(-c3ccc4ccccc4c3)nc(-c3cccc4oc5c(-c6ccccc6)c6ccccc6cc5c34)n2)cc1 ; c1ccc(-c2nc(-c3ccc4ccccc4c3)nc(-c3cccc4oc5c(-c6ccc7ccccc7c6)c6ccccc6cc5c34)n2)cc1]

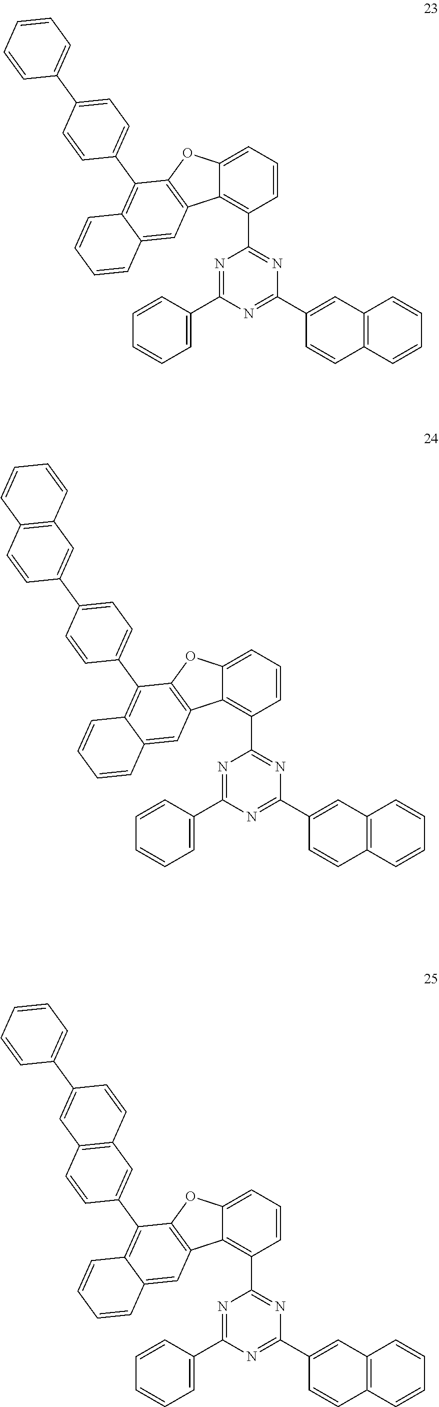 [SMILES: c1ccc(-c2ccc(-c3c4ccccc4cc4c3oc3cccc(-c5nc(-c6ccccc6)nc(-c6ccc7ccccc7c6)n5)c34)cc2)cc1 ; c1ccc(-c2nc(-c3ccc4ccccc4c3)nc(-c3cccc4oc5c(-c6ccc(-c7ccc8ccccc8c7)cc6)c6ccccc6cc5c34)n2)cc1 ; c1ccc(-c2ccc3cc(-c4c5ccccc5cc5c4oc4cccc(-c6nc(-c7ccccc7)nc(-c7ccc8ccccc8c7)n6)c45)ccc3c2)cc1]

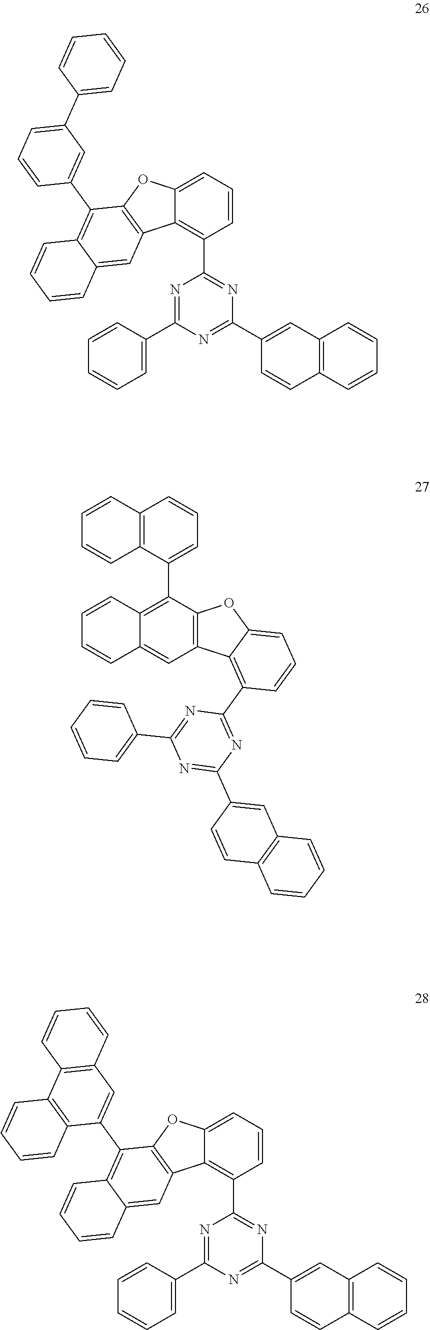 [SMILES: c1ccc(-c2cccc(-c3c4ccccc4cc4c3oc3cccc(-c5nc(-c6ccccc6)nc(-c6ccc7ccccc7c6)n5)c34)c2)cc1 ; c1ccc(-c2nc(-c3ccc4ccccc4c3)nc(-c3cccc4oc5c(-c6cccc7ccccc67)c6ccccc6cc5c34)n2)cc1 ; c1ccc(-c2nc(-c3ccc4ccccc4c3)nc(-c3cccc4oc5c(-c6cc7ccccc7c7ccccc67)c6ccccc6cc5c34)n2)cc1]

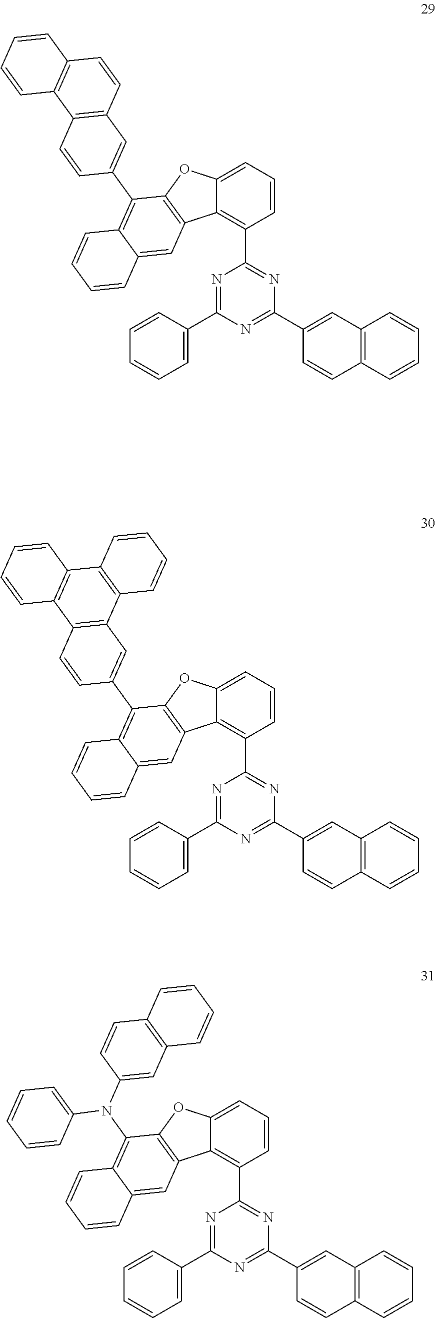 [SMILES: c1ccc(-c2nc(-c3ccc4ccccc4c3)nc(-c3cccc4oc5c(-c6ccc7c(ccc8ccccc87)c6)c6ccccc6cc5c34)n2)cc1 ; c1ccc(-c2nc(-c3ccc4ccccc4c3)nc(-c3cccc4oc5c(-c6ccc7c8ccccc8c8ccccc8c7c6)c6ccccc6cc5c34)n2)cc1 ; c1ccc(-c2nc(-c3ccc4ccccc4c3)nc(-c3cccc4oc5c(N(c6ccccc6)c6ccc7ccccc7c6)c6ccccc6cc5c34)n2)cc1]

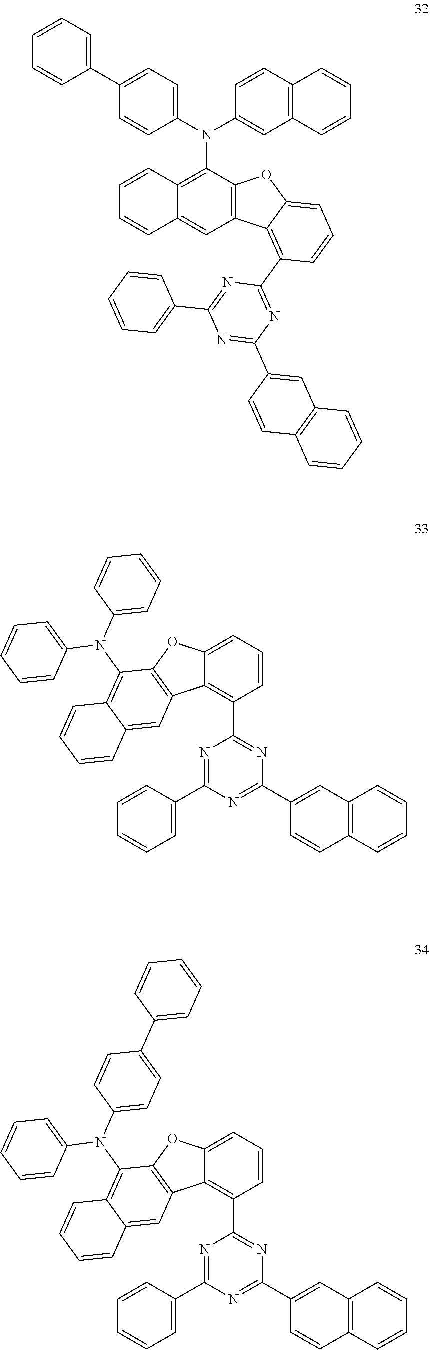 [SMILES: c1ccc(-c2ccc(N(c3ccc4ccccc4c3)c3c4ccccc4cc4c3oc3cccc(-c5nc(-c6ccccc6)nc(-c6ccc7ccccc7c6)n5)c34)cc2)cc1 ; c1ccc(-c2nc(-c3ccc4ccccc4c3)nc(-c3cccc4oc5c(N(c6ccccc6)c6ccccc6)c6ccccc6cc5c34)n2)cc1 ; c1ccc(-c2ccc(N(c3ccccc3)c3c4ccccc4cc4c3oc3cccc(-c5nc(-c6ccccc6)nc(-c6ccc7ccccc7c6)n5)c34)cc2)cc1]

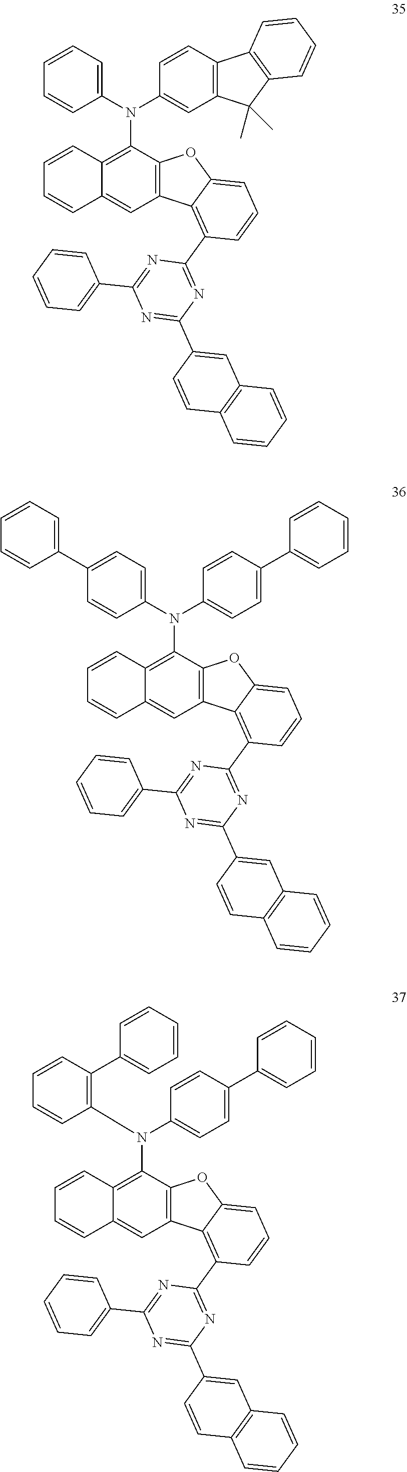 [SMILES: CC1(C)c2ccccc2-c2ccc(N(c3ccccc3)c3c4ccccc4cc4c3oc3cccc(-c5nc(-c6ccccc6)nc(-c6ccc7ccccc7c6)n5)c34)cc21 ; c1ccc(-c2ccc(N(c3ccc(-c4ccccc4)cc3)c3c4ccccc4cc4c3oc3cccc(-c5nc(-c6ccccc6)nc(-c6ccc7ccccc7c6)n5)c34)cc2)cc1 ; c1ccc(-c2ccc(N(c3ccccc3-c3ccccc3)c3c4ccccc4cc4c3oc3cccc(-c5nc(-c6ccccc6)nc(-c6ccc7ccccc7c6)n5)c34)cc2)cc1]

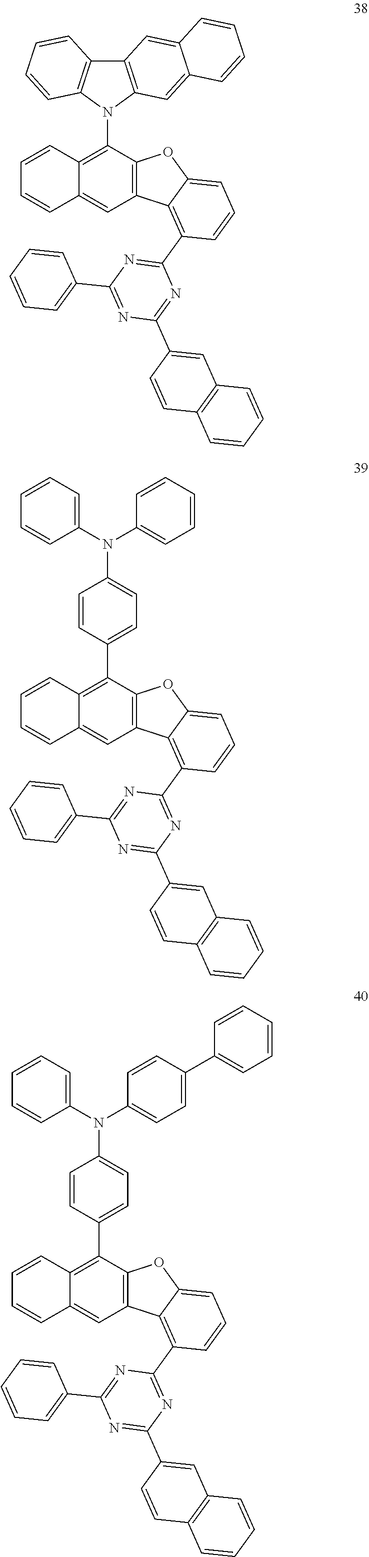

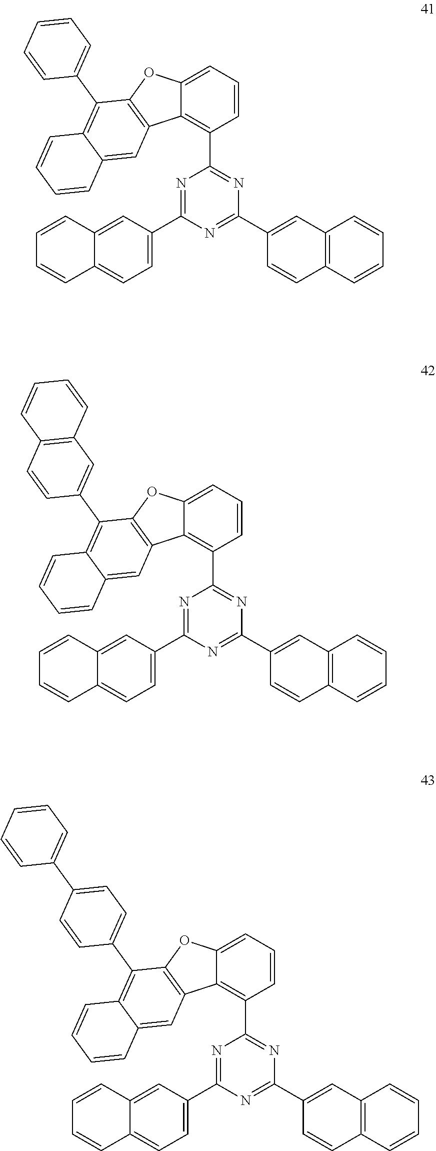 [SMILES: c1ccc(-c2c3ccccc3cc3c2oc2cccc(-c4nc(-c5ccc6ccccc6c5)nc(-c5ccc6ccccc6c5)n4)c23)cc1 ; c1ccc2cc(-c3nc(-c4ccc5ccccc5c4)nc(-c4cccc5oc6c(-c7ccc8ccccc8c7)c7ccccc7cc6c45)n3)ccc2c1 ; c1ccc(-c2ccc(-c3c4ccccc4cc4c3oc3cccc(-c5nc(-c6ccc7ccccc7c6)nc(-c6ccc7ccccc7c6)n5)c34)cc2)cc1]

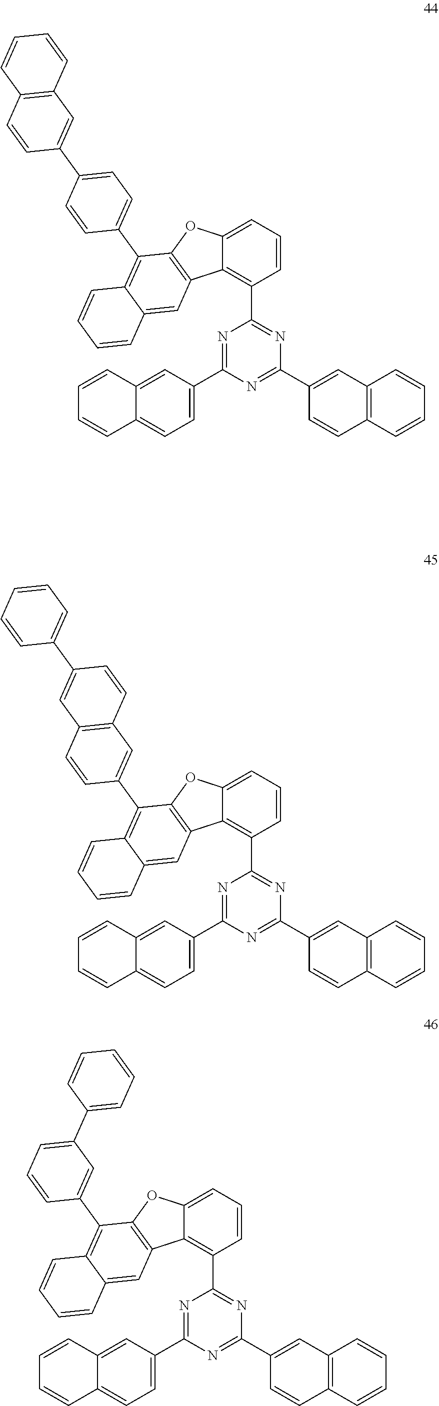 [SMILES: c1ccc2cc(-c3ccc(-c4c5ccccc5cc5c4oc4cccc(-c6nc(-c7ccc8ccccc8c7)nc(-c7ccc8ccccc8c7)n6)c45)cc3)ccc2c1 ; c1ccc(-c2ccc3cc(-c4c5ccccc5cc5c4oc4cccc(-c6nc(-c7ccc8ccccc8c7)nc(-c7ccc8ccccc8c7)n6)c45)ccc3c2)cc1 ; c1ccc(-c2cccc(-c3c4ccccc4cc4c3oc3cccc(-c5nc(-c6ccc7ccccc7c6)nc(-c6ccc7ccccc7c6)n5)c34)c2)cc1]

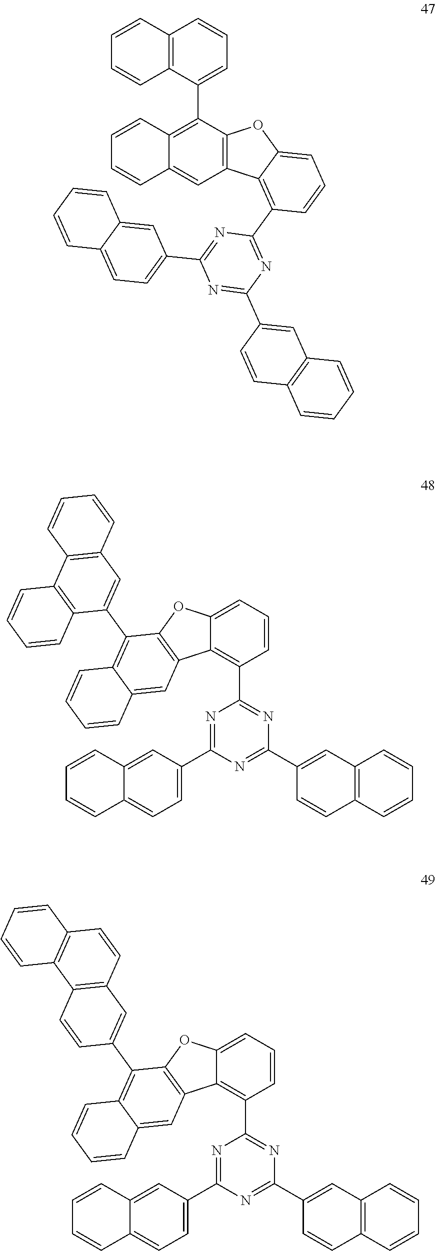 [SMILES: c1ccc2cc(-c3nc(-c4ccc5ccccc5c4)nc(-c4cccc5oc6c(-c7cccc8ccccc78)c7ccccc7cc6c45)n3)ccc2c1 ; c1ccc2cc(-c3nc(-c4ccc5ccccc5c4)nc(-c4cccc5oc6c(-c7cc8ccccc8c8ccccc78)c7ccccc7cc6c45)n3)ccc2c1 ; c1ccc2cc(-c3nc(-c4ccc5ccccc5c4)nc(-c4cccc5oc6c(-c7ccc8c(ccc9ccccc98)c7)c7ccccc7cc6c45)n3)ccc2c1]

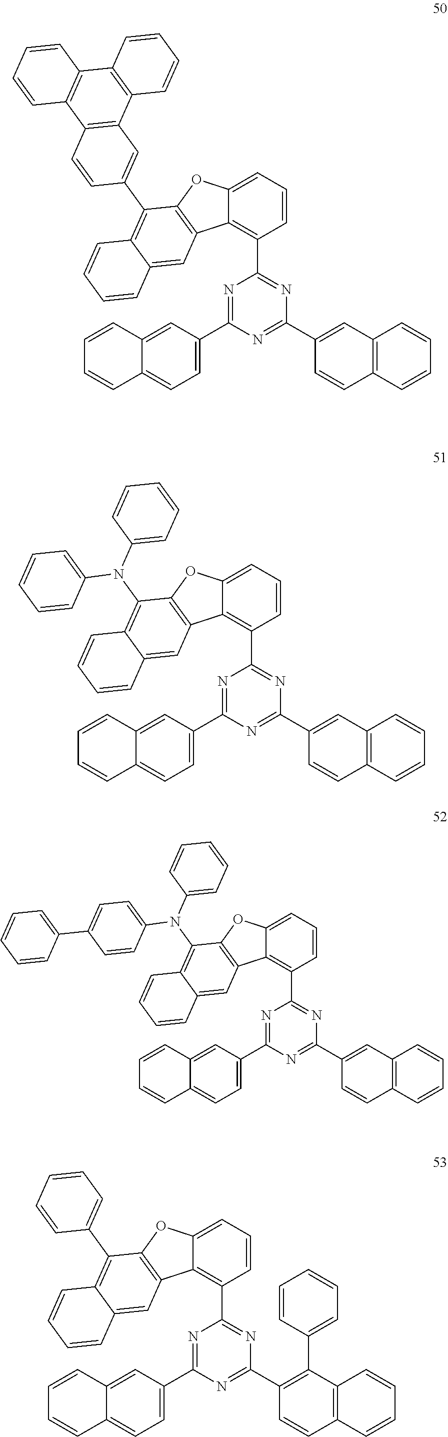 [SMILES: c1ccc2cc(-c3nc(-c4ccc5ccccc5c4)nc(-c4cccc5oc6c(-c7ccc8c9ccccc9c9ccccc9c8c7)c7ccccc7cc6c45)n3)ccc2c1 ; c1ccc(N(c2ccccc2)c2c3ccccc3cc3c2oc2cccc(-c4nc(-c5ccc6ccccc6c5)nc(-c5ccc6ccccc6c5)n4)c23)cc1 ; c1ccc(-c2ccc(N(c3ccccc3)c3c4ccccc4cc4c3oc3cccc(-c5nc(-c6ccc7ccccc7c6)nc(-c6ccc7ccccc7c6)n5)c34)cc2)cc1 ; c1ccc(-c2c(-c3nc(-c4ccc5ccccc5c4)nc(-c4cccc5oc6c(-c7ccccc7)c7ccccc7cc6c45)n3)ccc3ccccc23)cc1]

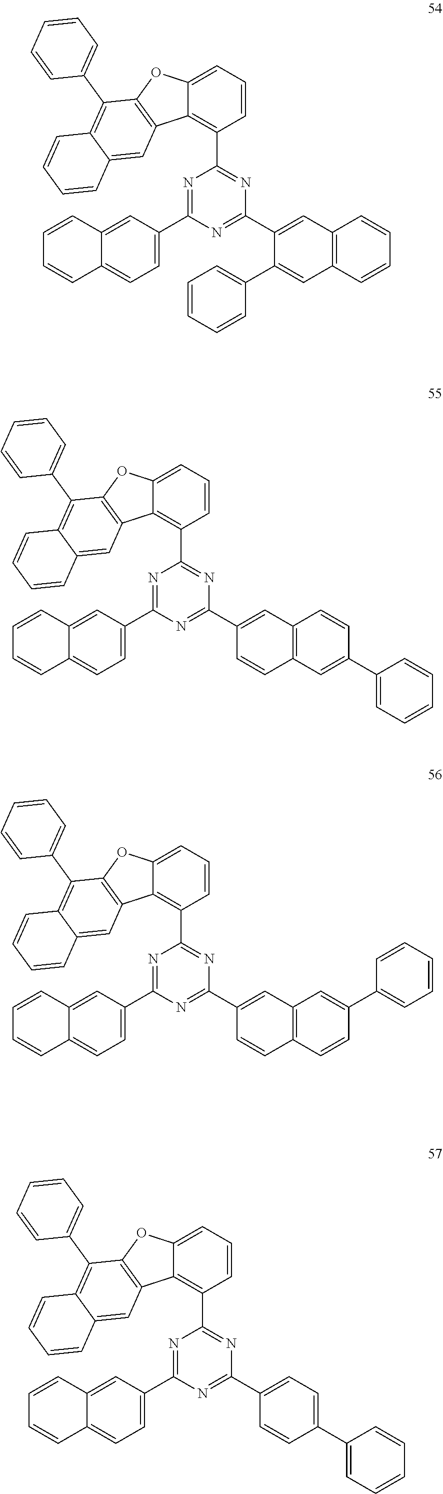 [SMILES: c1ccc(-c2cc3ccccc3cc2-c2nc(-c3ccc4ccccc4c3)nc(-c3cccc4oc5c(-c6ccccc6)c6ccccc6cc5c34)n2)cc1 ; c1ccc(-c2ccc3cc(-c4nc(-c5ccc6ccccc6c5)nc(-c5cccc6oc7c(-c8ccccc8)c8ccccc8cc7c56)n4)ccc3c2)cc1 ; c1ccc(-c2ccc3ccc(-c4nc(-c5ccc6ccccc6c5)nc(-c5cccc6oc7c(-c8ccccc8)c8ccccc8cc7c56)n4)cc3c2)cc1 ; c1ccc(-c2ccc(-c3nc(-c4ccc5ccccc5c4)nc(-c4cccc5oc6c(-c7ccccc7)c7ccccc7cc6c45)n3)cc2)cc1]

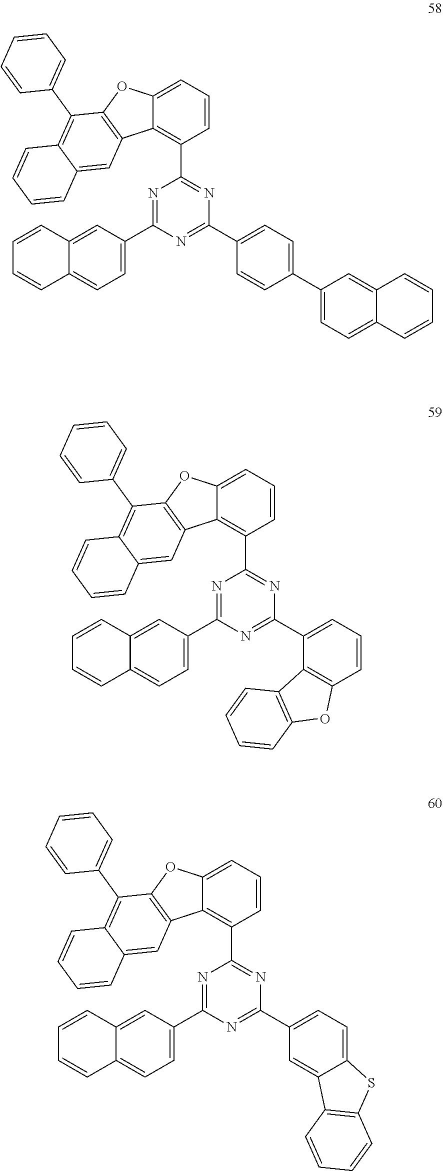 [SMILES: c1ccc(-c2c3ccccc3cc3c2oc2cccc(-c4nc(-c5ccc(-c6ccc7ccccc7c6)cc5)nc(-c5ccc6ccccc6c5)n4)c23)cc1 ; c1ccc(-c2c3ccccc3cc3c2oc2cccc(-c4nc(-c5ccc6ccccc6c5)nc(-c5cccc6oc7ccccc7c56)n4)c23)cc1 ; c1ccc(-c2c3ccccc3cc3c2oc2cccc(-c4nc(-c5ccc6ccccc6c5)nc(-c5ccc6sc7ccccc7c6c5)n4)c23)cc1]

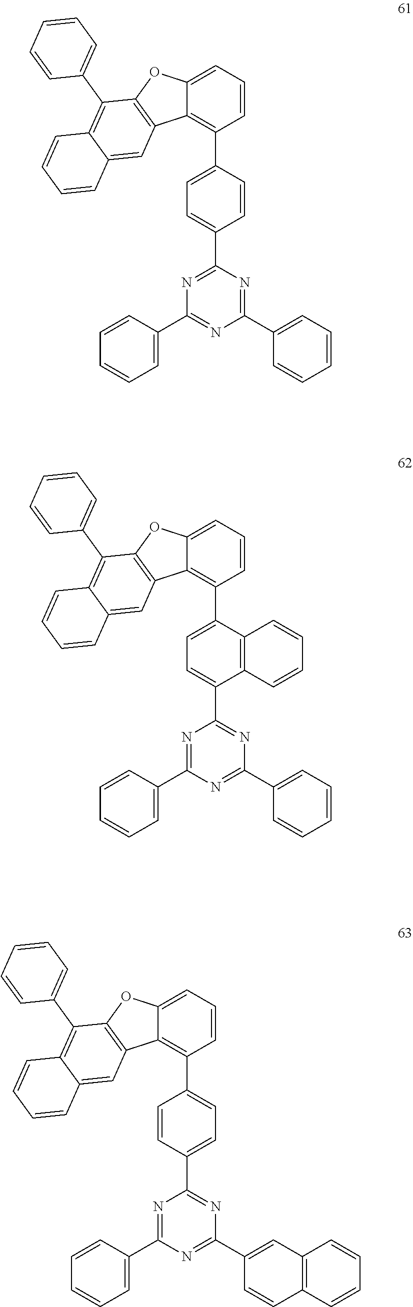 [SMILES: c1ccc(-c2nc(-c3ccccc3)nc(-c3ccc(-c4cccc5oc6c(-c7ccccc7)c7ccccc7cc6c45)cc3)n2)cc1 ; c1ccc(-c2nc(-c3ccccc3)nc(-c3ccc(-c4cccc5oc6c(-c7ccccc7)c7ccccc7cc6c45)c4ccccc34)n2)cc1 ; c1ccc(-c2nc(-c3ccc(-c4cccc5oc6c(-c7ccccc7)c7ccccc7cc6c45)cc3)nc(-c3ccc4ccccc4c3)n2)cc1]

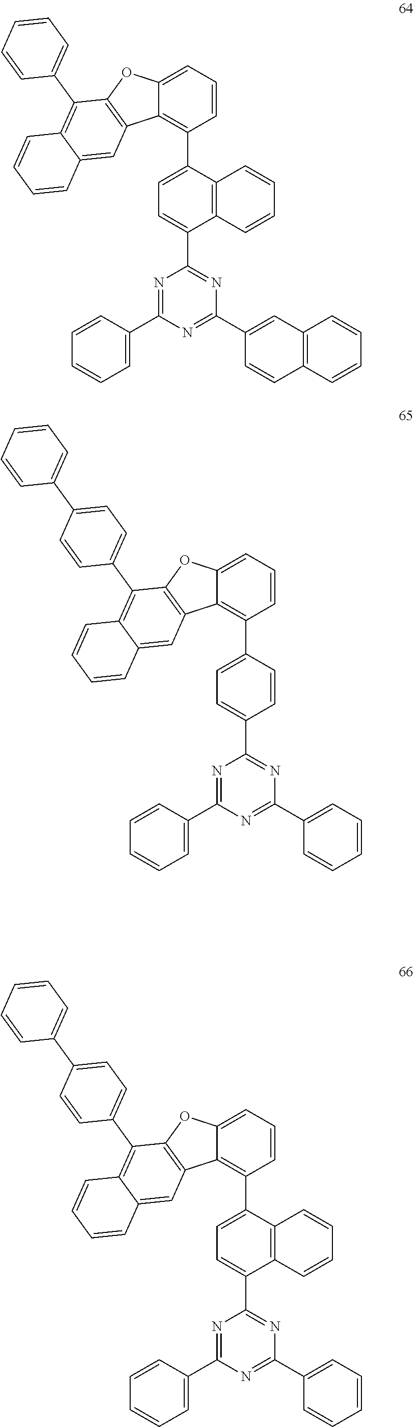 [SMILES: c1ccc(-c2nc(-c3ccc4ccccc4c3)nc(-c3ccc(-c4cccc5oc6c(-c7ccccc7)c7ccccc7cc6c45)c4ccccc34)n2)cc1 ; c1ccc(-c2ccc(-c3c4ccccc4cc4c3oc3cccc(-c5ccc(-c6nc(-c7ccccc7)nc(-c7ccccc7)n6)cc5)c34)cc2)cc1 ; c1ccc(-c2ccc(-c3c4ccccc4cc4c3oc3cccc(-c5ccc(-c6nc(-c7ccccc7)nc(-c7ccccc7)n6)c6ccccc56)c34)cc2)cc1]

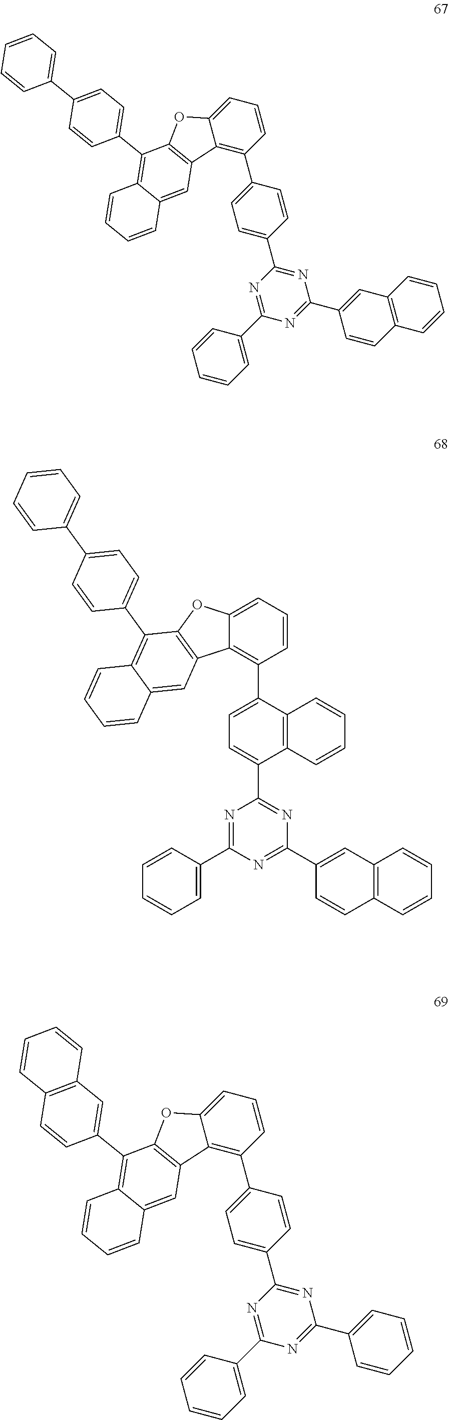 [SMILES: c1ccc(-c2ccc(-c3c4ccccc4cc4c3oc3cccc(-c5ccc(-c6nc(-c7ccccc7)nc(-c7ccc8ccccc8c7)n6)cc5)c34)cc2)cc1 ; c1ccc(-c2ccc(-c3c4ccccc4cc4c3oc3cccc(-c5ccc(-c6nc(-c7ccccc7)nc(-c7ccc8ccccc8c7)n6)c6ccccc56)c34)cc2)cc1 ; c1ccc(-c2nc(-c3ccccc3)nc(-c3ccc(-c4cccc5oc6c(-c7ccc8ccccc8c7)c7ccccc7cc6c45)cc3)n2)cc1]

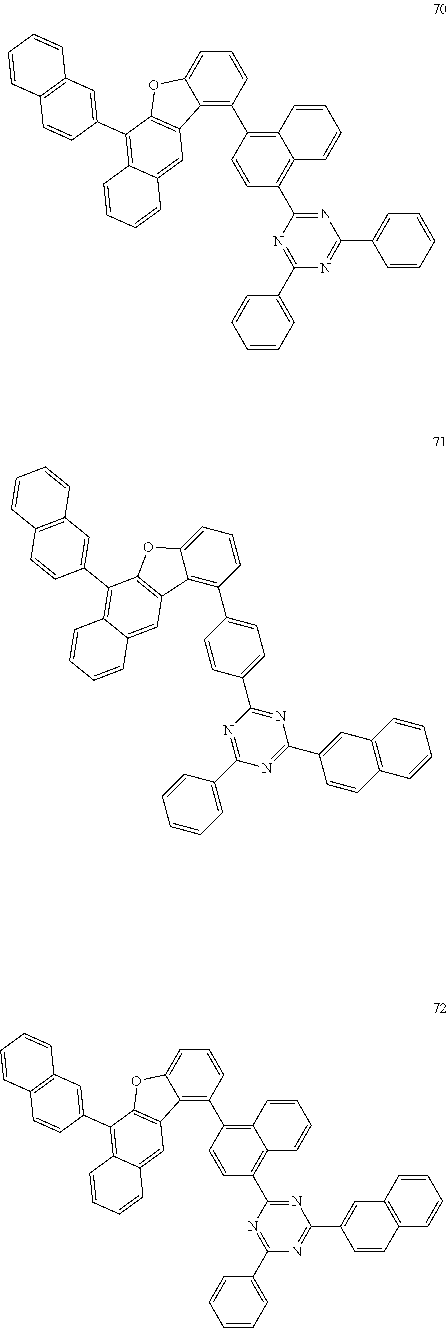 [SMILES: c1ccc(-c2nc(-c3ccccc3)nc(-c3ccc(-c4cccc5oc6c(-c7ccc8ccccc8c7)c7ccccc7cc6c45)c4ccccc34)n2)cc1 ; c1ccc(-c2nc(-c3ccc(-c4cccc5oc6c(-c7ccc8ccccc8c7)c7ccccc7cc6c45)cc3)nc(-c3ccc4ccccc4c3)n2)cc1 ; c1ccc(-c2nc(-c3ccc4ccccc4c3)nc(-c3ccc(-c4cccc5oc6c(-c7ccc8ccccc8c7)c7ccccc7cc6c45)c4ccccc34)n2)cc1]

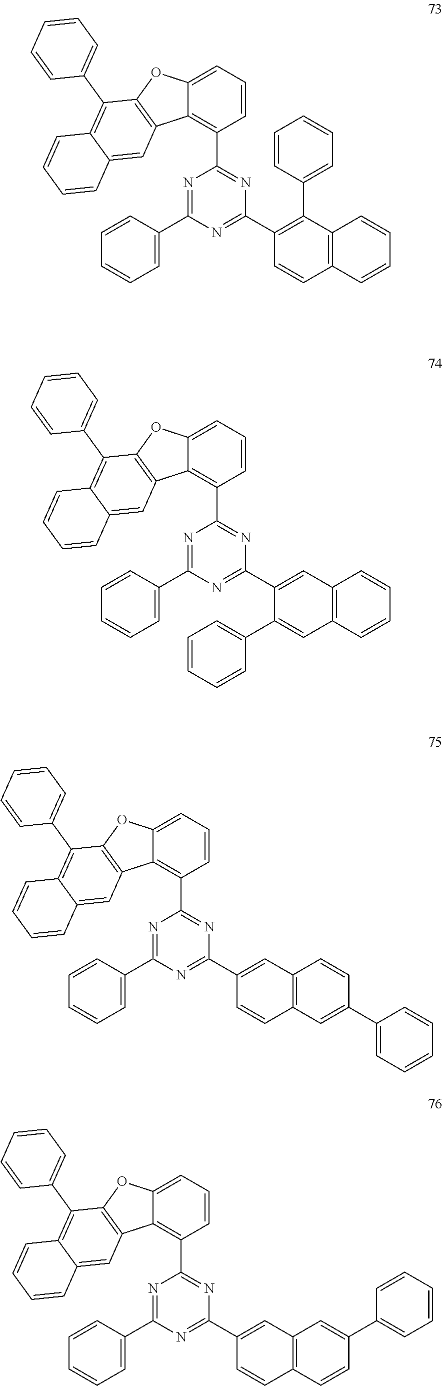 [SMILES: c1ccc(-c2nc(-c3ccc4ccccc4c3-c3ccccc3)nc(-c3cccc4oc5c(-c6ccccc6)c6ccccc6cc5c34)n2)cc1 ; c1ccc(-c2nc(-c3cc4ccccc4cc3-c3ccccc3)nc(-c3cccc4oc5c(-c6ccccc6)c6ccccc6cc5c34)n2)cc1 ; c1ccc(-c2ccc3cc(-c4nc(-c5ccccc5)nc(-c5cccc6oc7c(-c8ccccc8)c8ccccc8cc7c56)n4)ccc3c2)cc1 ; c1ccc(-c2ccc3ccc(-c4nc(-c5ccccc5)nc(-c5cccc6oc7c(-c8ccccc8)c8ccccc8cc7c56)n4)cc3c2)cc1]

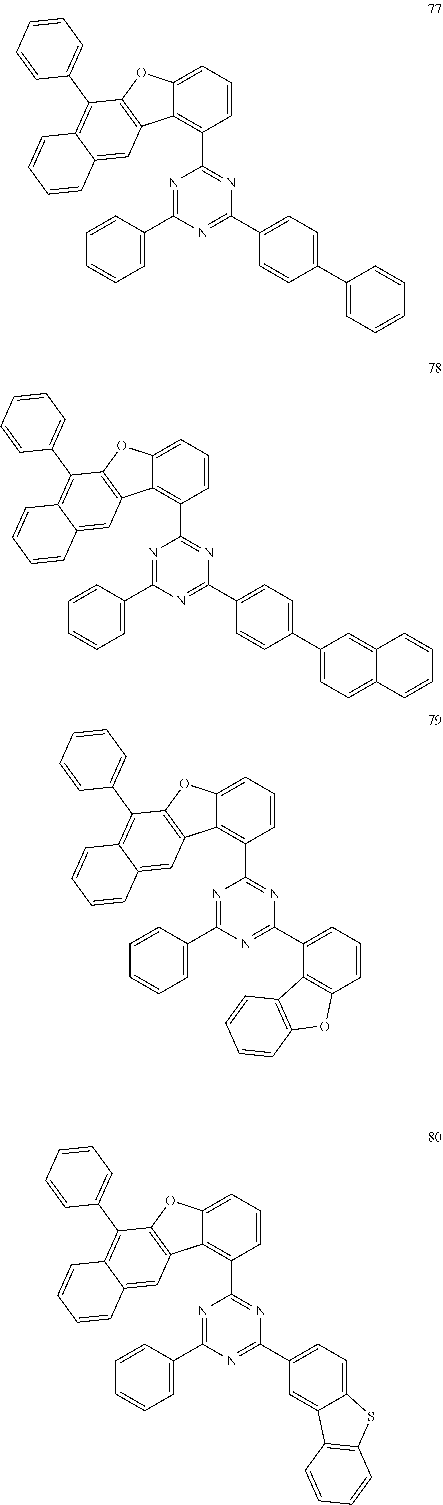 [SMILES: c1ccc(-c2ccc(-c3nc(-c4ccccc4)nc(-c4cccc5oc6c(-c7ccccc7)c7ccccc7cc6c45)n3)cc2)cc1 ; c1ccc(-c2nc(-c3ccc(-c4ccc5ccccc5c4)cc3)nc(-c3cccc4oc5c(-c6ccccc6)c6ccccc6cc5c34)n2)cc1 ; c1ccc(-c2nc(-c3cccc4oc5ccccc5c34)nc(-c3cccc4oc5c(-c6ccccc6)c6ccccc6cc5c34)n2)cc1 ; c1ccc(-c2nc(-c3ccc4sc5ccccc5c4c3)nc(-c3cccc4oc5c(-c6ccccc6)c6ccccc6cc5c34)n2)cc1]

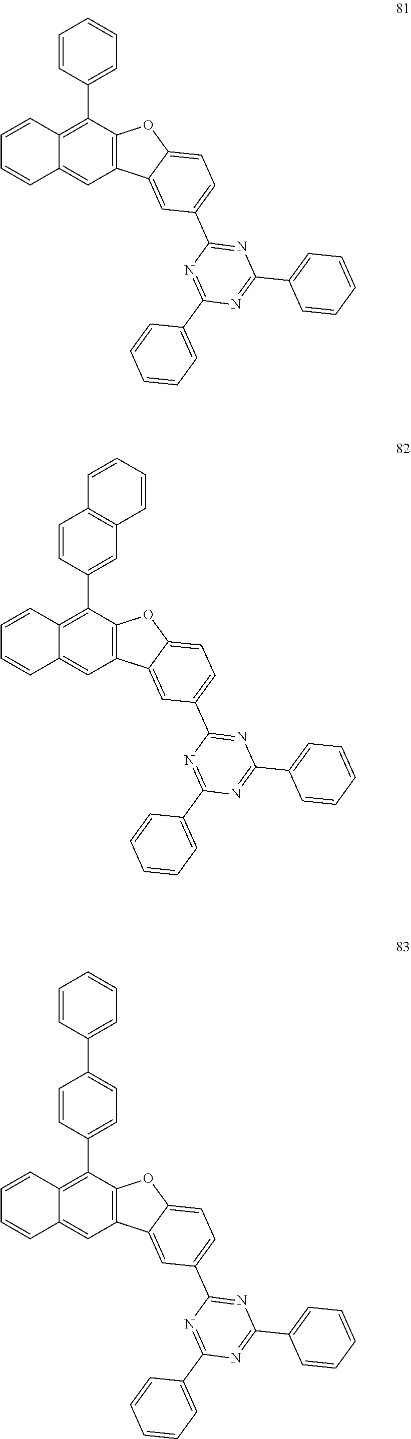 [SMILES: c1ccc(-c2nc(-c3ccccc3)nc(-c3ccc4oc5c(-c6ccccc6)c6ccccc6cc5c4c3)n2)cc1 ; c1ccc(-c2nc(-c3ccccc3)nc(-c3ccc4oc5c(-c6ccc7ccccc7c6)c6ccccc6cc5c4c3)n2)cc1 ; c1ccc(-c2ccc(-c3c4ccccc4cc4c3oc3ccc(-c5nc(-c6ccccc6)nc(-c6ccccc6)n5)cc34)cc2)cc1]

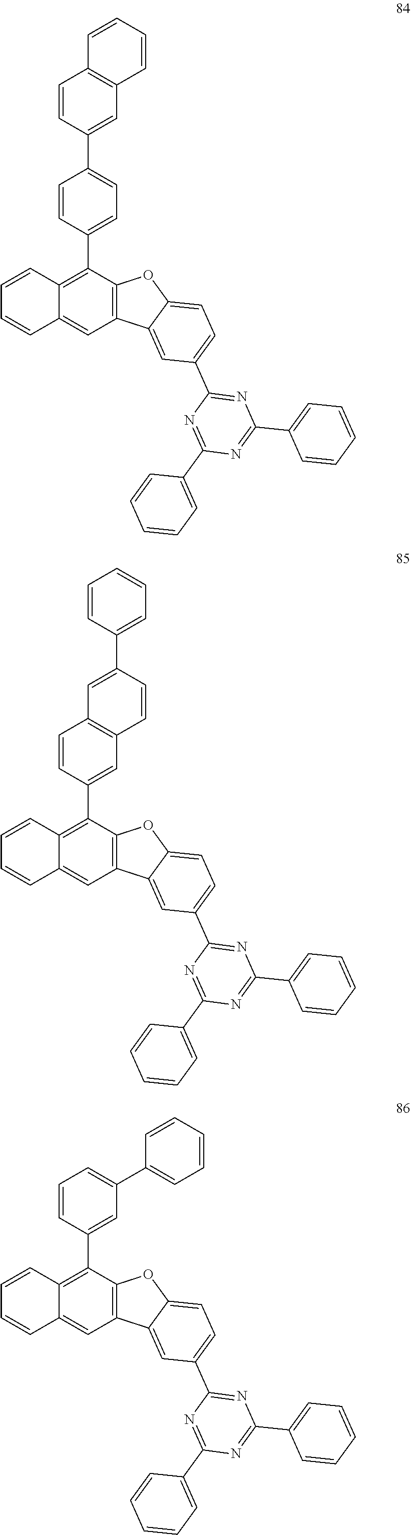

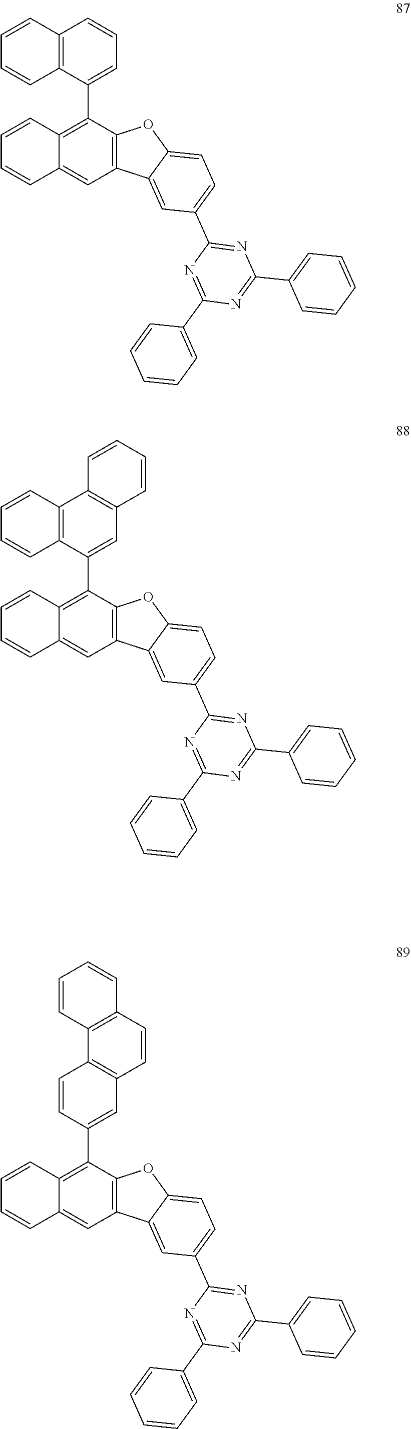 [SMILES: c1ccc(-c2nc(-c3ccccc3)nc(-c3ccc4oc5c(-c6cccc7ccccc67)c6ccccc6cc5c4c3)n2)cc1 ; c1ccc(-c2nc(-c3ccccc3)nc(-c3ccc4oc5c(-c6cc7ccccc7c7ccccc67)c6ccccc6cc5c4c3)n2)cc1 ; c1ccc(-c2nc(-c3ccccc3)nc(-c3ccc4oc5c(-c6ccc7c(ccc8ccccc87)c6)c6ccccc6cc5c4c3)n2)cc1]

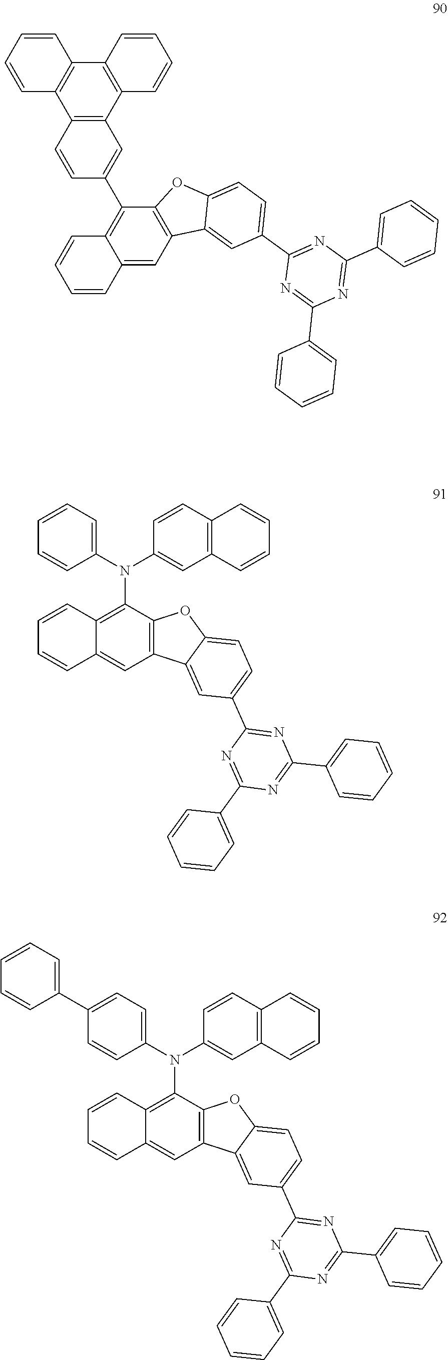 [SMILES: c1ccc(-c2nc(-c3ccccc3)nc(-c3ccc4oc5c(-c6ccc7c8ccccc8c8ccccc8c7c6)c6ccccc6cc5c4c3)n2)cc1 ; c1ccc(-c2nc(-c3ccccc3)nc(-c3ccc4oc5c(N(c6ccccc6)c6ccc7ccccc7c6)c6ccccc6cc5c4c3)n2)cc1 ; c1ccc(-c2ccc(N(c3ccc4ccccc4c3)c3c4ccccc4cc4c3oc3ccc(-c5nc(-c6ccccc6)nc(-c6ccccc6)n5)cc34)cc2)cc1]

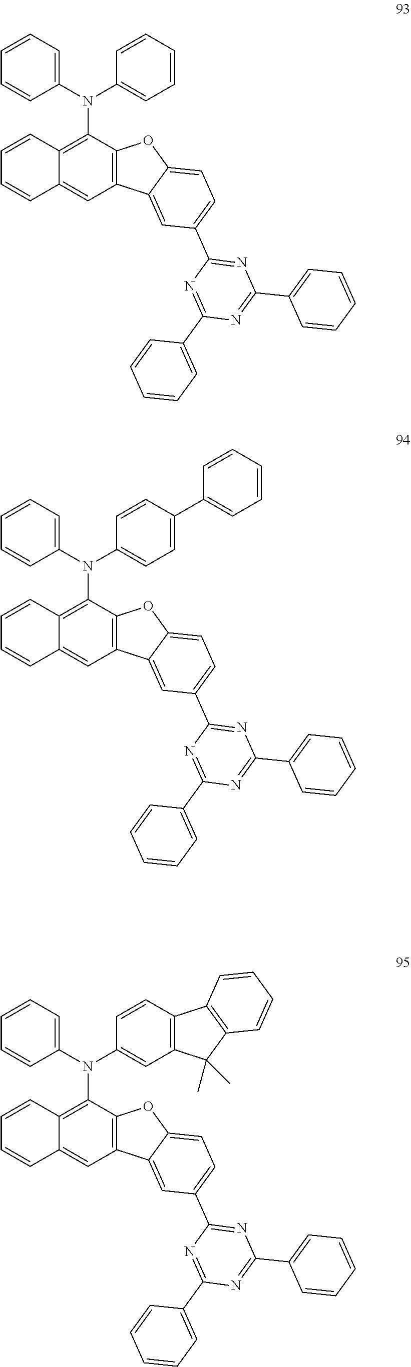 [SMILES: c1ccc(-c2nc(-c3ccccc3)nc(-c3ccc4oc5c(N(c6ccccc6)c6ccccc6)c6ccccc6cc5c4c3)n2)cc1 ; c1ccc(-c2ccc(N(c3ccccc3)c3c4ccccc4cc4c3oc3ccc(-c5nc(-c6ccccc6)nc(-c6ccccc6)n5)cc34)cc2)cc1 ; CC1(C)c2ccccc2-c2ccc(N(c3ccccc3)c3c4ccccc4cc4c3oc3ccc(-c5nc(-c6ccccc6)nc(-c6ccccc6)n5)cc34)cc21]

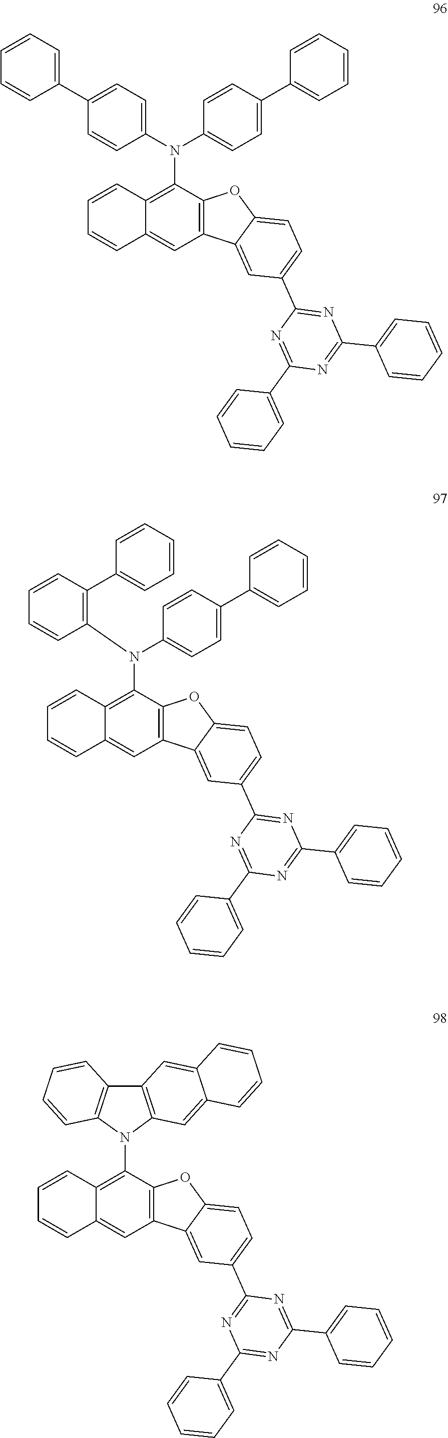 [SMILES: c1ccc(-c2ccc(N(c3ccc(-c4ccccc4)cc3)c3c4ccccc4cc4c3oc3ccc(-c5nc(-c6ccccc6)nc(-c6ccccc6)n5)cc34)cc2)cc1 ; c1ccc(-c2ccc(N(c3ccccc3-c3ccccc3)c3c4ccccc4cc4c3oc3ccc(-c5nc(-c6ccccc6)nc(-c6ccccc6)n5)cc34)cc2)cc1 ; c1ccc(-c2nc(-c3ccccc3)nc(-c3ccc4oc5c(-n6c7ccccc7c7cc8ccccc8cc76)c6ccccc6cc5c4c3)n2)cc1]

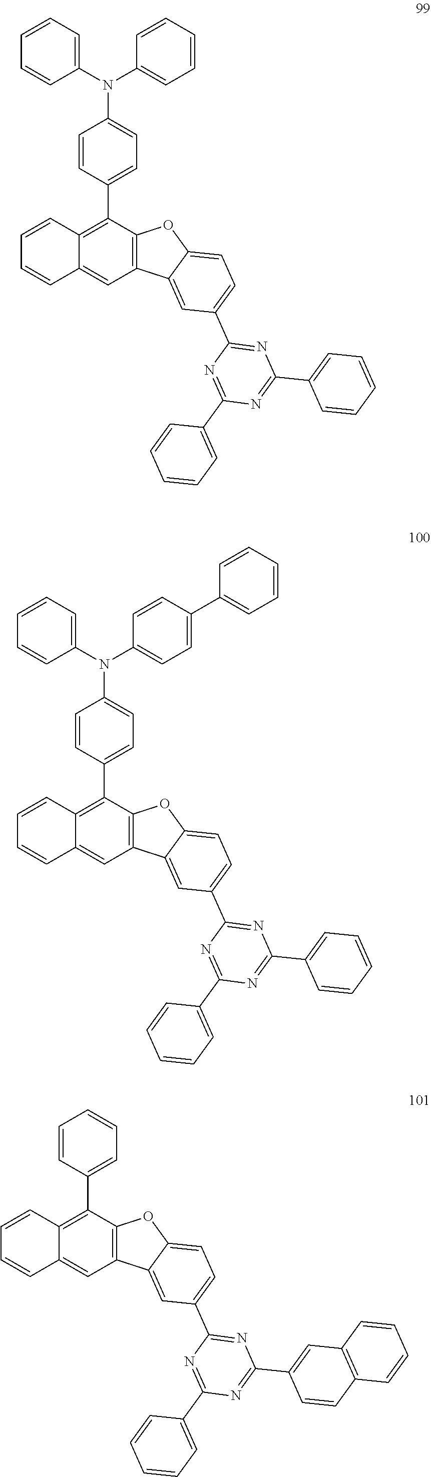 [SMILES: c1ccc(-c2nc(-c3ccccc3)nc(-c3ccc4oc5c(-c6ccc(N(c7ccccc7)c7ccccc7)cc6)c6ccccc6cc5c4c3)n2)cc1 ; c1ccc(-c2ccc(N(c3ccccc3)c3ccc(-c4c5ccccc5cc5c4oc4ccc(-c6nc(-c7ccccc7)nc(-c7ccccc7)n6)cc45)cc3)cc2)cc1 ; c1ccc(-c2nc(-c3ccc4ccccc4c3)nc(-c3ccc4oc5c(-c6ccccc6)c6ccccc6cc5c4c3)n2)cc1]

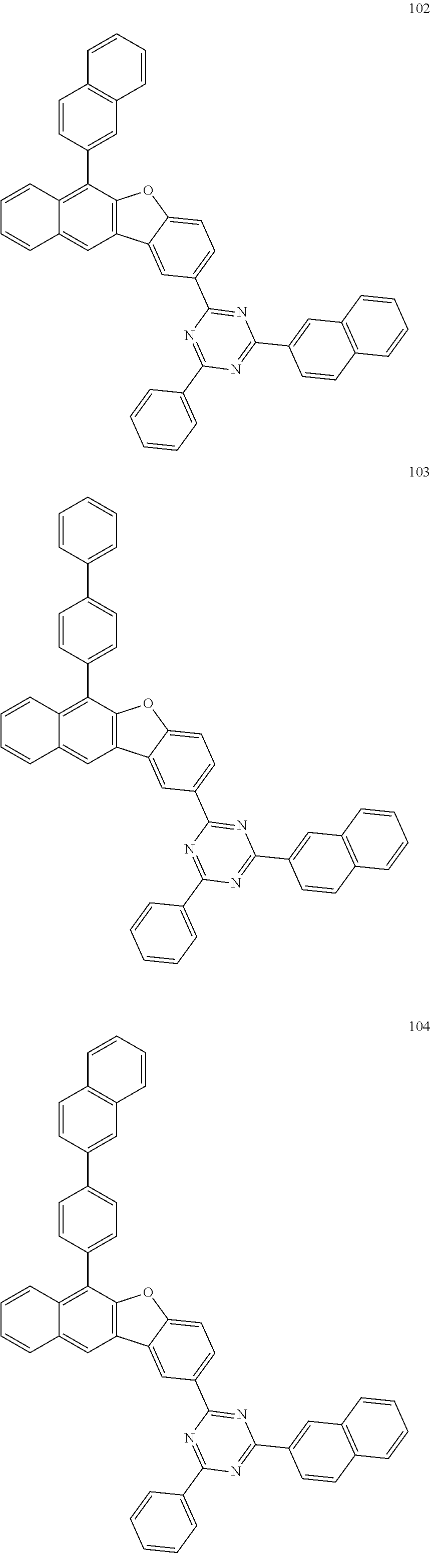 [SMILES: c1ccc(-c2nc(-c3ccc4ccccc4c3)nc(-c3ccc4oc5c(-c6ccc7ccccc7c6)c6ccccc6cc5c4c3)n2)cc1 ; c1ccc(-c2ccc(-c3c4ccccc4cc4c3oc3ccc(-c5nc(-c6ccccc6)nc(-c6ccc7ccccc7c6)n5)cc34)cc2)cc1 ; c1ccc(-c2nc(-c3ccc4ccccc4c3)nc(-c3ccc4oc5c(-c6ccc(-c7ccc8ccccc8c7)cc6)c6ccccc6cc5c4c3)n2)cc1]

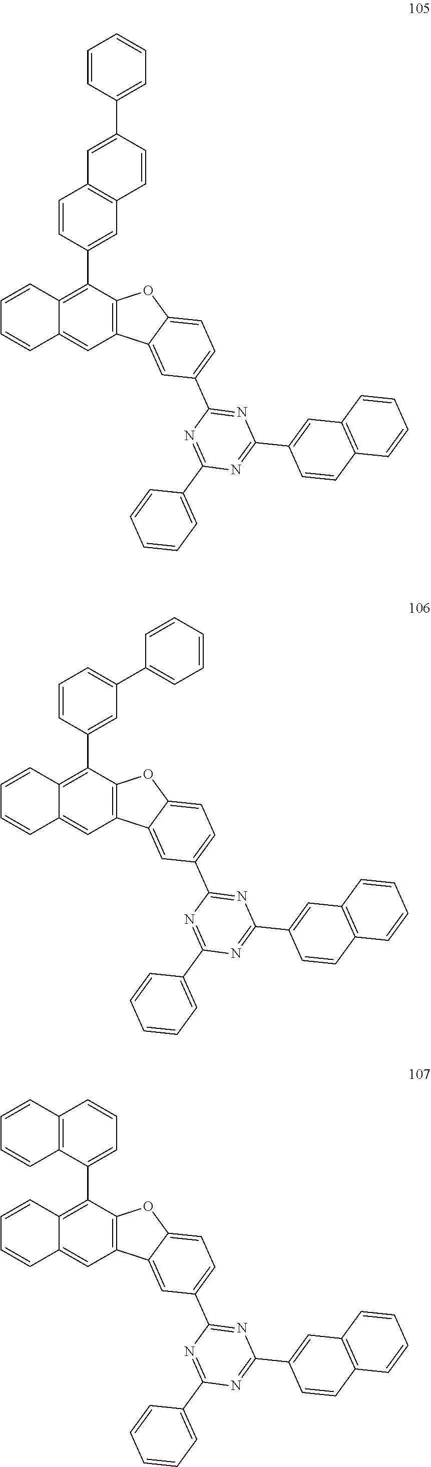 [SMILES: c1ccc(-c2ccc3cc(-c4c5ccccc5cc5c4oc4ccc(-c6nc(-c7ccccc7)nc(-c7ccc8ccccc8c7)n6)cc45)ccc3c2)cc1 ; c1ccc(-c2cccc(-c3c4ccccc4cc4c3oc3ccc(-c5nc(-c6ccccc6)nc(-c6ccc7ccccc7c6)n5)cc34)c2)cc1 ; c1ccc(-c2nc(-c3ccc4ccccc4c3)nc(-c3ccc4oc5c(-c6cccc7ccccc67)c6ccccc6cc5c4c3)n2)cc1]

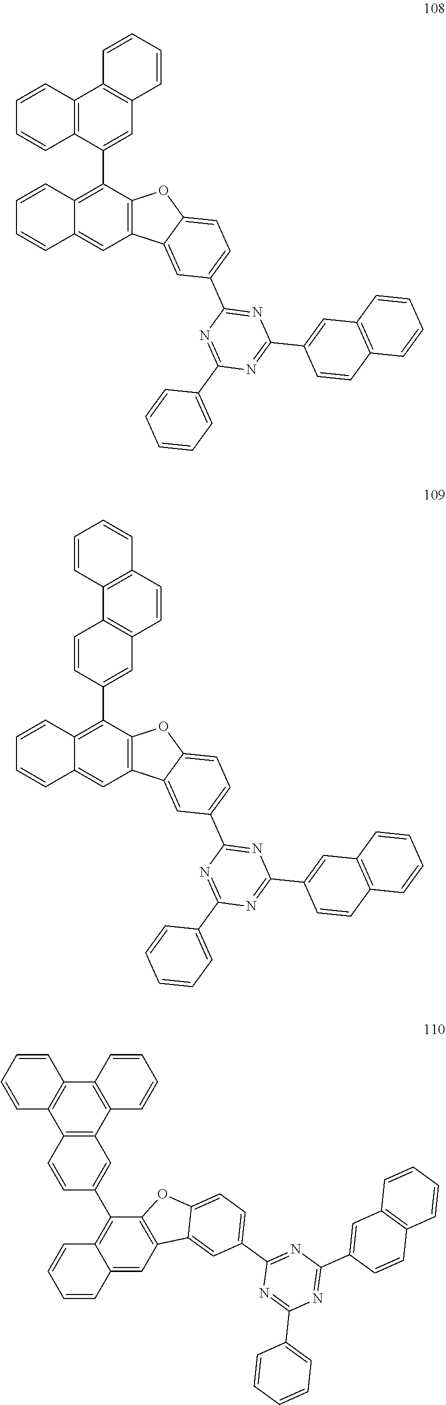 [SMILES: c1ccc(-c2nc(-c3ccc4ccccc4c3)nc(-c3ccc4oc5c(-c6cc7ccccc7c7ccccc67)c6ccccc6cc5c4c3)n2)cc1 ; c1ccc(-c2nc(-c3ccc4ccccc4c3)nc(-c3ccc4oc5c(-c6ccc7c(ccc8ccccc87)c6)c6ccccc6cc5c4c3)n2)cc1 ; c1ccc(-c2nc(-c3ccc4ccccc4c3)nc(-c3ccc4oc5c(-c6ccc7c8ccccc8c8ccccc8c7c6)c6ccccc6cc5c4c3)n2)cc1]

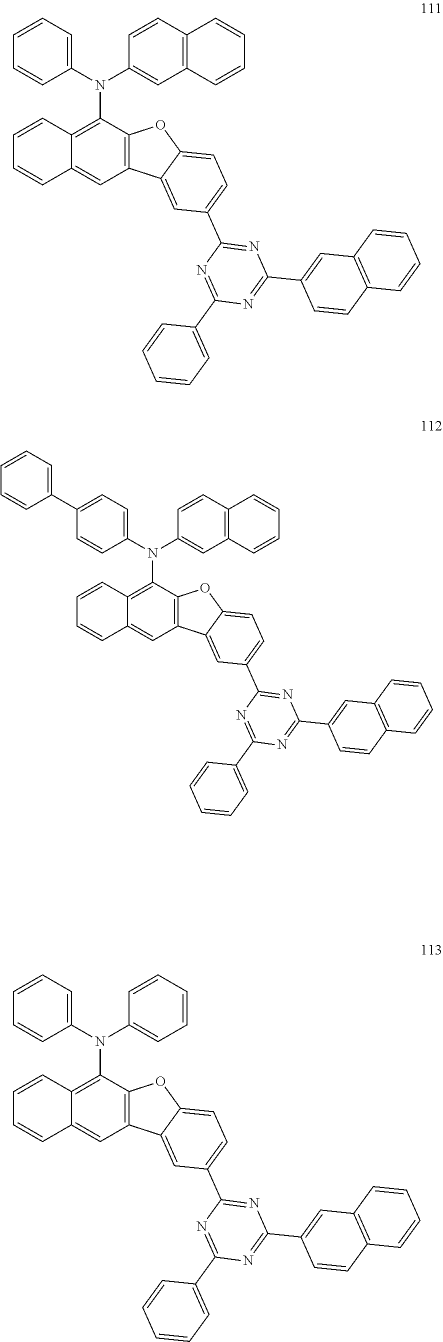 [SMILES: c1ccc(-c2nc(-c3ccc4ccccc4c3)nc(-c3ccc4oc5c(N(c6ccccc6)c6ccc7ccccc7c6)c6ccccc6cc5c4c3)n2)cc1 ; c1ccc(-c2ccc(N(c3ccc4ccccc4c3)c3c4ccccc4cc4c3oc3ccc(-c5nc(-c6ccccc6)nc(-c6ccc7ccccc7c6)n5)cc34)cc2)cc1 ; c1ccc(-c2nc(-c3ccc4ccccc4c3)nc(-c3ccc4oc5c(N(c6ccccc6)c6ccccc6)c6ccccc6cc5c4c3)n2)cc1]

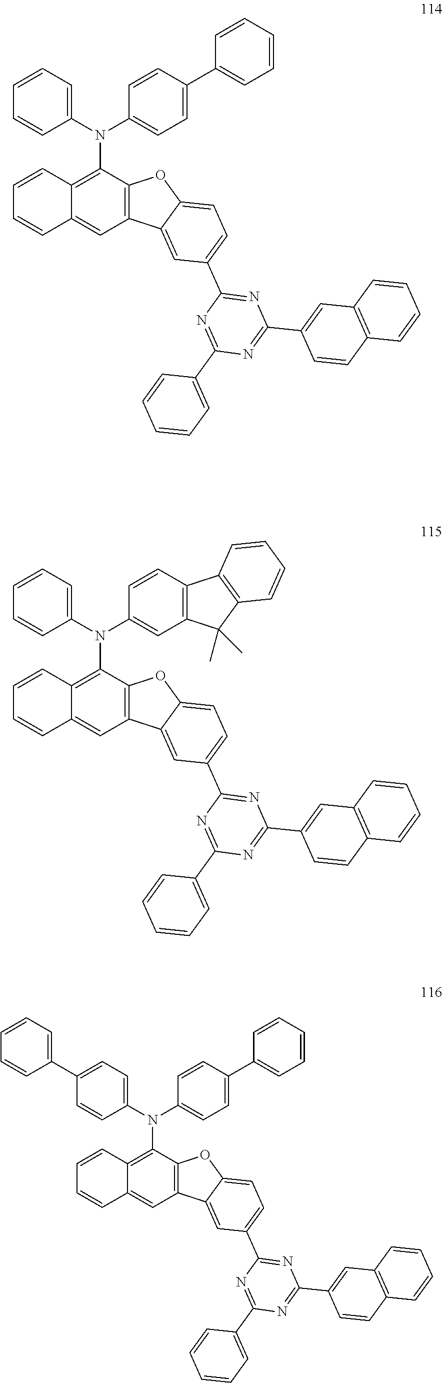 [SMILES: c1ccc(-c2ccc(N(c3ccccc3)c3c4ccccc4cc4c3oc3ccc(-c5nc(-c6ccccc6)nc(-c6ccc7ccccc7c6)n5)cc34)cc2)cc1 ; CC1(C)c2ccccc2-c2ccc(N(c3ccccc3)c3c4ccccc4cc4c3oc3ccc(-c5nc(-c6ccccc6)nc(-c6ccc7ccccc7c6)n5)cc34)cc21 ; c1ccc(-c2ccc(N(c3ccc(-c4ccccc4)cc3)c3c4ccccc4cc4c3oc3ccc(-c5nc(-c6ccccc6)nc(-c6ccc7ccccc7c6)n5)cc34)cc2)cc1]

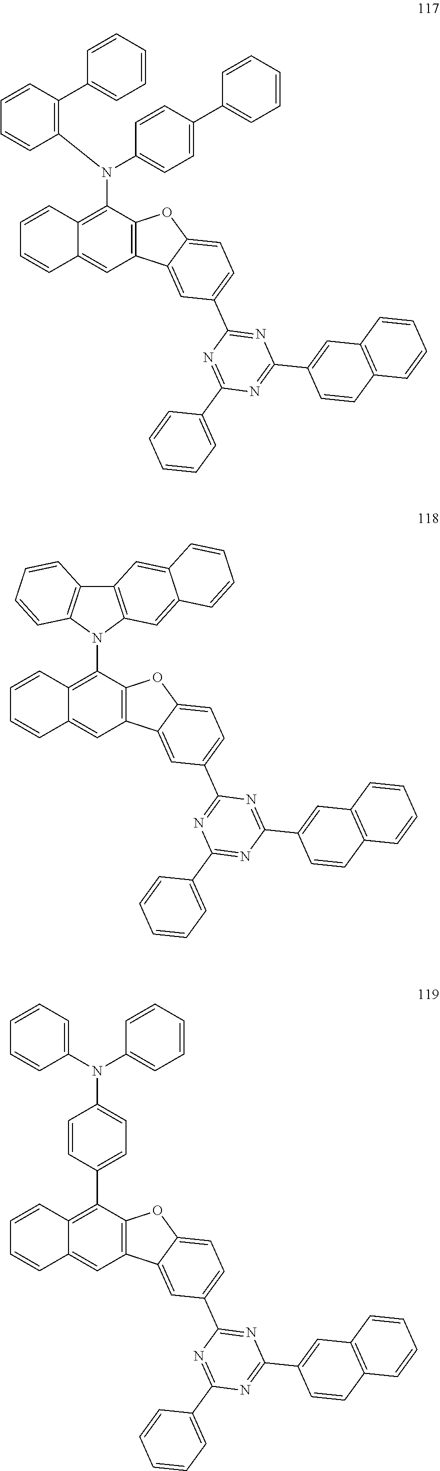 [SMILES: c1ccc(-c2ccc(N(c3ccccc3-c3ccccc3)c3c4ccccc4cc4c3oc3ccc(-c5nc(-c6ccccc6)nc(-c6ccc7ccccc7c6)n5)cc34)cc2)cc1 ; c1ccc(-c2nc(-c3ccc4ccccc4c3)nc(-c3ccc4oc5c(-n6c7ccccc7c7cc8ccccc8cc76)c6ccccc6cc5c4c3)n2)cc1 ; c1ccc(-c2nc(-c3ccc4ccccc4c3)nc(-c3ccc4oc5c(-c6ccc(N(c7ccccc7)c7ccccc7)cc6)c6ccccc6cc5c4c3)n2)cc1]

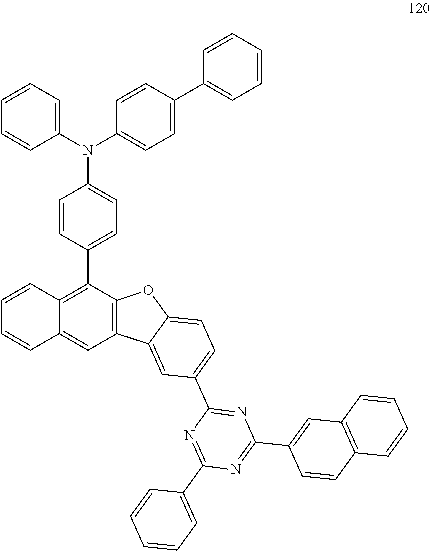 [SMILES: c1ccc(-c2ccc(N(c3ccccc3)c3ccc(-c4c5ccccc5cc5c4oc4ccc(-c6nc(-c7ccccc7)nc(-c7ccc8ccccc8c7)n6)cc45)cc3)cc2)cc1]

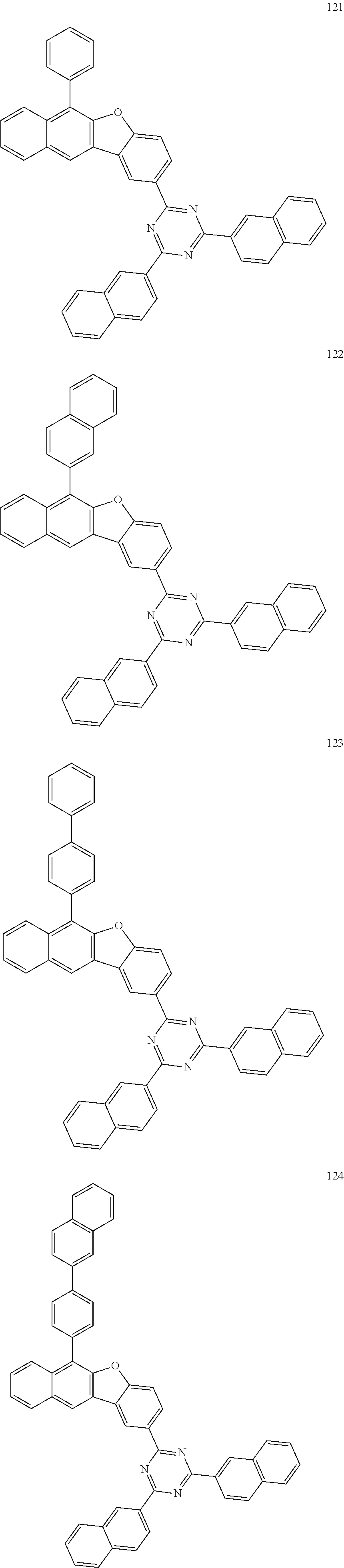

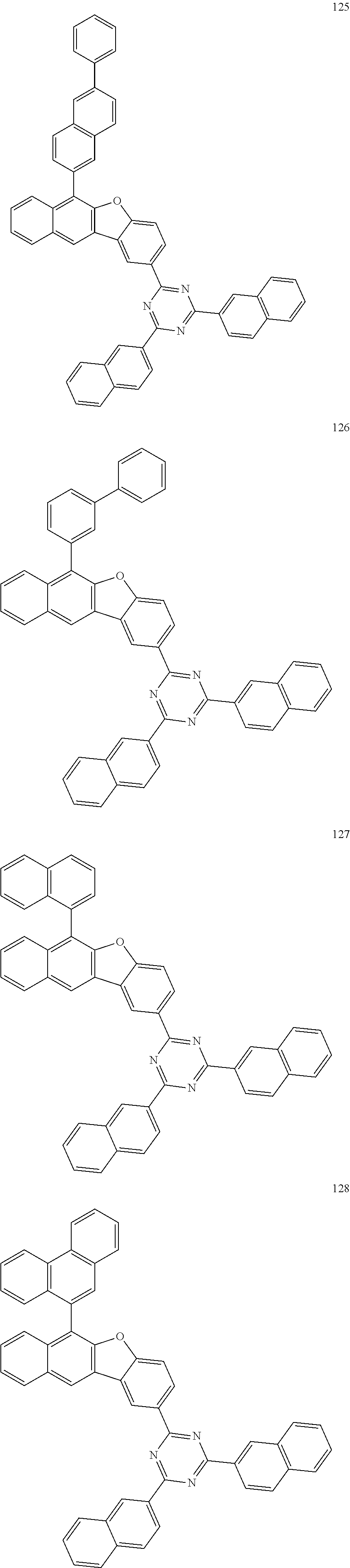

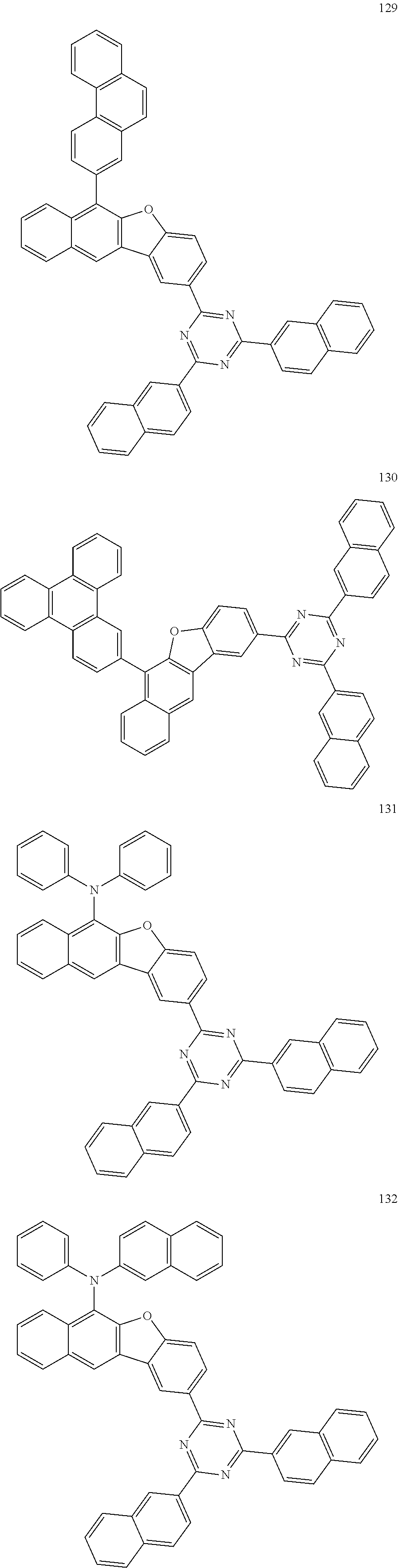 [SMILES: c1ccc2cc(-c3nc(-c4ccc5ccccc5c4)nc(-c4ccc5oc6c(-c7ccc8c(ccc9ccccc98)c7)c7ccccc7cc6c5c4)n3)ccc2c1 ; c1ccc2cc(-c3nc(-c4ccc5ccccc5c4)nc(-c4ccc5oc6c(-c7ccc8c9ccccc9c9ccccc9c8c7)c7ccccc7cc6c5c4)n3)ccc2c1 ; c1ccc(N(c2ccccc2)c2c3ccccc3cc3c2oc2ccc(-c4nc(-c5ccc6ccccc6c5)nc(-c5ccc6ccccc6c5)n4)cc23)cc1 ; c1ccc(N(c2ccc3ccccc3c2)c2c3ccccc3cc3c2oc2ccc(-c4nc(-c5ccc6ccccc6c5)nc(-c5ccc6ccccc6c5)n4)cc23)cc1]

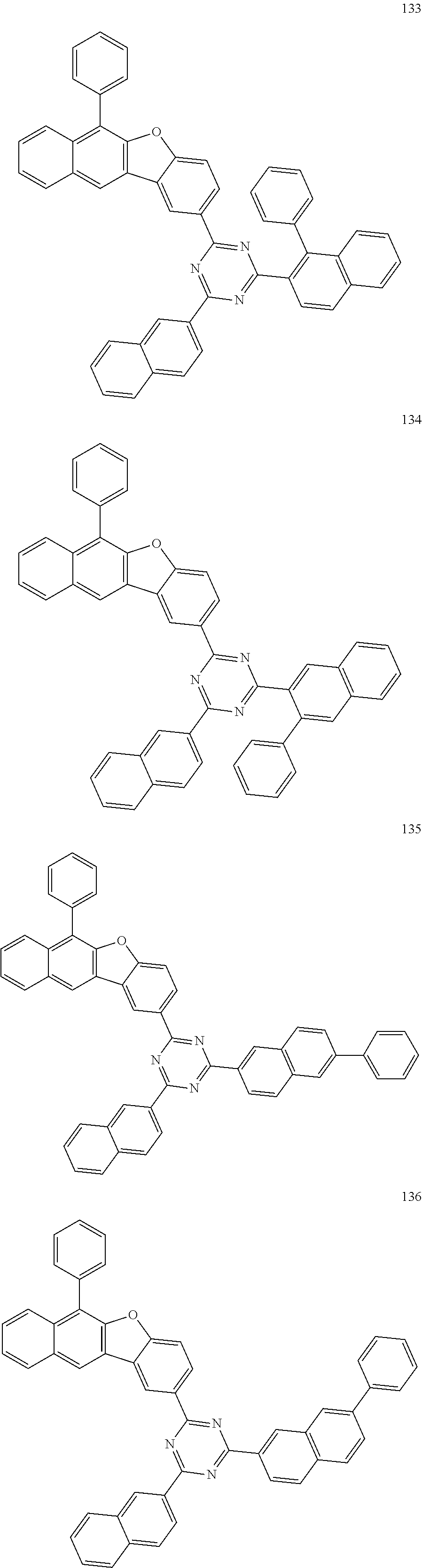 [SMILES: c1ccc(-c2c(-c3nc(-c4ccc5ccccc5c4)nc(-c4ccc5oc6c(-c7ccccc7)c7ccccc7cc6c5c4)n3)ccc3ccccc23)cc1 ; c1ccc(-c2cc3ccccc3cc2-c2nc(-c3ccc4ccccc4c3)nc(-c3ccc4oc5c(-c6ccccc6)c6ccccc6cc5c4c3)n2)cc1 ; c1ccc(-c2ccc3cc(-c4nc(-c5ccc6ccccc6c5)nc(-c5ccc6oc7c(-c8ccccc8)c8ccccc8cc7c6c5)n4)ccc3c2)cc1 ; c1ccc(-c2ccc3ccc(-c4nc(-c5ccc6ccccc6c5)nc(-c5ccc6oc7c(-c8ccccc8)c8ccccc8cc7c6c5)n4)cc3c2)cc1]

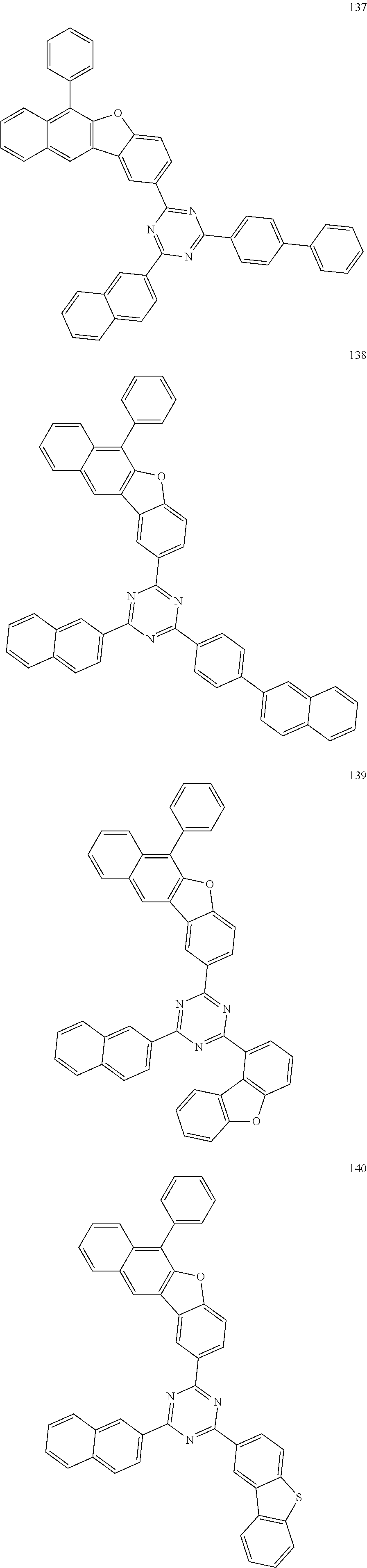

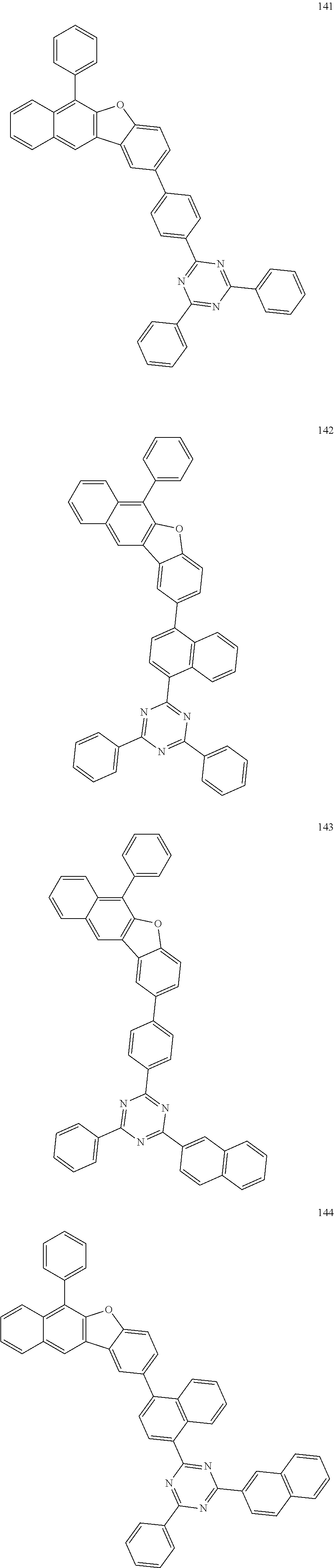 [SMILES: c1ccc(-c2nc(-c3ccccc3)nc(-c3ccc(-c4ccc5oc6c(-c7ccccc7)c7ccccc7cc6c5c4)cc3)n2)cc1 ; c1ccc(-c2nc(-c3ccccc3)nc(-c3ccc(-c4ccc5oc6c(-c7ccccc7)c7ccccc7cc6c5c4)c4ccccc34)n2)cc1 ; c1ccc(-c2nc(-c3ccc(-c4ccc5oc6c(-c7ccccc7)c7ccccc7cc6c5c4)cc3)nc(-c3ccc4ccccc4c3)n2)cc1 ; c1ccc(-c2nc(-c3ccc4ccccc4c3)nc(-c3ccc(-c4ccc5oc6c(-c7ccccc7)c7ccccc7cc6c5c4)c4ccccc34)n2)cc1]

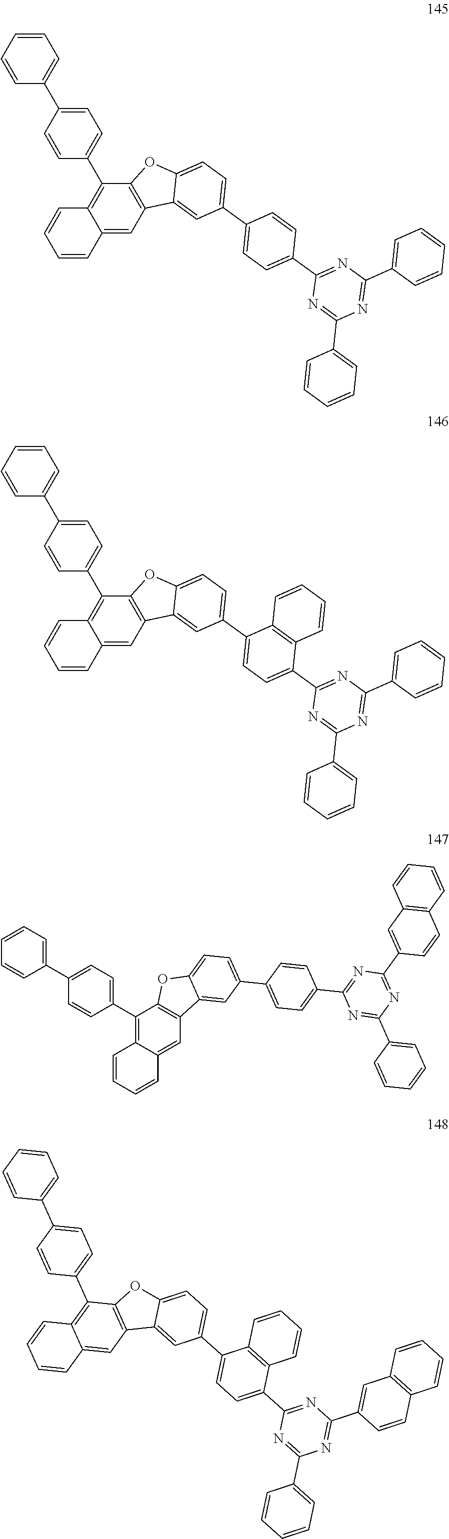 [SMILES: c1ccc(-c2ccc(-c3c4ccccc4cc4c3oc3ccc(-c5ccc(-c6nc(-c7ccccc7)nc(-c7ccccc7)n6)cc5)cc34)cc2)cc1 ; c1ccc(-c2ccc(-c3c4ccccc4cc4c3oc3ccc(-c5ccc(-c6nc(-c7ccccc7)nc(-c7ccccc7)n6)c6ccccc56)cc34)cc2)cc1 ; c1ccc(-c2ccc(-c3c4ccccc4cc4c3oc3ccc(-c5ccc(-c6nc(-c7ccccc7)nc(-c7ccc8ccccc8c7)n6)cc5)cc34)cc2)cc1 ; c1ccc(-c2ccc(-c3c4ccccc4cc4c3oc3ccc(-c5ccc(-c6nc(-c7ccccc7)nc(-c7ccc8ccccc8c7)n6)c6ccccc56)cc34)cc2)cc1]

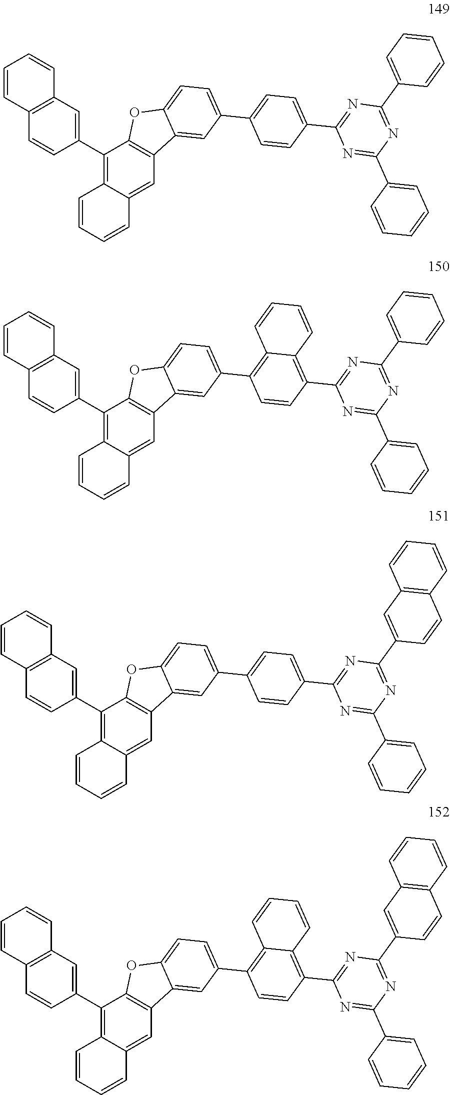 [SMILES: c1ccc(-c2nc(-c3ccccc3)nc(-c3ccc(-c4ccc5oc6c(-c7ccc8ccccc8c7)c7ccccc7cc6c5c4)cc3)n2)cc1 ; c1ccc(-c2nc(-c3ccccc3)nc(-c3ccc(-c4ccc5oc6c(-c7ccc8ccccc8c7)c7ccccc7cc6c5c4)c4ccccc34)n2)cc1 ; c1ccc(-c2nc(-c3ccc(-c4ccc5oc6c(-c7ccc8ccccc8c7)c7ccccc7cc6c5c4)cc3)nc(-c3ccc4ccccc4c3)n2)cc1 ; c1ccc(-c2nc(-c3ccc4ccccc4c3)nc(-c3ccc(-c4ccc5oc6c(-c7ccc8ccccc8c7)c7ccccc7cc6c5c4)c4ccccc34)n2)cc1]

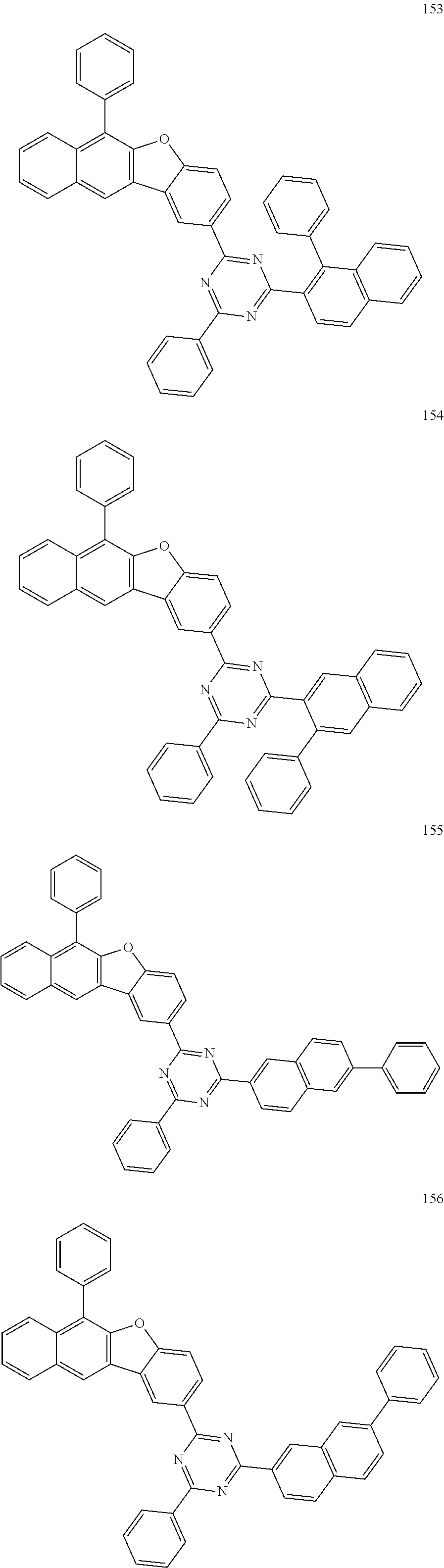 [SMILES: c1ccc(-c2nc(-c3ccc4oc5c(-c6ccccc6)c6ccccc6cc5c4c3)nc(-c3ccc4ccccc4c3-c3ccccc3)n2)cc1 ; c1ccc(-c2nc(-c3ccc4oc5c(-c6ccccc6)c6ccccc6cc5c4c3)nc(-c3cc4ccccc4cc3-c3ccccc3)n2)cc1 ; c1ccc(-c2ccc3cc(-c4nc(-c5ccccc5)nc(-c5ccc6oc7c(-c8ccccc8)c8ccccc8cc7c6c5)n4)ccc3c2)cc1 ; c1ccc(-c2ccc3ccc(-c4nc(-c5ccccc5)nc(-c5ccc6oc7c(-c8ccccc8)c8ccccc8cc7c6c5)n4)cc3c2)cc1]

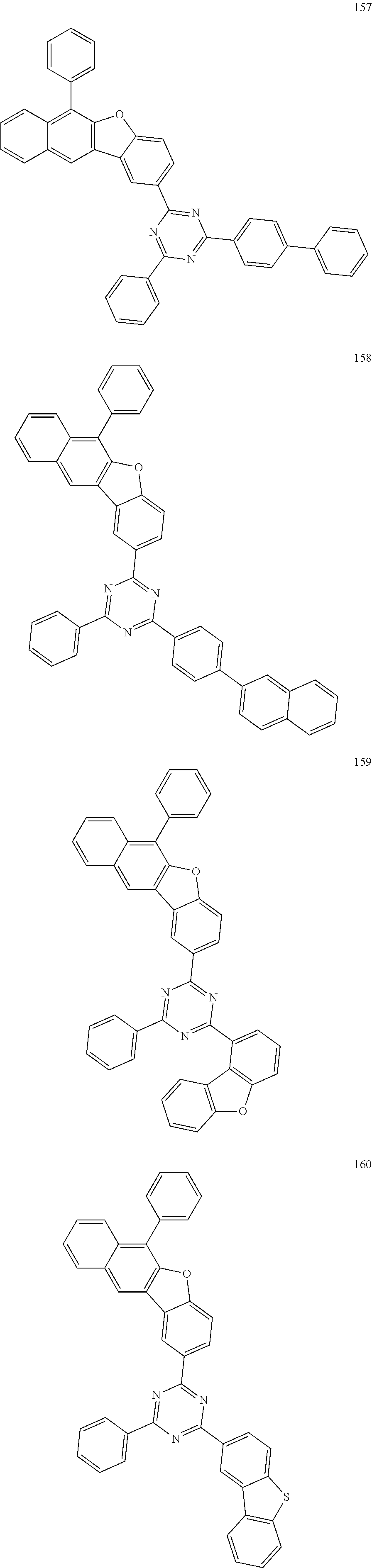 [SMILES: c1ccc(-c2ccc(-c3nc(-c4ccccc4)nc(-c4ccc5oc6c(-c7ccccc7)c7ccccc7cc6c5c4)n3)cc2)cc1 ; c1ccc(-c2nc(-c3ccc(-c4ccc5ccccc5c4)cc3)nc(-c3ccc4oc5c(-c6ccccc6)c6ccccc6cc5c4c3)n2)cc1 ; c1ccc(-c2nc(-c3ccc4oc5c(-c6ccccc6)c6ccccc6cc5c4c3)nc(-c3cccc4oc5ccccc5c34)n2)cc1 ; c1ccc(-c2nc(-c3ccc4oc5c(-c6ccccc6)c6ccccc6cc5c4c3)nc(-c3ccc4sc5ccccc5c4c3)n2)cc1]

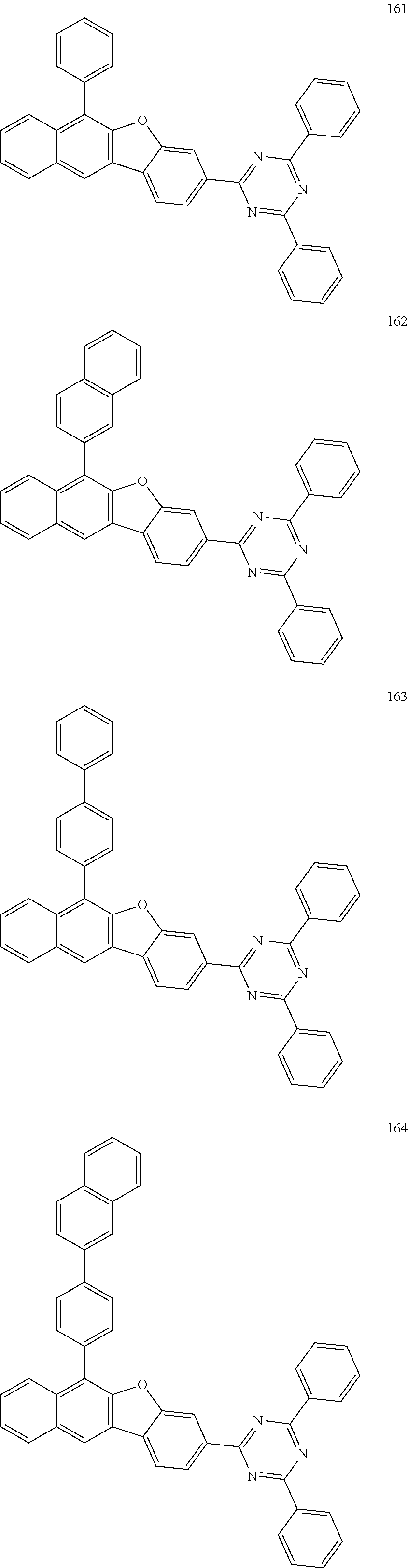 [SMILES: c1ccc(-c2nc(-c3ccccc3)nc(-c3ccc4c(c3)oc3c(-c5ccccc5)c5ccccc5cc34)n2)cc1 ; c1ccc(-c2nc(-c3ccccc3)nc(-c3ccc4c(c3)oc3c(-c5ccc6ccccc6c5)c5ccccc5cc34)n2)cc1 ; c1ccc(-c2ccc(-c3c4ccccc4cc4c3oc3cc(-c5nc(-c6ccccc6)nc(-c6ccccc6)n5)ccc34)cc2)cc1 ; c1ccc(-c2nc(-c3ccccc3)nc(-c3ccc4c(c3)oc3c(-c5ccc(-c6ccc7ccccc7c6)cc5)c5ccccc5cc34)n2)cc1]

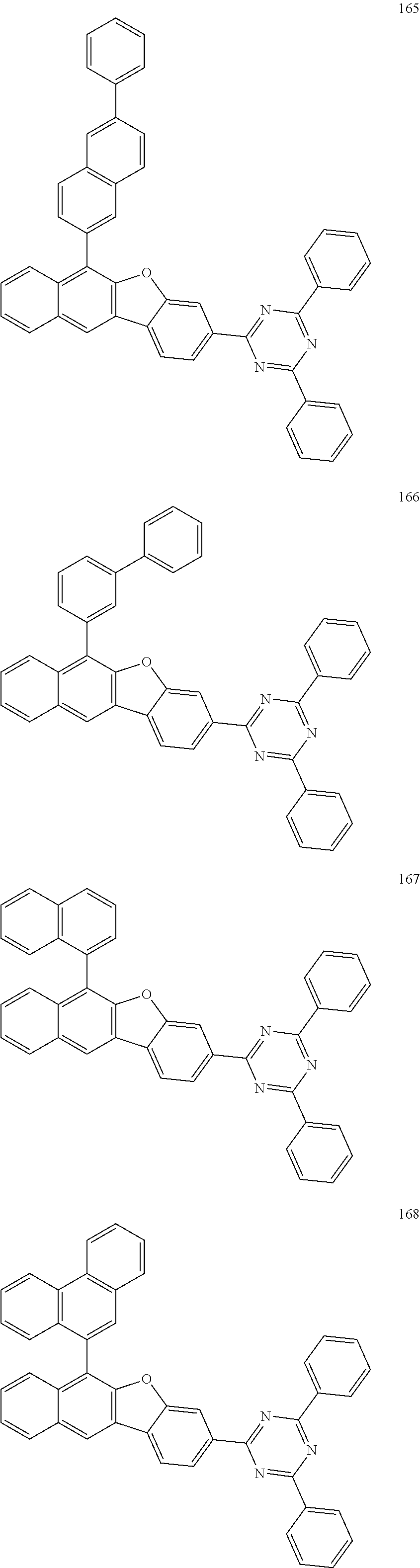 [SMILES: c1ccc(-c2ccc3cc(-c4c5ccccc5cc5c4oc4cc(-c6nc(-c7ccccc7)nc(-c7ccccc7)n6)ccc45)ccc3c2)cc1 ; c1ccc(-c2cccc(-c3c4ccccc4cc4c3oc3cc(-c5nc(-c6ccccc6)nc(-c6ccccc6)n5)ccc34)c2)cc1 ; c1ccc(-c2nc(-c3ccccc3)nc(-c3ccc4c(c3)oc3c(-c5cccc6ccccc56)c5ccccc5cc34)n2)cc1 ; c1ccc(-c2nc(-c3ccccc3)nc(-c3ccc4c(c3)oc3c(-c5cc6ccccc6c6ccccc56)c5ccccc5cc34)n2)cc1]

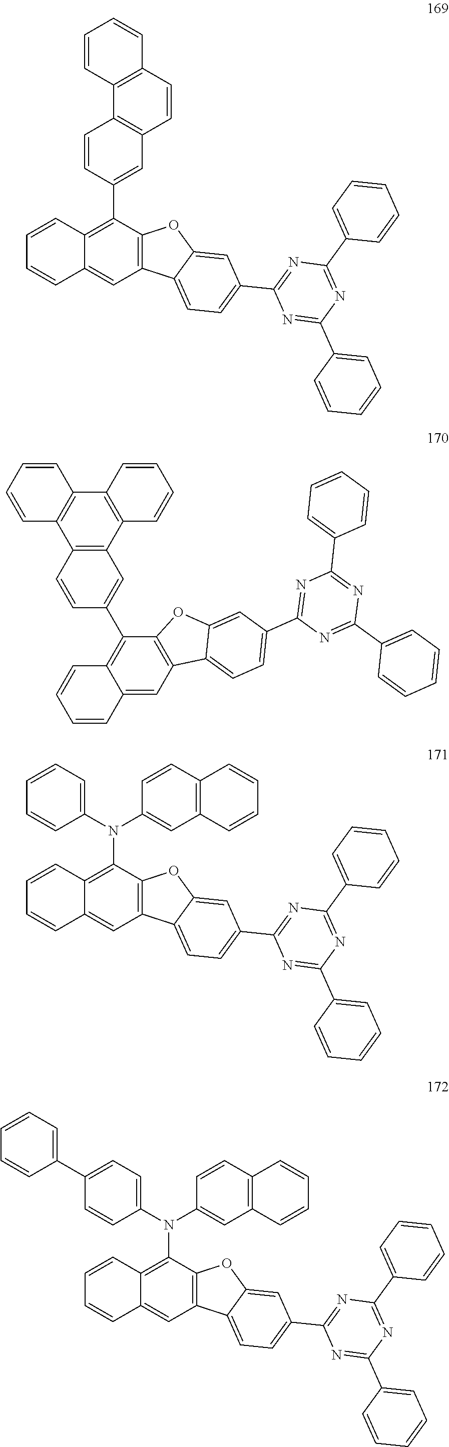

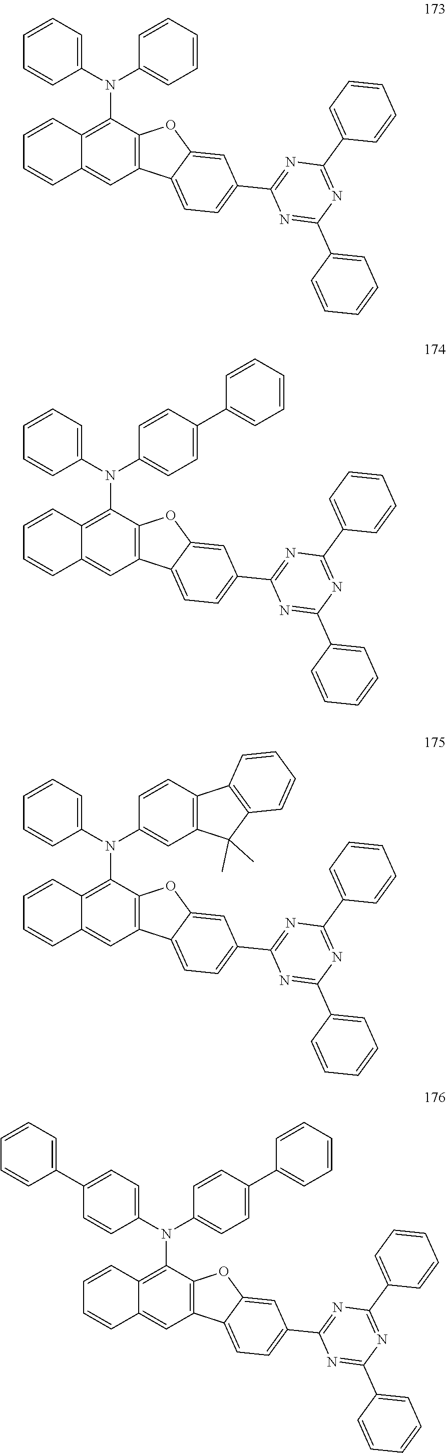 [SMILES: c1ccc(-c2nc(-c3ccccc3)nc(-c3ccc4c(c3)oc3c(N(c5ccccc5)c5ccccc5)c5ccccc5cc34)n2)cc1 ; c1ccc(-c2ccc(N(c3ccccc3)c3c4ccccc4cc4c3oc3cc(-c5nc(-c6ccccc6)nc(-c6ccccc6)n5)ccc34)cc2)cc1 ; CC1(C)c2ccccc2-c2ccc(N(c3ccccc3)c3c4ccccc4cc4c3oc3cc(-c5nc(-c6ccccc6)nc(-c6ccccc6)n5)ccc34)cc21 ; c1ccc(-c2ccc(N(c3ccc(-c4ccccc4)cc3)c3c4ccccc4cc4c3oc3cc(-c5nc(-c6ccccc6)nc(-c6ccccc6)n5)ccc34)cc2)cc1]

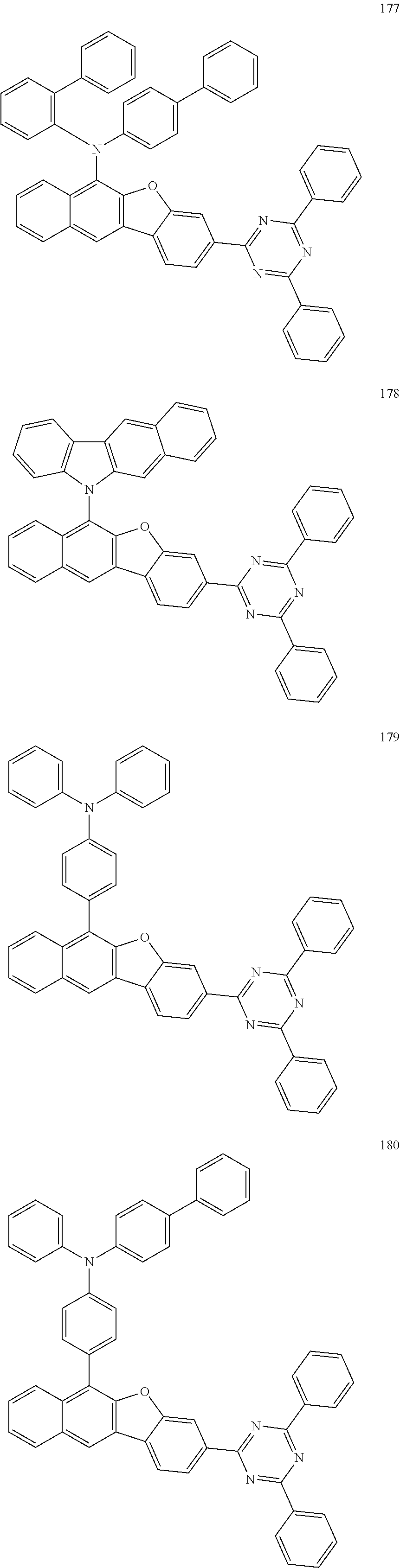 [SMILES: c1ccc(-c2ccc(N(c3ccccc3-c3ccccc3)c3c4ccccc4cc4c3oc3cc(-c5nc(-c6ccccc6)nc(-c6ccccc6)n5)ccc34)cc2)cc1 ; c1ccc(-c2nc(-c3ccccc3)nc(-c3ccc4c(c3)oc3c(-n5c6ccccc6c6cc7ccccc7cc65)c5ccccc5cc34)n2)cc1 ; c1ccc(-c2nc(-c3ccccc3)nc(-c3ccc4c(c3)oc3c(-c5ccc(N(c6ccccc6)c6ccccc6)cc5)c5ccccc5cc34)n2)cc1 ; c1ccc(-c2ccc(N(c3ccccc3)c3ccc(-c4c5ccccc5cc5c4oc4cc(-c6nc(-c7ccccc7)nc(-c7ccccc7)n6)ccc45)cc3)cc2)cc1]

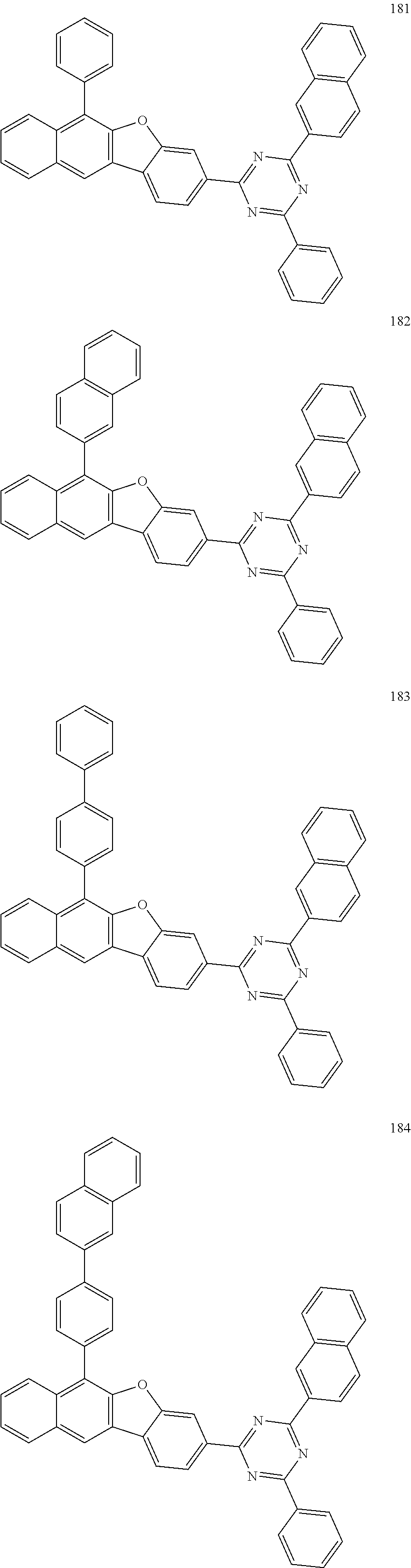 [SMILES: c1ccc(-c2nc(-c3ccc4ccccc4c3)nc(-c3ccc4c(c3)oc3c(-c5ccccc5)c5ccccc5cc34)n2)cc1 ; c1ccc(-c2nc(-c3ccc4ccccc4c3)nc(-c3ccc4c(c3)oc3c(-c5ccc6ccccc6c5)c5ccccc5cc34)n2)cc1 ; c1ccc(-c2ccc(-c3c4ccccc4cc4c3oc3cc(-c5nc(-c6ccccc6)nc(-c6ccc7ccccc7c6)n5)ccc34)cc2)cc1 ; c1ccc(-c2nc(-c3ccc4ccccc4c3)nc(-c3ccc4c(c3)oc3c(-c5ccc(-c6ccc7ccccc7c6)cc5)c5ccccc5cc34)n2)cc1]

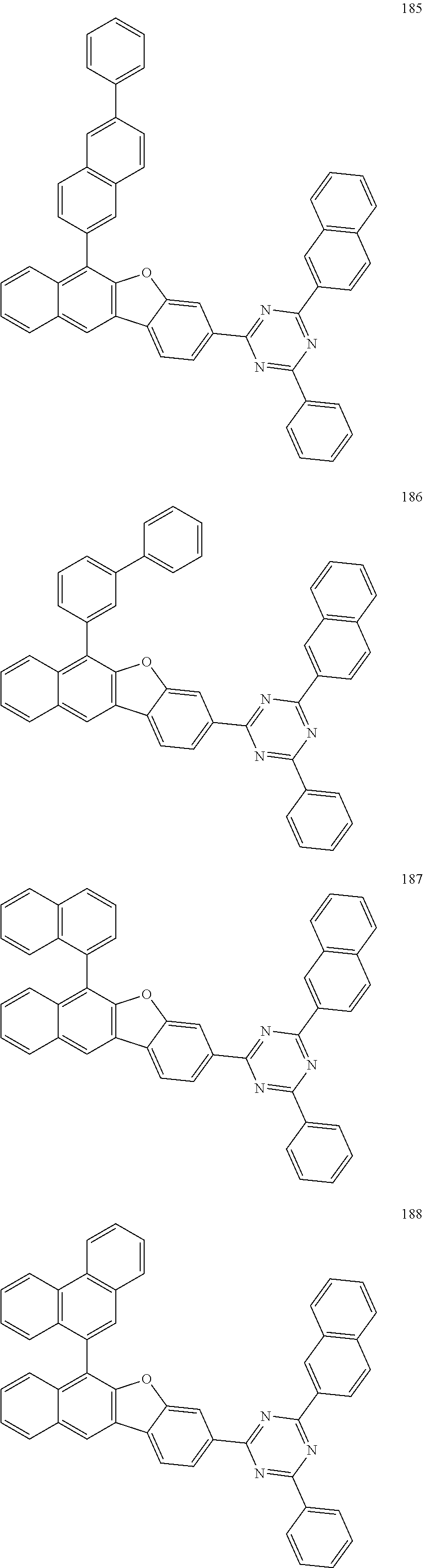

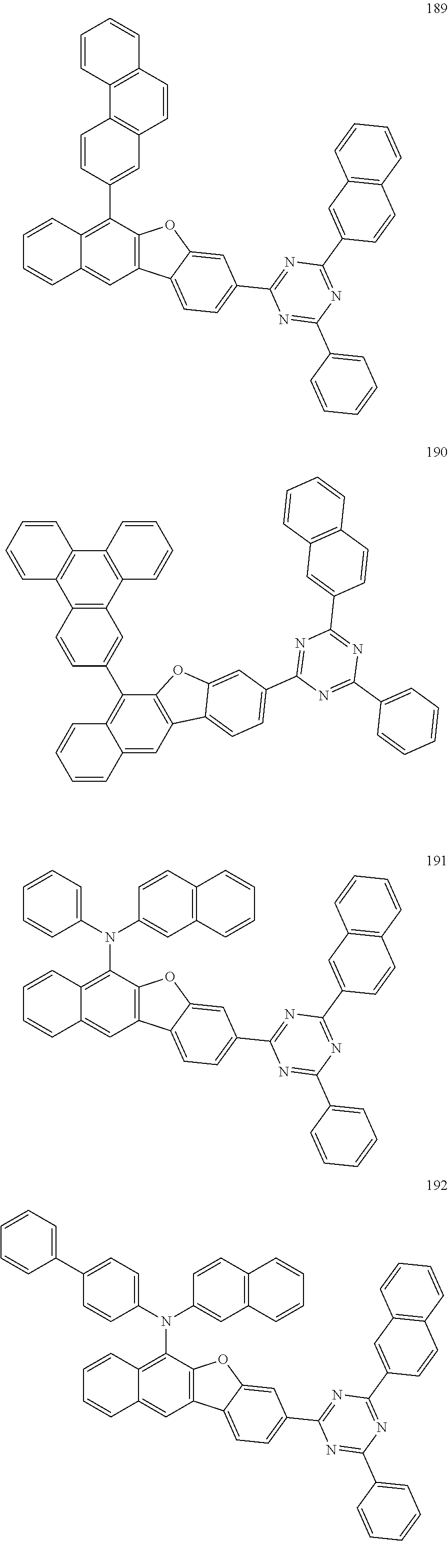 [SMILES: c1ccc(-c2nc(-c3ccc4ccccc4c3)nc(-c3ccc4c(c3)oc3c(-c5ccc6c(ccc7ccccc76)c5)c5ccccc5cc34)n2)cc1 ; c1ccc(-c2nc(-c3ccc4ccccc4c3)nc(-c3ccc4c(c3)oc3c(-c5ccc6c7ccccc7c7ccccc7c6c5)c5ccccc5cc34)n2)cc1 ; c1ccc(-c2nc(-c3ccc4ccccc4c3)nc(-c3ccc4c(c3)oc3c(N(c5ccccc5)c5ccc6ccccc6c5)c5ccccc5cc34)n2)cc1 ; c1ccc(-c2ccc(N(c3ccc4ccccc4c3)c3c4ccccc4cc4c3oc3cc(-c5nc(-c6ccccc6)nc(-c6ccc7ccccc7c6)n5)ccc34)cc2)cc1]

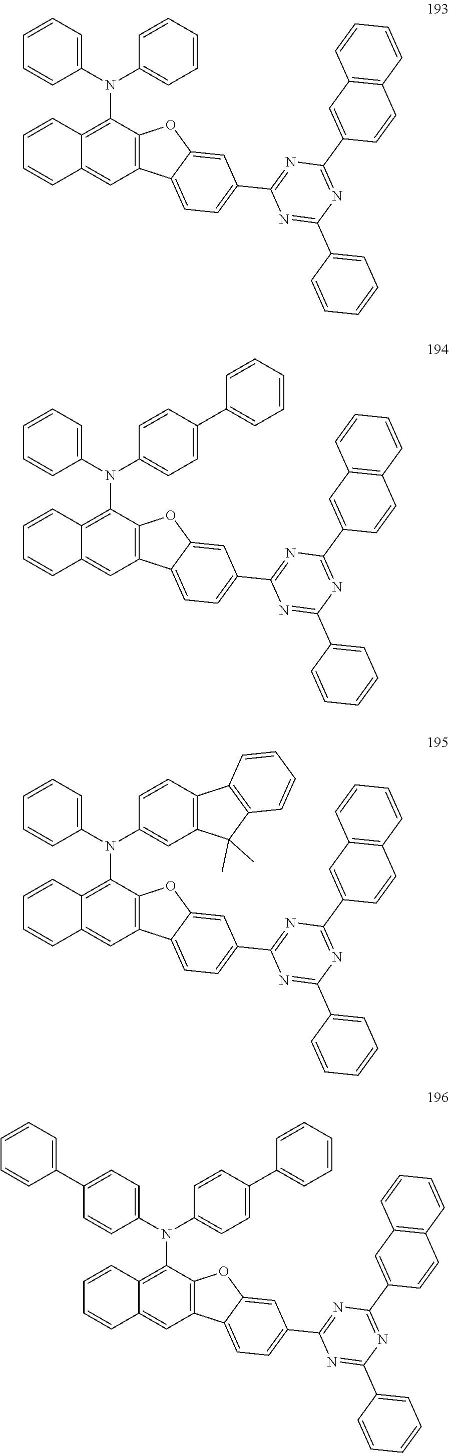 [SMILES: c1ccc(-c2nc(-c3ccc4ccccc4c3)nc(-c3ccc4c(c3)oc3c(N(c5ccccc5)c5ccccc5)c5ccccc5cc34)n2)cc1 ; c1ccc(-c2ccc(N(c3ccccc3)c3c4ccccc4cc4c3oc3cc(-c5nc(-c6ccccc6)nc(-c6ccc7ccccc7c6)n5)ccc34)cc2)cc1 ; CC1(C)c2ccccc2-c2ccc(N(c3ccccc3)c3c4ccccc4cc4c3oc3cc(-c5nc(-c6ccccc6)nc(-c6ccc7ccccc7c6)n5)ccc34)cc21 ; c1ccc(-c2ccc(N(c3ccc(-c4ccccc4)cc3)c3c4ccccc4cc4c3oc3cc(-c5nc(-c6ccccc6)nc(-c6ccc7ccccc7c6)n5)ccc34)cc2)cc1]

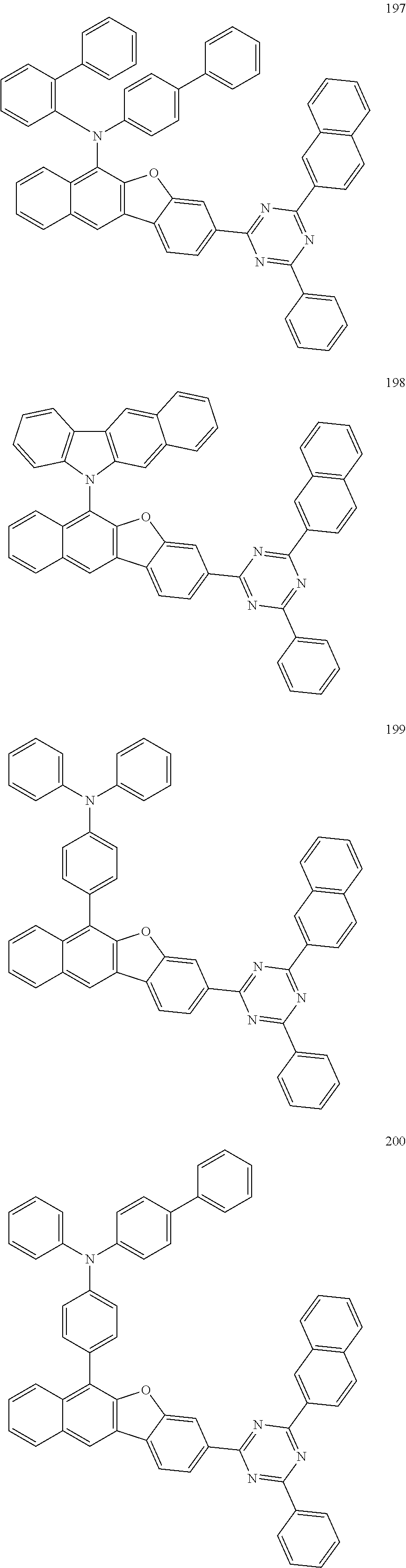 [SMILES: c1ccc(-c2ccc(N(c3ccccc3-c3ccccc3)c3c4ccccc4cc4c3oc3cc(-c5nc(-c6ccccc6)nc(-c6ccc7ccccc7c6)n5)ccc34)cc2)cc1 ; c1ccc(-c2nc(-c3ccc4ccccc4c3)nc(-c3ccc4c(c3)oc3c(-n5c6ccccc6c6cc7ccccc7cc65)c5ccccc5cc34)n2)cc1 ; c1ccc(-c2nc(-c3ccc4ccccc4c3)nc(-c3ccc4c(c3)oc3c(-c5ccc(N(c6ccccc6)c6ccccc6)cc5)c5ccccc5cc34)n2)cc1 ; c1ccc(-c2ccc(N(c3ccccc3)c3ccc(-c4c5ccccc5cc5c4oc4cc(-c6nc(-c7ccccc7)nc(-c7ccc8ccccc8c7)n6)ccc45)cc3)cc2)cc1]

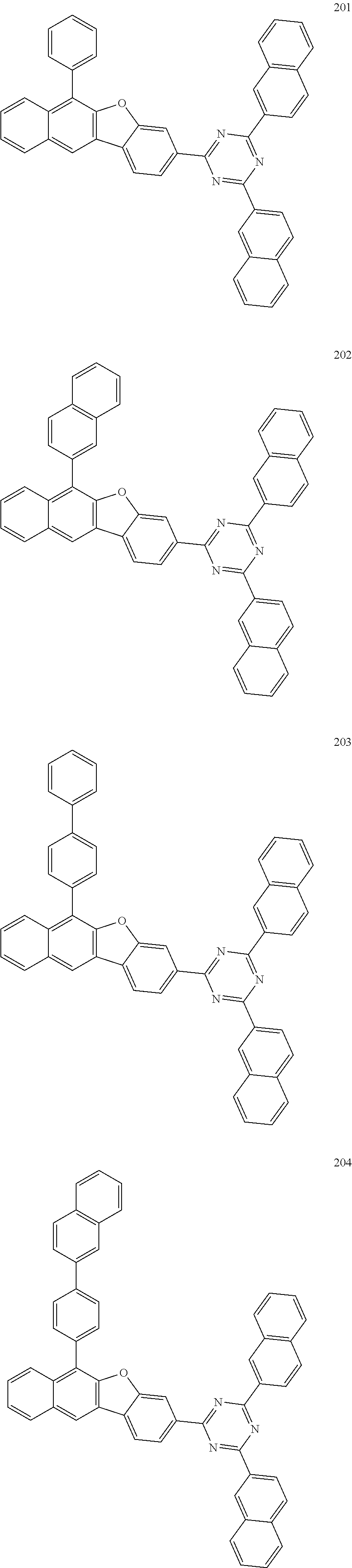 [SMILES: c1ccc(-c2c3ccccc3cc3c2oc2cc(-c4nc(-c5ccc6ccccc6c5)nc(-c5ccc6ccccc6c5)n4)ccc23)cc1 ; c1ccc2cc(-c3nc(-c4ccc5ccccc5c4)nc(-c4ccc5c(c4)oc4c(-c6ccc7ccccc7c6)c6ccccc6cc45)n3)ccc2c1 ; c1ccc(-c2ccc(-c3c4ccccc4cc4c3oc3cc(-c5nc(-c6ccc7ccccc7c6)nc(-c6ccc7ccccc7c6)n5)ccc34)cc2)cc1 ; c1ccc2cc(-c3ccc(-c4c5ccccc5cc5c4oc4cc(-c6nc(-c7ccc8ccccc8c7)nc(-c7ccc8ccccc8c7)n6)ccc45)cc3)ccc2c1]

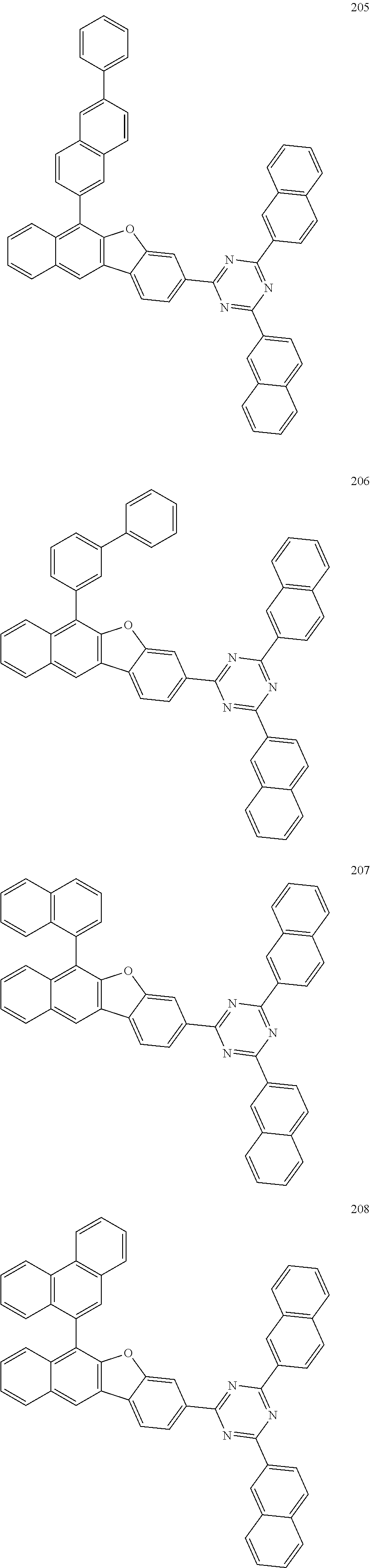 [SMILES: c1ccc(-c2ccc3cc(-c4c5ccccc5cc5c4oc4cc(-c6nc(-c7ccc8ccccc8c7)nc(-c7ccc8ccccc8c7)n6)ccc45)ccc3c2)cc1 ; c1ccc(-c2cccc(-c3c4ccccc4cc4c3oc3cc(-c5nc(-c6ccc7ccccc7c6)nc(-c6ccc7ccccc7c6)n5)ccc34)c2)cc1 ; c1ccc2cc(-c3nc(-c4ccc5ccccc5c4)nc(-c4ccc5c(c4)oc4c(-c6cccc7ccccc67)c6ccccc6cc45)n3)ccc2c1 ; c1ccc2cc(-c3nc(-c4ccc5ccccc5c4)nc(-c4ccc5c(c4)oc4c(-c6cc7ccccc7c7ccccc67)c6ccccc6cc45)n3)ccc2c1]

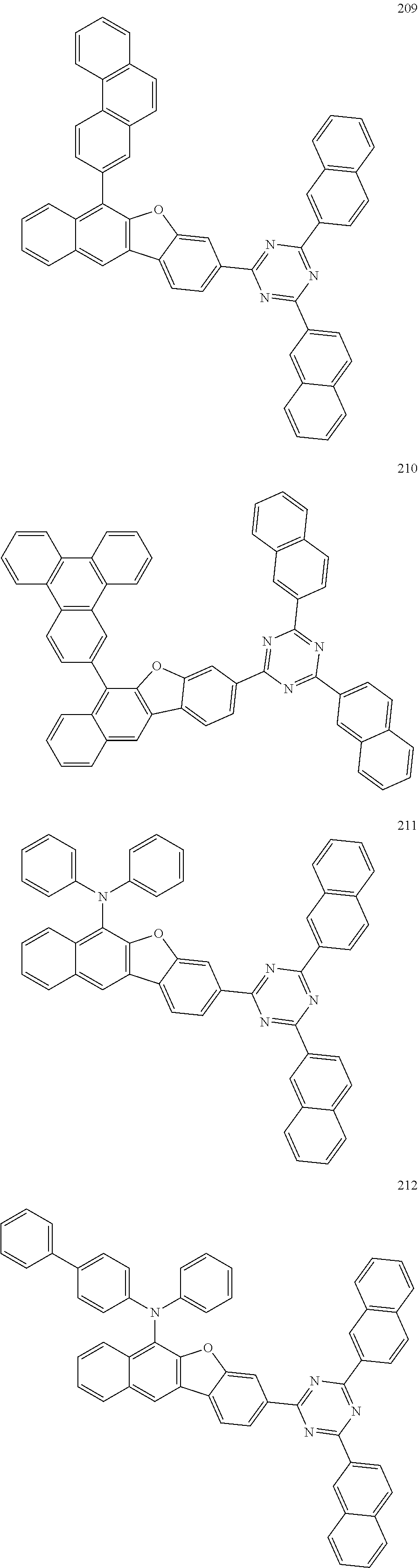 [SMILES: c1ccc2cc(-c3nc(-c4ccc5ccccc5c4)nc(-c4ccc5c(c4)oc4c(-c6ccc7c(ccc8ccccc87)c6)c6ccccc6cc45)n3)ccc2c1 ; c1ccc2cc(-c3nc(-c4ccc5ccccc5c4)nc(-c4ccc5c(c4)oc4c(-c6ccc7c8ccccc8c8ccccc8c7c6)c6ccccc6cc45)n3)ccc2c1 ; c1ccc(N(c2ccccc2)c2c3ccccc3cc3c2oc2cc(-c4nc(-c5ccc6ccccc6c5)nc(-c5ccc6ccccc6c5)n4)ccc23)cc1 ; c1ccc(-c2ccc(N(c3ccccc3)c3c4ccccc4cc4c3oc3cc(-c5nc(-c6ccc7ccccc7c6)nc(-c6ccc7ccccc7c6)n5)ccc34)cc2)cc1]

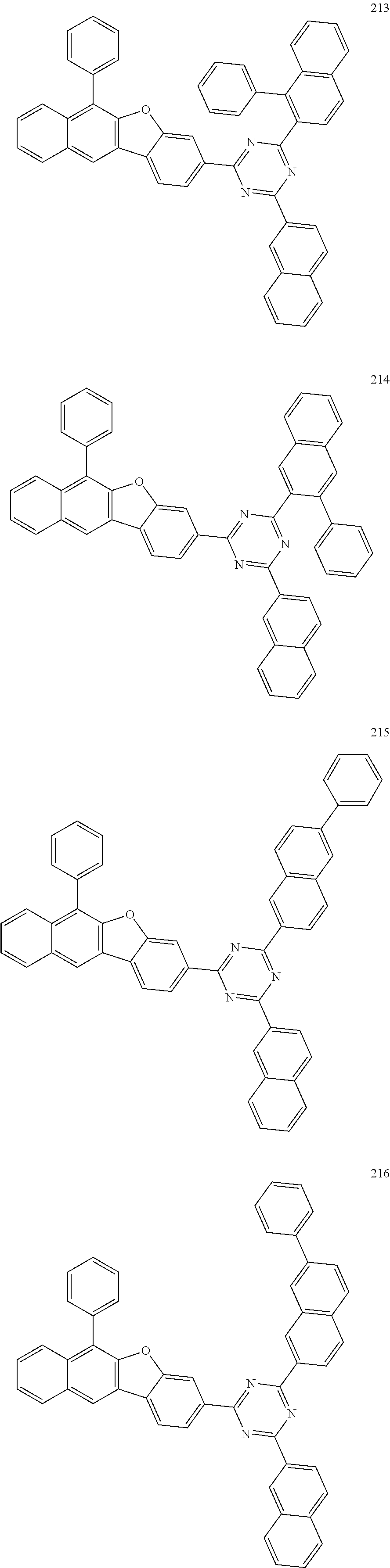 [SMILES: c1ccc(-c2c(-c3nc(-c4ccc5ccccc5c4)nc(-c4ccc5c(c4)oc4c(-c6ccccc6)c6ccccc6cc45)n3)ccc3ccccc23)cc1 ; c1ccc(-c2cc3ccccc3cc2-c2nc(-c3ccc4ccccc4c3)nc(-c3ccc4c(c3)oc3c(-c5ccccc5)c5ccccc5cc34)n2)cc1 ; c1ccc(-c2ccc3cc(-c4nc(-c5ccc6ccccc6c5)nc(-c5ccc6c(c5)oc5c(-c7ccccc7)c7ccccc7cc56)n4)ccc3c2)cc1 ; c1ccc(-c2ccc3ccc(-c4nc(-c5ccc6ccccc6c5)nc(-c5ccc6c(c5)oc5c(-c7ccccc7)c7ccccc7cc56)n4)cc3c2)cc1]

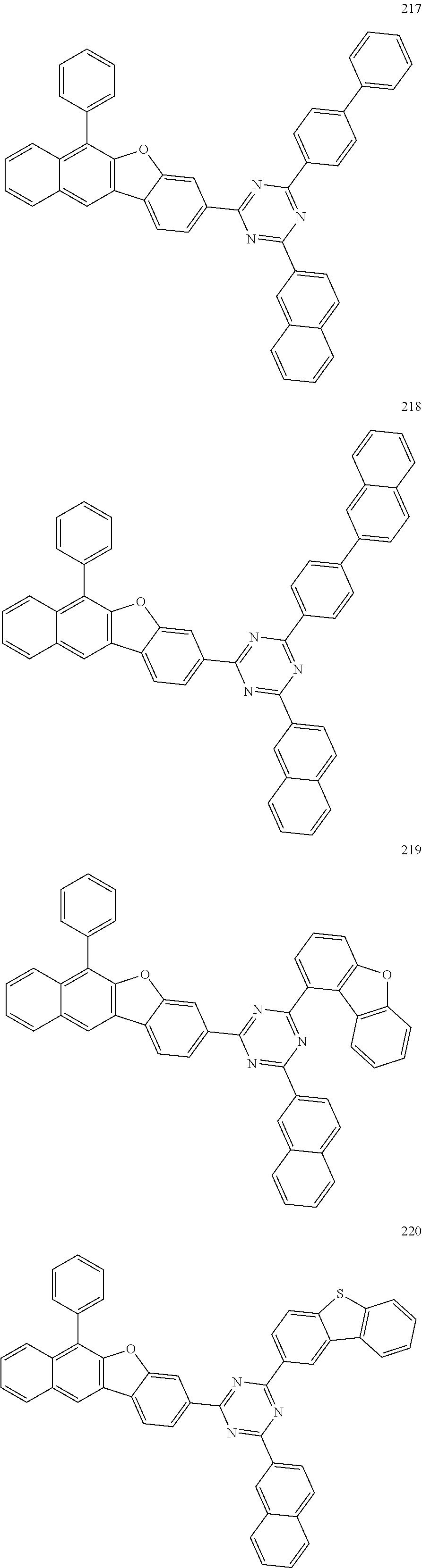 [SMILES: c1ccc(-c2ccc(-c3nc(-c4ccc5ccccc5c4)nc(-c4ccc5c(c4)oc4c(-c6ccccc6)c6ccccc6cc45)n3)cc2)cc1 ; c1ccc(-c2c3ccccc3cc3c2oc2cc(-c4nc(-c5ccc(-c6ccc7ccccc7c6)cc5)nc(-c5ccc6ccccc6c5)n4)ccc23)cc1 ; c1ccc(-c2c3ccccc3cc3c2oc2cc(-c4nc(-c5ccc6ccccc6c5)nc(-c5cccc6oc7ccccc7c56)n4)ccc23)cc1 ; c1ccc(-c2c3ccccc3cc3c2oc2cc(-c4nc(-c5ccc6ccccc6c5)nc(-c5ccc6sc7ccccc7c6c5)n4)ccc23)cc1]

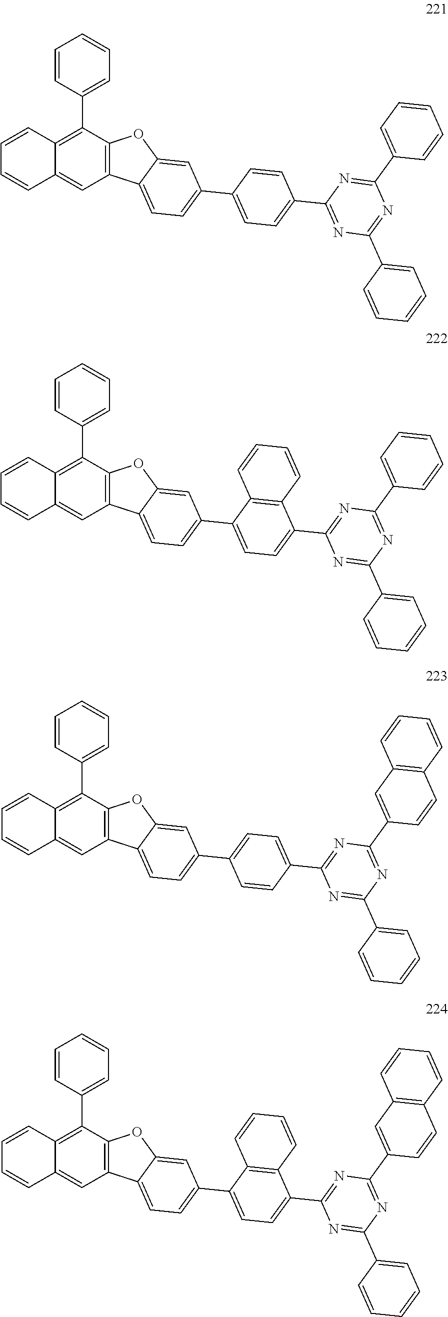 [SMILES: c1ccc(-c2nc(-c3ccccc3)nc(-c3ccc(-c4ccc5c(c4)oc4c(-c6ccccc6)c6ccccc6cc45)cc3)n2)cc1 ; c1ccc(-c2nc(-c3ccccc3)nc(-c3ccc(-c4ccc5c(c4)oc4c(-c6ccccc6)c6ccccc6cc45)c4ccccc34)n2)cc1 ; c1ccc(-c2nc(-c3ccc(-c4ccc5c(c4)oc4c(-c6ccccc6)c6ccccc6cc45)cc3)nc(-c3ccc4ccccc4c3)n2)cc1 ; c1ccc(-c2nc(-c3ccc4ccccc4c3)nc(-c3ccc(-c4ccc5c(c4)oc4c(-c6ccccc6)c6ccccc6cc45)c4ccccc34)n2)cc1]

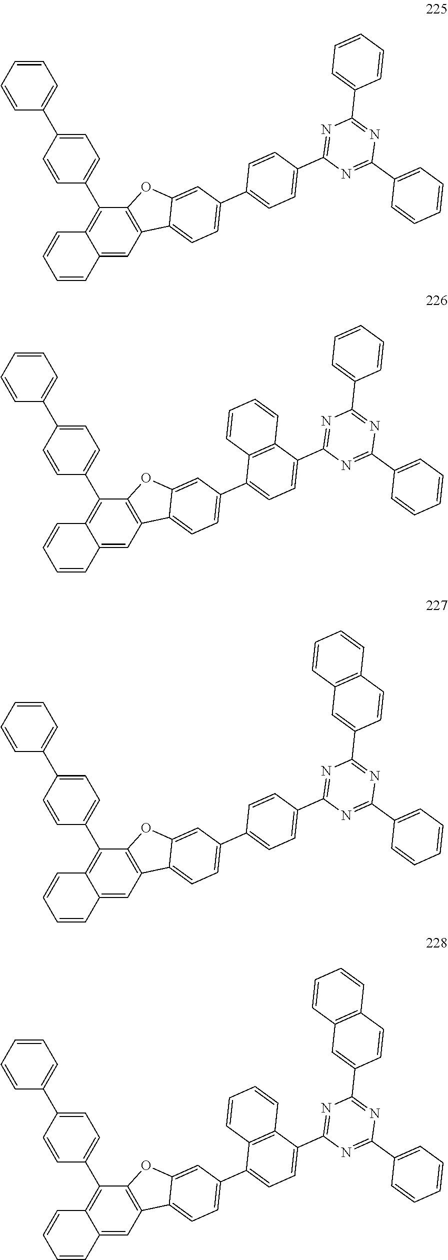 [SMILES: c1ccc(-c2ccc(-c3c4ccccc4cc4c3oc3cc(-c5ccc(-c6nc(-c7ccccc7)nc(-c7ccccc7)n6)cc5)ccc34)cc2)cc1 ; c1ccc(-c2ccc(-c3c4ccccc4cc4c3oc3cc(-c5ccc(-c6nc(-c7ccccc7)nc(-c7ccccc7)n6)c6ccccc56)ccc34)cc2)cc1 ; c1ccc(-c2ccc(-c3c4ccccc4cc4c3oc3cc(-c5ccc(-c6nc(-c7ccccc7)nc(-c7ccc8ccccc8c7)n6)cc5)ccc34)cc2)cc1 ; c1ccc(-c2ccc(-c3c4ccccc4cc4c3oc3cc(-c5ccc(-c6nc(-c7ccccc7)nc(-c7ccc8ccccc8c7)n6)c6ccccc56)ccc34)cc2)cc1]

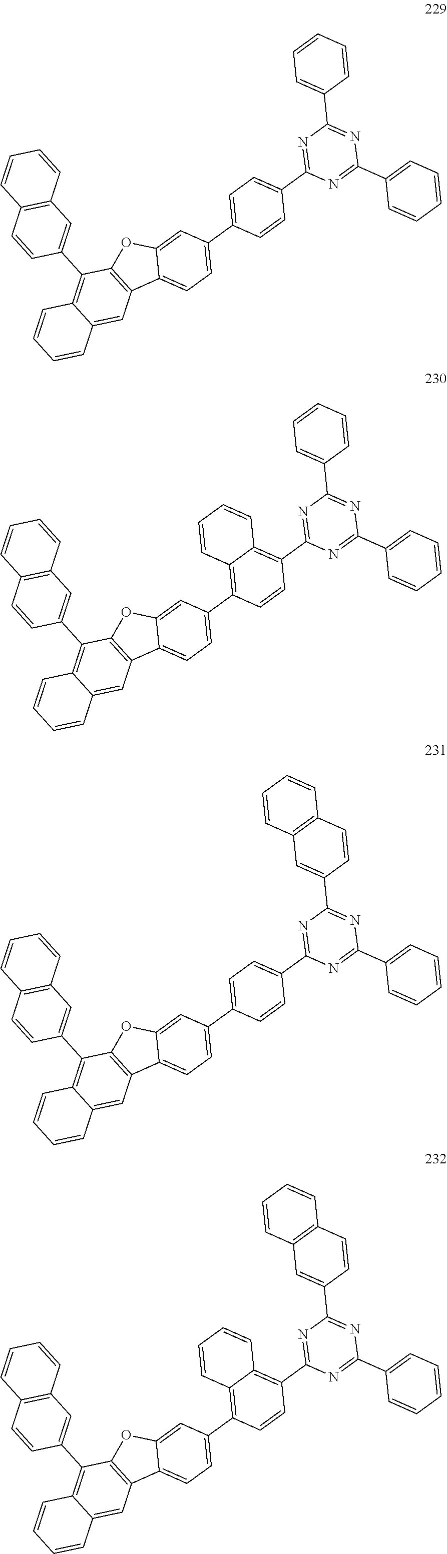 [SMILES: c1ccc(-c2nc(-c3ccccc3)nc(-c3ccc(-c4ccc5c(c4)oc4c(-c6ccc7ccccc7c6)c6ccccc6cc45)cc3)n2)cc1 ; c1ccc(-c2nc(-c3ccccc3)nc(-c3ccc(-c4ccc5c(c4)oc4c(-c6ccc7ccccc7c6)c6ccccc6cc45)c4ccccc34)n2)cc1 ; c1ccc(-c2nc(-c3ccc(-c4ccc5c(c4)oc4c(-c6ccc7ccccc7c6)c6ccccc6cc45)cc3)nc(-c3ccc4ccccc4c3)n2)cc1 ; c1ccc(-c2nc(-c3ccc4ccccc4c3)nc(-c3ccc(-c4ccc5c(c4)oc4c(-c6ccc7ccccc7c6)c6ccccc6cc45)c4ccccc34)n2)cc1]

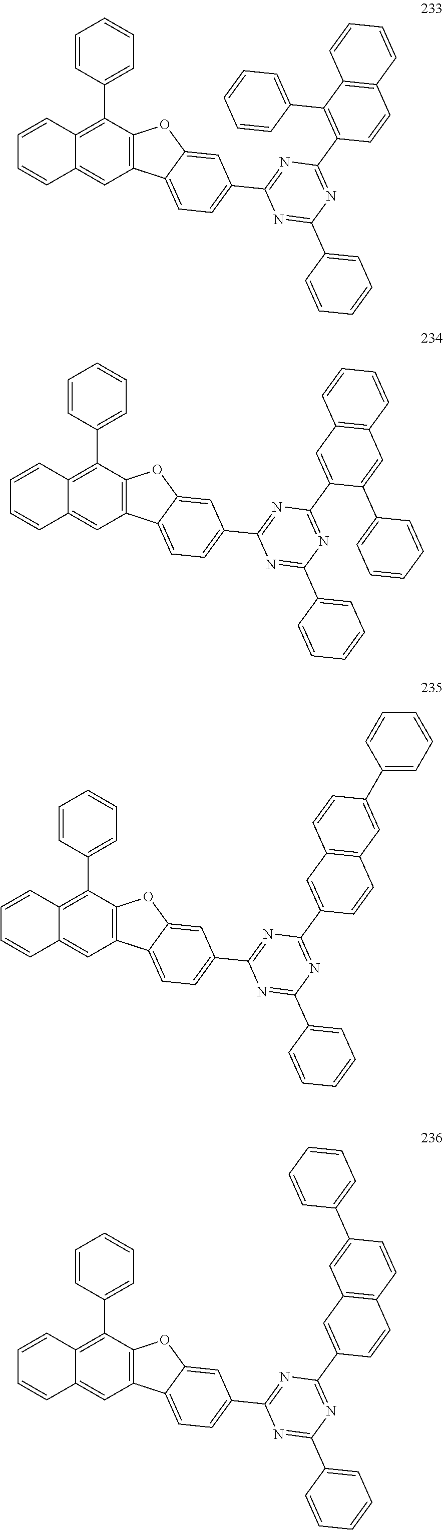 [SMILES: c1ccc(-c2nc(-c3ccc4c(c3)oc3c(-c5ccccc5)c5ccccc5cc34)nc(-c3ccc4ccccc4c3-c3ccccc3)n2)cc1 ; c1ccc(-c2nc(-c3ccc4c(c3)oc3c(-c5ccccc5)c5ccccc5cc34)nc(-c3cc4ccccc4cc3-c3ccccc3)n2)cc1 ; c1ccc(-c2ccc3cc(-c4nc(-c5ccccc5)nc(-c5ccc6c(c5)oc5c(-c7ccccc7)c7ccccc7cc56)n4)ccc3c2)cc1 ; c1ccc(-c2ccc3ccc(-c4nc(-c5ccccc5)nc(-c5ccc6c(c5)oc5c(-c7ccccc7)c7ccccc7cc56)n4)cc3c2)cc1]

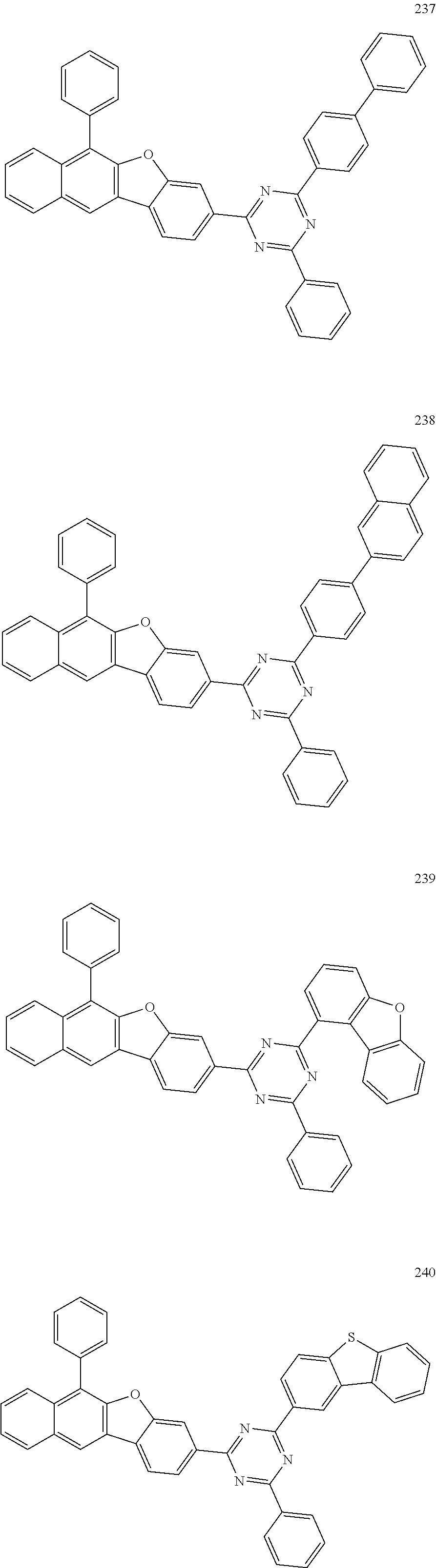 [SMILES: c1ccc(-c2ccc(-c3nc(-c4ccccc4)nc(-c4ccc5c(c4)oc4c(-c6ccccc6)c6ccccc6cc45)n3)cc2)cc1 ; c1ccc(-c2nc(-c3ccc(-c4ccc5ccccc5c4)cc3)nc(-c3ccc4c(c3)oc3c(-c5ccccc5)c5ccccc5cc34)n2)cc1 ; c1ccc(-c2nc(-c3ccc4c(c3)oc3c(-c5ccccc5)c5ccccc5cc34)nc(-c3cccc4oc5ccccc5c34)n2)cc1 ; c1ccc(-c2nc(-c3ccc4c(c3)oc3c(-c5ccccc5)c5ccccc5cc34)nc(-c3ccc4sc5ccccc5c4c3)n2)cc1]

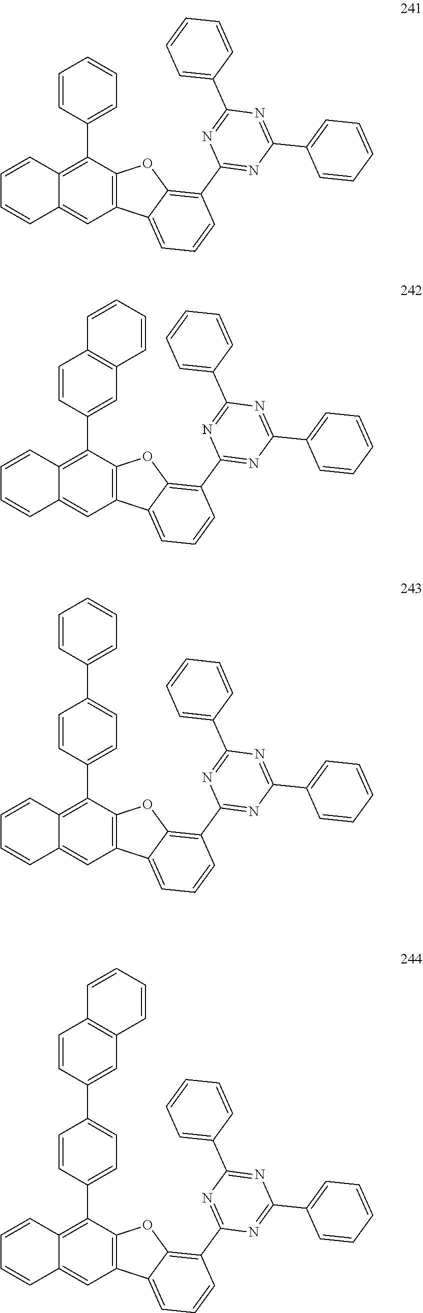

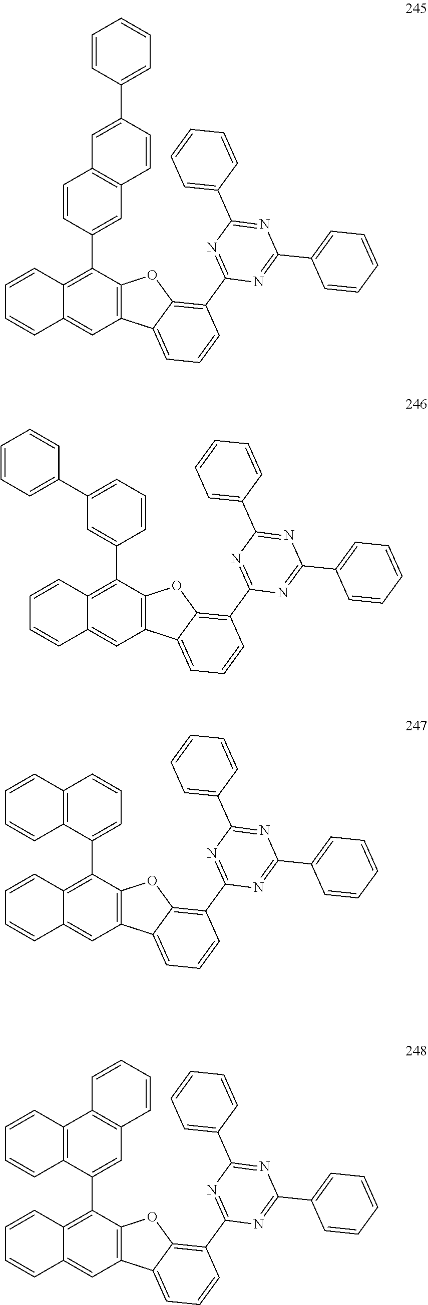 [SMILES: c1ccc(-c2ccc3cc(-c4c5ccccc5cc5c4oc4c(-c6nc(-c7ccccc7)nc(-c7ccccc7)n6)cccc45)ccc3c2)cc1 ; c1ccc(-c2cccc(-c3c4ccccc4cc4c3oc3c(-c5nc(-c6ccccc6)nc(-c6ccccc6)n5)cccc34)c2)cc1 ; c1ccc(-c2nc(-c3ccccc3)nc(-c3cccc4c3oc3c(-c5cccc6ccccc56)c5ccccc5cc34)n2)cc1 ; c1ccc(-c2nc(-c3ccccc3)nc(-c3cccc4c3oc3c(-c5cc6ccccc6c6ccccc56)c5ccccc5cc34)n2)cc1]

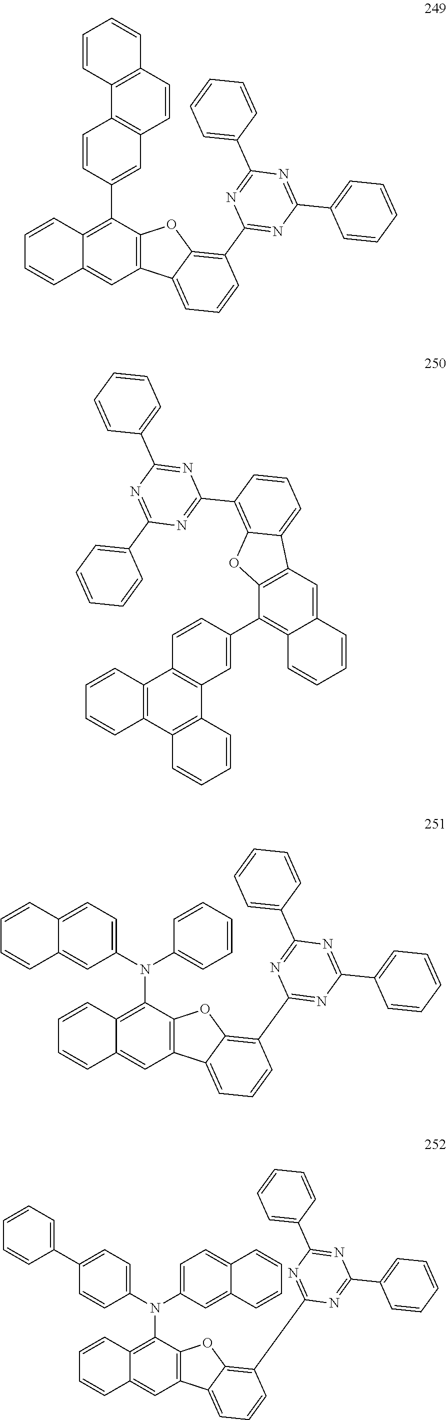 [SMILES: c1ccc(-c2nc(-c3ccccc3)nc(-c3cccc4c3oc3c(-c5ccc6c(ccc7ccccc76)c5)c5ccccc5cc34)n2)cc1 ; c1ccc(-c2nc(-c3ccccc3)nc(-c3cccc4c3oc3c(-c5ccc6c7ccccc7c7ccccc7c6c5)c5ccccc5cc34)n2)cc1 ; c1ccc(-c2nc(-c3ccccc3)nc(-c3cccc4c3oc3c(N(c5ccccc5)c5ccc6ccccc6c5)c5ccccc5cc34)n2)cc1 ; c1ccc(-c2ccc(N(c3ccc4ccccc4c3)c3c4ccccc4cc4c3oc3c(-c5nc(-c6ccccc6)nc(-c6ccccc6)n5)cccc34)cc2)cc1]

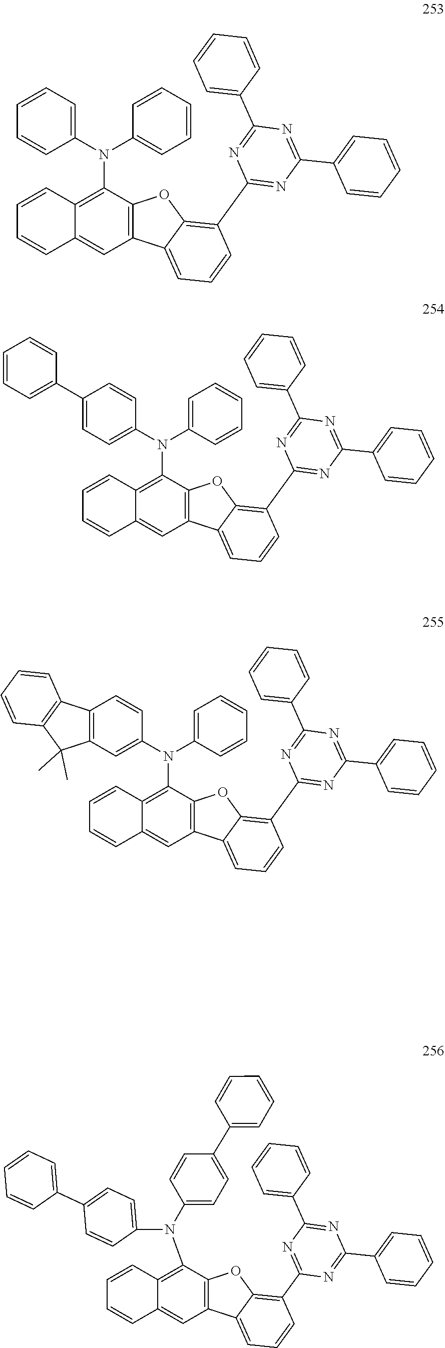 [SMILES: c1ccc(-c2nc(-c3ccccc3)nc(-c3cccc4c3oc3c(N(c5ccccc5)c5ccccc5)c5ccccc5cc34)n2)cc1 ; c1ccc(-c2ccc(N(c3ccccc3)c3c4ccccc4cc4c3oc3c(-c5nc(-c6ccccc6)nc(-c6ccccc6)n5)cccc34)cc2)cc1 ; CC1(C)c2ccccc2-c2ccc(N(c3ccccc3)c3c4ccccc4cc4c3oc3c(-c5nc(-c6ccccc6)nc(-c6ccccc6)n5)cccc34)cc21 ; c1ccc(-c2ccc(N(c3ccc(-c4ccccc4)cc3)c3c4ccccc4cc4c3oc3c(-c5nc(-c6ccccc6)nc(-c6ccccc6)n5)cccc34)cc2)cc1]

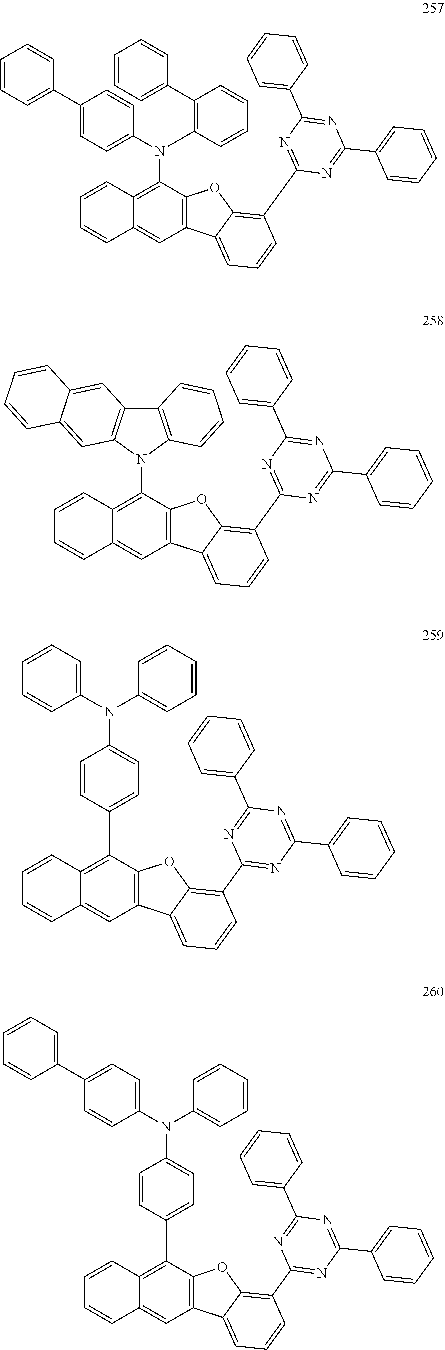 [SMILES: c1ccc(-c2ccc(N(c3ccccc3-c3ccccc3)c3c4ccccc4cc4c3oc3c(-c5nc(-c6ccccc6)nc(-c6ccccc6)n5)cccc34)cc2)cc1 ; c1ccc(-c2nc(-c3ccccc3)nc(-c3cccc4c3oc3c(-n5c6ccccc6c6cc7ccccc7cc65)c5ccccc5cc34)n2)cc1 ; c1ccc(-c2nc(-c3ccccc3)nc(-c3cccc4c3oc3c(-c5ccc(N(c6ccccc6)c6ccccc6)cc5)c5ccccc5cc34)n2)cc1 ; c1ccc(-c2ccc(N(c3ccccc3)c3ccc(-c4c5ccccc5cc5c4oc4c(-c6nc(-c7ccccc7)nc(-c7ccccc7)n6)cccc45)cc3)cc2)cc1]

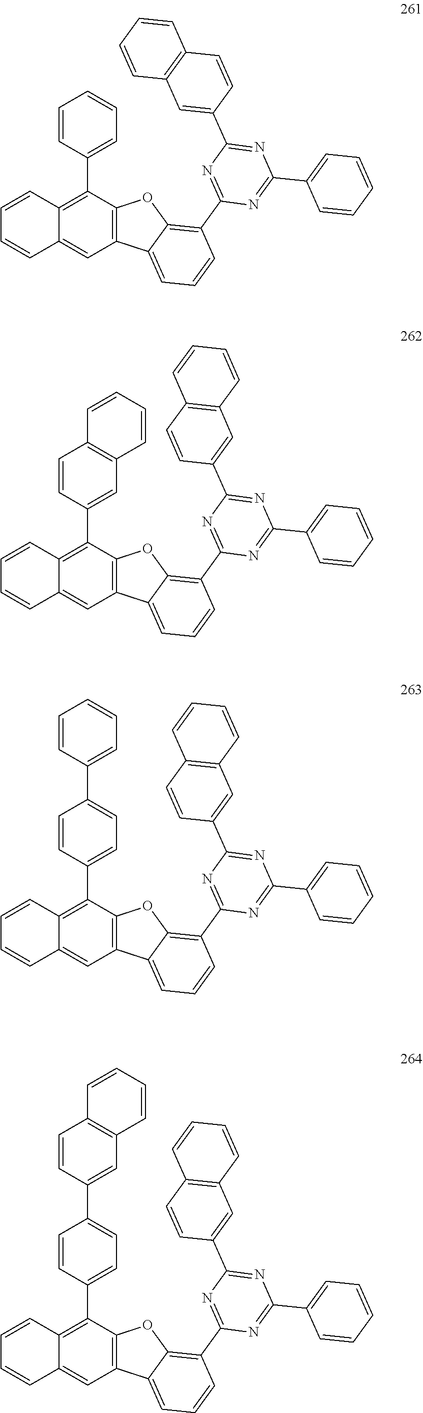 [SMILES: c1ccc(-c2nc(-c3ccc4ccccc4c3)nc(-c3cccc4c3oc3c(-c5ccccc5)c5ccccc5cc34)n2)cc1 ; c1ccc(-c2nc(-c3ccc4ccccc4c3)nc(-c3cccc4c3oc3c(-c5ccc6ccccc6c5)c5ccccc5cc34)n2)cc1 ; c1ccc(-c2ccc(-c3c4ccccc4cc4c3oc3c(-c5nc(-c6ccccc6)nc(-c6ccc7ccccc7c6)n5)cccc34)cc2)cc1 ; c1ccc(-c2nc(-c3ccc4ccccc4c3)nc(-c3cccc4c3oc3c(-c5ccc(-c6ccc7ccccc7c6)cc5)c5ccccc5cc34)n2)cc1]

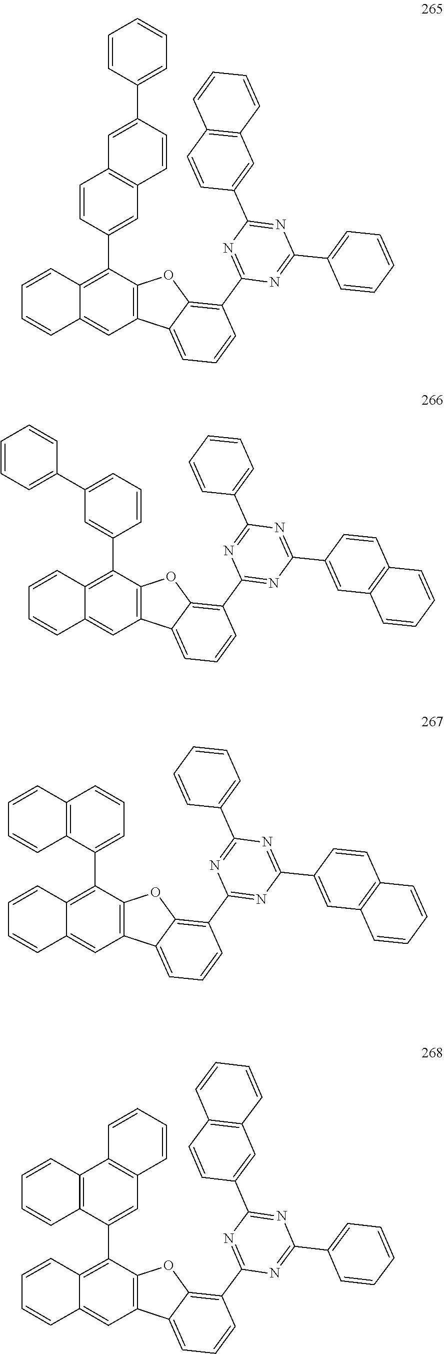 [SMILES: c1ccc(-c2ccc3cc(-c4c5ccccc5cc5c4oc4c(-c6nc(-c7ccccc7)nc(-c7ccc8ccccc8c7)n6)cccc45)ccc3c2)cc1 ; c1ccc(-c2cccc(-c3c4ccccc4cc4c3oc3c(-c5nc(-c6ccccc6)nc(-c6ccc7ccccc7c6)n5)cccc34)c2)cc1 ; c1ccc(-c2nc(-c3ccc4ccccc4c3)nc(-c3cccc4c3oc3c(-c5cccc6ccccc56)c5ccccc5cc34)n2)cc1 ; c1ccc(-c2nc(-c3ccc4ccccc4c3)nc(-c3cccc4c3oc3c(-c5cc6ccccc6c6ccccc56)c5ccccc5cc34)n2)cc1]

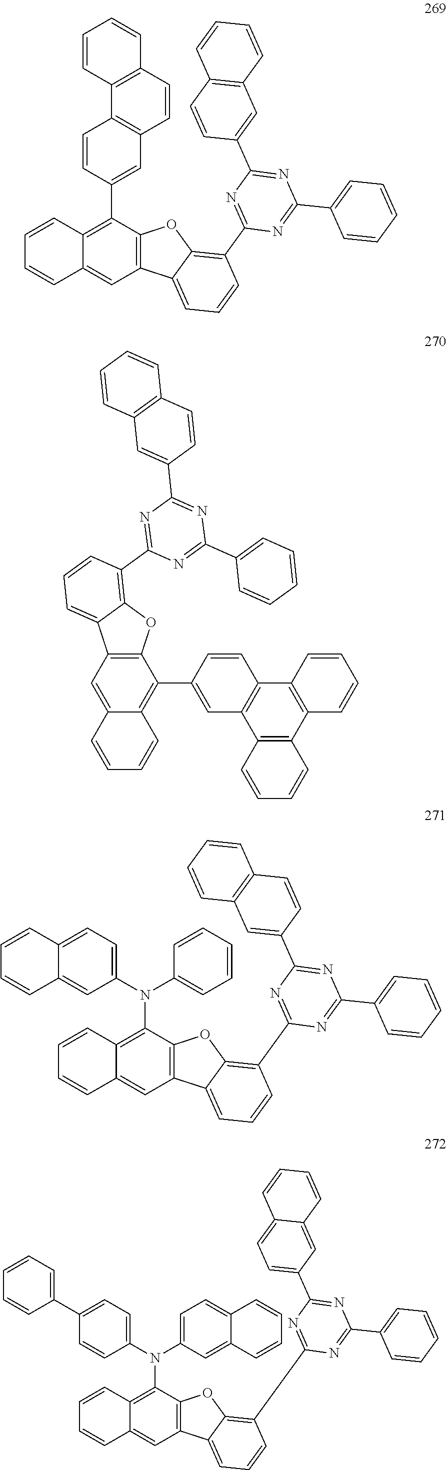 [SMILES: c1ccc(-c2nc(-c3ccc4ccccc4c3)nc(-c3cccc4c3oc3c(-c5ccc6c(ccc7ccccc76)c5)c5ccccc5cc34)n2)cc1 ; c1ccc(-c2nc(-c3ccc4ccccc4c3)nc(-c3cccc4c3oc3c(-c5ccc6c7ccccc7c7ccccc7c6c5)c5ccccc5cc34)n2)cc1 ; c1ccc(-c2nc(-c3ccc4ccccc4c3)nc(-c3cccc4c3oc3c(N(c5ccccc5)c5ccc6ccccc6c5)c5ccccc5cc34)n2)cc1 ; c1ccc(-c2ccc(N(c3ccc4ccccc4c3)c3c4ccccc4cc4c3oc3c(-c5nc(-c6ccccc6)nc(-c6ccc7ccccc7c6)n5)cccc34)cc2)cc1]

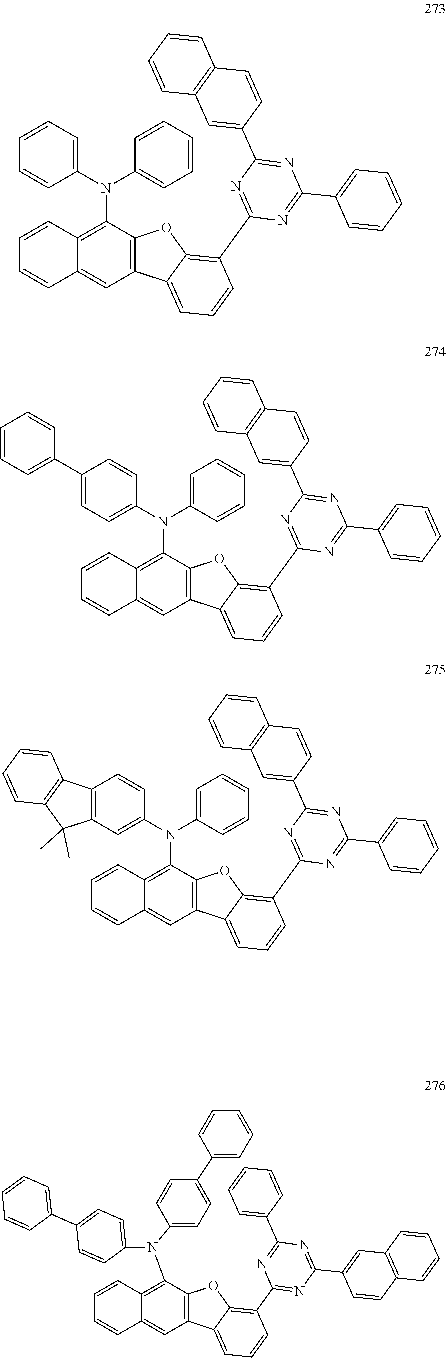 [SMILES: c1ccc(-c2nc(-c3ccc4ccccc4c3)nc(-c3cccc4c3oc3c(N(c5ccccc5)c5ccccc5)c5ccccc5cc34)n2)cc1 ; c1ccc(-c2ccc(N(c3ccccc3)c3c4ccccc4cc4c3oc3c(-c5nc(-c6ccccc6)nc(-c6ccc7ccccc7c6)n5)cccc34)cc2)cc1 ; CC1(C)c2ccccc2-c2ccc(N(c3ccccc3)c3c4ccccc4cc4c3oc3c(-c5nc(-c6ccccc6)nc(-c6ccc7ccccc7c6)n5)cccc34)cc21 ; c1ccc(-c2ccc(N(c3ccc(-c4ccccc4)cc3)c3c4ccccc4cc4c3oc3c(-c5nc(-c6ccccc6)nc(-c6ccc7ccccc7c6)n5)cccc34)cc2)cc1]

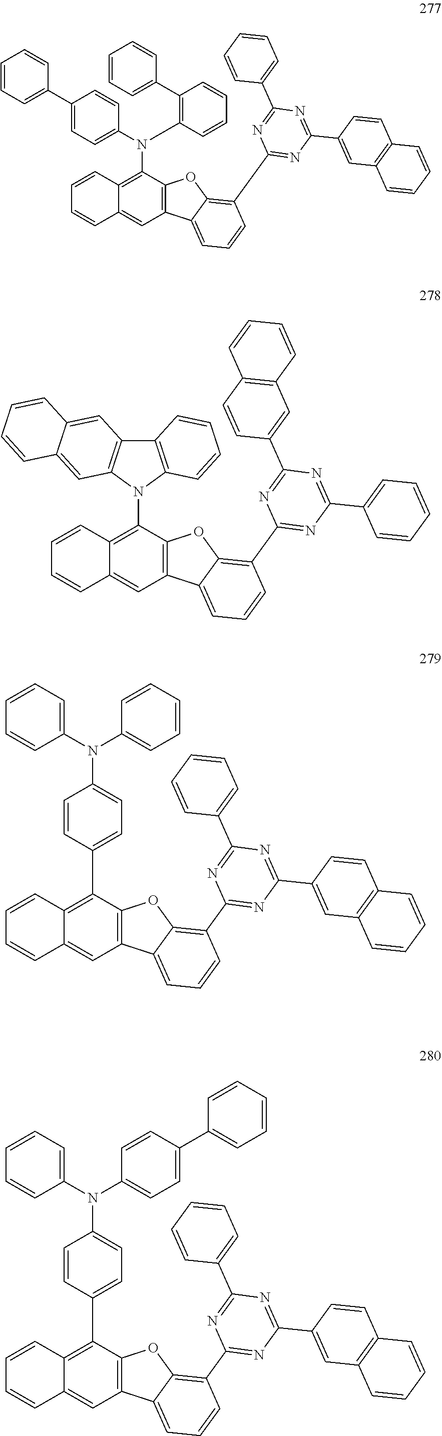 [SMILES: c1ccc(-c2ccc(N(c3ccccc3-c3ccccc3)c3c4ccccc4cc4c3oc3c(-c5nc(-c6ccccc6)nc(-c6ccc7ccccc7c6)n5)cccc34)cc2)cc1 ; c1ccc(-c2nc(-c3ccc4ccccc4c3)nc(-c3cccc4c3oc3c(-n5c6ccccc6c6cc7ccccc7cc65)c5ccccc5cc34)n2)cc1 ; c1ccc(-c2nc(-c3ccc4ccccc4c3)nc(-c3cccc4c3oc3c(-c5ccc(N(c6ccccc6)c6ccccc6)cc5)c5ccccc5cc34)n2)cc1 ; c1ccc(-c2ccc(N(c3ccccc3)c3ccc(-c4c5ccccc5cc5c4oc4c(-c6nc(-c7ccccc7)nc(-c7ccc8ccccc8c7)n6)cccc45)cc3)cc2)cc1]

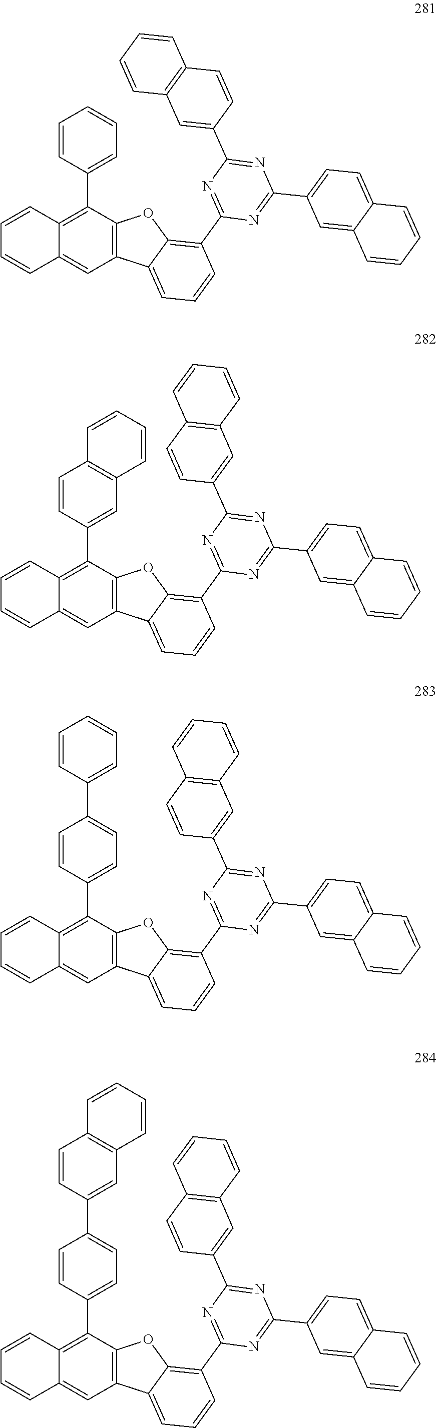 [SMILES: c1ccc(-c2c3ccccc3cc3c2oc2c(-c4nc(-c5ccc6ccccc6c5)nc(-c5ccc6ccccc6c5)n4)cccc23)cc1 ; c1ccc2cc(-c3nc(-c4ccc5ccccc5c4)nc(-c4cccc5c4oc4c(-c6ccc7ccccc7c6)c6ccccc6cc45)n3)ccc2c1 ; c1ccc(-c2ccc(-c3c4ccccc4cc4c3oc3c(-c5nc(-c6ccc7ccccc7c6)nc(-c6ccc7ccccc7c6)n5)cccc34)cc2)cc1 ; c1ccc2cc(-c3ccc(-c4c5ccccc5cc5c4oc4c(-c6nc(-c7ccc8ccccc8c7)nc(-c7ccc8ccccc8c7)n6)cccc45)cc3)ccc2c1]

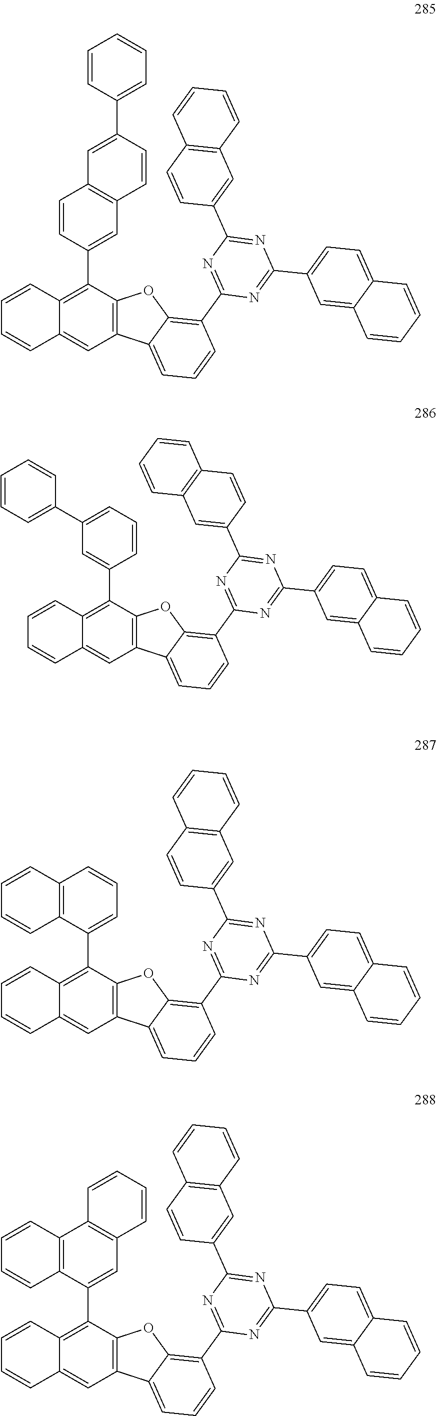 [SMILES: c1ccc(-c2ccc3cc(-c4c5ccccc5cc5c4oc4c(-c6nc(-c7ccc8ccccc8c7)nc(-c7ccc8ccccc8c7)n6)cccc45)ccc3c2)cc1 ; c1ccc(-c2cccc(-c3c4ccccc4cc4c3oc3c(-c5nc(-c6ccc7ccccc7c6)nc(-c6ccc7ccccc7c6)n5)cccc34)c2)cc1 ; c1ccc2cc(-c3nc(-c4ccc5ccccc5c4)nc(-c4cccc5c4oc4c(-c6cccc7ccccc67)c6ccccc6cc45)n3)ccc2c1 ; c1ccc2cc(-c3nc(-c4ccc5ccccc5c4)nc(-c4cccc5c4oc4c(-c6cc7ccccc7c7ccccc67)c6ccccc6cc45)n3)ccc2c1]

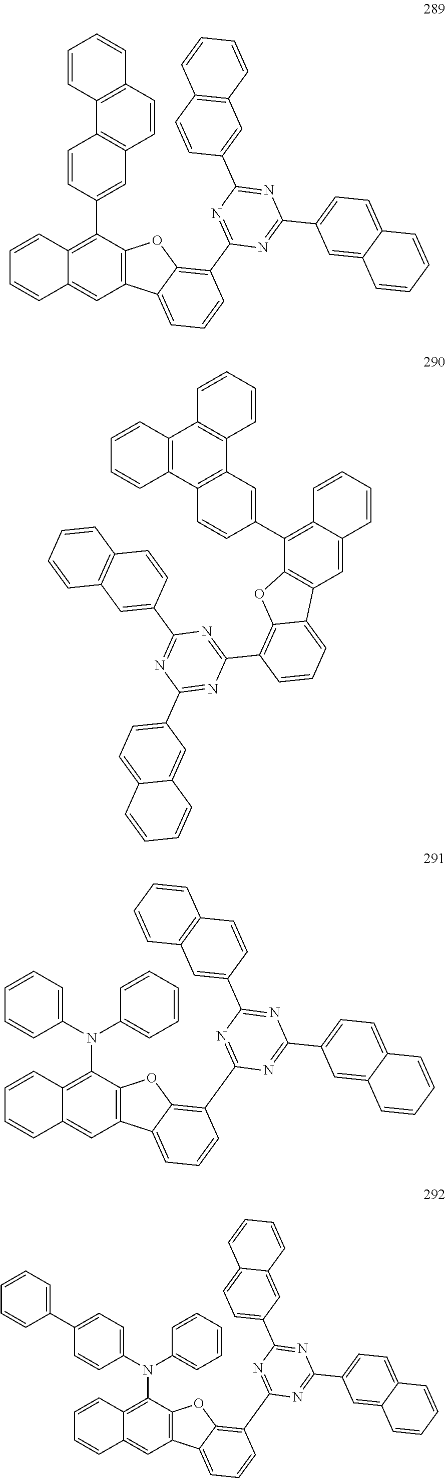

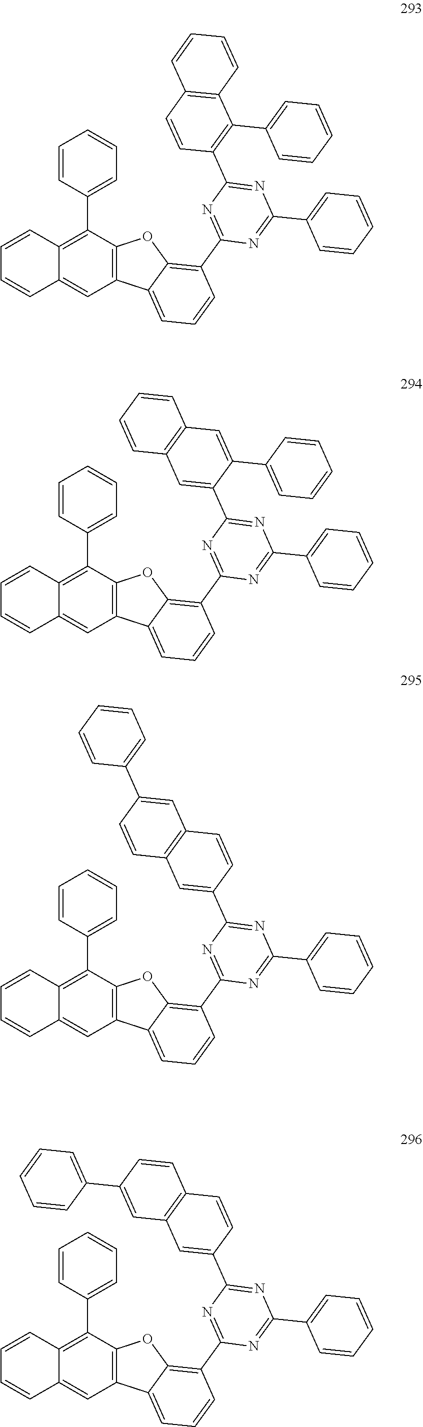 [SMILES: c1ccc(-c2nc(-c3ccc4ccccc4c3-c3ccccc3)nc(-c3cccc4c3oc3c(-c5ccccc5)c5ccccc5cc34)n2)cc1 ; c1ccc(-c2nc(-c3cc4ccccc4cc3-c3ccccc3)nc(-c3cccc4c3oc3c(-c5ccccc5)c5ccccc5cc34)n2)cc1 ; c1ccc(-c2ccc3cc(-c4nc(-c5ccccc5)nc(-c5cccc6c5oc5c(-c7ccccc7)c7ccccc7cc56)n4)ccc3c2)cc1 ; c1ccc(-c2ccc3ccc(-c4nc(-c5ccccc5)nc(-c5cccc6c5oc5c(-c7ccccc7)c7ccccc7cc56)n4)cc3c2)cc1]

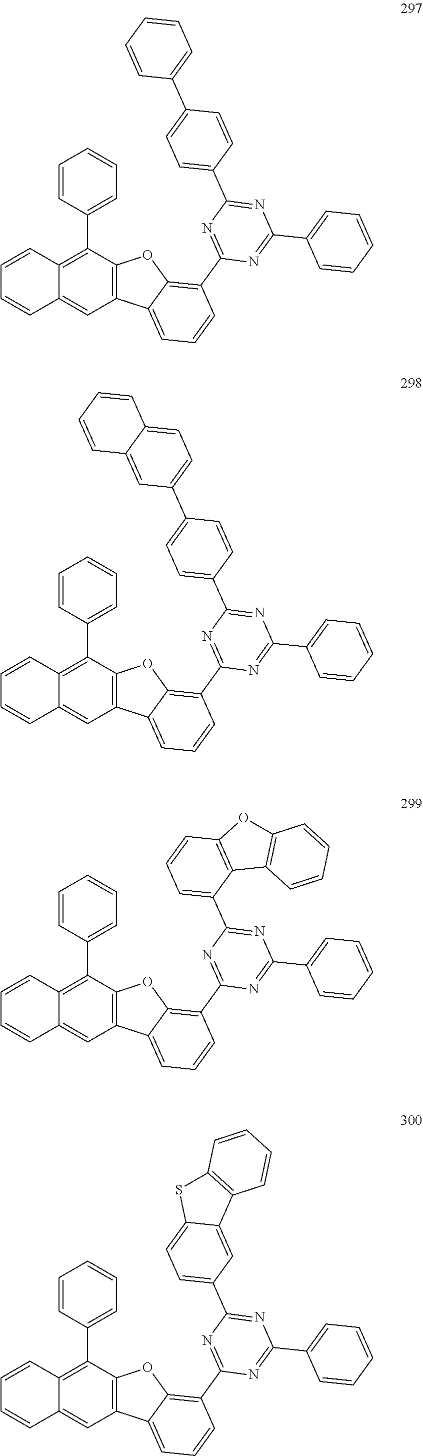 [SMILES: c1ccc(-c2ccc(-c3nc(-c4ccccc4)nc(-c4cccc5c4oc4c(-c6ccccc6)c6ccccc6cc45)n3)cc2)cc1 ; c1ccc(-c2nc(-c3ccc(-c4ccc5ccccc5c4)cc3)nc(-c3cccc4c3oc3c(-c5ccccc5)c5ccccc5cc34)n2)cc1 ; c1ccc(-c2nc(-c3cccc4c3oc3c(-c5ccccc5)c5ccccc5cc34)nc(-c3cccc4oc5ccccc5c34)n2)cc1 ; c1ccc(-c2nc(-c3ccc4sc5ccccc5c4c3)nc(-c3cccc4c3oc3c(-c5ccccc5)c5ccccc5cc34)n2)cc1]

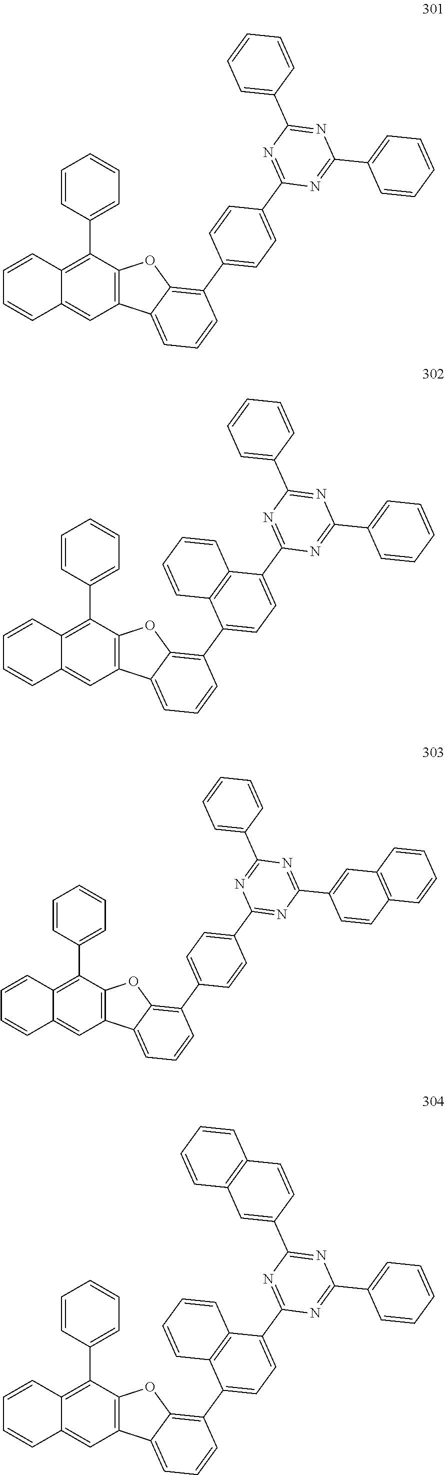 [SMILES: c1ccc(-c2nc(-c3ccccc3)nc(-c3ccc(-c4cccc5c4oc4c(-c6ccccc6)c6ccccc6cc45)cc3)n2)cc1 ; c1ccc(-c2nc(-c3ccccc3)nc(-c3ccc(-c4cccc5c4oc4c(-c6ccccc6)c6ccccc6cc45)c4ccccc34)n2)cc1 ; c1ccc(-c2nc(-c3ccc(-c4cccc5c4oc4c(-c6ccccc6)c6ccccc6cc45)cc3)nc(-c3ccc4ccccc4c3)n2)cc1 ; c1ccc(-c2nc(-c3ccc4ccccc4c3)nc(-c3ccc(-c4cccc5c4oc4c(-c6ccccc6)c6ccccc6cc45)c4ccccc34)n2)cc1]

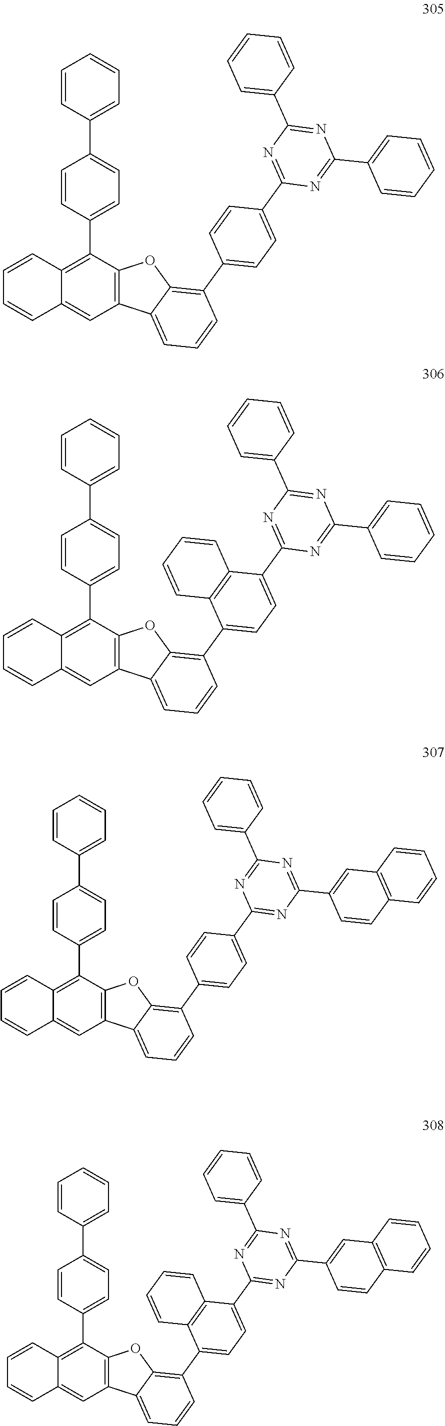 [SMILES: c1ccc(-c2ccc(-c3c4ccccc4cc4c3oc3c(-c5ccc(-c6nc(-c7ccccc7)nc(-c7ccccc7)n6)cc5)cccc34)cc2)cc1 ; c1ccc(-c2ccc(-c3c4ccccc4cc4c3oc3c(-c5ccc(-c6nc(-c7ccccc7)nc(-c7ccccc7)n6)c6ccccc56)cccc34)cc2)cc1 ; c1ccc(-c2ccc(-c3c4ccccc4cc4c3oc3c(-c5ccc(-c6nc(-c7ccccc7)nc(-c7ccc8ccccc8c7)n6)cc5)cccc34)cc2)cc1 ; c1ccc(-c2ccc(-c3c4ccccc4cc4c3oc3c(-c5ccc(-c6nc(-c7ccccc7)nc(-c7ccc8ccccc8c7)n6)c6ccccc56)cccc34)cc2)cc1]

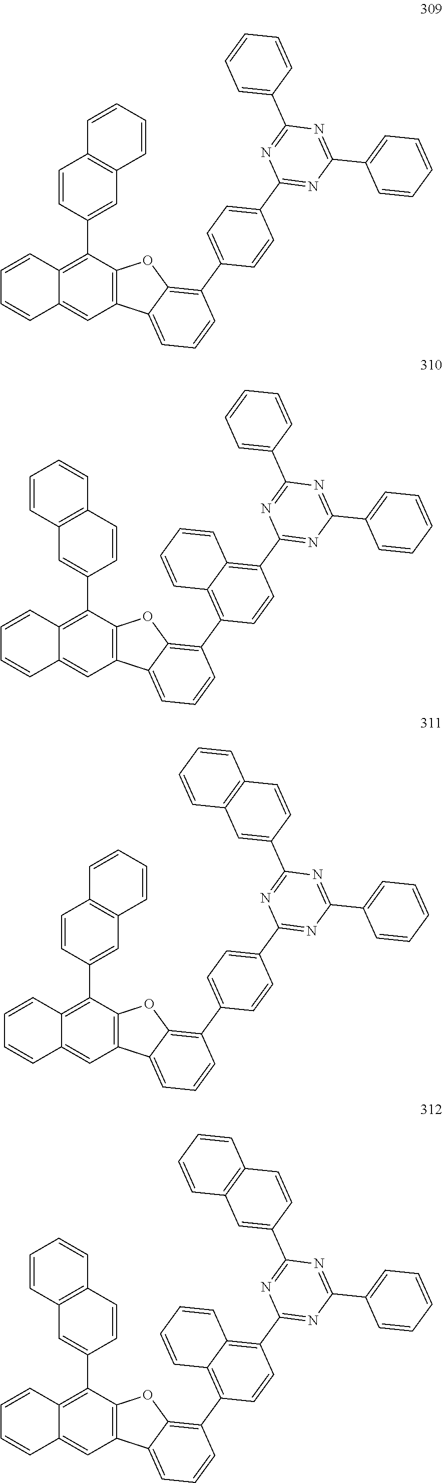

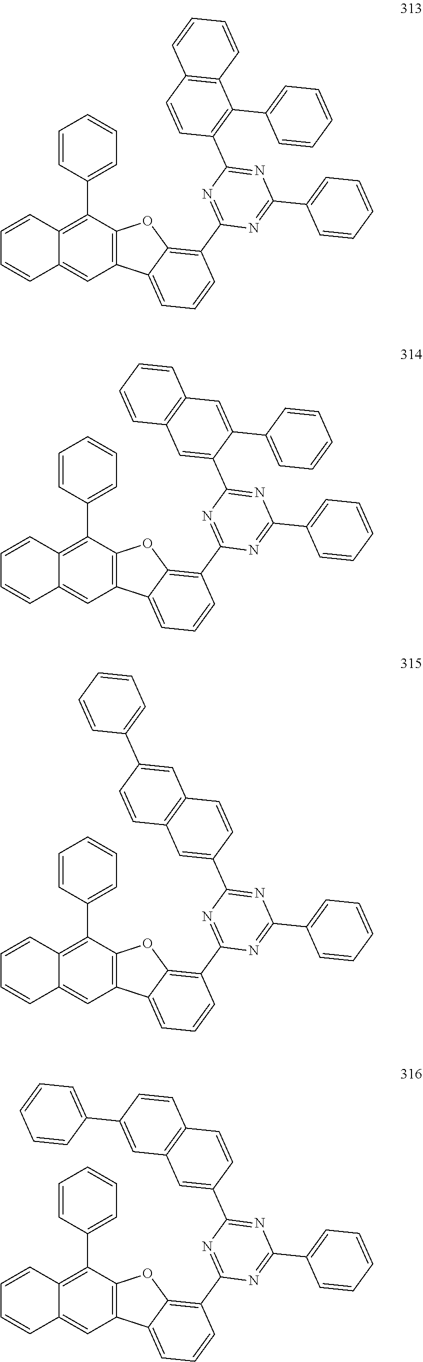 [SMILES: c1ccc(-c2nc(-c3ccc4ccccc4c3-c3ccccc3)nc(-c3cccc4c3oc3c(-c5ccccc5)c5ccccc5cc34)n2)cc1 ; c1ccc(-c2nc(-c3cc4ccccc4cc3-c3ccccc3)nc(-c3cccc4c3oc3c(-c5ccccc5)c5ccccc5cc34)n2)cc1 ; c1ccc(-c2ccc3cc(-c4nc(-c5ccccc5)nc(-c5cccc6c5oc5c(-c7ccccc7)c7ccccc7cc56)n4)ccc3c2)cc1 ; c1ccc(-c2ccc3ccc(-c4nc(-c5ccccc5)nc(-c5cccc6c5oc5c(-c7ccccc7)c7ccccc7cc56)n4)cc3c2)cc1]

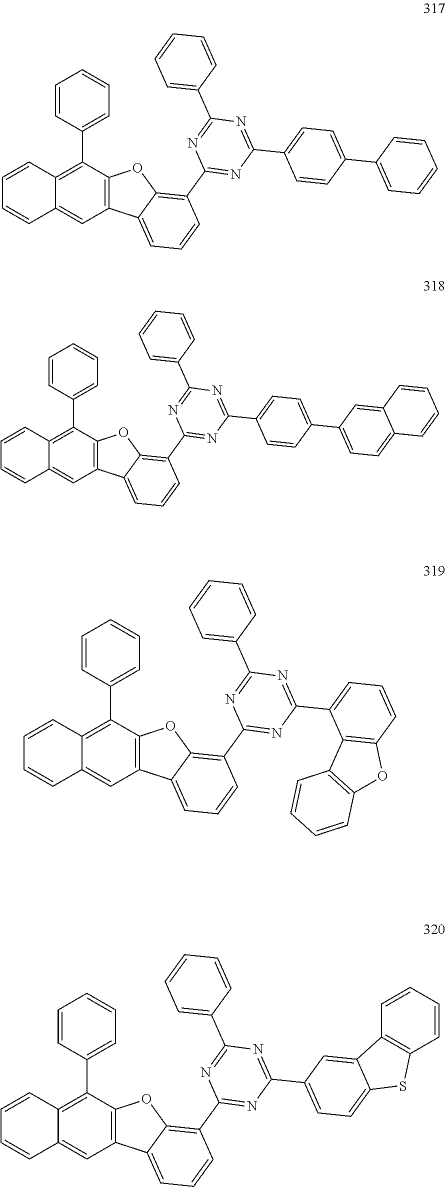 [SMILES: c1ccc(-c2ccc(-c3nc(-c4ccccc4)nc(-c4cccc5c4oc4c(-c6ccccc6)c6ccccc6cc45)n3)cc2)cc1 ; c1ccc(-c2nc(-c3ccc(-c4ccc5ccccc5c4)cc3)nc(-c3cccc4c3oc3c(-c5ccccc5)c5ccccc5cc34)n2)cc1 ; c1ccc(-c2nc(-c3cccc4c3oc3c(-c5ccccc5)c5ccccc5cc34)nc(-c3cccc4oc5ccccc5c34)n2)cc1 ; c1ccc(-c2nc(-c3ccc4sc5ccccc5c4c3)nc(-c3cccc4c3oc3c(-c5ccccc5)c5ccccc5cc34)n2)cc1]

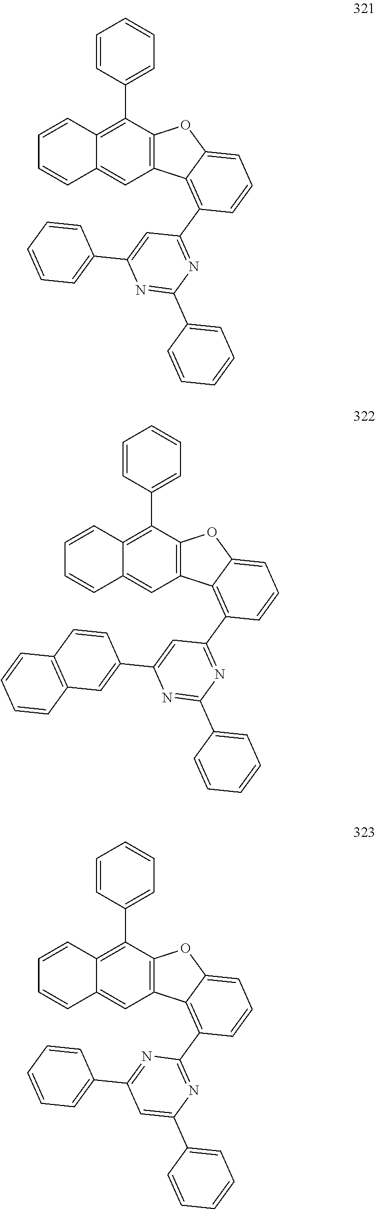 [SMILES: c1ccc(-c2cc(-c3cccc4oc5c(-c6ccccc6)c6ccccc6cc5c34)nc(-c3ccccc3)n2)cc1 ; c1ccc(-c2nc(-c3ccc4ccccc4c3)cc(-c3cccc4oc5c(-c6ccccc6)c6ccccc6cc5c34)n2)cc1 ; c1ccc(-c2cc(-c3ccccc3)nc(-c3cccc4oc5c(-c6ccccc6)c6ccccc6cc5c34)n2)cc1]

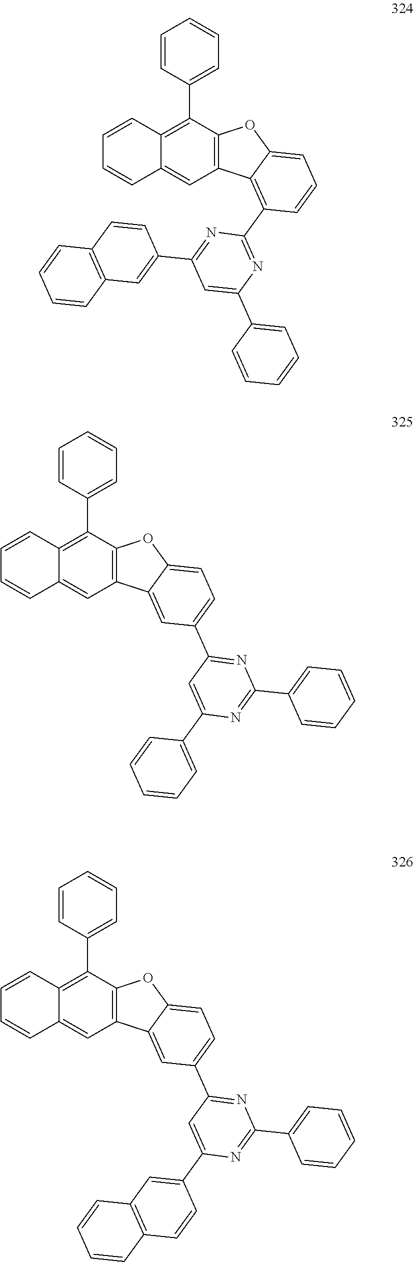 [SMILES: c1ccc(-c2cc(-c3ccc4ccccc4c3)nc(-c3cccc4oc5c(-c6ccccc6)c6ccccc6cc5c34)n2)cc1 ; c1ccc(-c2cc(-c3ccc4oc5c(-c6ccccc6)c6ccccc6cc5c4c3)nc(-c3ccccc3)n2)cc1 ; c1ccc(-c2nc(-c3ccc4ccccc4c3)cc(-c3ccc4oc5c(-c6ccccc6)c6ccccc6cc5c4c3)n2)cc1]

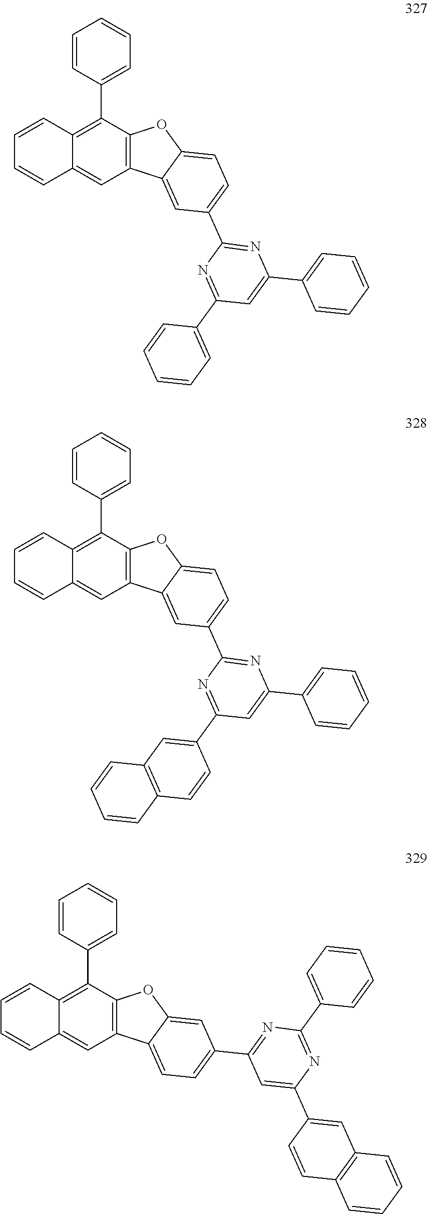 [SMILES: c1ccc(-c2cc(-c3ccccc3)nc(-c3ccc4oc5c(-c6ccccc6)c6ccccc6cc5c4c3)n2)cc1 ; c1ccc(-c2cc(-c3ccc4ccccc4c3)nc(-c3ccc4oc5c(-c6ccccc6)c6ccccc6cc5c4c3)n2)cc1 ; c1ccc(-c2nc(-c3ccc4ccccc4c3)cc(-c3ccc4c(c3)oc3c(-c5ccccc5)c5ccccc5cc34)n2)cc1]

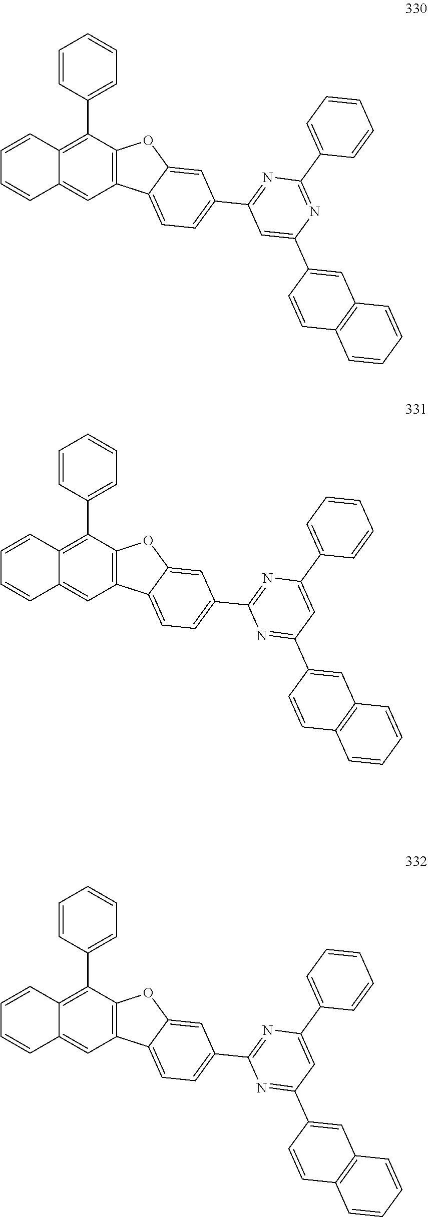 [SMILES: c1ccc(-c2nc(-c3ccc4ccccc4c3)cc(-c3ccc4c(c3)oc3c(-c5ccccc5)c5ccccc5cc34)n2)cc1 ; c1ccc(-c2cc(-c3ccc4ccccc4c3)nc(-c3ccc4c(c3)oc3c(-c5ccccc5)c5ccccc5cc34)n2)cc1 ; c1ccc(-c2cc(-c3ccc4ccccc4c3)nc(-c3ccc4c(c3)oc3c(-c5ccccc5)c5ccccc5cc34)n2)cc1]

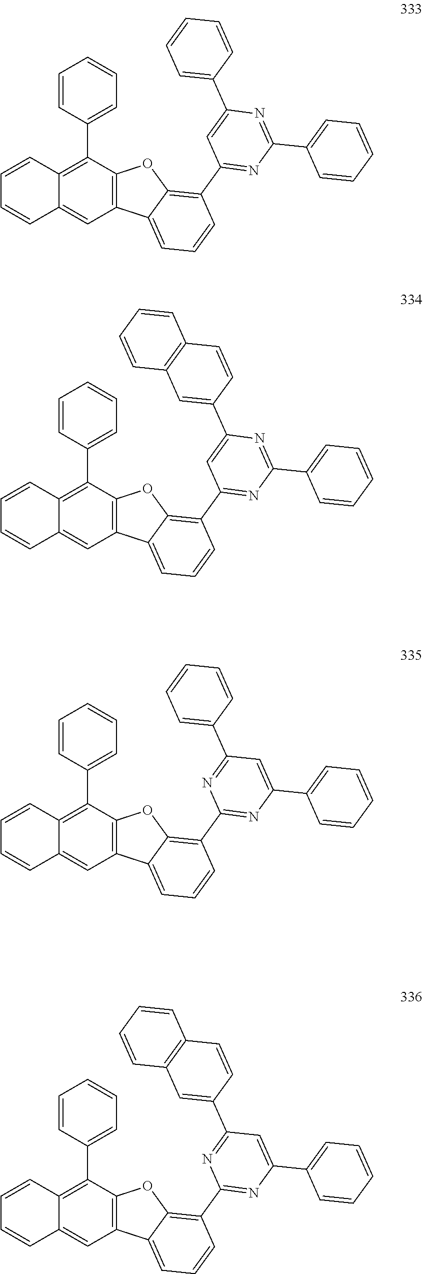 [SMILES: c1ccc(-c2cc(-c3cccc4c3oc3c(-c5ccccc5)c5ccccc5cc34)nc(-c3ccccc3)n2)cc1 ; c1ccc(-c2nc(-c3ccc4ccccc4c3)cc(-c3cccc4c3oc3c(-c5ccccc5)c5ccccc5cc34)n2)cc1 ; c1ccc(-c2cc(-c3ccccc3)nc(-c3cccc4c3oc3c(-c5ccccc5)c5ccccc5cc34)n2)cc1 ; c1ccc(-c2cc(-c3ccc4ccccc4c3)nc(-c3cccc4c3oc3c(-c5ccccc5)c5ccccc5cc34)n2)cc1]

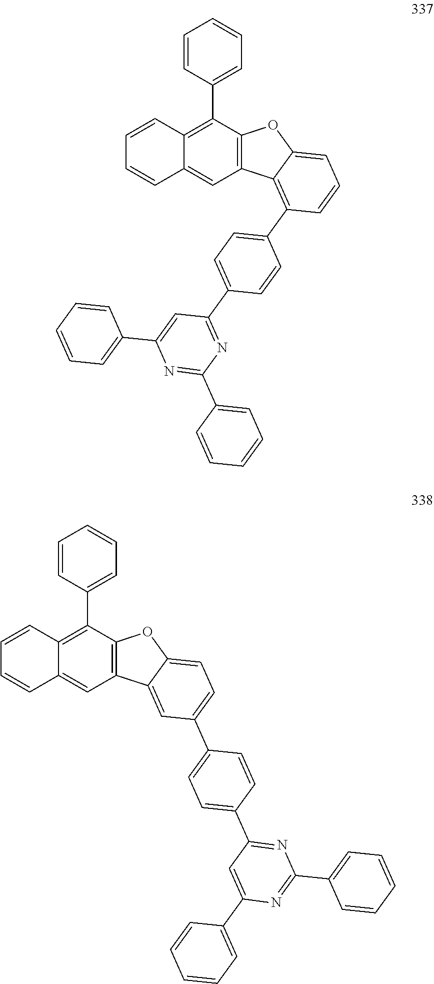 [SMILES: c1ccc(-c2cc(-c3ccc(-c4cccc5oc6c(-c7ccccc7)c7ccccc7cc6c45)cc3)nc(-c3ccccc3)n2)cc1 ; c1ccc(-c2cc(-c3ccc(-c4ccc5oc6c(-c7ccccc7)c7ccccc7cc6c5c4)cc3)nc(-c3ccccc3)n2)cc1]

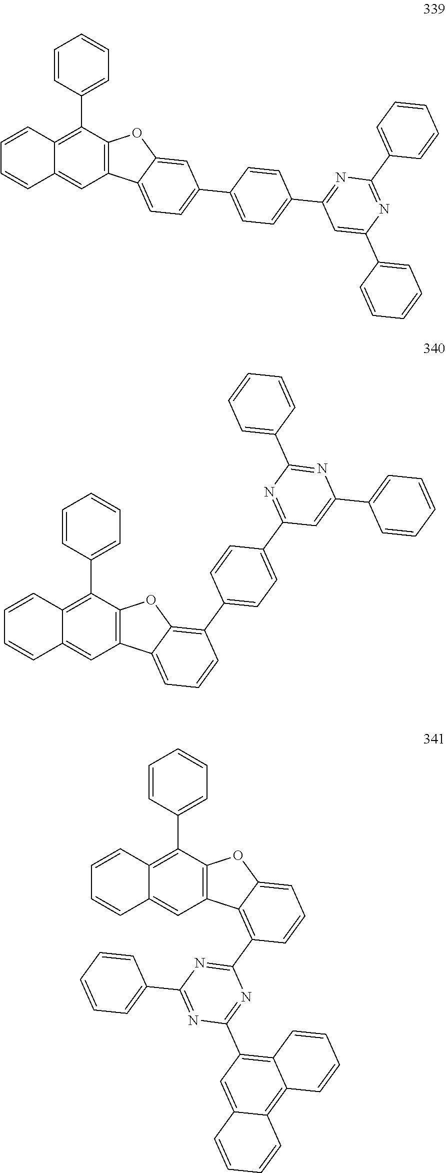 [SMILES: c1ccc(-c2cc(-c3ccc(-c4ccc5c(c4)oc4c(-c6ccccc6)c6ccccc6cc45)cc3)nc(-c3ccccc3)n2)cc1 ; c1ccc(-c2cc(-c3ccc(-c4cccc5c4oc4c(-c6ccccc6)c6ccccc6cc45)cc3)nc(-c3ccccc3)n2)cc1 ; c1ccc(-c2nc(-c3cc4ccccc4c4ccccc34)nc(-c3cccc4oc5c(-c6ccccc6)c6ccccc6cc5c34)n2)cc1]

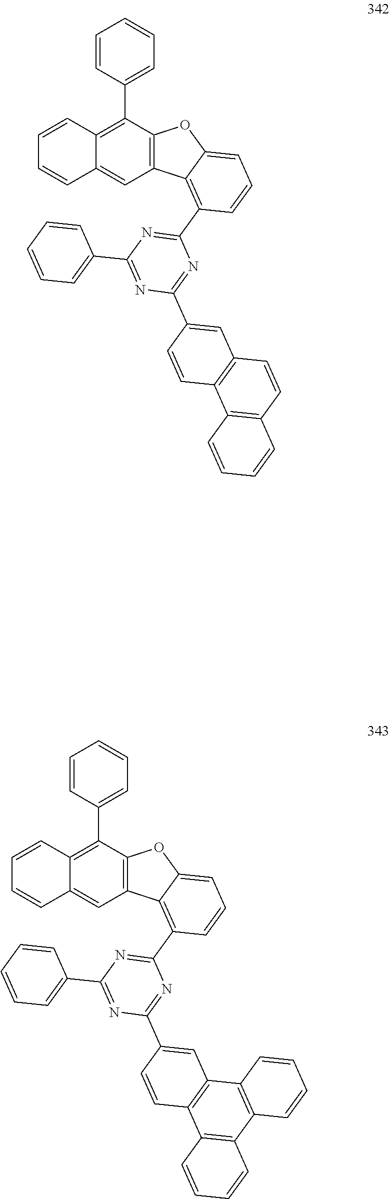 [SMILES: c1ccc(-c2nc(-c3ccc4c(ccc5ccccc54)c3)nc(-c3cccc4oc5c(-c6ccccc6)c6ccccc6cc5c34)n2)cc1 ; c1ccc(-c2nc(-c3ccc4c5ccccc5c5ccccc5c4c3)nc(-c3cccc4oc5c(-c6ccccc6)c6ccccc6cc5c34)n2)cc1]

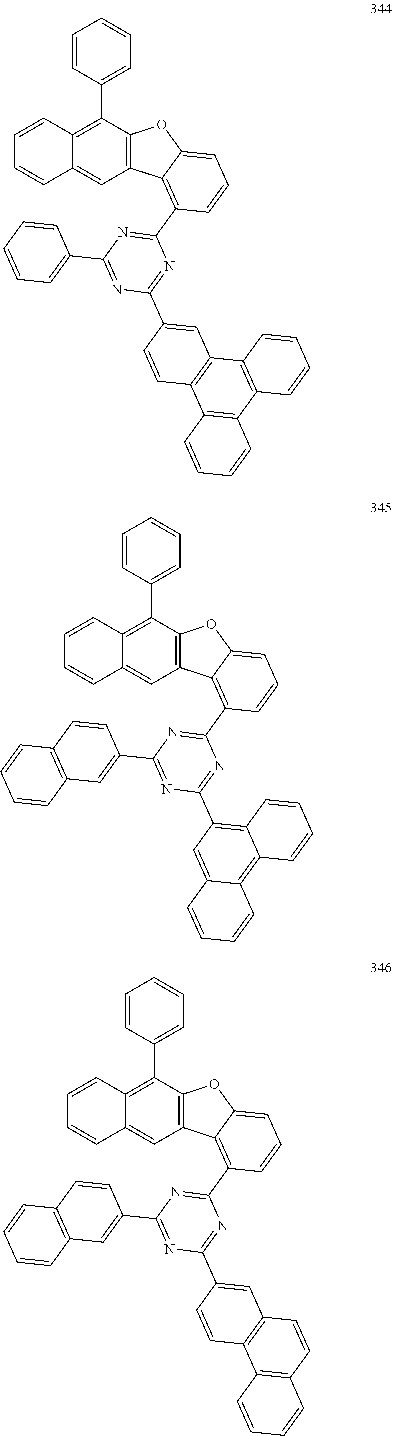 [SMILES: c1ccc(-c2nc(-c3ccc4c5ccccc5c5ccccc5c4c3)nc(-c3cccc4oc5c(-c6ccccc6)c6ccccc6cc5c34)n2)cc1 ; c1ccc(-c2c3ccccc3cc3c2oc2cccc(-c4nc(-c5ccc6ccccc6c5)nc(-c5cc6ccccc6c6ccccc56)n4)c23)cc1 ; c1ccc(-c2c3ccccc3cc3c2oc2cccc(-c4nc(-c5ccc6ccccc6c5)nc(-c5ccc6c(ccc7ccccc76)c5)n4)c23)cc1]

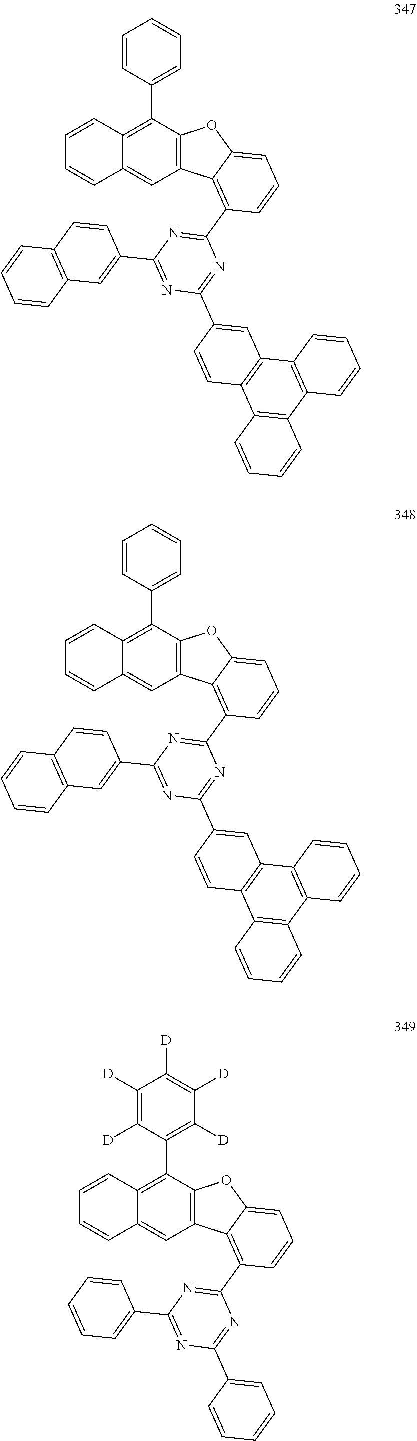 [SMILES: c1ccc(-c2c3ccccc3cc3c2oc2cccc(-c4nc(-c5ccc6ccccc6c5)nc(-c5ccc6c7ccccc7c7ccccc7c6c5)n4)c23)cc1 ; c1ccc(-c2c3ccccc3cc3c2oc2cccc(-c4nc(-c5ccc6ccccc6c5)nc(-c5ccc6c7ccccc7c7ccccc7c6c5)n4)c23)cc1 ; [2H]c1c([2H])c([2H])c(-c2c3ccccc3cc3c2oc2cccc(-c4nc(-c5ccccc5)nc(-c5ccccc5)n4)c23)c([2H])c1[2H]]

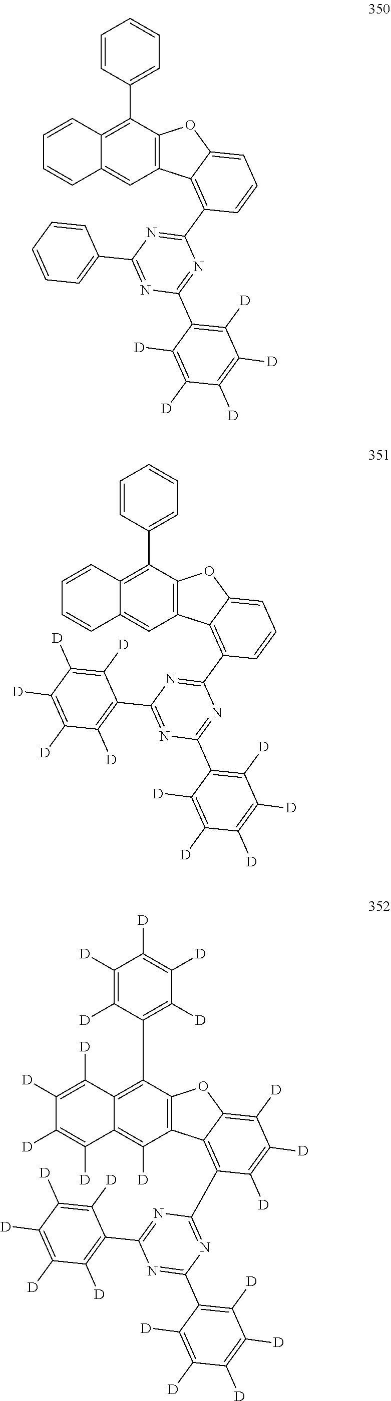 [SMILES: [2H]c1c([2H])c([2H])c(-c2nc(-c3ccccc3)nc(-c3cccc4oc5c(-c6ccccc6)c6ccccc6cc5c34)n2)c([2H])c1[2H] ; [2H]c1c([2H])c([2H])c(-c2nc(-c3c([2H])c([2H])c([2H])c([2H])c3[2H])nc(-c3cccc4oc5c(-c6ccccc6)c6ccccc6cc5c34)n2)c([2H])c1[2H] ; [2H]c1c([2H])c([2H])c(-c2nc(-c3c([2H])c([2H])c([2H])c([2H])c3[2H])nc(-c3c([2H])c([2H])c([2H])c4oc5c(-c6c([2H])c([2H])c([2H])c([2H])c6[2H])c6c([2H])c([2H])c([2H])c([2H])c6c([2H])c5c34)n2)c([2H])c1[2H]]

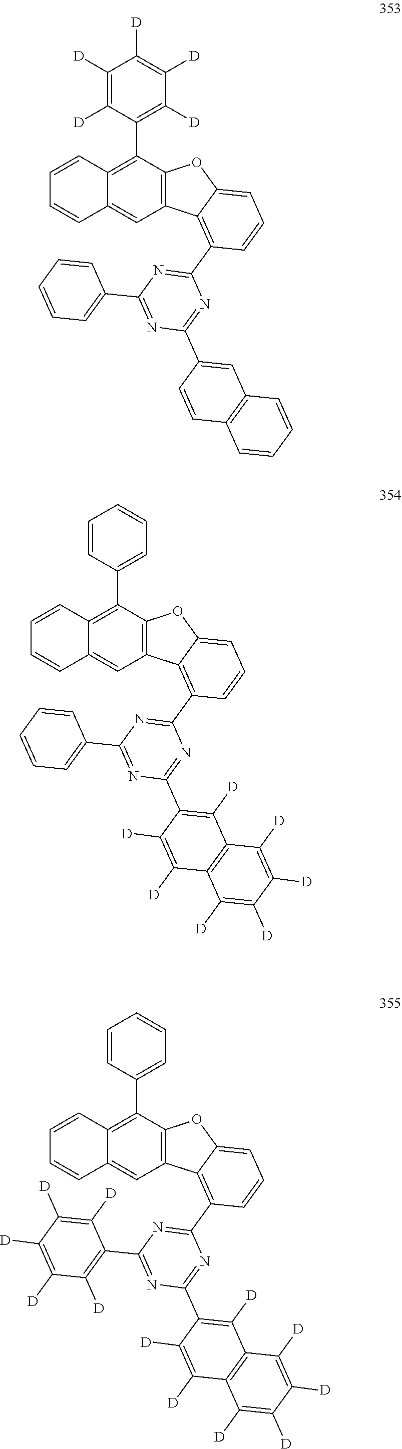 [SMILES: [2H]c1c([2H])c([2H])c(-c2c3ccccc3cc3c2oc2cccc(-c4nc(-c5ccccc5)nc(-c5ccc6ccccc6c5)n4)c23)c([2H])c1[2H] ; [2H]c1c([2H])c([2H])c2c([2H])c(-c3nc(-c4ccccc4)nc(-c4cccc5oc6c(-c7ccccc7)c7ccccc7cc6c45)n3)c([2H])c([2H])c2c1[2H] ; [2H]c1c([2H])c([2H])c(-c2nc(-c3c([2H])c([2H])c4c([2H])c([2H])c([2H])c([2H])c4c3[2H])nc(-c3cccc4oc5c(-c6ccccc6)c6ccccc6cc5c34)n2)c([2H])c1[2H]]

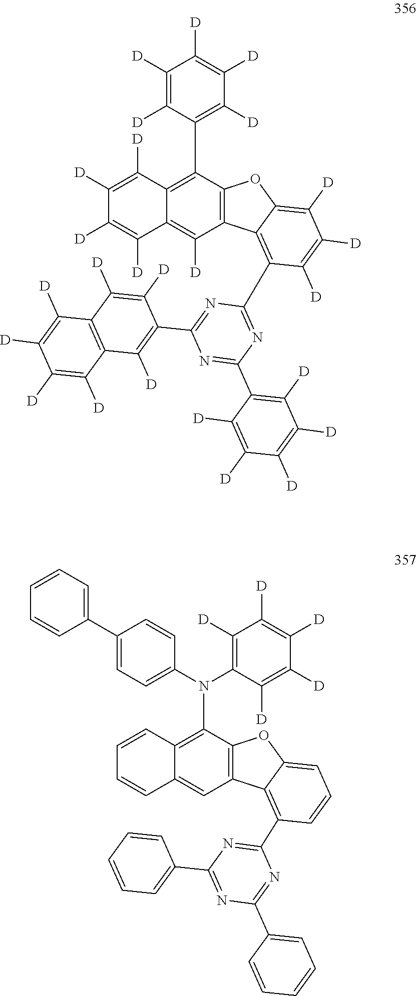 [SMILES: [2H]c1c([2H])c([2H])c(-c2nc(-c3c([2H])c([2H])c4c([2H])c([2H])c([2H])c([2H])c4c3[2H])nc(-c3c([2H])c([2H])c([2H])c4oc5c(-c6c([2H])c([2H])c([2H])c([2H])c6[2H])c6c([2H])c([2H])c([2H])c([2H])c6c([2H])c5c34)n2)c([2H])c1[2H] ; [2H]c1c([2H])c([2H])c(N(c2ccc(-c3ccccc3)cc2)c2c3ccccc3cc3c2oc2cccc(-c4nc(-c5ccccc5)nc(-c5ccccc5)n4)c23)c([2H])c1[2H]]

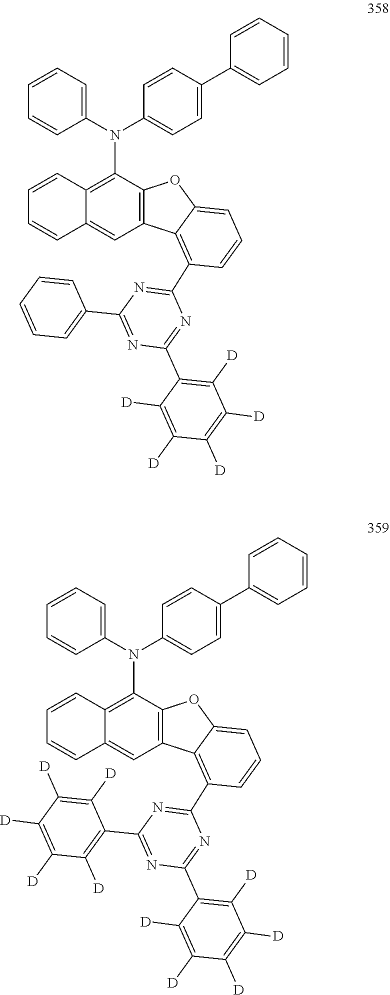 [SMILES: [2H]c1c([2H])c([2H])c(-c2nc(-c3ccccc3)nc(-c3cccc4oc5c(N(c6ccccc6)c6ccc(-c7ccccc7)cc6)c6ccccc6cc5c34)n2)c([2H])c1[2H] ; [2H]c1c([2H])c([2H])c(-c2nc(-c3c([2H])c([2H])c([2H])c([2H])c3[2H])nc(-c3cccc4oc5c(N(c6ccccc6)c6ccc(-c7ccccc7)cc6)c6ccccc6cc5c34)n2)c([2H])c1[2H]]

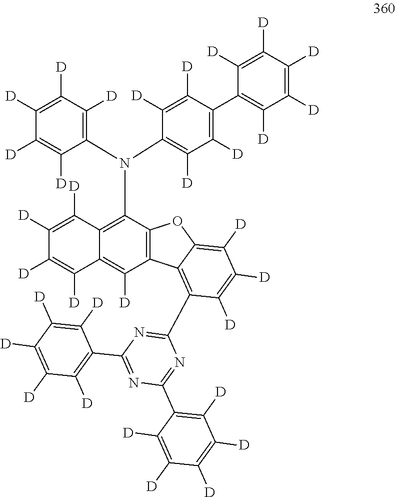 [SMILES: [2H]c1c([2H])c([2H])c(-c2nc(-c3c([2H])c([2H])c([2H])c([2H])c3[2H])nc(-c3c([2H])c([2H])c([2H])c4oc5c(N(c6c([2H])c([2H])c([2H])c([2H])c6[2H])c6c([2H])c([2H])c(-c7c([2H])c([2H])c([2H])c([2H])c7[2H])c([2H])c6[2H])c6c([2H])c([2H])c([2H])c([2H])c6c([2H])c5c34)n2)c([2H])c1[2H]]

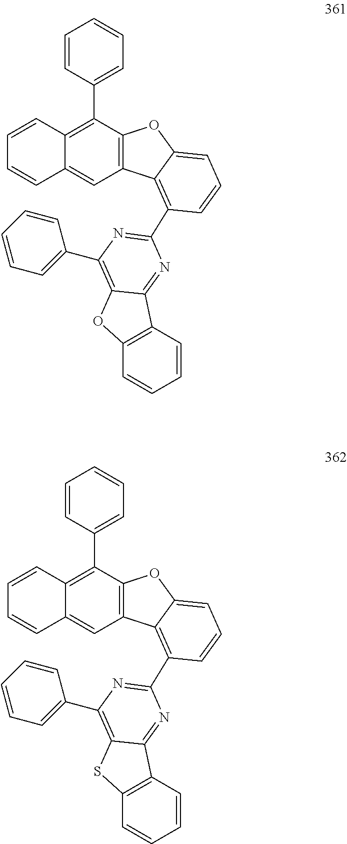 [SMILES: c1ccc(-c2c3ccccc3cc3c2oc2cccc(-c4nc(-c5ccccc5)c5oc6ccccc6c5n4)c23)cc1 ; c1ccc(-c2c3ccccc3cc3c2oc2cccc(-c4nc(-c5ccccc5)c5sc6ccccc6c5n4)c23)cc1]

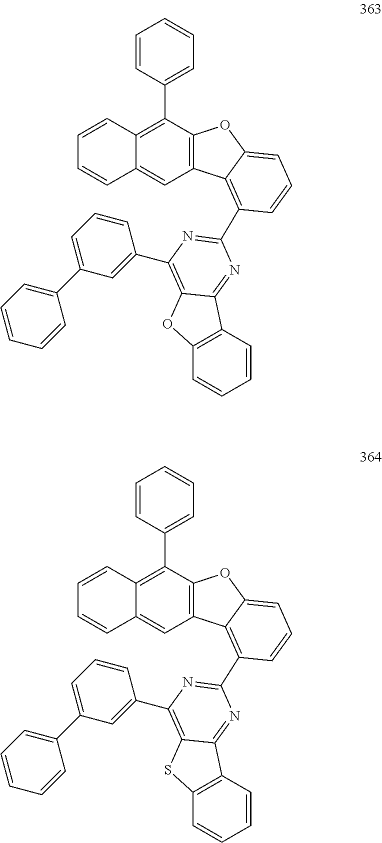 [SMILES: c1ccc(-c2cccc(-c3nc(-c4cccc5oc6c(-c7ccccc7)c7ccccc7cc6c45)nc4c3oc3ccccc34)c2)cc1 ; c1ccc(-c2cccc(-c3nc(-c4cccc5oc6c(-c7ccccc7)c7ccccc7cc6c45)nc4c3sc3ccccc34)c2)cc1]

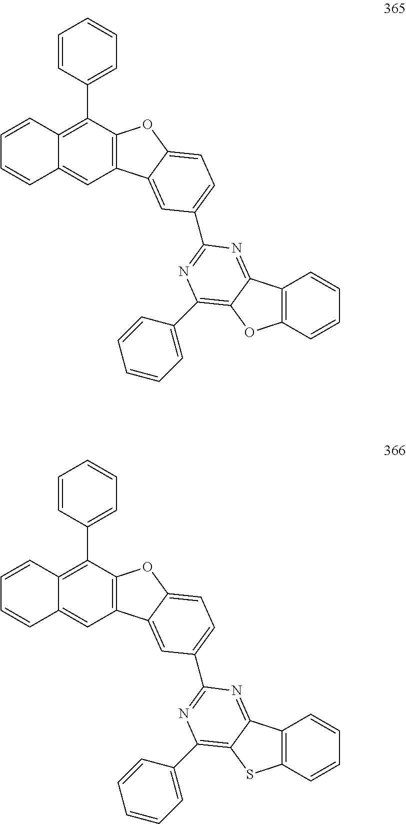 [SMILES: c1ccc(-c2c3ccccc3cc3c2oc2ccc(-c4nc(-c5ccccc5)c5oc6ccccc6c5n4)cc23)cc1 ; c1ccc(-c2c3ccccc3cc3c2oc2ccc(-c4nc(-c5ccccc5)c5sc6ccccc6c5n4)cc23)cc1]

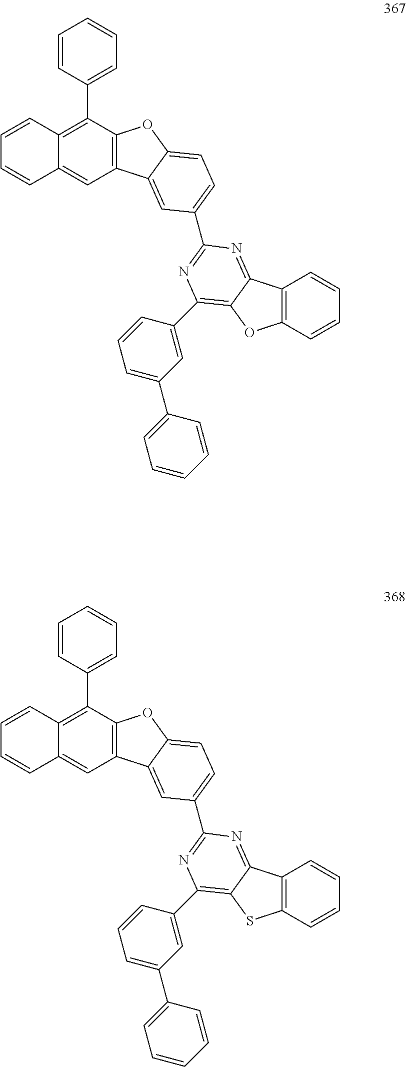 [SMILES: c1ccc(-c2cccc(-c3nc(-c4ccc5oc6c(-c7ccccc7)c7ccccc7cc6c5c4)nc4c3oc3ccccc34)c2)cc1 ; c1ccc(-c2cccc(-c3nc(-c4ccc5oc6c(-c7ccccc7)c7ccccc7cc6c5c4)nc4c3sc3ccccc34)c2)cc1]

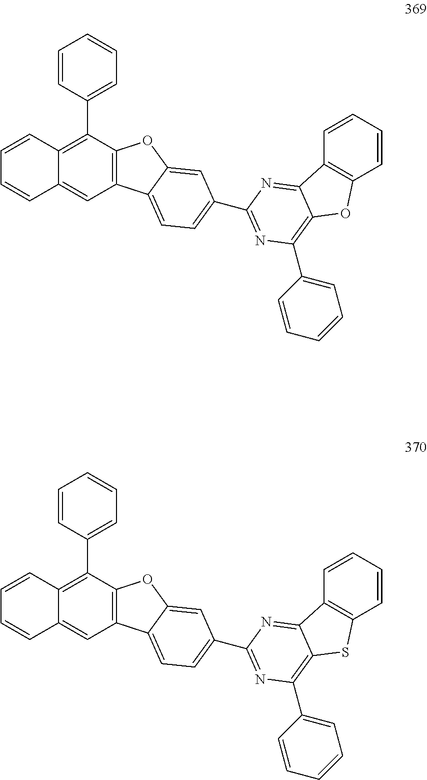 [SMILES: c1ccc(-c2c3ccccc3cc3c2oc2cc(-c4nc(-c5ccccc5)c5oc6ccccc6c5n4)ccc23)cc1 ; c1ccc(-c2c3ccccc3cc3c2oc2cc(-c4nc(-c5ccccc5)c5sc6ccccc6c5n4)ccc23)cc1]

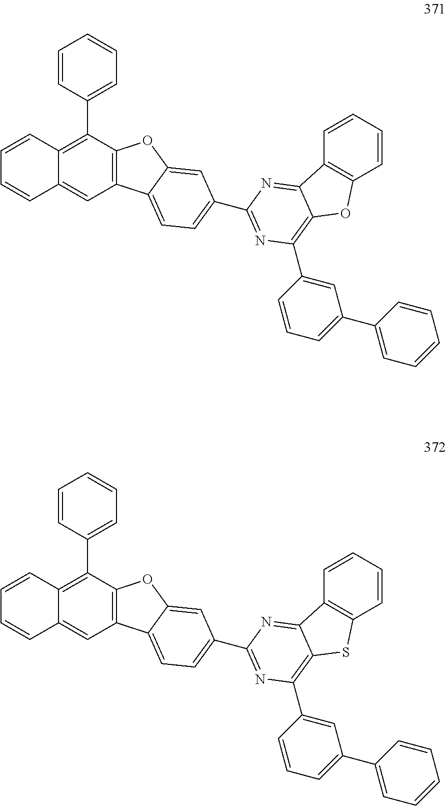 [SMILES: c1ccc(-c2cccc(-c3nc(-c4ccc5c(c4)oc4c(-c6ccccc6)c6ccccc6cc45)nc4c3oc3ccccc34)c2)cc1 ; c1ccc(-c2cccc(-c3nc(-c4ccc5c(c4)oc4c(-c6ccccc6)c6ccccc6cc45)nc4c3sc3ccccc34)c2)cc1]

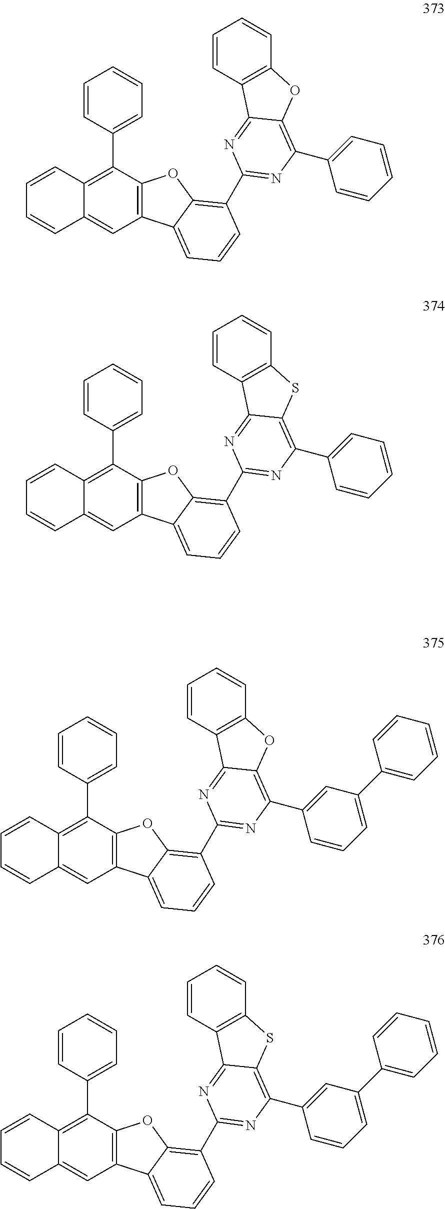 [SMILES: c1ccc(-c2c3ccccc3cc3c2oc2c(-c4nc(-c5ccccc5)c5oc6ccccc6c5n4)cccc23)cc1 ; c1ccc(-c2c3ccccc3cc3c2oc2c(-c4nc(-c5ccccc5)c5sc6ccccc6c5n4)cccc23)cc1 ; c1ccc(-c2cccc(-c3nc(-c4cccc5c4oc4c(-c6ccccc6)c6ccccc6cc45)nc4c3oc3ccccc34)c2)cc1 ; c1ccc(-c2cccc(-c3nc(-c4cccc5c4oc4c(-c6ccccc6)c6ccccc6cc45)nc4c3sc3ccccc34)c2)cc1]

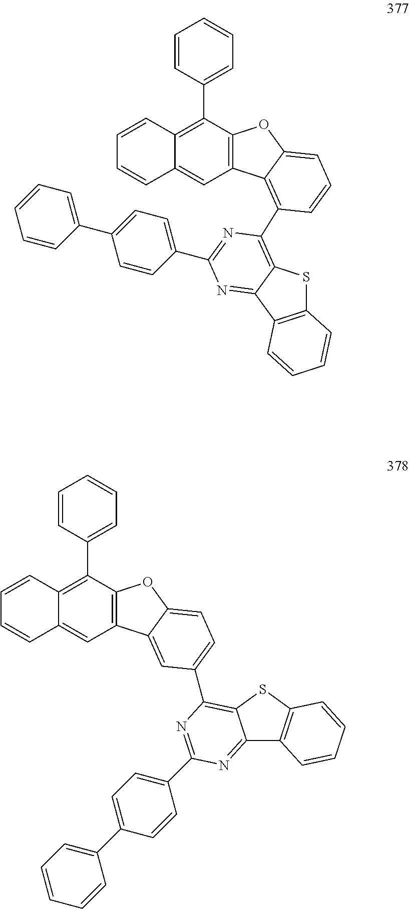 [SMILES: c1ccc(-c2ccc(-c3nc(-c4cccc5oc6c(-c7ccccc7)c7ccccc7cc6c45)c4sc5ccccc5c4n3)cc2)cc1 ; c1ccc(-c2ccc(-c3nc(-c4ccc5oc6c(-c7ccccc7)c7ccccc7cc6c5c4)c4sc5ccccc5c4n3)cc2)cc1]

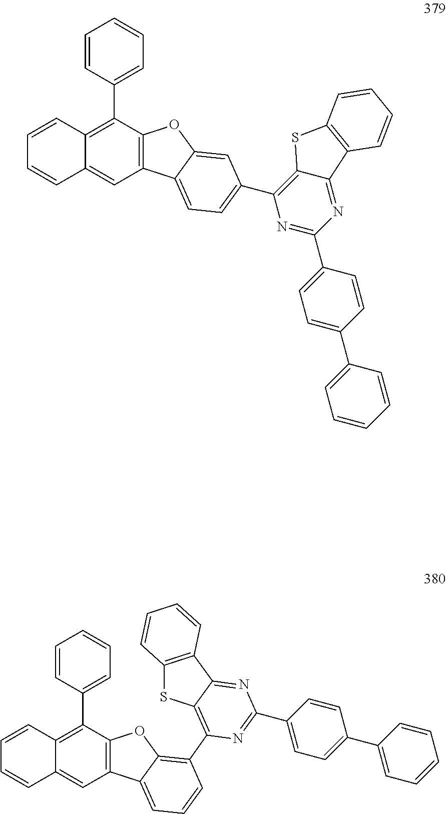 [SMILES: c1ccc(-c2ccc(-c3nc(-c4ccc5c(c4)oc4c(-c6ccccc6)c6ccccc6cc45)c4sc5ccccc5c4n3)cc2)cc1 ; c1ccc(-c2ccc(-c3nc(-c4cccc5c4oc4c(-c6ccccc6)c6ccccc6cc45)c4sc5ccccc5c4n3)cc2)cc1]

Further, various substituents may be introduced into the structure of Chemical Formula 1 to synthesize a compound having inherent characteristics of a substituent introduced. For example, it is possible to synthesize a material which satisfies conditions required for each organic material layer by introducing a substituent usually used for a hole injection layer material, a hole transport layer material, a light emitting layer material, an electron transport layer material, and a charge generation layer material, which are used for preparing an organic light emitting device, into the core structure.

In addition, it is possible to finely adjust an energy band-gap by introducing various substituents into the structure of Chemical Formula 1, and meanwhile, it is possible to improve characteristics at the interface between organic materials and diversify the use of the material.

In another exemplary embodiment of the present specification, provided is an organic light emitting device including: a first electrode; a second electrode; and an organic material layer having one or more layers provided between the first electrode and the second electrode, in which one or more layers of the organic material layer include one or more heterocyclic compounds of Chemical Formula 1.

In an exemplary embodiment of the present specification, the organic material layer includes a light emitting layer, and the light emitting layer may include one or more of the heterocyclic compounds.

In an exemplary embodiment of the present specification, the organic material layer includes a light emitting layer, and the light emitting layer may include one of the heterocyclic compounds.

In an exemplary embodiment of the present specification, the organic material layer includes a light emitting layer, and when the light emitting layer includes a host, the host may include one or more of the heterocyclic compounds.

In an exemplary embodiment of the present specification, the organic material layer includes a light emitting layer, the light emitting layer includes a host, and the host may include one of the heterocyclic compounds.

In an exemplary embodiment of the present specification, the organic material layer includes a light emitting layer, and the light emitting layer may include the heterocyclic compound as an N-type host.

In an exemplary embodiment of the present specification, an organic material layer including the heterocyclic compound may further include a compound of the following Chemical Formula 2 or 3.

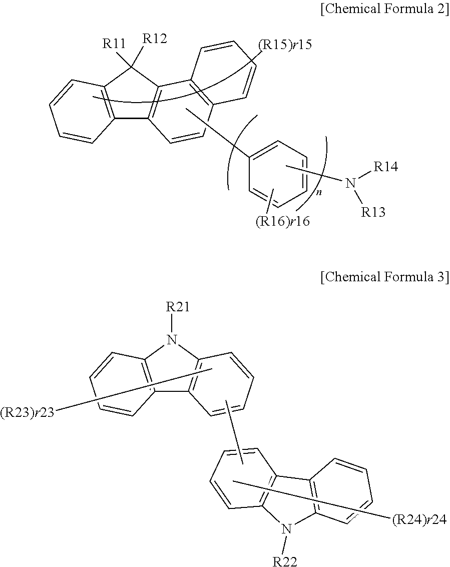

In Chemical Formulae 2 and 3,
R11 to R14 and R21 to R24 are each independently hydrogen; deuterium; a substituted or unsubstituted alkyl group having 1 to 60 carbon atoms; a substituted or unsubstituted aryl group having 6 to 60 carbon atoms; or a substituted or unsubstituted heteroaryl group having 2 to 60 carbon atoms,
R15 and R16 are each independently hydrogen; or deuterium,
n is 0 or 1,
r15 is an integer from 0 to 9,
r16 is an integer from 0 to 4,
r23 and r24 are each an integer from 0 to 7, and
when r15, r16, r23 and r24 are each 2 or higher, substituents in the parenthesis are the same as or different from each other.

In an exemplary embodiment of the present specification, an organic material layer including the heterocyclic compound may further include the compound of Chemical Formula 2 or 3 as a P-type host.

In an exemplary embodiment of the present specification, an organic material layer including the heterocyclic compound may further include the compound of Chemical Formula 2.

In an exemplary embodiment of the present specification, when n is 0,

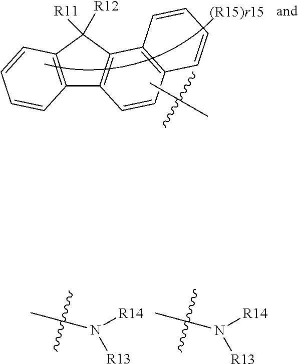

are directly bonded, and when n is 1,

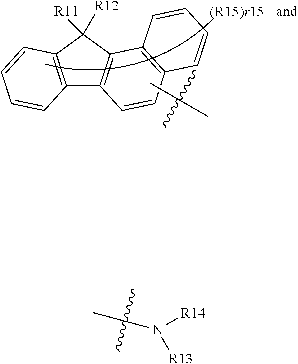

are bonded through a phenylene group, and the phenylene group may be substituted with deuterium.

In an exemplary embodiment of the present specification, Chemical Formula 2 may be represented by the following Chemical Formula 2-1 or 2-2.

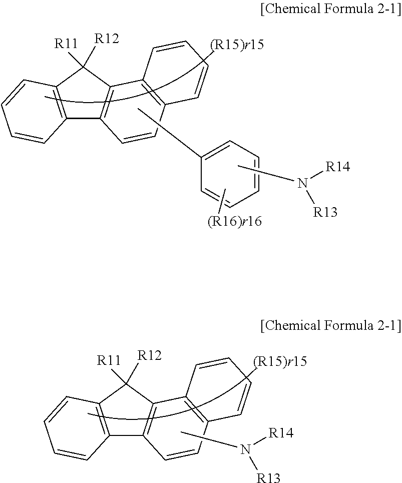

In Chemical Formulae 2-1 and 2-2, the definition of each substituent is the same as that in Chemical Formula 2.

In an exemplary embodiment of the present specification, R11 and R12 may be each independently a substituted or unsubstituted alkyl group having 1 to 60 carbon atoms; or a substituted or unsubstituted aryl group having 6 to 60 carbon atoms.

In an exemplary embodiment of the present specification, R11 and R12 may be each independently a substituted or unsubstituted alkyl group having 1 to 20 carbon atoms; or a substituted or unsubstituted aryl group having 6 to 30 carbon atoms.

In an exemplary embodiment of the present specification, R11 and R12 may be each independently a substituted or unsubstituted alkyl group having 1 to 10 carbon atoms; or a substituted or unsubstituted aryl group having 6 to 20 carbon atoms.

In an exemplary embodiment of the present specification, R11 and R12 may be each independently a substituted or unsubstituted methyl group; or a substituted or unsubstituted phenyl group.

In an exemplary embodiment of the present specification, R11 and R12 may be each independently a methyl group; or a phenyl group.

In an exemplary embodiment of the present specification, R11 and R12 may be each independently a substituted or unsubstituted alkyl group having 1 to 20 carbon atoms.

In an exemplary embodiment of the present specification, R13 and R14 may be each independently a substituted or unsubstituted aryl group having 6 to 60 carbon atoms; or a substituted or unsubstituted heteroaryl group having 2 to 60 carbon atoms.

In an exemplary embodiment of the present specification, R13 and R14 may be each independently a substituted or unsubstituted aryl group having 6 to 30 carbon atoms; or a substituted or unsubstituted heteroaryl group having 2 to 30 carbon atoms.

In an exemplary embodiment of the present specification, R13 and R14 may be each independently a substituted or unsubstituted phenyl group; a substituted or unsubstituted biphenyl group; a substituted or unsubstituted terphenyl group; a substituted or unsubstituted naphthyl group; a substituted or unsubstituted fluorenyl group; a substituted or unsubstituted dibenzofuran group; or a substituted or unsubstituted dibenzothiophene group.

In an exemplary embodiment of the present specification, R13 and R14 may be each independently a substituted or unsubstituted biphenyl group; a substituted or unsubstituted terphenyl group; a substituted or unsubstituted fluorenyl group; a substituted or unsubstituted dibenzofuran group; or a substituted or unsubstituted dibenzothiophene group.

In an exemplary embodiment of the present specification, R13 and R14 may be each independently a biphenyl group; a terphenyl group; a fluorenyl group which is unsubstituted or substituted with an alkyl group or an aryl group; a dibenzofuran group; or a dibenzothiophene group.

In an exemplary embodiment of the present specification, Chemical Formula 2 may be represented by any one of the following compounds.

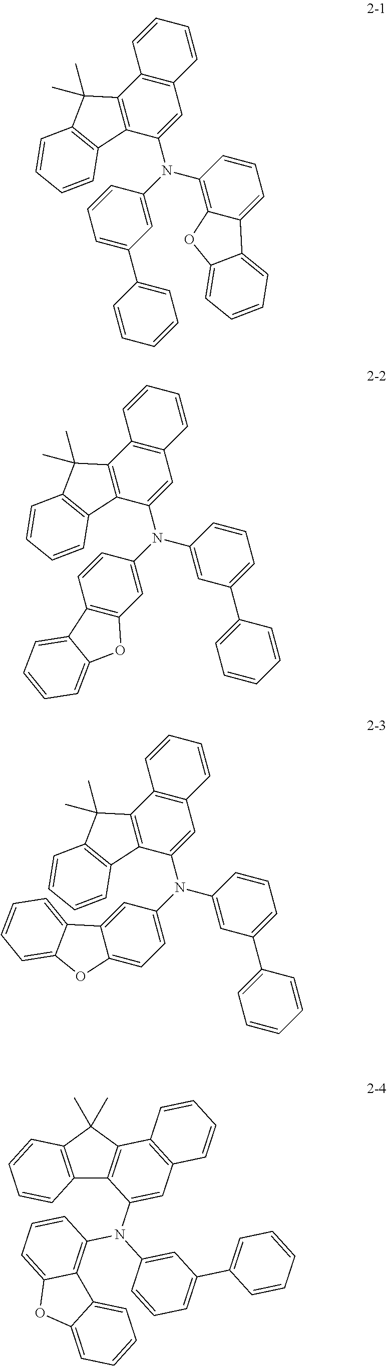

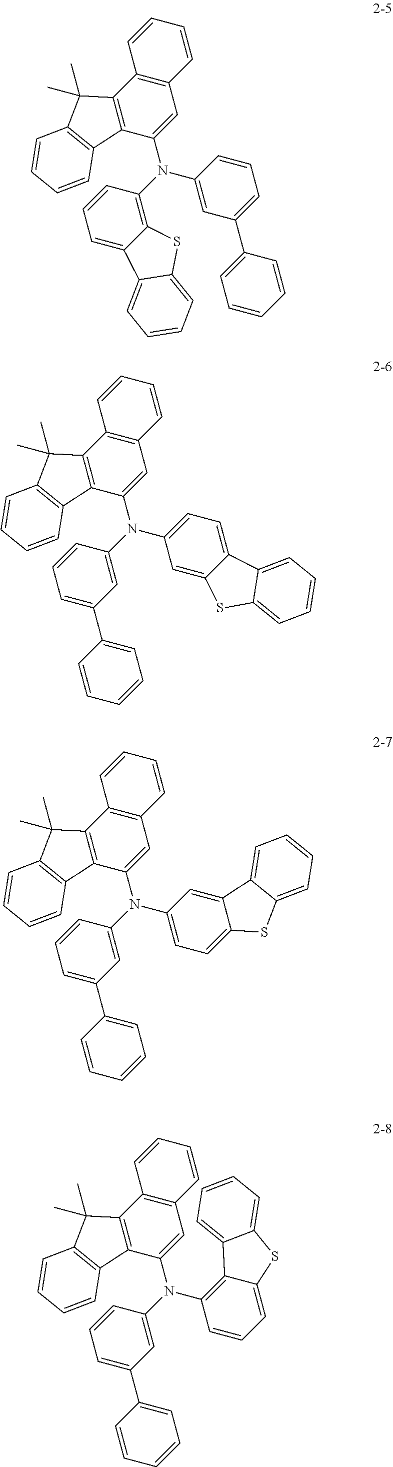 [SMILES: CC1(C)c2ccccc2-c2c(N(c3cccc(-c4ccccc4)c3)c3cccc4c3sc3ccccc34)cc3ccccc3c21 ; CC1(C)c2ccccc2-c2c(N(c3cccc(-c4ccccc4)c3)c3ccc4c(c3)sc3ccccc34)cc3ccccc3c21 ; CC1(C)c2ccccc2-c2c(N(c3cccc(-c4ccccc4)c3)c3ccc4sc5ccccc5c4c3)cc3ccccc3c21 ; CC1(C)c2ccccc2-c2c(N(c3cccc(-c4ccccc4)c3)c3cccc4sc5ccccc5c34)cc3ccccc3c21]

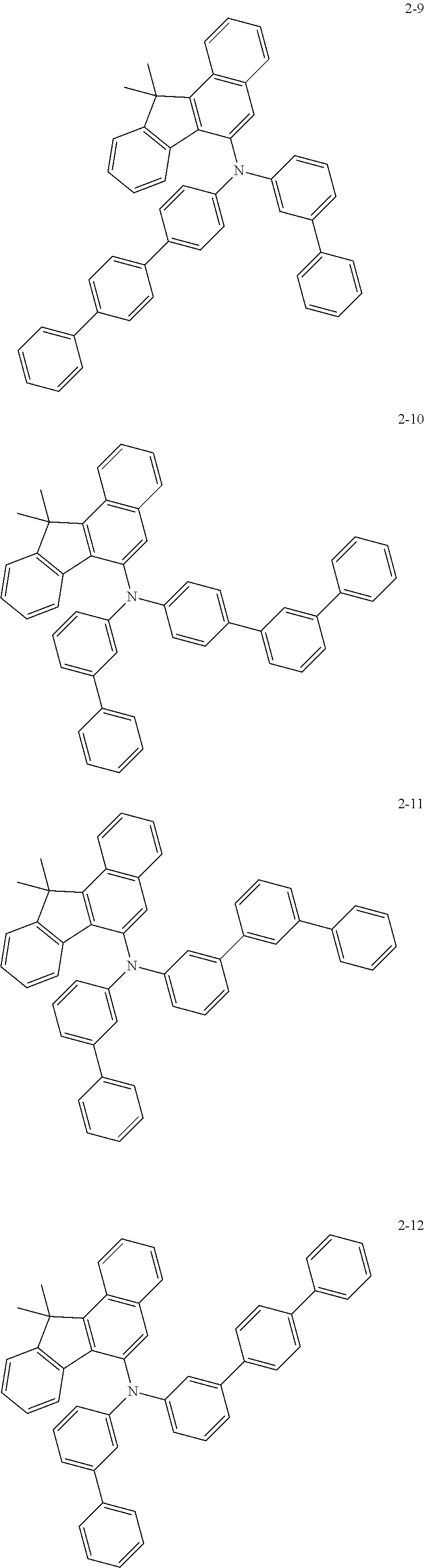

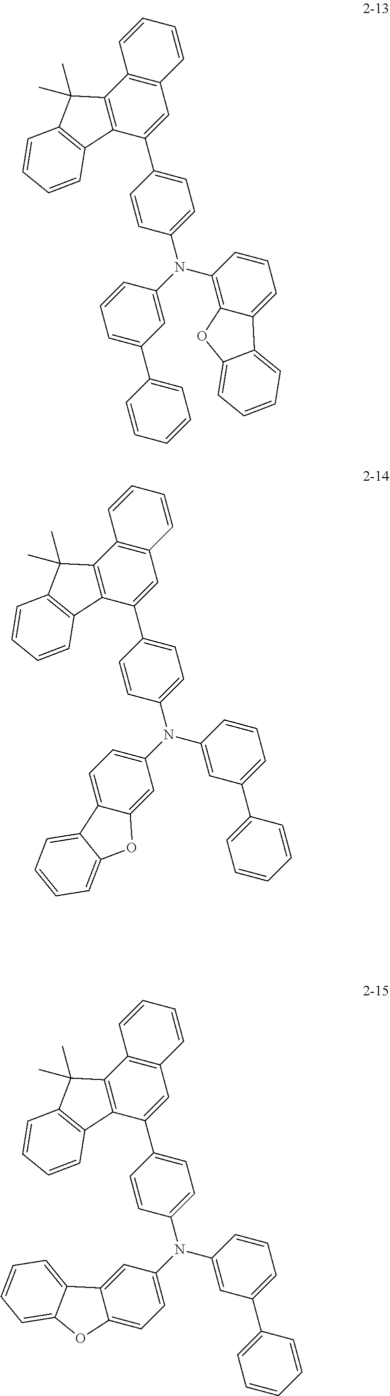 [SMILES: CC1(C)c2ccccc2-c2c(-c3ccc(N(c4cccc(-c5ccccc5)c4)c4cccc5c4oc4ccccc45)cc3)cc3ccccc3c21 ; CC1(C)c2ccccc2-c2c(-c3ccc(N(c4cccc(-c5ccccc5)c4)c4ccc5c(c4)oc4ccccc45)cc3)cc3ccccc3c21 ; CC1(C)c2ccccc2-c2c(-c3ccc(N(c4cccc(-c5ccccc5)c4)c4ccc5oc6ccccc6c5c4)cc3)cc3ccccc3c21]

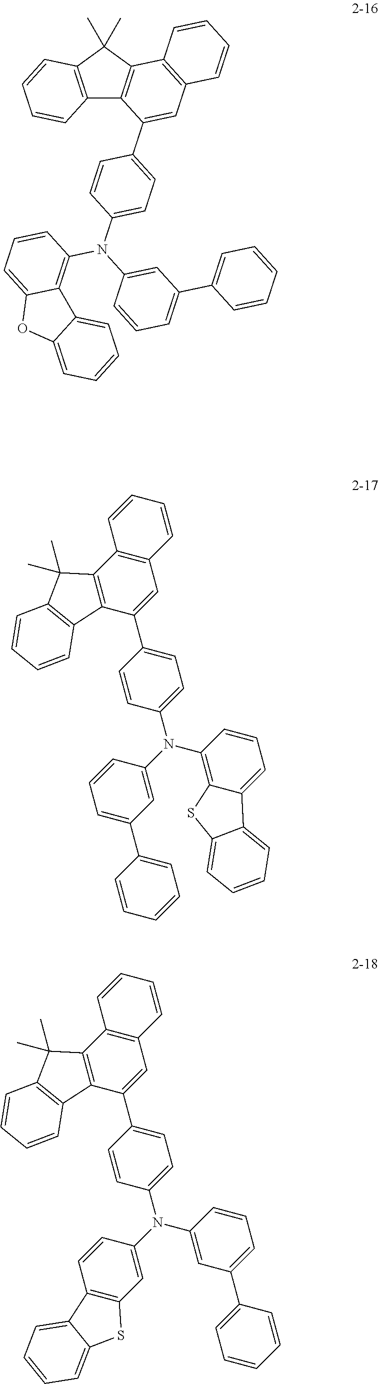 [SMILES: CC1(C)c2ccccc2-c2c(-c3ccc(N(c4cccc(-c5ccccc5)c4)c4cccc5oc6ccccc6c45)cc3)cc3ccccc3c21 ; CC1(C)c2ccccc2-c2c(-c3ccc(N(c4cccc(-c5ccccc5)c4)c4cccc5c4sc4ccccc45)cc3)cc3ccccc3c21 ; CC1(C)c2ccccc2-c2c(-c3ccc(N(c4cccc(-c5ccccc5)c4)c4ccc5c(c4)sc4ccccc45)cc3)cc3ccccc3c21]

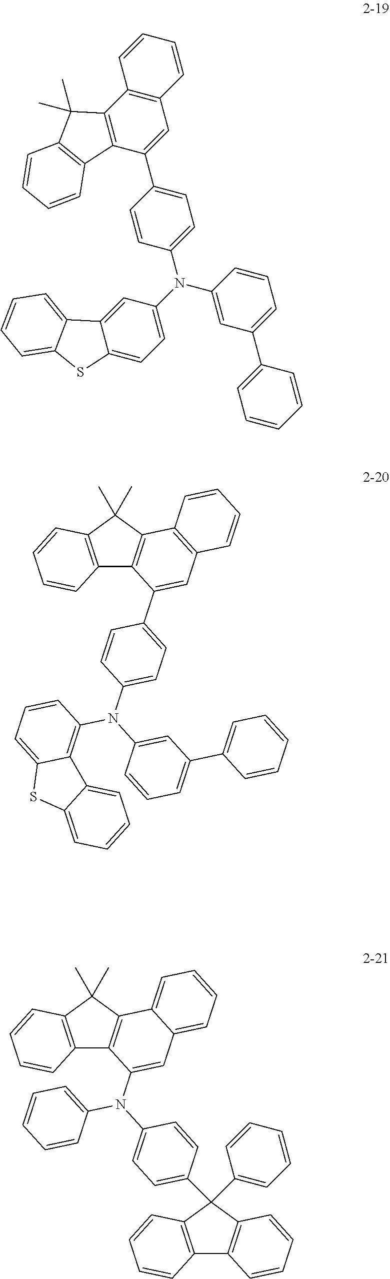 [SMILES: CC1(C)c2ccccc2-c2c(-c3ccc(N(c4cccc(-c5ccccc5)c4)c4ccc5sc6ccccc6c5c4)cc3)cc3ccccc3c21 ; CC1(C)c2ccccc2-c2c(-c3ccc(N(c4cccc(-c5ccccc5)c4)c4cccc5sc6ccccc6c45)cc3)cc3ccccc3c21 ; CC1(C)c2ccccc2-c2c(N(c3ccccc3)c3ccc(C4(c5ccccc5)c5ccccc5-c5ccccc54)cc3)cc3ccccc3c21]

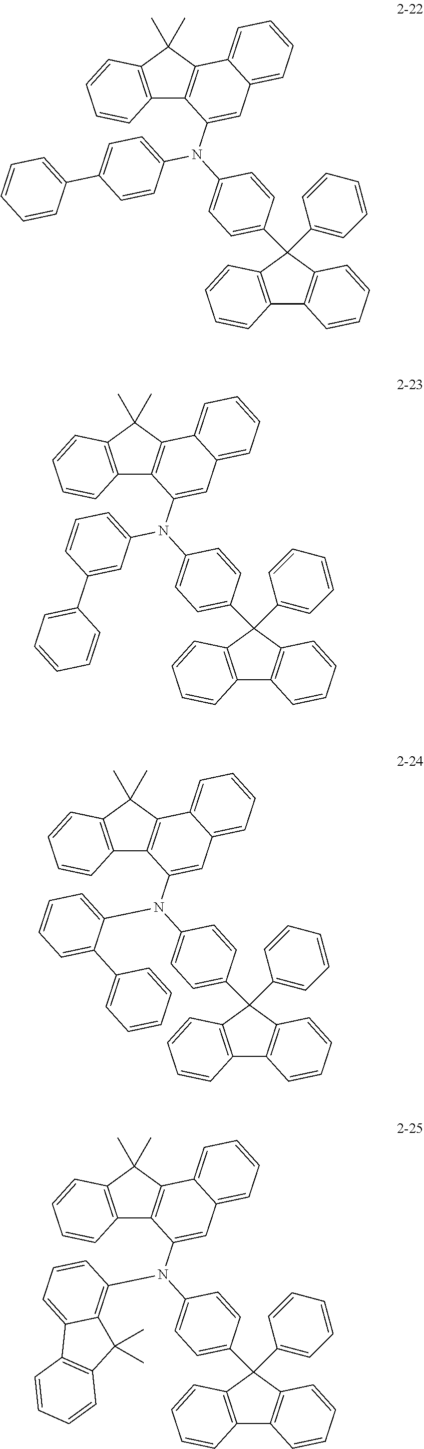 [SMILES: CC1(C)c2ccccc2-c2c(N(c3ccc(-c4ccccc4)cc3)c3ccc(C4(c5ccccc5)c5ccccc5-c5ccccc54)cc3)cc3ccccc3c21 ; CC1(C)c2ccccc2-c2c(N(c3ccc(C4(c5ccccc5)c5ccccc5-c5ccccc54)cc3)c3cccc(-c4ccccc4)c3)cc3ccccc3c21 ; CC1(C)c2ccccc2-c2c(N(c3ccc(C4(c5ccccc5)c5ccccc5-c5ccccc54)cc3)c3ccccc3-c3ccccc3)cc3ccccc3c21 ; CC1(C)c2ccccc2-c2cccc(N(c3ccc(C4(c5ccccc5)c5ccccc5-c5ccccc54)cc3)c3cc4ccccc4c4c3-c3ccccc3C4(C)C)c21]

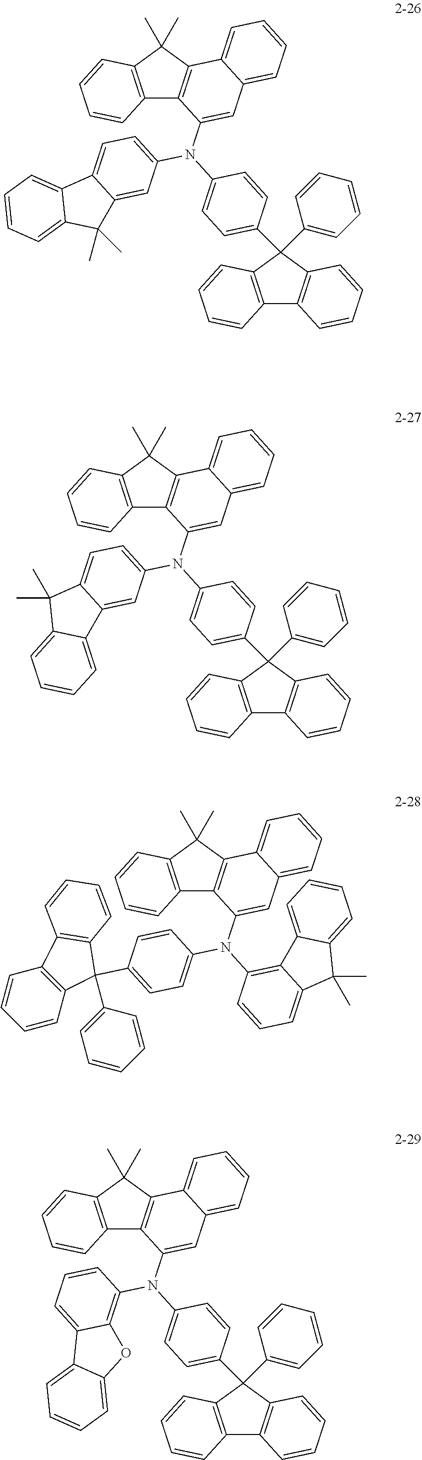 [SMILES: CC1(C)c2ccccc2-c2ccc(N(c3ccc(C4(c5ccccc5)c5ccccc5-c5ccccc54)cc3)c3cc4ccccc4c4c3-c3ccccc3C4(C)C)cc21 ; CC1(C)c2ccccc2-c2cc(N(c3ccc(C4(c5ccccc5)c5ccccc5-c5ccccc54)cc3)c3cc4ccccc4c4c3-c3ccccc3C4(C)C)ccc21 ; CC1(C)c2ccccc2-c2c(N(c3ccc(C4(c5ccccc5)c5ccccc5-c5ccccc54)cc3)c3cc4ccccc4c4c3-c3ccccc3C4(C)C)cccc21 ; CC1(C)c2ccccc2-c2c(N(c3ccc(C4(c5ccccc5)c5ccccc5-c5ccccc54)cc3)c3cccc4c3oc3ccccc34)cc3ccccc3c21]

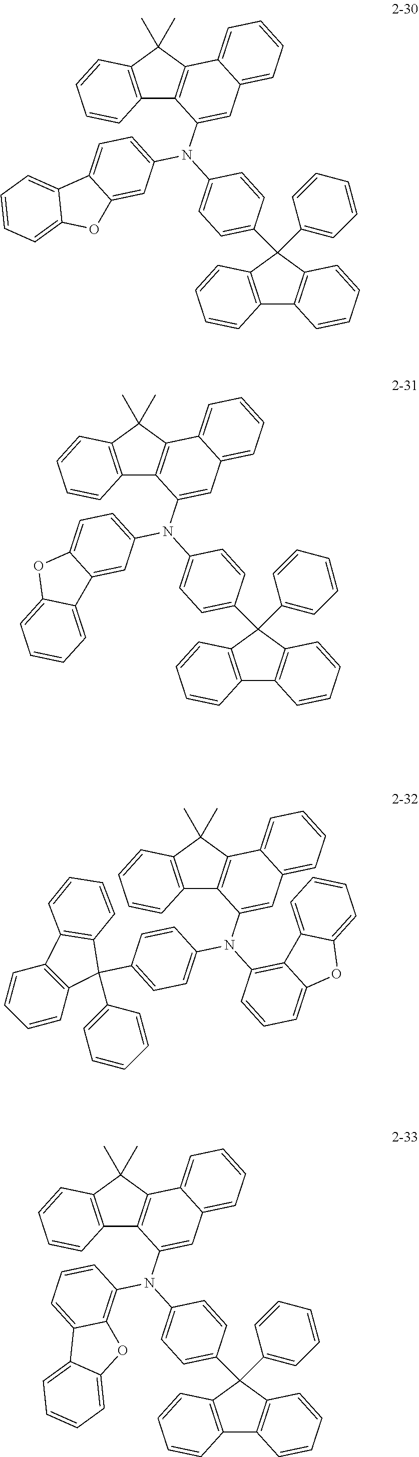 [SMILES: CC1(C)c2ccccc2-c2c(N(c3ccc(C4(c5ccccc5)c5ccccc5-c5ccccc54)cc3)c3ccc4c(c3)oc3ccccc34)cc3ccccc3c21 ; CC1(C)c2ccccc2-c2c(N(c3ccc(C4(c5ccccc5)c5ccccc5-c5ccccc54)cc3)c3ccc4oc5ccccc5c4c3)cc3ccccc3c21 ; CC1(C)c2ccccc2-c2c(N(c3ccc(C4(c5ccccc5)c5ccccc5-c5ccccc54)cc3)c3cccc4oc5ccccc5c34)cc3ccccc3c21 ; CC1(C)c2ccccc2-c2c(N(c3ccc(C4(c5ccccc5)c5ccccc5-c5ccccc54)cc3)c3cccc4c3oc3ccccc34)cc3ccccc3c21]

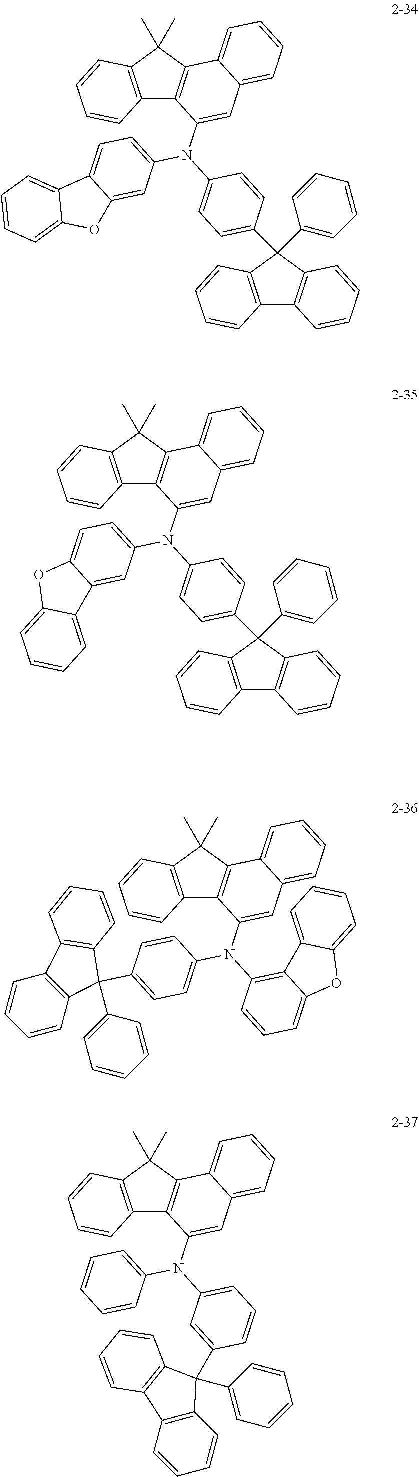 [SMILES: CC1(C)c2ccccc2-c2c(N(c3ccc(C4(c5ccccc5)c5ccccc5-c5ccccc54)cc3)c3ccc4c(c3)oc3ccccc34)cc3ccccc3c21 ; CC1(C)c2ccccc2-c2c(N(c3ccc(C4(c5ccccc5)c5ccccc5-c5ccccc54)cc3)c3ccc4oc5ccccc5c4c3)cc3ccccc3c21 ; CC1(C)c2ccccc2-c2c(N(c3ccc(C4(c5ccccc5)c5ccccc5-c5ccccc54)cc3)c3cccc4oc5ccccc5c34)cc3ccccc3c21 ; CC1(C)c2ccccc2-c2c(N(c3ccccc3)c3cccc(C4(c5ccccc5)c5ccccc5-c5ccccc54)c3)cc3ccccc3c21]

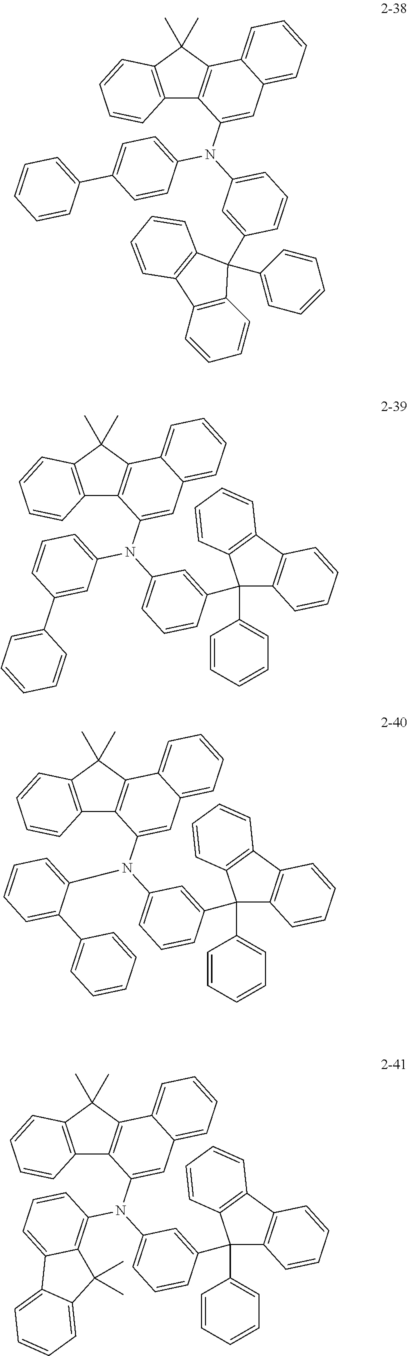 [SMILES: CC1(C)c2ccccc2-c2c(N(c3ccc(-c4ccccc4)cc3)c3cccc(C4(c5ccccc5)c5ccccc5-c5ccccc54)c3)cc3ccccc3c21 ; CC1(C)c2ccccc2-c2c(N(c3cccc(-c4ccccc4)c3)c3cccc(C4(c5ccccc5)c5ccccc5-c5ccccc54)c3)cc3ccccc3c21 ; CC1(C)c2ccccc2-c2c(N(c3cccc(C4(c5ccccc5)c5ccccc5-c5ccccc54)c3)c3ccccc3-c3ccccc3)cc3ccccc3c21 ; CC1(C)c2ccccc2-c2cccc(N(c3cccc(C4(c5ccccc5)c5ccccc5-c5ccccc54)c3)c3cc4ccccc4c4c3-c3ccccc3C4(C)C)c21]

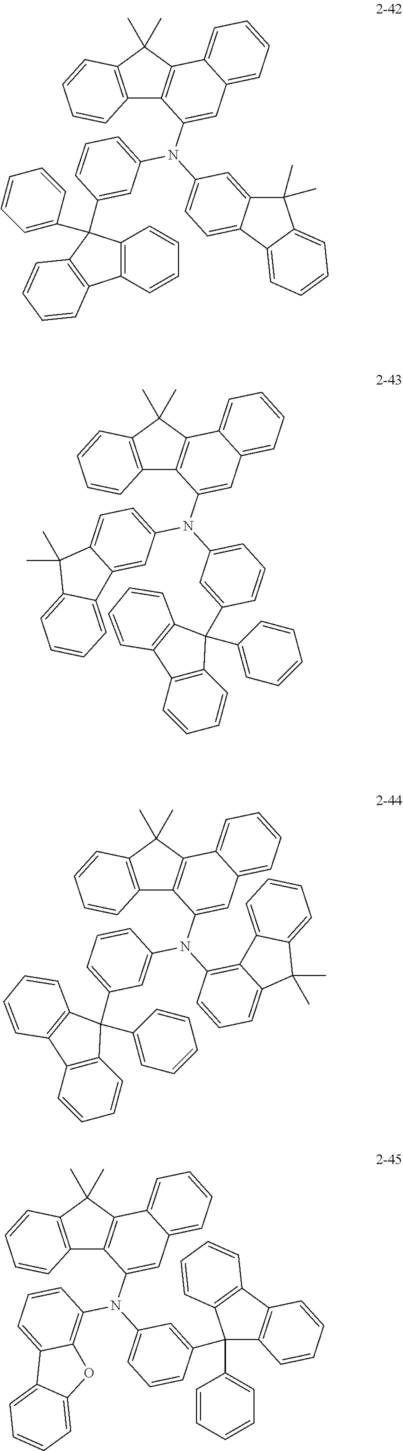

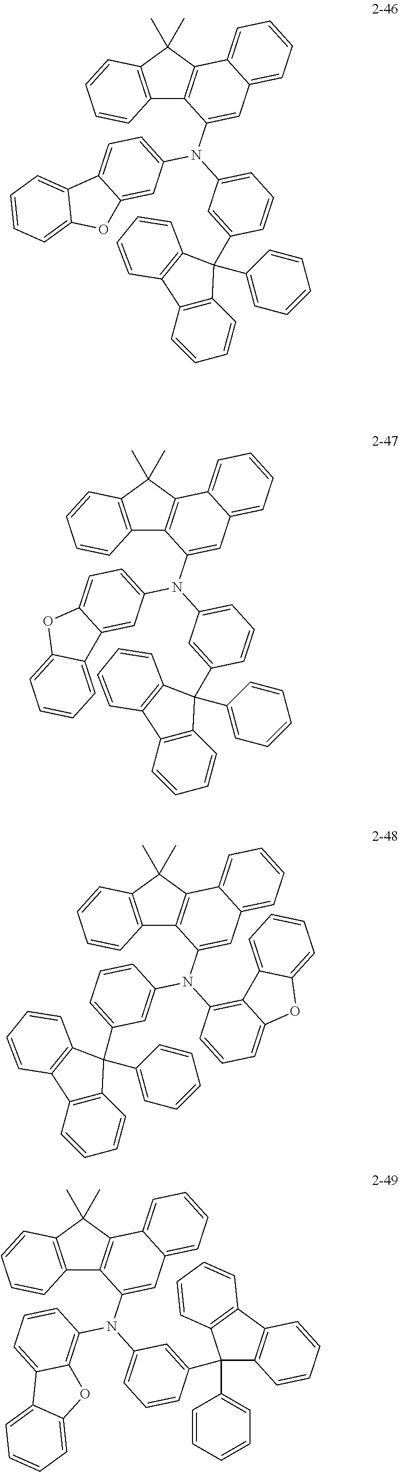

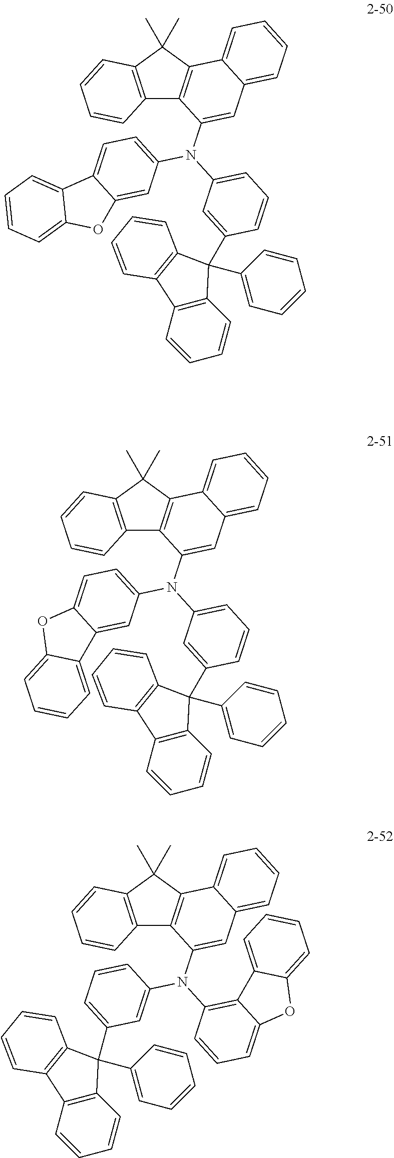 [SMILES: CC1(C)c2ccccc2-c2c(N(c3cccc(C4(c5ccccc5)c5ccccc5-c5ccccc54)c3)c3ccc4c(c3)oc3ccccc34)cc3ccccc3c21 ; CC1(C)c2ccccc2-c2c(N(c3cccc(C4(c5ccccc5)c5ccccc5-c5ccccc54)c3)c3ccc4oc5ccccc5c4c3)cc3ccccc3c21 ; CC1(C)c2ccccc2-c2c(N(c3cccc(C4(c5ccccc5)c5ccccc5-c5ccccc54)c3)c3cccc4oc5ccccc5c34)cc3ccccc3c21]

In an exemplary embodiment of the present specification, an organic material layer including the heterocyclic compound may further include the compound of Chemical Formula 3.

In an exemplary embodiment of the present specification, R21 and R22 may be each independently a substituted or unsubstituted aryl group having 6 to 60 carbon atoms; or a substituted or unsubstituted heteroaryl group having 2 to 60 carbon atoms.

In an exemplary embodiment of the present specification, R21 and R22 may be each independently a substituted or unsubstituted aryl group having 6 to 30 carbon atoms; or a substituted or unsubstituted heteroaryl group having 2 to 30 carbon atoms.

In an exemplary embodiment of the present specification, R21 and R22 may be each independently a substituted or unsubstituted phenyl group; a substituted or unsubstituted biphenyl group; a substituted or unsubstituted terphenyl group; a substituted or unsubstituted naphthyl group; a substituted or unsubstituted dibenzofuran group; or a substituted or unsubstituted dibenzothiophene group.

In an exemplary embodiment of the present specification, R23 and R24 may be each independently hydrogen; deuterium; or a substituted or unsubstituted aryl group having 6 to 60 carbon atoms.

In an exemplary embodiment of the present specification, R23 and R24 may be each independently hydrogen; deuterium; or a substituted or unsubstituted aryl group having 6 to 30 carbon atoms.

In an exemplary embodiment of the present specification, R23 and R24 may be each independently hydrogen; deuterium; or a substituted or unsubstituted aryl group having 6 to 15 carbon atoms.

In an exemplary embodiment of the present specification, R23 and R24 may be each independently hydrogen; deuterium; or a substituted or unsubstituted phenyl group.

In an exemplary embodiment of the present specification, r23 and r24 are each an integer from 0 to 7, and when r23 and r24 are 2 or higher, substituents in the parenthesis are the same as or different from each other.

In an exemplary embodiment of the present specification, when r23 or r24 are each 0, this means that all positions where R23 or R24 may be substituted are hydrogen.

In an exemplary embodiment of the present specification, Chemical Formula 3 may be represented by any one of the following compounds.

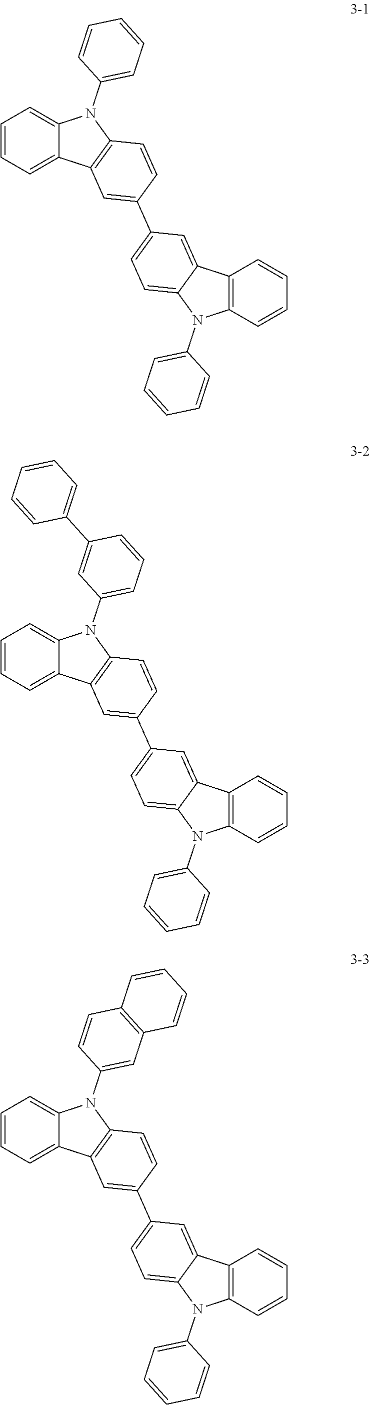

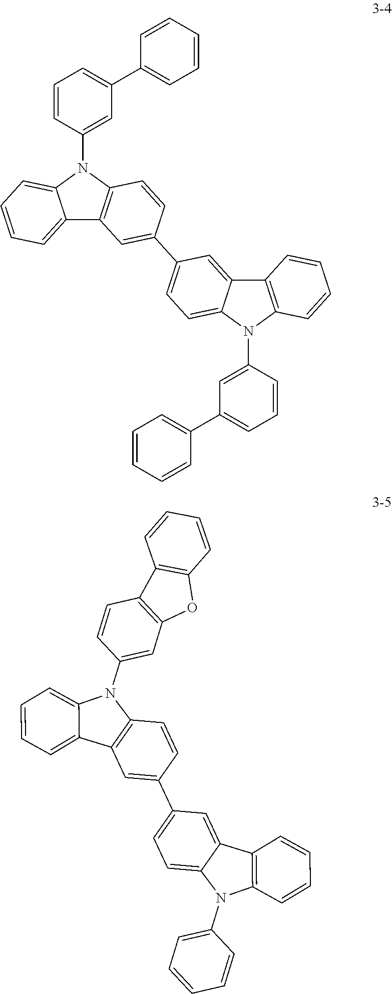

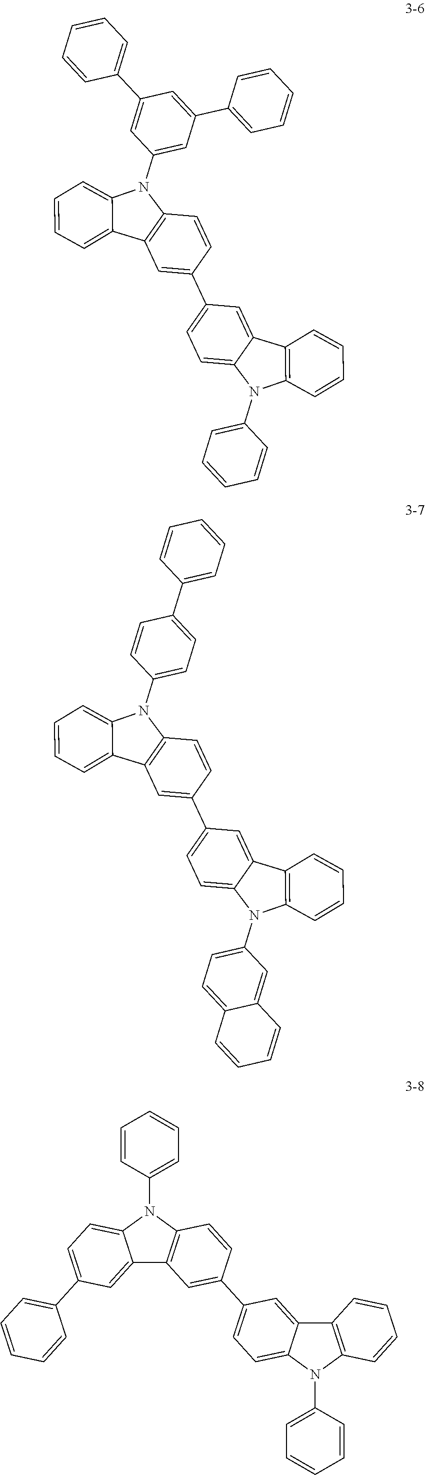

The organic material layer of the organic light emitting device of the present invention may be composed of a single-layered structure, but may be composed of a multi-layered structure in which two or more organic material layers are stacked. For example, the organic light emitting device of the present invention may have a structure including a hole injection layer, a hole transport layer, a light emitting layer, an electron transport layer, an electron injection layer, and the like as organic material layers. However, the structure of the organic light emitting device is not limited thereto, and may include a fewer number of organic material layers.

In an exemplary embodiment of the present specification, the first electrode may be a positive electrode, and the second electrode may be a negative electrode.

In another exemplary embodiment of the present specification, the first electrode may be a negative electrode, and the second electrode may be a positive electrode.

The organic light emitting device according to an exemplary embodiment of the present specification may be manufactured by typical methods and materials for manufacturing an organic light emitting device, except that an organic material layer having one or more layers is formed by using the heterocyclic compound of the above-described Chemical Formula 1.

The heterocyclic compound of Chemical Formula 1 may be formed as an organic material layer by not only a vacuum deposition method, but also a solution application method when an organic light emitting device is manufactured. Here, the solution application method means spin coating, dip coating, inkjet printing, screen printing, a spray method, roll coating, and the like, but is not limited thereto.

In an exemplary embodiment of the present specification, the organic light emitting device may be a blue organic light emitting device, and the heterocyclic compound of Chemical Formula 1 may be used as a material for the blue organic light emitting device. For example, the heterocyclic compound of Chemical Formula 1 may be included in a hole transport layer, an electron blocking layer or a light emitting layer of the blue organic light emitting device.

In another exemplary embodiment of the present specification, the organic light emitting device may be a green organic light emitting device, and the heterocyclic compound of Chemical Formula 1 may be used as a material for the green organic light emitting device. For example, the heterocyclic compound of Chemical Formula 1 may be included in a hole transport layer, an electron blocking layer or a light emitting layer of the green organic light emitting device.

In still another exemplary embodiment of the present specification, the organic light emitting device may be a red organic light emitting device, and the heterocyclic compound of Chemical Formula 1 may be used as a material for the red organic light emitting device. For example, the heterocyclic compound of Chemical Formula 1 may be included in a hole transport layer, an electron blocking layer or a light emitting layer of the red organic light emitting device.

The organic light emitting device of the present invention may further include one or more layers selected from the group consisting of a light emitting layer, a hole injection layer, a hole transport layer, an electron injection layer, an electron transport layer, an electron blocking layer, and a hole blocking layer.

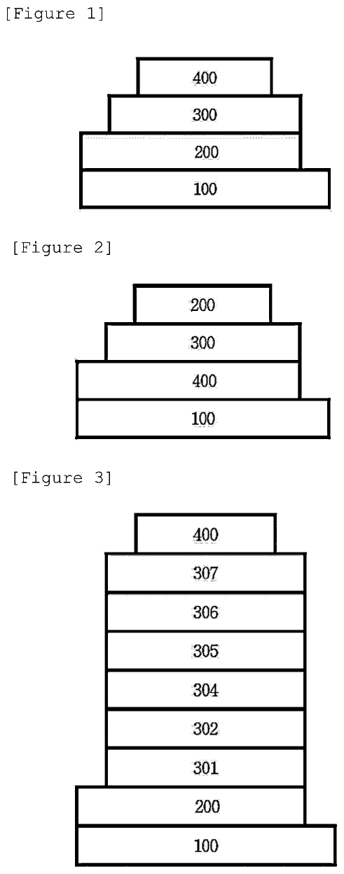

FIGS. 1 to 4 exemplify the stacking sequence of the electrodes and the organic material layer of the organic light emitting device according to an exemplary embodiment of the present specification. However, the scope of the present application is not intended to be limited by these drawings, and the structure of the organic light emitting device known in the art may also be applied to the present application.

According to FIG. 1, an organic light emitting device in which a positive electrode 200, an organic material layer 300, and a negative electrode 400 are sequentially stacked on a substrate 100 is illustrated. However, the organic light emitting device is not limited only to such a structure, and as in FIG. 2, an organic light emitting device in which a negative electrode, an organic material layer, and a positive electrode are sequentially stacked on a substrate may also be implemented.

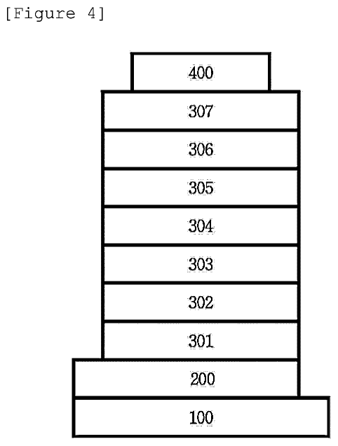

FIGS. 3 and 4 exemplify a case where an organic material layer is a multilayer. The organic light emitting device according to FIG. 3 includes a hole injection layer 301, a hole transport layer 302, a light emitting layer 304, a hole blocking layer 305, an electron transport layer 306 and an electron injection layer 307, and the organic light emitting device according to FIG. 4 includes a hole injection layer 301, a hole transport layer 302, a hole blocking layer 303, a light emitting layer 304, a hole blocking layer 305, an electron transport layer 306 and an electron injection layer 307. However, the scope of the present application is not limited by the stacking structure as described above, and if necessary, the other layers except for the light emitting layer may be omitted, and another necessary functional layer may be further added.

An organic material layer including the heterocyclic compound of Chemical Formula 1 may additionally include other materials, if necessary.

In the organic light emitting device according to an exemplary embodiment of the present specification, materials other than the heterocyclic compound of Chemical Formula 1 will be exemplified below, but these materials are illustrative only and are not for limiting the scope of the present application, and may be replaced with materials publicly known in the art.

As a positive electrode material, materials having a relatively high work function may be used, and a transparent conductive oxide, a metal or a conductive polymer, and the like may be used. Specific examples of the positive electrode material include: a metal such as vanadium, chromium, copper, zinc, and gold, or an alloy thereof; a metal oxide such as zinc oxide, indium oxide, indium tin oxide (ITO), and indium zinc oxide (IZO); a combination of a metal and an oxide, such as ZnO:Al or SnO₂:Sb; a conductive polymer such as poly(3-methylthiophene), poly[3,4-(ethylene-1,2-dioxy)thiophene] (PEDOT), polypyrrole, and polyaniline; and the like, but are not limited thereto.

As a negative electrode material, materials having a relatively low work function may be used, and a metal, a metal oxide, or a conductive polymer, and the like may be used. Specific examples of the negative electrode material include: a metal such as magnesium, calcium, sodium, potassium, titanium, indium, yttrium, lithium, gadolinium, aluminum, silver, tin, and lead, or an alloy thereof; a multi-layer structured material, such as LiF/Al or LiO₂/Al; and the like, but are not limited thereto.

As a hole injection material, a publicly-known hole injection material may also be used, and it is possible to use, for example, a phthalocyanine compound such as copper phthalocyanine disclosed in U.S. Pat. No. 4,356,429 or starburst-type amine derivatives described in the document [Advanced Material, 6, p. 677 (1994)], for example, tris(4-carbazoyl-9-ylphenyl)amine (TCTA), 4,4′,4″-tri[phenyl(m-tolyl)amino]triphenylamine (m-MTDATA), 1,3,5-tris[4-(3-methylphenylphenylamino)phenyl]benzene (m-MTDAPB), polyaniline/dodecylbenzenesulfonic acid or poly(3,4-ethylenedioxythiophene)/poly(4-styrenesulfonate), which is a soluble conductive polymer, polyaniline/camphor sulfonic acid or polyaniline/poly(4-styrenesulfonate), and the like.

As a hole transport material, a pyrazoline derivative, an arylamine-based derivative, a stilbene derivative, a triphenyldiamine derivative, and the like may be used, and a low-molecular weight or polymer material may also be used.

As an electron transport material, it is possible to use an oxadiazole derivative, anthraquinodimethane and a derivative thereof, benzoquinone and a derivative thereof, naphthoquinone and a derivative thereof, anthraquinone and a derivative thereof, tetracyanoanthraquinodimethane and a derivative thereof, a fluorenone derivative, diphenyldicyanoethylene and a derivative thereof, a diphenoquinone derivative, a metal complex of 8-hydroxyquinoline and a derivative thereof, and the like, and a low-molecular weight material and a polymer material may also be used.

As an electron injection material, for example, LiF is representatively used in the art, but the present application is not limited thereto.

As a light emitting material, a red, green, or blue light emitting material may be used, and if necessary, two or more light emitting materials may be mixed and used. In this case, two or more light emitting materials are deposited or used as an individual supply source, or pre-mixed to be deposited and used as one supply source. Further, a fluorescent material may also be used as the light emitting material, but may also be used as a phosphorescent material. As the light emitting material, it is also possible to use alone a material which emits light by combining holes and electrons each injected from a positive electrode and a negative electrode, but materials in which a host material and a dopant material are involved in light emission together may also be used.

When hosts of the light emitting material are mixed and used, the same series of hosts may also be mixed and used, and different series of hosts may also be mixed and used. For example, any two or more materials from N-type host materials or P-type host materials may be selected and used as a host material for a light emitting layer.

The organic light emitting device according to an exemplary embodiment of the present specification may be a top emission type, a bottom emission type, or a dual emission type according to the material to be used.

The heterocyclic compound according to an exemplary embodiment of the present specification may act even in organic electronic devices including organic solar cells, organic photoconductors, organic transistors, and the like, based on the principle similar to those applied to organic light emitting devices.

In addition, it is possible to finely adjust an energy band-gap by introducing various substituents into the structure of Chemical Formula 1, and meanwhile, it is possible to improve characteristics at the interface between organic materials and diversify the use of the material.

In an exemplary embodiment of the present specification, provided is a composition for forming an organic material layer, including the heterocyclic compound.

In an exemplary embodiment of the present specification, the composition for forming an organic material layer may further include the compound of Chemical Formula 2 or 3.

In an exemplary embodiment of the present specification, the composition for forming an organic material layer may include the heterocyclic compound and the compound of Chemical Formula 2 or 3 at a weight ratio of 1:10 to 10:1.

In an exemplary embodiment of the present specification, provided is a method for manufacturing an organic light emitting device, the method including: preparing a substrate; forming a first electrode on the substrate; forming an organic material layer having one or more layers on the first electrode; and forming a second electrode on the organic material layer, in which the forming of the organic material layer includes forming the organic material layer having one or more layers by using the composition for forming an organic material layer, including the heterocyclic compound.

In an exemplary embodiment of the present specification, provided is a method for manufacturing an organic light emitting device, the method including: preparing a substrate; forming a first electrode on the substrate; forming an organic material layer having one or more layers on the first electrode; and forming a second electrode on the organic material layer, in which the forming of the organic material layer includes forming the organic material layer having one or more layers by using the composition for forming an organic material layer, including the heterocyclic compound and the compound of Chemical Formula 2 or 3.

In an exemplary embodiment of the present specification, provided is a method for manufacturing an organic light emitting device, in which the forming of the organic material layer forms the organic material layer by pre-mixing the heterocyclic compound represented by Chemical Formula 1 and the compound represented by Chemical Formula 2 or 3, and using a thermal vacuum deposition method.

The pre-mixing means that before the heterocyclic compound represented by Chemical Formula 1 and the compound represented by Chemical Formula 2 or 3 are deposited onto an organic material layer, the materials are first mixed and the mixture is contained in one common container and mixed.

The pre-mixed material may be referred to as a composition for forming an organic material layer according to an exemplary embodiment of the present specification.

Hereinafter, the present specification will be described in more detail through Examples, but these Examples are provided only for exemplifying the present application, and are not intended to limit the scope of the present application.

### Preparation Examples

### <Preparation Example 1> Preparation of Compound 4

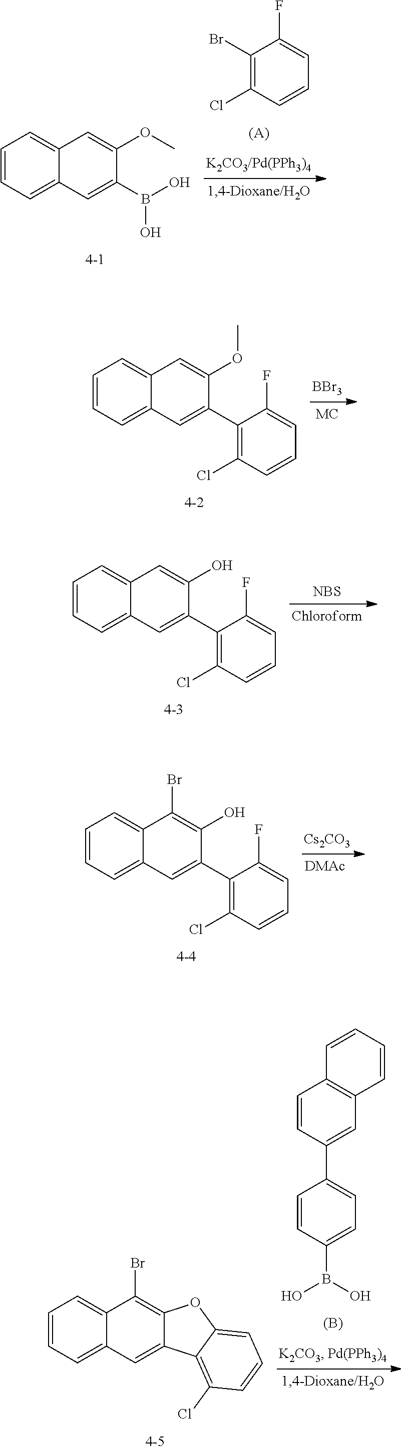

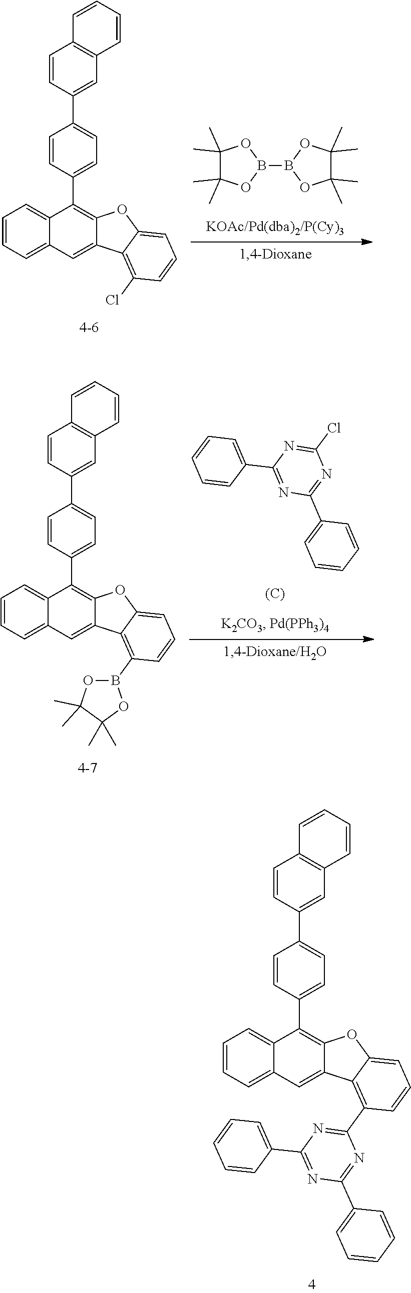

### 1) Preparation of Compound 4-2

1,4-Dioxane (200 ml) and water (40 ml) were put into Compound 4-1 (20 g, 0.099 mol, 1 eq), 2-bromo-1-chloro-3-fluorobenzene (A) (22.8 g, 0.109 mol, 1.1 eq), K₂CO₃ (34.2 g, 0.248 mol, 2.5 eq) and Pd(PPh₃)₄(tetrakis(triphenylphosphine)palladium(0)) (5.7 g, 0.005 mol, 0.05 eq), and the resulting mixture was stirred at 100° C. for 8 hours. After the reaction was terminated by adding water thereto, extraction was performed using methylene chloride (MC) and water. Thereafter, moisture was removed with MgSO₄. The residue was separated by silica gel column to obtain 21 g of Compound 4-2 with a yield of 74%.

### 2) Preparation of Compound 4-3

After Compound 4-2 (21 g, 0.073 mol, 1 eq) was dissolved in MC (210 ml), BBr₃ (36.7 g, 0.146 mol, 2 eq) was slowly added dropwise thereto at 0° C. After the temperature was increased to RT (room temperature), the mixture was stirred for 6 hours. After the reaction was terminated by adding water, extraction was performed using MC and water. Thereafter, moisture was removed with MgSO₄. The residue was separated by silica gel column to obtain 18 g of Compound 4-3 with a yield of 90%.

### 3) Preparation of Compound 4-4

Chloroform (180 ml) was put into Compound 4-3 (18 g, 0.066 mol, 1 eq) and N-bromosuccinimide (NBS) (12.9 g, 0.073 mol, 1.1 q), and the resulting mixture was stirred at RT for 12 hours. After the reaction was terminated by adding water, extraction was performed using MC and water. Thereafter, moisture was removed with MgSO₄. The residue was separated by silica gel column to obtain 18 g of Compound 4-4 with a yield of 78%.

### 4) Preparation of Compound 4-5

Chloroform (180 ml) was put into Compound 4-4 (18 g, 0.066 mol, 1 eq) and NBS (12.9 g, 0.073 mol, 1.1 eq), and the resulting mixture was stirred at 140° C. for 6 hours. After the reaction was terminated by adding water, extraction was performed using MC and water. Thereafter, moisture was removed with MgSO₄. The residue was separated by silica gel column to obtain 13 g of Compound 4-5 with a yield of 77%.

### 5) Preparation of Compound 4-6

1,4-Dioxane (130 ml) and water (35 ml) were put into Compound 4-5 (13 g, 0.039 mol, 1 eq), (4-(naphthalen-2-yl)phenyl)boronic acid (B) (10.7 g, 0.034 mol, 1.1 eq), K₂CO₃ (13.5 g, 0.098 mol, 2.5 eq) and Pd(PPh₃)₄ (2.3 g, 0.002 mol, 0.05 eq), and the resulting mixture was stirred at 100° C. for 8 hours. After the reaction was terminated by adding water, extraction was performed using MC and water. Thereafter, moisture was removed with MgSO₄. The residue was separated by silica gel column to obtain 14 g of Compound 4-6 with a yield of 79%.

### 6) Preparation of Compound 4-7

1,4-Dioxane (140 ml) was put into Compound 4-6 (14 g, 0.031 mol, eq), 4,4,4′,4′,5,5,5′,5′-octamethyl-2,2′-bi(1,3,2-dioxaborolane) (11.7 g, 0.046 mol, 1.5 eq), KOAc (9 g, 0.092 mol, 3 eq), Pd(dba)₂(bis(dibenzylideneacetone)palladium(0)) (1.8 g, 0.003 mol, 0.1 eq) and P(Cy)₃ (tricyclohexylphosphine) (1.7 g, 0.006 mol, 0.2 eq), and the resulting mixture was stirred at 100° C. for 8 hours. After the reaction was terminated by adding water, extraction was performed using MC and water. Thereafter, moisture was removed with MgSO₄. The residue was separated by silica gel column to obtain 10 g of Compound 4-7 with a yield of 59%.

### 7) Preparation of Compound 4

1,4-Dioxane (100 ml) and water (20 ml) were put into Compound 4-7 (10 g, 0.018 mol, 1 eq), 2-chloro-4,6-diphenyl-1,3,5-triazine (C) (5.4 g, 0.020 mol, 1.1 eq), K₂CO₃ (6.3 g, 0.046 mol, 2.5 eq) and Pd(PPh₃)₄ (1 g, 0.0009 mol, 0.05 eq), and the resulting mixture was stirred at 100° C. for 6 hours. After the reaction was terminated by adding water, extraction was performed using MC and water. Thereafter, moisture was removed with MgSO₄. The residue was separated by silica gel column to obtain 8 g of Compound 4 with a yield of 67%.

Compounds were synthesized in the same manner as in Preparation Example 1, except that Intermediates A, B and C of the following Table 1 were used instead of (A), (B) and (C) in Preparation Example 1.

TABLE 1

| Com- | | | |
|---|---|---|---|
| pound | | | |
| No. | Intermediate A | Intermediate B | Intermediate C |
| 1 | 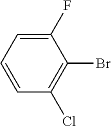 | 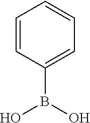 | 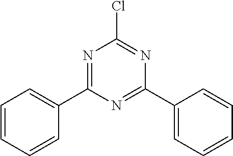 [SMILES: Clc1nc(-c2ccccc2)nc(-c2ccccc2)n1] |
| 9 | 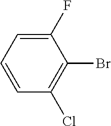 | 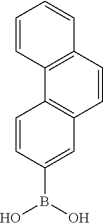 [SMILES: OB(O)c1ccc2c(ccc3ccccc32)c1] | 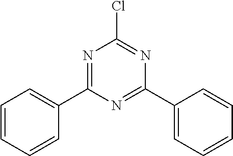 [SMILES: Clc1nc(-c2ccccc2)nc(-c2ccccc2)n1] |
| 21 | 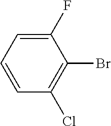 | 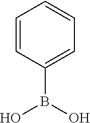 | 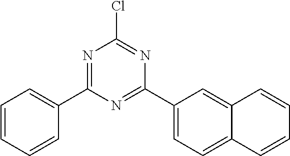 [SMILES: Clc1nc(-c2ccccc2)nc(-c2ccc3ccccc3c2)n1] |
| 26 | 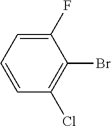 | 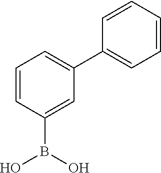 [SMILES: OB(O)c1cccc(-c2ccccc2)c1] | 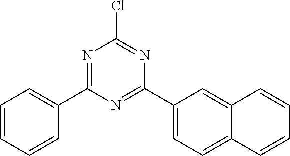 [SMILES: Clc1nc(-c2ccccc2)nc(-c2ccc3ccccc3c2)n1] |
| 41 | 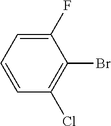 | 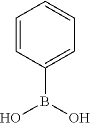 | 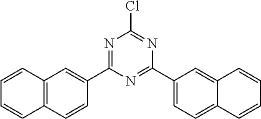 [SMILES: Clc1nc(-c2ccc3ccccc3c2)nc(-c2ccc3ccccc3c2)n1] |
| 78 | 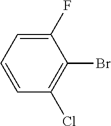 | 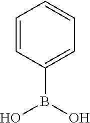 | 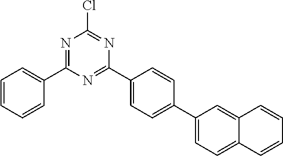 [SMILES: Clc1nc(-c2ccccc2)nc(-c2ccc(-c3ccc4ccccc4c3)cc2)n1] |
| 80 | 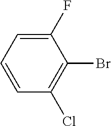 | 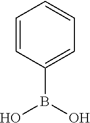 | 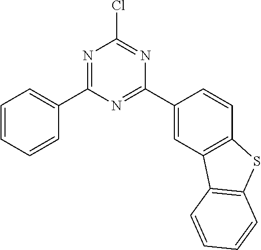 [SMILES: Clc1nc(-c2ccccc2)nc(-c2ccc3sc4ccccc4c3c2)n1] |
| 90 | 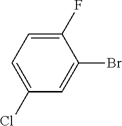 | 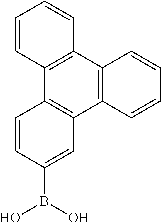 [SMILES: OB(O)c1ccc2c3ccccc3c3ccccc3c2c1] | 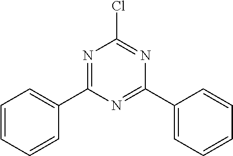 [SMILES: Clc1nc(-c2ccccc2)nc(-c2ccccc2)n1] |
| 101 | 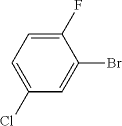 | 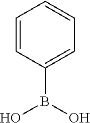 | 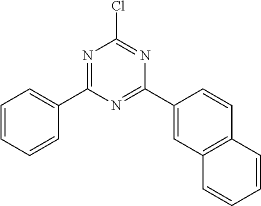 [SMILES: Clc1nc(-c2ccccc2)nc(-c2ccc3ccccc3c2)n1] |
| 119 | 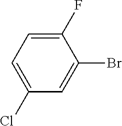 | 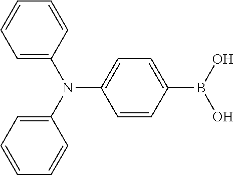 [SMILES: OB(O)c1ccc(N(c2ccccc2)c2ccccc2)cc1] | 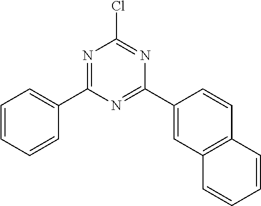 [SMILES: Clc1nc(-c2ccccc2)nc(-c2ccc3ccccc3c2)n1] |
| 137 | 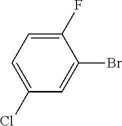 | 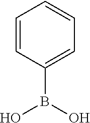 | 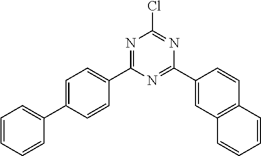 [SMILES: Clc1nc(-c2ccc(-c3ccccc3)cc2)nc(-c2ccc3ccccc3c2)n1] |
| 144 | 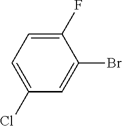 | 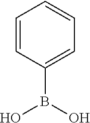 | 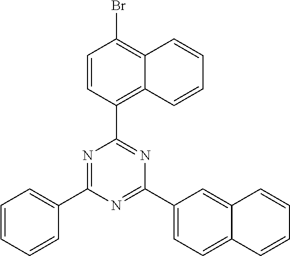 [SMILES: Brc1ccc(-c2nc(-c3ccccc3)nc(-c3ccc4ccccc4c3)n2)c2ccccc12] |
| 160 | 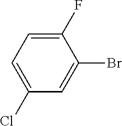 | 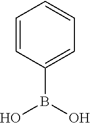 | 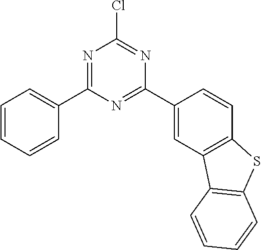 [SMILES: Clc1nc(-c2ccccc2)nc(-c2ccc3sc4ccccc4c3c2)n1] |
| 163 | 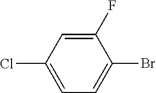 | 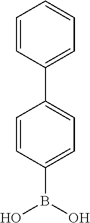 [SMILES: OB(O)c1ccc(-c2ccccc2)cc1] | 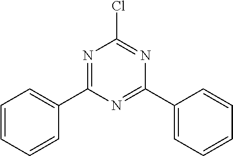 [SMILES: Clc1nc(-c2ccccc2)nc(-c2ccccc2)n1] |
| 200 | 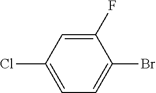 | 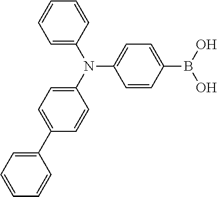 [SMILES: OB(O)c1ccc(N(c2ccccc2)c2ccc(-c3ccccc3)cc2)cc1] | 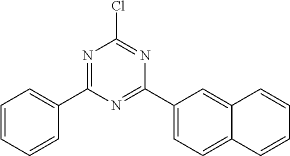 [SMILES: Clc1nc(-c2ccccc2)nc(-c2ccc3ccccc3c2)n1] |
| 205 | 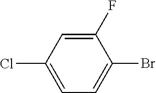 | 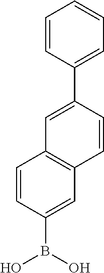 [SMILES: OB(O)c1ccc2cc(-c3ccccc3)ccc2c1] | 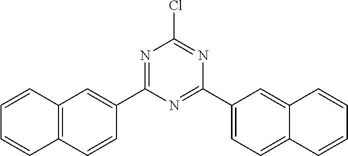 [SMILES: Clc1nc(-c2ccc3ccccc3c2)nc(-c2ccc3ccccc3c2)n1] |
| 237 | 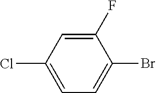 | 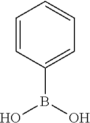 | 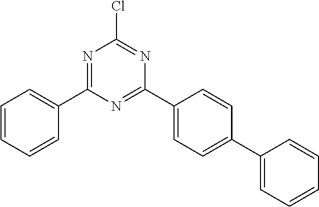 [SMILES: Clc1nc(-c2ccccc2)nc(-c2ccc(-c3ccccc3)cc2)n1] |
| 240 | 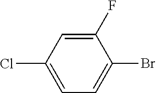 | 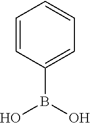 | 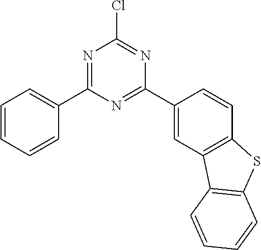 [SMILES: Clc1nc(-c2ccccc2)nc(-c2ccc3sc4ccccc4c3c2)n1] |
| 241 | 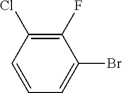 | 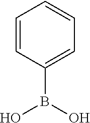 | 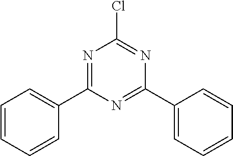 [SMILES: Clc1nc(-c2ccccc2)nc(-c2ccccc2)n1] |
| 262 | 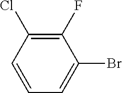 | 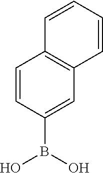 [SMILES: OB(O)c1ccc2ccccc2c1] | 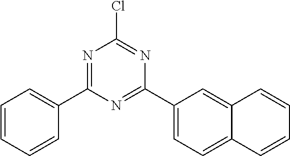 [SMILES: Clc1nc(-c2ccccc2)nc(-c2ccc3ccccc3c2)n1] |
| 295 | 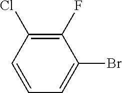 | 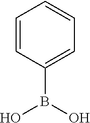 | 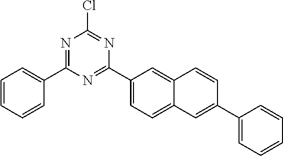 [SMILES: Clc1nc(-c2ccccc2)nc(-c2ccc3cc(-c4ccccc4)ccc3c2)n1] |
| 302 | 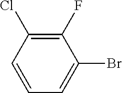 | 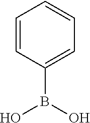 | 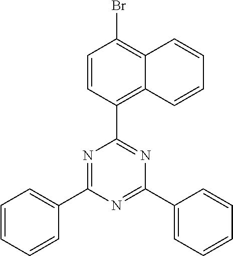 [SMILES: Brc1ccc(-c2nc(-c3ccccc3)nc(-c3ccccc3)n2)c2ccccc12] |
| 324 | 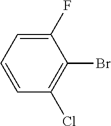 | 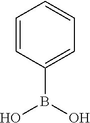 | 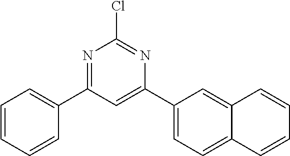 [SMILES: Clc1nc(-c2ccccc2)cc(-c2ccc3ccccc3c2)n1] |
| 341 | 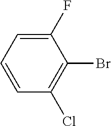 | 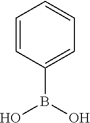 | 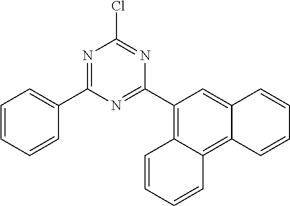 [SMILES: Clc1nc(-c2ccccc2)nc(-c2cc3ccccc3c3ccccc23)n1] |
| 353 | 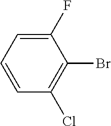 | 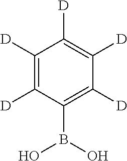 [SMILES: [2H]c1c([2H])c([2H])c(B(O)O)c([2H])c1[2H]] | 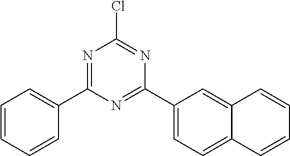 [SMILES: Clc1nc(-c2ccccc2)nc(-c2ccc3ccccc3c2)n1] |
| 375 | 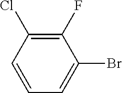 | 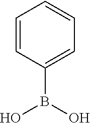 | 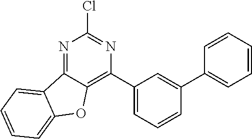 [SMILES: Clc1nc(-c2cccc(-c3ccccc3)c2)c2oc3ccccc3c2n1] |
| 378 | 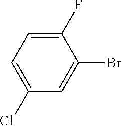 | 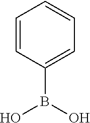 | 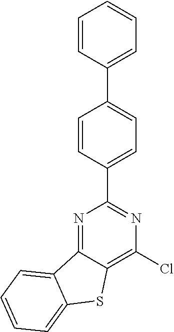 [SMILES: Clc1nc(-c2ccc(-c3ccccc3)cc2)nc2c1sc1ccccc12] |

### <Preparation Example 2> Preparation of Compound 356

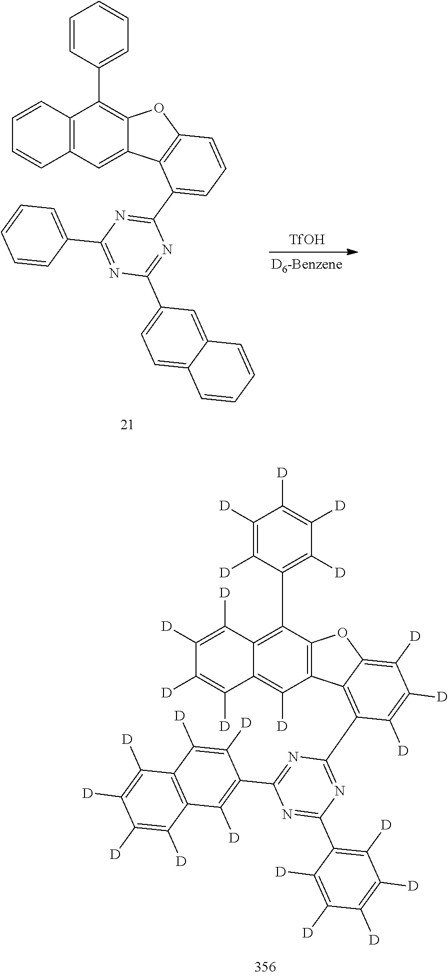

After Compound 21 (8 g, 0.014 mol, 1 eq) prepared in Preparation Example 1, TfOH (3.1 g, 0.021 mol, 1.5 eq) and D₆-benzene (80 ml) were put into a container, the resulting mixture was stirred at 100° C. for 8 hours. After the reaction was terminated by adding water, extraction was performed using MC and water. Thereafter, moisture was removed with MgSO₄. The residue was separated with silica gel column to obtain 7 g of Compound 356 with a yield of 84%.

### <Preparation Example 3> Preparation of Compound 178

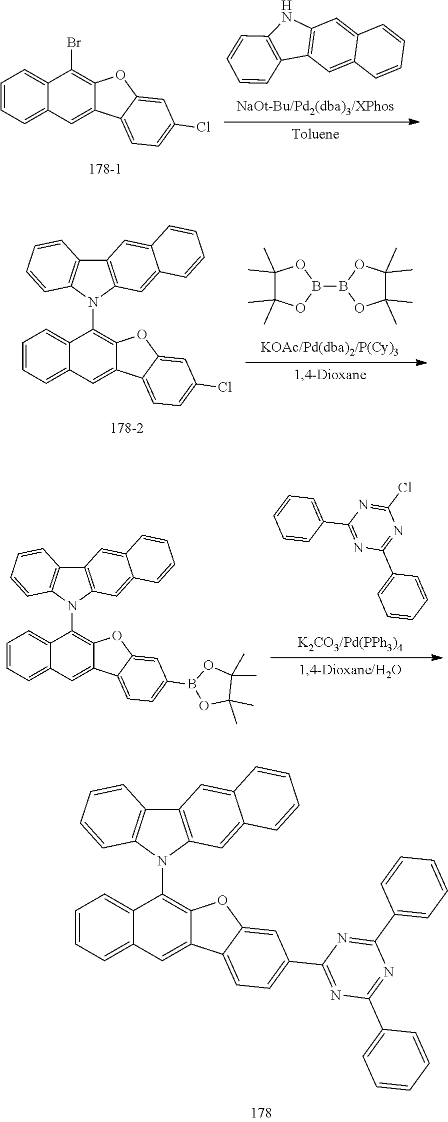

### 1) Preparation of Compound 178-2

Toluene (120 ml) was put into Compound 178-1 (12 g, 0.036 mol, 1 eq), 5H-benzo[b]carbazole (8.6 g, 0.040 mol, 1.1 eq), NaOtBu (5.2 g, 0.054 mol, 1.5 eq), Pd₂(dba)₃ (3.3 g, 0.004 mol, 0.1 eq) and XPhos (3.2 g, 0.007 mol, 0.2 eq), and the resulting mixture was stirred at 100° C. for 12 hours. After the reaction was terminated by adding water, extraction was performed using MC and water. Thereafter, moisture was removed with MgSO₄. The residue was separated by silica gel column to obtain 10 g of Compound 178-2 with a yield of 59%.

### 2) Preparation of Compound 178-3

1,4-Dioxane (150 ml) was put into Compound 178-2 (10 g, 0.021 mol, 1 eq), 4,4,4′,4′,5,5,5′,5′-octamethyl-2,2′-bi(1,3,2-dioxaborolane) (8.1 g, 0.032 mol, 1.5 eq), KOAc (6.3 g, 0.064 mol, 3 eq), Pd(dba)₂ (1.2 g, 0.002 mol, 0.1 eq) and P(Cy)₃ (1.2 g, 0.004 mol, 0.2 eq), and the resulting mixture was stirred at 100° C. for 9 hours. After the reaction was terminated by adding water, extraction was performed using MC and water. Thereafter, moisture was removed with MgSO₄. The residue was separated by silica gel column to obtain 8 g of Compound 178-3 with a yield of 67%.

### 3) Preparation of Compound 178

1,4-Dioxane (100 ml) and water (20 ml) were put into Compound 178-3 (8 g, 0.014 mol, 1 eq), 2-chloro-4,6-diphenyl-1,3,5-triazine (4.2 g, 0.016 mol, 1.1 eq), K₂CO₃ (4.9 g, 0.036 mol, 2.5 eq) and Pd(PPh₃)₄ (0.8 g, 0.0007 mol, 0.05 eq), and the resulting mixture was stirred at 100° C. for 6 hours. After the reaction was terminated by adding water, extraction was performed using MC and water. Thereafter, moisture was removed with MgSO₄. The residue was separated by silica gel column to obtain 7 g of Compound 178 with a yield of 74%.

Compounds were prepared in the same manner as in the Preparation Examples, and the synthesis confirmation results thereof are shown in the following Tables 2 and 3. Table 2 shows the measured values of ¹H NMR (CDCl₃, 200 MHZ), and Table 3 shows the measured values of field desorption mass spectrometry (FD-MS).

TABLE 2

| Compound | ¹H NMR (CDCl₃, 200 MHz) |
|---|---|
| 1 | δ = 8.55 (1H, d), 8.28 (4H, m), 8.08 (1H, d), 7.75 (1H, |
| | d), 7.62~7.41 (16H, m) |
| 4 | δ = 8.55 (1H, d), 8.28 (4H, m), 8.00 (4H, m), 7.75 (2H, |
| | m), 7.62~7.41 (14H, m), 7.25 (4H, m) |
| 9 | δ = 8.99 (1H, d), 8.93 (1H, d), 8.55 (1H, d), 8.34 (1H, |
| | s), 8,28 (4H, m), 8.12 (3H, m), 7.71 (5H, m), 7.45 (11H, |
| | m) |
| 21 | δ = 9.09 (1H, s), 8.49 (2H, m), 8.28 (2H, m), 8.00 (4H, |
| | m), 7.75 (1H, d), 7.62~7.41 (15H, m) |
| 26 | δ = 9.09 (1H, s), 8.55 (2H, m), 8.28 (2H, m), 7.92 (4H, |
| | m), 7.70 (2H, m), 7.62~7.41 (18H, m) |
| 41 | δ = 9.09 (2H, m), 8.49 (3H, m), 8.00 (7H, m), 7.75 (1H, |
| | d), 7.62~7.41 (14H, m) |
| 78 | δ = 8.55 (1H, d), 8.28 (2H, m), 7.85 (6H, m), 7.73 (2H, |
| | m), 7.62~7.41 (16H, m), 7.25 (2H, m) |
| 80 | δ = 8.55 (1H, d), 8.45 (1H, d), 8.28 (2H, m), 8.08 (1H, |
| | d), 7.98 (2H, m), 7.80 (3H, m), 7.62~7.41 (15H, m) |
| 90 | δ = 9.15 (1H, d), 8.93 (2H, m), 8.55 (1H, d), 8,28 (4H, |
| | m), 8.08 (5H, m), 7.88 (5H, m), 7.72 (2H, m), 7.45 (9H, |
| | m) |
| 101 | δ = 9.09 (1H, s), 8.49 (2H, m), 8.28 (2H, m), 8.00 (4H, |
| | m), 7.81 (1H, d), 7.72 (2H, m), 7.55 (13H, m) |
| 119 | δ = 9.09 (1H, s), 8.49 (2H, m), 8.28 (2H, m), 8.00 (4H, |
| | m), 7.81 (1H, d), 7.72 (2H, m), 7.55 (10H, m), 7.20 (4H, |
| | m), 6.81 (2H, m), 6.69 (6H, m) |
| 137 | δ = 9.09 (1H, s), 8.55 (2H, m), 8.08 (3H, m), 7.92 (4H, |
| | m), 7.71 (2H, m), 7.59~7.41 (15H, m), 7.25 (2H, m) |
| 144 | δ = 9.09 (1H, s), 8.49 (4H, m), 8.28 (2H, m), 8.01 (6H, |
| | m), 7.81 (1H, d), 7.72 (2H, m), 7.59~7.41 (15H, m) |
| 160 | δ = 9.55 (1H, d), 8.45 (1H, d), 8.28 (2H, m), 8.08 (1H, |
| | d), 7.98 (2H, m), 7.86 (3H, m), 7.71 (2H, m), |
| | 7.55~7.41 (13H, m) |
| 163 | δ = 8.55 (1H, d), 8.28 (4H, m), 8.08 (1H, d), 7.95 (1H, |
| | d), 7.75 (1H, d), 7.64 (1H, s), 7.55~7.41 (14H, m), |
| | 7.25 (4H, m) |
| 178 | δ = 8.55 (1H, d), 8.28 (4H, m), 8.16 (4H, m), 7.95 (2H, |
| | m), 7.67 (5H, m), 7.55~25 (12H, m) |
| 200 | δ = 9.09 (1H, s), 8.55 (2H, m), 8.28 (2H, m), 8.00 (5H, |
| | m), 7.75 (1H, d), 7.64~7.41 (18H, m), 7.20 (2H, m), |
| | 6.81 (1H, t), 6.69 (6H, m) |
| 205 | δ = 9.09 (2H, m), 8.49 (3H, m), 7.92 (10H, m), 7.73 (3H, |
| | m), 7.64~7.41 (15H, m) |
| 237 | δ = 8.55 (1H, d), 8.28 (2H, m), 8.08 (1H, d), 7.95 (1H, |
| | d), 7.85 (2H, m), 7.75 (1H, d), 7.64 (1H, s), |
| | 7.55~7.41 (16H, m), 7.25 (2H, m) |
| 240 | δ = 8.66 (1H, d), 8.45 (1H, d), 8.28 (2H, m), 8.08 (1H, |
| | d), 8.00 (3H, m), 7.80 (3H, m), 7.64 (1H, s), |
| | 7.55~7.41(13H, m) |
| 241 | δ = 8.55 (1H, d), 8.28 (4H, m), 8.08 (1H, d), 7.81 (2H, |
| | m), 7.55~7.38 (15H, m) |
| 262 | δ = 9.09 (1H, s), 8.55 (2H, m), 8.28 (2H, m), 7.85 (9H, |
| | m), 7.73 (1H, d), 7.59~7.38 (12H, m) |
| 295 | δ = 9.09 (1H, s), 8.49 (2H, m), 8.28 (2H, m), 8.08 (1H, |
| | d), 7,85 (4H, m), 7.73 (1H, d), 7.55~7.38 (18H, m) |
| 302 | δ = 8.55 (3H, m), 8.28 (4H, m), 8.08 (1H, d), 8.01 (2H, |
| | m), 7.85 (2H, m), 7.55~7.38 (17H, m) |
| 324 | δ = 8.55 (1H, d), 8.34 (1H, s), 8.23 (1H, s), 8.00 (5H, |
| | m), 7.75 (3H, m), 7.62~7.41 (15H, m) |
| 341 | δ = 8.93 (2H, m), 8.55 (1H, d), 8,28 (2H, m), 8.12 (3H, |
| | m), 7.82 (6H, m), 7.62~7.41 (13H, m) |
| 353 | δ = 9.09 (1H, s), 8.55 (2H, m), 8.28 (2H, m), 7.92 (4H, |
| | m), 7,75 (1H, d), 7.62~7.41 (10H, m) |
| 356 | δ = — |
| 375 | δ = 8.55 (1H, d), 8.08 (1H, d), 7.66 (6H, m), |
| | 7.57~7.32 (18H, m) |
| 378 | δ = 8.55 (1H, d), 7.98 (3H, m), 7.81 (3H, m), 7.72 (2H, |
| | m), 7.55~7.41 (15H, m), 7.25 (2H, m) |

TABLE 3

| Compound | FD-MS |
|---|---|
| 1 | m/z = 525.18 |
| 4 | m/z = 651.23 |
| 9 | m/z = 625.22 |
| 21 | m/z = 575.20 |
| 26 | m/z = 651.23 |
| 41 | m/z = 625.22 |
| 78 | m/z = 651.23 |
| 80 | m/z = 631.17 |
| 90 | m/z = 675.23 |
| 101 | m/z = 575.20 |
| 119 | m/z = 742.27 |
| 137 | m/z = 651.23 |
| 144 | m/z = 701.25 |
| 160 | m/z = 631.17 |
| 163 | m/z = 601.22 |
| 178 | m/z = 664.23 |
| 200 | m/z = 818.30 |
| 205 | m/z = 751.26 |
| 237 | m/z = 601.22 |
| 240 | m/z = 631.17 |
| 241 | m/z = 525.18 |
| 262 | m/z = 625.22 |
| 295 | m/z = 651.23 |
| 302 | m/z = 651.23 |
| 324 | m/z = 574.20 |
| 341 | m/z = 625.22 |
| 353 | m/z = 580.23 |
| 356 | m/z = 600.36 |
| 375 | m/z = 614.20 |
| 378 | m/z = 630.18 |

### Experimental Examples

### Experimental Example 1

### 1) Manufacture of Organic Light Emitting Device

A glass substrate, in which indium tin oxide (ITO) was thinly coated to have a thickness of 1,500 Å, was ultrasonically distilled water. When the washing with distilled water was finished, the glass substrate was ultrasonically washed with a solvent such as acetone, methanol, and isopropyl alcohol, dried and then subjected to ultraviolet ozone (UVO) treatment for 5 minutes using UV in an ultraviolet (UV) washing machine. Thereafter, the substrate was transferred to a plasma washing machine (PT), and then was subjected to plasma treatment in a vacuum state for an ITO work function and in order to remove a residual film, and was transferred to a thermal deposition apparatus for organic deposition.

As the common layers, the hole injection layer 4,4′,4″-tris[2-naphthyl(phenyl)amino]triphenylamine (2-TNATA) and the hole transport layer N,N′-di(1-naphthyl)-N,N′-diphenyl-(1,1′-biphenyl)-4,4′-diamine (NPB) were formed on the ITO transparent electrode (positive electrode).

A light emitting layer was thermally vacuum deposited thereon as follows. The light emitting layer was deposited to have a thickness of 400 Å by depositing the compound described in the following Table 4 as a red host and doping the host with an Ir compound at 3 wt % using (piq)₂(Ir)_{(acac)} as a red phosphorescent dopant. Thereafter, Bphen as a hole blocking layer was deposited to have a thickness of 30 Å, and Alq₃ as an electron transport layer was deposited to have a thickness of 250 Å thereon. Finally, lithium fluoride (LiF) was deposited to have a thickness of 10 Å on the electron transport layer to form an electron injection layer, and then an aluminum (Al) negative electrode was deposited to have a thickness of 1,200 Å on the electron injection layer to form a negative electrode, thereby manufacturing an organic electroluminescence device.

Meanwhile, all the organic compounds required for manufacturing an OLED device were subjected to vacuum sublimed purification under 10-8 to 10-6 torr for each material, and used for the manufacture of OLED.

### 2) Evaluation of Organic Light Emitting Device

For the organic electroluminescence device manufactured as described above, electroluminescence (EL) characteristics were measured by M7000 manufactured by McScience Inc., and based on the measurement result thereof, service life (T90) was measured by a service life measurement device (M6000) manufactured by McScience Inc., when the reference luminance was 6,000 cd/m. The results of measuring the driving voltage, light emitting efficiency, color coordinate (CIE) and service life of the organic light emitting device manufactured according to the present invention are shown in the following Table 4.

TABLE 4

| | | | Light | | |
|---|---|---|---|---|---|
| | | Driving | emitting | | Service |
| | | voltage | efficiency | CIE | life |
| | Compound | (V) | (cd/A) | (x, y) | (T90) |
| Example 1 | 1 | 5.1 | 52.8 | (0.68, 0.32) | 101 |
| Example 2 | 4 | 5.1 | 52.5 | (0.68, 0.32) | 110 |
| Example 3 | 9 | 5.1 | 50.8 | (0.68, 0.32) | 112 |
| Example 4 | 21 | 5.0 | 51.7 | (0.68, 0.32) | 107 |
| Example 5 | 26 | 5.0 | 50.9 | (0.68, 0.32) | 105 |
| Example 6 | 41 | 5.1 | 51.5 | (0.68, 0.32) | 114 |
| Example 7 | 78 | 5.0 | 53.5 | (0.68, 0.32) | 109 |
| Example 8 | 80 | 5.1 | 52.6 | (0.68, 0.32) | 117 |
| Example 9 | 90 | 5.0 | 51.2 | (0.68, 0.32) | 106 |
| Example 10 | 101 | 5.1 | 52.7 | (0.68, 0.32) | 113 |
| Example 11 | 119 | 5.3 | 49.9 | (0.68, 0.32) | 100 |
| Example 12 | 137 | 5.0 | 53.4 | (0.68, 0.32) | 125 |
| Example 13 | 144 | 5.4 | 46.7 | (0.68, 0.32) | 84 |
| Example 14 | 160 | 5.1 | 53.2 | (0.68, 0.32) | 107 |
| Example 15 | 163 | 5.1 | 52.0 | (0.68, 0.32) | 123 |
| Example 16 | 178 | 5.3 | 49.7 | (0.68, 0.32) | 98 |
| Example 17 | 200 | 5.2 | 51.6 | (0.68, 0.32) | 120 |
| Example 18 | 205 | 5.0 | 52.3 | (0.68, 0.32) | 105 |
| Example 19 | 237 | 5.1 | 53.7 | (0.68, 0.32) | 118 |
| Example 20 | 240 | 5.1 | 52.3 | (0.68, 0.32) | 126 |
| Example 21 | 241 | 5.3 | 48.1 | (0.68, 0.32) | 99 |
| Example 22 | 262 | 5.3 | 48.5 | (0.68, 0.32) | 97 |
| Example 23 | 295 | 5.3 | 47.9 | (0.68, 0.32) | 95 |
| Example 24 | 302 | 5.4 | 46.1 | (0.68, 0.32) | 80 |
| Example 25 | 324 | 5.1 | 51.5 | (0.68, 0.32) | 110 |
| Example 26 | 341 | 5.1 | 52.0 | (0.68, 0.32) | 103 |
| Example 27 | 353 | 5.1 | 51.4 | (0.68, 0.32) | 119 |
| Example 28 | 356 | 5.0 | 53.3 | (0.68, 0.32) | 125 |
| Example 29 | 375 | 5.3 | 49.0 | (0.68, 0.32) | 95 |
| Example 30 | 378 | 5.1 | 52.4 | (0.68, 0.32) | 117 |
| Comparative | H1 | 5.8 | 44.5 | (0.68, 0.32) | 63 |
| Example 1 | | | | | |
| Comparative | H2 | 5.9 | 43.9 | (0.68, 0.32) | 65 |
| Example 2 | | | | | |
| Comparative | H3 | 5.9 | 44.6 | (0.68, 0.32) | 61 |
| Example 3 | | | | | |
| Comparative | H4 | 6.0 | 44.1 | (0.68, 0.32) | 70 |
| Example 4 | | | | | |
| Comparative | H5 | 6.1 | 42.2 | (0.68, 0.32) | 54 |
| Example 5 | | | | | |
| Comparative | H6 | 6.1 | 42.0 | (0.68, 0.32) | 51 |
| Example 6 | | | | | |
| Comparative | H7 | 6.1 | 42.4 | (0.68, 0.32) | 67 |
| Example 7 | | | | | |
| Comparative | H8 | 6.3 | 41.7 | (0.68, 0.32) | 60 |
| Example 8 | | | | | |
| Comparative | H9 | 5.8 | 45.5 | (0.68, 0.32) | 71 |
| Example 9 | | | | | |
| Comparative | H10 | 5.8 | 45.0 | (0.68, 0.32) | 70 |
| Example 10 | | | | | |
| Comparative | H11 | 6.1 | 44.8 | (0.68, 0.32) | 62 |
| Example 11 | | | | | |
| Comparative | H12 | 5.8 | 45.1 | (0.68, 0.32) | 73 |
| Example 12 | | | | | |

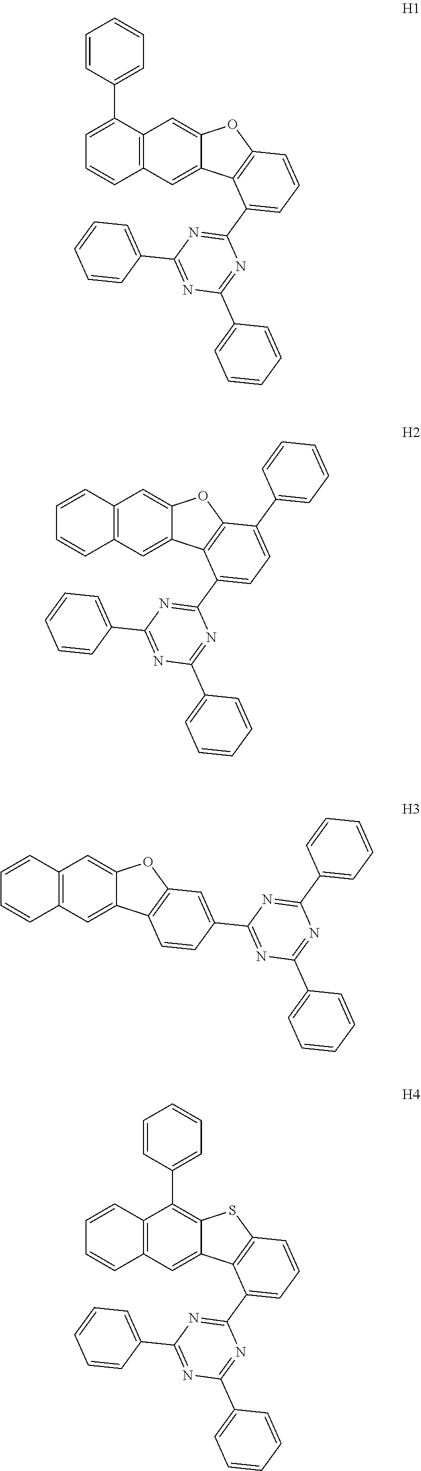 [SMILES: c1ccc(-c2nc(-c3ccccc3)nc(-c3cccc4oc5cc6c(-c7ccccc7)cccc6cc5c34)n2)cc1 ; c1ccc(-c2nc(-c3ccccc3)nc(-c3ccc(-c4ccccc4)c4oc5cc6ccccc6cc5c34)n2)cc1 ; c1ccc(-c2nc(-c3ccccc3)nc(-c3ccc4c(c3)oc3cc5ccccc5cc34)n2)cc1 ; c1ccc(-c2nc(-c3ccccc3)nc(-c3cccc4sc5c(-c6ccccc6)c6ccccc6cc5c34)n2)cc1]

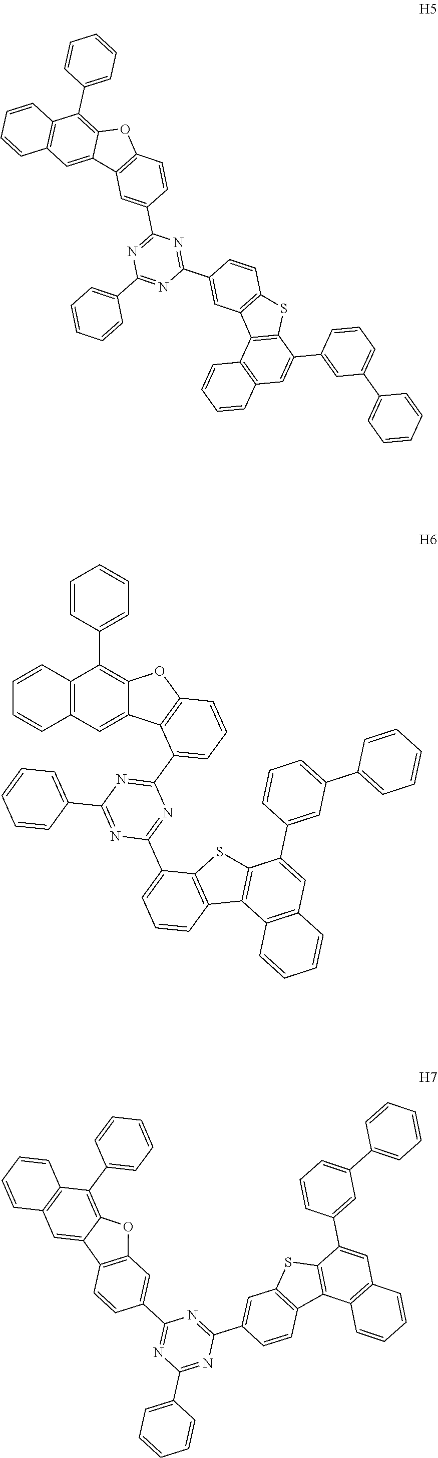 [SMILES: c1ccc(-c2cccc(-c3cc4ccccc4c4c3sc3ccc(-c5nc(-c6ccccc6)nc(-c6ccc7oc8c(-c9ccccc9)c9ccccc9cc8c7c6)n5)cc34)c2)cc1 ; c1ccc(-c2cccc(-c3cc4ccccc4c4c3sc3c(-c5nc(-c6ccccc6)nc(-c6cccc7oc8c(-c9ccccc9)c9ccccc9cc8c67)n5)cccc34)c2)cc1 ; c1ccc(-c2cccc(-c3cc4ccccc4c4c3sc3cc(-c5nc(-c6ccccc6)nc(-c6ccc7c(c6)oc6c(-c8ccccc8)c8ccccc8cc67)n5)ccc34)c2)cc1]

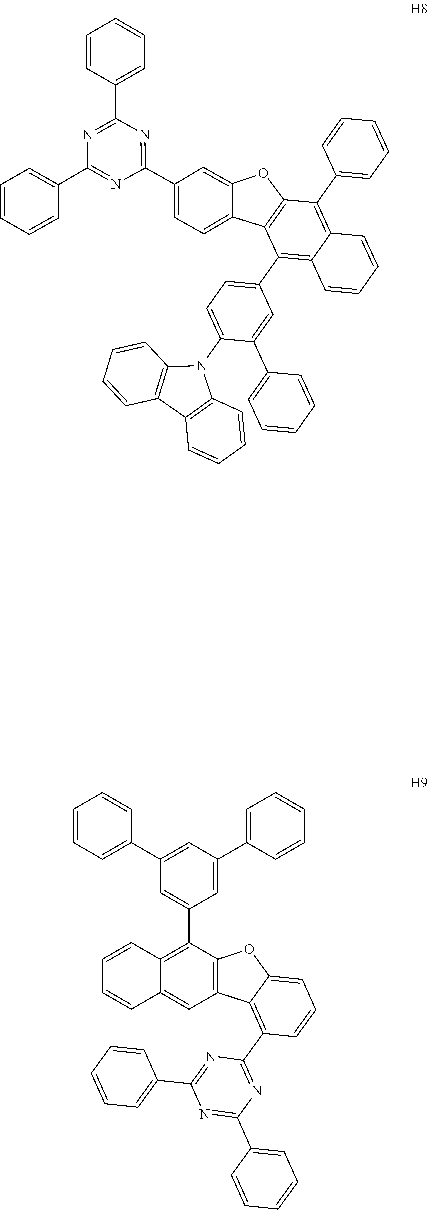 [SMILES: c1ccc(-c2nc(-c3ccccc3)nc(-c3ccc4c(c3)oc3c(-c5ccccc5)c5ccccc5c(-c5ccc(-n6c7ccccc7c7ccccc76)c(-c6ccccc6)c5)c34)n2)cc1 ; c1ccc(-c2cc(-c3ccccc3)cc(-c3c4ccccc4cc4c3oc3cccc(-c5nc(-c6ccccc6)nc(-c6ccccc6)n5)c34)c2)cc1]

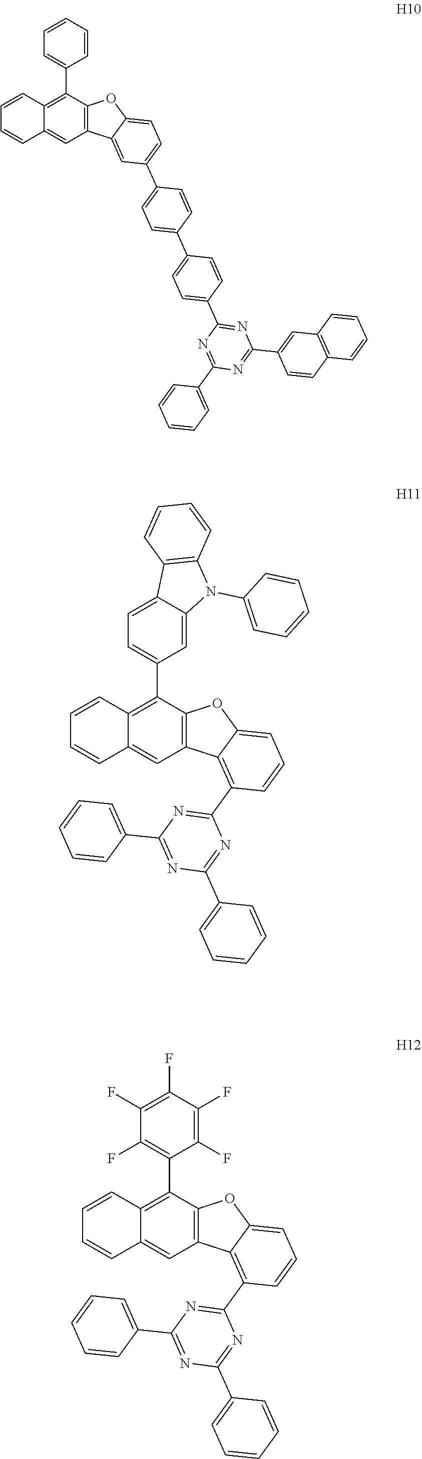 [SMILES: c1ccc(-c2nc(-c3ccc(-c4ccc(-c5ccc6oc7c(-c8ccccc8)c8ccccc8cc7c6c5)cc4)cc3)nc(-c3ccc4ccccc4c3)n2)cc1 ; c1ccc(-c2nc(-c3ccccc3)nc(-c3cccc4oc5c(-c6ccc7c8ccccc8n(-c8ccccc8)c7c6)c6ccccc6cc5c34)n2)cc1 ; Fc1c(F)c(F)c(-c2c3ccccc3cc3c2oc2cccc(-c4nc(-c5ccccc5)nc(-c5ccccc5)n4)c23)c(F)c1F]

As can be seen from the results in Table 4, it could be confirmed that the organic light emitting device using a host material of the present invention has a low driving voltage and remarkably improved light emitting efficiency and service life compared to the Comparative Examples. The heterocyclic compound according to the present application has a molecular weight and a band gap suitable for use in the light emitting layer of the organic light emitting device while having high thermal stability. A suitable molecular weight facilitates the formation of the light emitting layer of the organic light emitting device, and a suitable band gap prevents the electrons and holes of the light emitting layer from flowing out to help form an effective recombination zone. In addition, a heterocyclic compound having transfer properties of electrons substituted at an appropriate position solves a hole blocking phenomenon caused by a dopant more than a compound substituted at other positions, and as can be seen from the above device evaluation, it is determined that the compound of the present invention brought about excellence in all aspects of driving, efficiency and service life compared to the Comparative Examples.

Specifically, Example 1 and Comparative Examples 1 and 2 differ only in the substitution position of the phenyl group, and it can be seen when the phenyl group is substituted at position 6 as in the present invention, the driving voltage is lower, the light emitting efficiency is higher, and the service life is longer than when the phenyl group is substituted at other positions.

Furthermore, Example 1 and Comparative Example 4 have different core structures, and it can be seen that when the compound of the present invention including naphthobenzofuran rather than including naphthobenzothiophene is used in an organic light emitting device, all of the driving voltage, light emitting efficiency and service life are excellent.

Comparative Examples 5 to 7 include naphthobenzothiophene as a substituent of triazine, and it can be confirmed that when Comparative Examples 5 to 7 are compared to Examples 14, 8 and 20, respectively, the compound of the present invention including dibenzothiophene instead of naphthobenzothiophene provides the excellent performances of low driving voltage, high light emitting efficiency and long service life as a material for an organic light emitting device.

In Comparative Example 8, naphthobenzofuran has three substituents, and it can be seen that the driving voltage, light emitting efficiency and service life all deteriorated compared to when the compound of the present invention having two substituents is used.

Comparative Example 10 is an organic light emitting device using a compound including a biphenylene group as a linker between naphthobenzofuran and an azine group, and it can be confirmed that when Comparative Example 10 is compared to Example 10 which does not include a linker, the driving voltage is higher, the light emitting efficiency is lower, and particularly, the service life of Example 10 is 1.6-fold longer than that of Comparative Example 10.

Finally, in Comparative Examples 9, 11 and 12, compounds different from the characteristics of (L1) l1-Z in Chemical Formula 1 of the present invention are used, and it can be seen that when Comparative Examples 9, 11 and 12 are compared to Example 1, Example 1 is better than Comparative Examples 9, 11 and 12 in terms of driving voltage, light emitting efficiency, and service life.

### Experimental Example 2

### 1) Manufacture of Organic Light Emitting Device

A glass substrate, in which indium tin oxide (ITO) was thinly coated to have a thickness of 1,500 Å, was ultrasonically washed with distilled water. When the washing with distilled water was finished, the glass substrate was ultrasonically washed with a solvent such as acetone, methanol, and isopropyl alcohol, dried and then subjected to ultraviolet ozone (UVO) treatment for 5 minutes using UV in an ultraviolet (UV) washing machine. Thereafter, the substrate was transferred to a plasma washing machine (PT), and then was subjected to plasma treatment in a vacuum state for an ITO work function and in order to remove a residual film, and was transferred to a thermal deposition apparatus for organic deposition.

A hole injection layer 4,4′,4″-tris[2-naphthyl(phenyl)amino]triphenylamine (2-TNATA), a hole transport layer N,N′-di(1-naphthyl)-N,N′-diphenyl-(1,1′-biphenyl)-4,4′-diamine (NPB) and an electron blocking layer cyclohexylidenebis[N,N-bis(4-methylphenyl)benzenamine] (TAPC), which are common layers, were formed on the ITO transparent electrode (positive electrode).

A light emitting layer was thermally vacuum deposited thereon as follows. The light emitting layer was deposited to have a thickness of 400 Å by depositing two types of the compounds described in the following Table 5 at the weight ratio described in the following Table 5 from a single supply source and doping the host with an Ir compound at 3 wt % using (piq)₂(Ir)(acac) as a red phosphorescent dopant. Thereafter, Bphen as a hole blocking layer was deposited to have a thickness of 30 Å, and TPBI as an electron transport layer was deposited to have a thickness of 250 Å thereon. Finally, lithium fluoride (LiF) was deposited to have a thickness of 10 Å on the electron transport layer to form an electron injection layer, and then an aluminum (Al) negative electrode was deposited to have a thickness of 1,200 Å on the electron injection layer to form a negative electrode, thereby manufacturing an organic electroluminescence device.

Meanwhile, all the organic compounds required for manufacturing an OLED device were subjected to vacuum sublimed purification under 10-8 to 10-6 torr for each material, and used for the manufacture of OLED.

### 2) Evaluation of Organic Light Emitting Device

For the organic electroluminescence device manufactured as described above, electroluminescence (EL) characteristics were measured by M7000 manufactured by McScience Inc., and based on the measurement result thereof, service life (T90) was measured by a service life measurement device (M6000) manufactured by McScience Inc., when the reference luminance was 6,000 cd/m². The results of measuring the driving voltage, light emitting efficiency, color coordinate (CIE) and service life of the organic light emitting device manufactured according to the present invention are shown in the following Table 5.

TABLE 5

| | | | Driving | Light emitting | | Service |
|---|---|---|---|---|---|---|
| | Compound | Ratio | voltage | efficiency | CIE | life |
| | (P:N) | (P:N) | (V) | (cd/A) | (x, y) | (T90) |
| Example 31 | 2-6:1 | 1:3 | 4.7 | 53.3 | (0.68, 0.32) | 138 |
| Example 32 | 2-6:1 | 1:2 | 4.6 | 54.2 | (0.68, 0.32) | 150 |
| Example 33 | 2-6:1 | 1:1 | 4.5 | 55.1 | (0.68, 0.32) | 152 |
| Example 34 | 2-6:1 | 2:1 | 4.6 | 53.7 | (0.68, 0.32) | 149 |
| Example 35 | 2-6:1 | 3:1 | 4.7 | 52.2 | (0.68, 0.32) | 140 |
| Example 36 | 2-15:4 | 1:1 | 4.5 | 55.5 | (0.68, 0.32) | 151 |
| Example 37 | 2-8:9 | 1:1 | 4.6 | 54.1 | (0.68, 0.32) | 150 |
| Example 38 | 2-11:21 | 1:1 | 4.6 | 54.4 | (0.68, 0.32) | 149 |
| Example 39 | 2-12:26 | 1:1 | 4.5 | 55.7 | (0.68, 0.32) | 156 |
| Example 40 | 2-24:41 | 1:1 | 4.4 | 56.0 | (0.68, 0.32) | 154 |
| Example 41 | 2-4:78 | 1:1 | 4.5 | 53.2 | (0.68, 0.32) | 145 |
| Example 42 | 2-30:80 | 1:1 | 4.5 | 54.7 | (0.68, 0.32) | 161 |
| Example 43 | 2-3:90 | 1:1 | 4.6 | 55.5 | (0.68, 0.32) | 155 |
| Example 44 | 2-33:101 | 1:1 | 4.7 | 53.9 | (0.68, 0.32) | 148 |
| Example 45 | 2-15:119 | 1:1 | 4.9 | 51.3 | (0.68, 0.32) | 121 |
| Example 46 | 2-42:137 | 1:1 | 4.6 | 53.8 | (0.68, 0.32) | 143 |
| Example 47 | 3-2:144 | 1:1 | 5.2 | 49.6 | (0.68, 0.32) | 101 |
| Example 48 | 2-51:160 | 1:1 | 4.5 | 55.9 | (0.68, 0.32) | 151 |
| Example 49 | 2-27:163 | 1:1 | 4.5 | 53.8 | (0.68, 0.32) | 154 |
| Example 50 | 3-7:178 | 1:1 | 5.0 | 51.8 | (0.68, 0.32) | 123 |
| Example 51 | 2-2:200 | 1:1 | 4.5 | 54.4 | (0.68, 0.32) | 169 |
| Example 52 | 2-8:205 | 1:1 | 4.5 | 54.9 | (0.68, 0.32) | 159 |
| Example 53 | 2-7:237 | 1:1 | 4.6 | 53.3 | (0.68, 0.32) | 157 |
| Example 54 | 2-26:240 | 1:1 | 4.5 | 53.4 | (0.68, 0.32) | 166 |
| Example 55 | 2-43:241 | 1:1 | 5.0 | 50.9 | (0.68, 0.32) | 129 |
| Example 56 | 2-25:262 | 1:1 | 5.0 | 50.5 | (0.68, 0.32) | 121 |
| Example 57 | 2-5:295 | 1:1 | 5.0 | 50.8 | (0.68, 0.32) | 126 |
| Example 58 | 2-16:302 | 1:1 | 5.2 | 49.1 | (0.86, 0.32) | 102 |
| Example 59 | 2-19:324 | 1:1 | 4.6 | 53.1 | (0.68, 0.32) | 147 |
| Example 60 | 2-27:341 | 1:1 | 4.6 | 54.0 | (0.68, 0.32) | 154 |
| Example 61 | 2-51:353 | 1:1 | 4.6 | 53.0 | (0.68, 0.32) | 141 |
| Example 62 | 2-40:356 | 1:1 | 4.7 | 53.8 | (0.68, 0.32) | 147 |
| Example 63 | 2-37:375 | 1:1 | 5.0 | 51.5 | (0.68, 0.32) | 125 |
| Example 64 | 2-25:378 | 1:1 | 4.7 | 54.3 | (0.68, 0.32) | 140 |
| Comparative | 2-8:H1 | 1:1 | 5.5 | 48.3 | (0.68, 0.32) | 91 |
| Example 13 | | | | | | |
| Comparative | 2-41:H2 | 1:1 | 5.5 | 47.9 | (0.68, 0.32) | 95 |
| Example 14 | | | | | | |
| Comparative | 2-37:H3 | 1:1 | 5.5 | 48.9 | (0.68, 0.32) | 92 |
| Example 15 | | | | | | |
| Comparative | 2-47:H4 | 1:1 | 5.6 | 48.4 | (0.68, 0.32) | 96 |
| Example 16 | | | | | | |
| Comparative | 2-39:H5 | 1:1 | 5.7 | 46.3 | (0.68, 0.32) | 80 |
| Example 17 | | | | | | |
| Comparative | 2-23:H6 | 1:1 | 5.7 | 46.6 | (0.68, 0.32) | 82 |
| Example 18 | | | | | | |
| Comparative | 2-15:H7 | 1:1 | 5.7 | 46.1 | (0.68, 0.32) | 84 |
| Example 19 | | | | | | |
| Comparative | 2-52:H8 | 1:1 | 5.7 | 46.8 | (0.68, 0.32) | 80 |
| Example 20 | | | | | | |
| Comparative | 2-45:H9 | 1:1 | 5.5 | 48.9 | (0.68, 0.32) | 93 |
| Example 21 | | | | | | |
| Comparative | 2-5:H10 | 1:1 | 5.5 | 48.3 | (0.68, 0.32) | 95 |
| Example 22 | | | | | | |
| Comparative | 2-16:H11 | 1:1 | 5.6 | 47.7 | (0.68, 0.32) | 88 |
| Example 23 | | | | | | |
| Comparative | 2-7:H12 | 1:1 | 5.5 | 48.0 | (0.68, 0.32) | 93 |
| Example 24 | | | | | | |

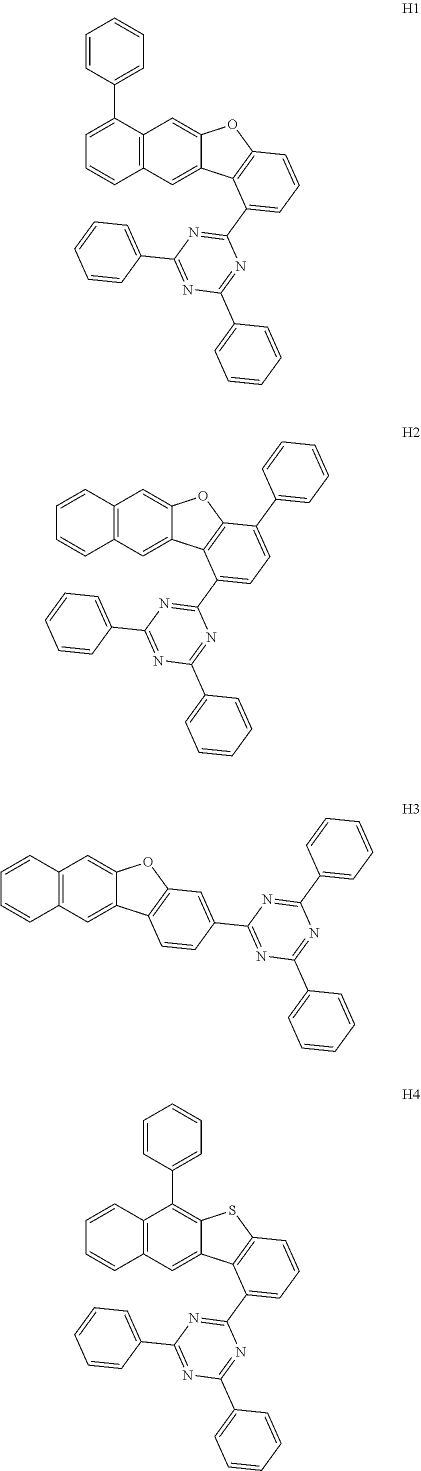 [SMILES: c1ccc(-c2nc(-c3ccccc3)nc(-c3cccc4oc5cc6c(-c7ccccc7)cccc6cc5c34)n2)cc1 ; c1ccc(-c2nc(-c3ccccc3)nc(-c3ccc(-c4ccccc4)c4oc5cc6ccccc6cc5c34)n2)cc1 ; c1ccc(-c2nc(-c3ccccc3)nc(-c3ccc4c(c3)oc3cc5ccccc5cc34)n2)cc1 ; c1ccc(-c2nc(-c3ccccc3)nc(-c3cccc4sc5c(-c6ccccc6)c6ccccc6cc5c34)n2)cc1]

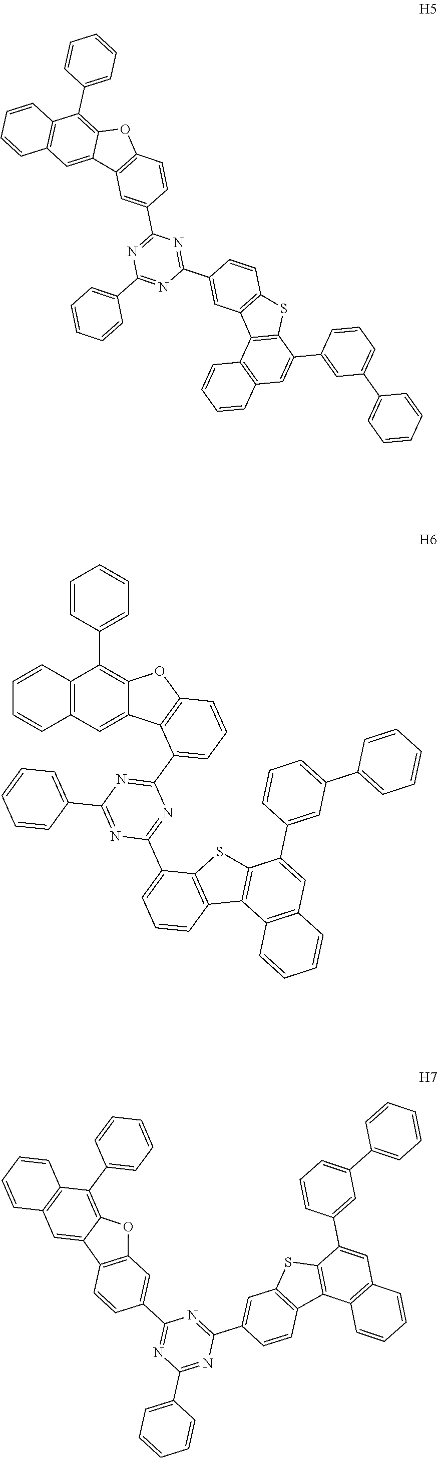 [SMILES: c1ccc(-c2cccc(-c3cc4ccccc4c4c3sc3ccc(-c5nc(-c6ccccc6)nc(-c6ccc7oc8c(-c9ccccc9)c9ccccc9cc8c7c6)n5)cc34)c2)cc1 ; c1ccc(-c2cccc(-c3cc4ccccc4c4c3sc3c(-c5nc(-c6ccccc6)nc(-c6cccc7oc8c(-c9ccccc9)c9ccccc9cc8c67)n5)cccc34)c2)cc1 ; c1ccc(-c2cccc(-c3cc4ccccc4c4c3sc3cc(-c5nc(-c6ccccc6)nc(-c6ccc7c(c6)oc6c(-c8ccccc8)c8ccccc8cc67)n5)ccc34)c2)cc1]

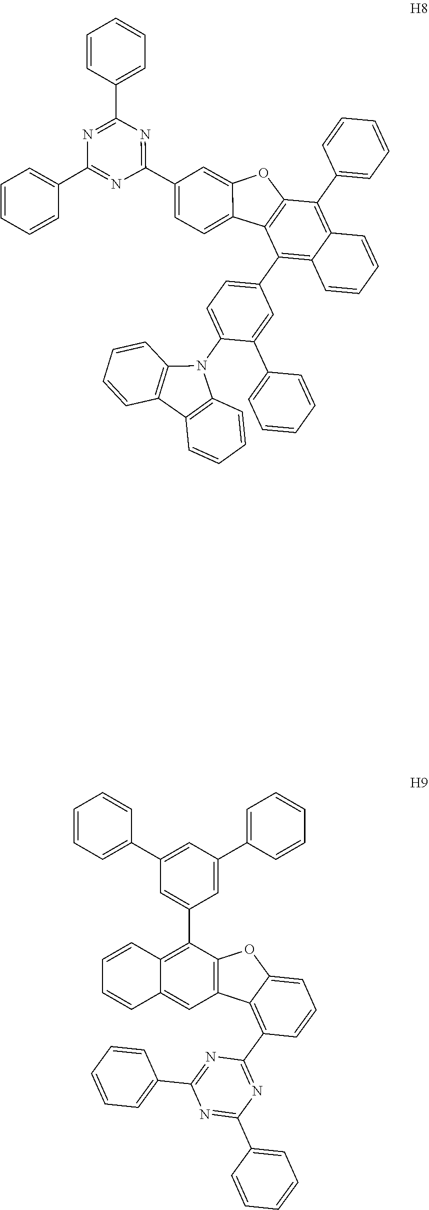 [SMILES: c1ccc(-c2nc(-c3ccccc3)nc(-c3ccc4c(c3)oc3c(-c5ccccc5)c5ccccc5c(-c5ccc(-n6c7ccccc7c7ccccc76)c(-c6ccccc6)c5)c34)n2)cc1 ; c1ccc(-c2cc(-c3ccccc3)cc(-c3c4ccccc4cc4c3oc3cccc(-c5nc(-c6ccccc6)nc(-c6ccccc6)n5)c34)c2)cc1]

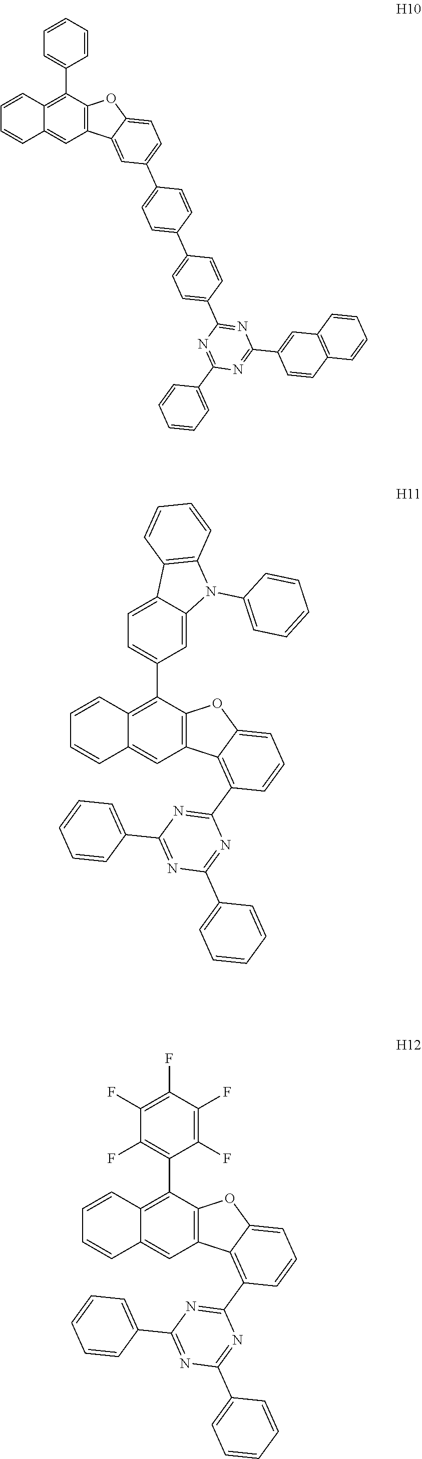 [SMILES: c1ccc(-c2nc(-c3ccc(-c4ccc(-c5ccc6oc7c(-c8ccccc8)c8ccccc8cc7c6c5)cc4)cc3)nc(-c3ccc4ccccc4c3)n2)cc1 ; c1ccc(-c2nc(-c3ccccc3)nc(-c3cccc4oc5c(-c6ccc7c8ccccc8n(-c8ccccc8)c7c6)c6ccccc6cc5c34)n2)cc1 ; Fc1c(F)c(F)c(-c2c3ccccc3cc3c2oc2cccc(-c4nc(-c5ccccc5)nc(-c5ccccc5)n4)c23)c(F)c1F]

In Table 5, P means a P-type host compound, and N means an N-type host compound. The compound of the present invention was used as the N-type host compound, and the P-type host compound was selected from the following compounds and used.

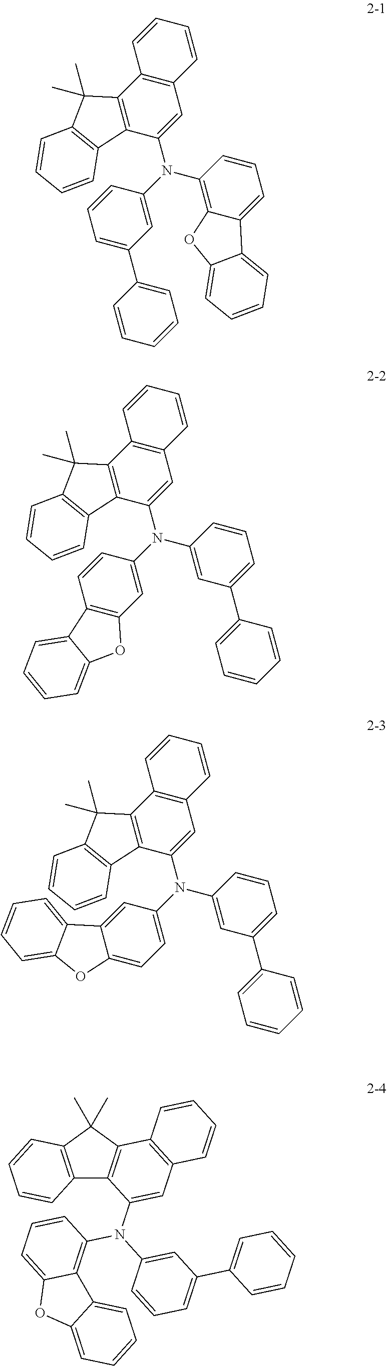

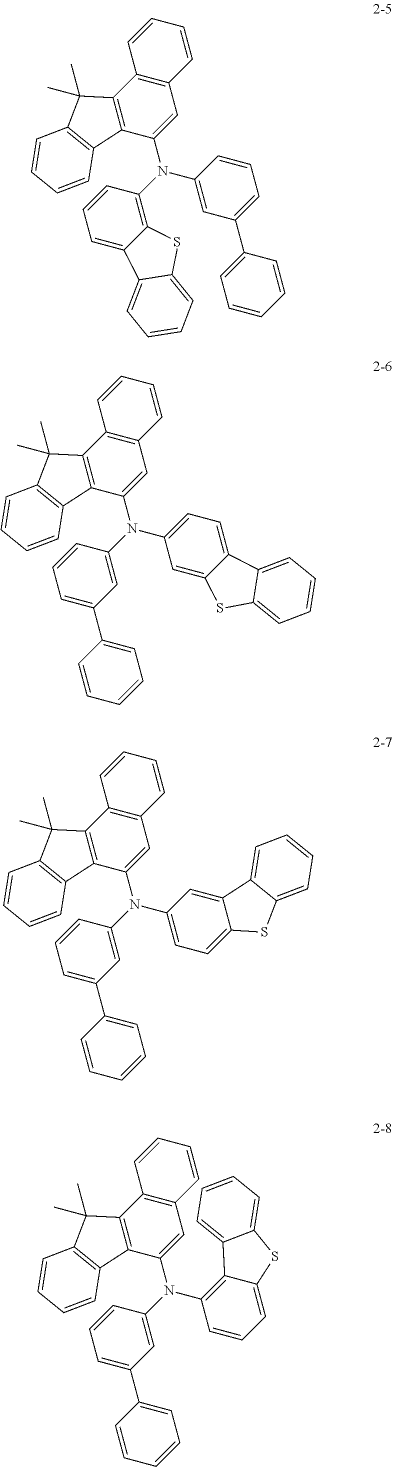 [SMILES: CC1(C)c2ccccc2-c2c(N(c3cccc(-c4ccccc4)c3)c3cccc4c3sc3ccccc34)cc3ccccc3c21 ; CC1(C)c2ccccc2-c2c(N(c3cccc(-c4ccccc4)c3)c3ccc4c(c3)sc3ccccc34)cc3ccccc3c21 ; CC1(C)c2ccccc2-c2c(N(c3cccc(-c4ccccc4)c3)c3ccc4sc5ccccc5c4c3)cc3ccccc3c21 ; CC1(C)c2ccccc2-c2c(N(c3cccc(-c4ccccc4)c3)c3cccc4sc5ccccc5c34)cc3ccccc3c21]

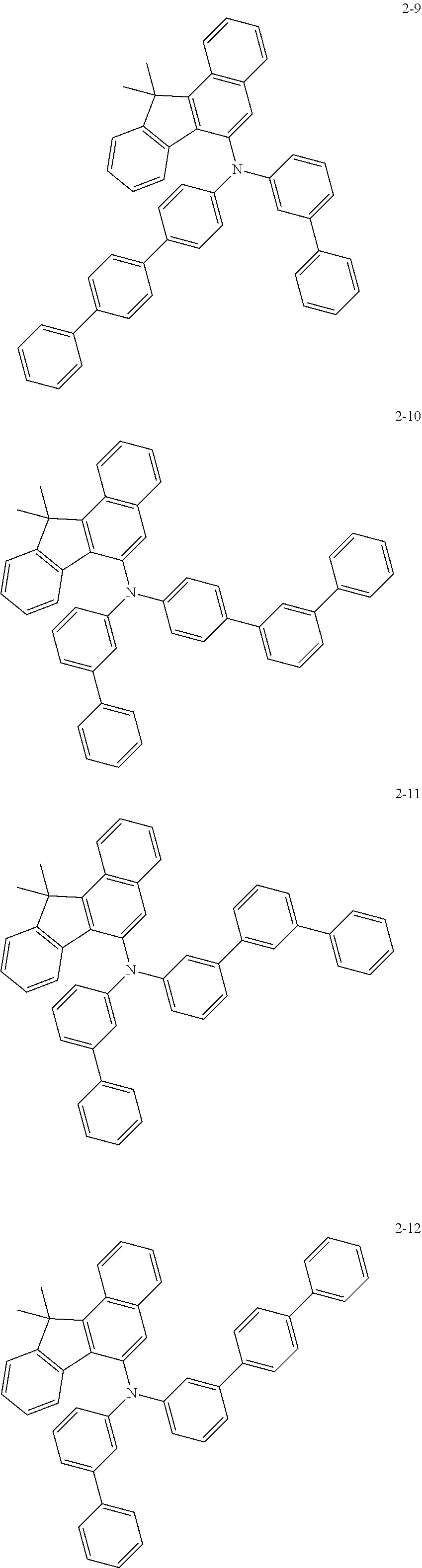 [SMILES: CC1(C)c2ccccc2-c2c(N(c3ccc(-c4ccc(-c5ccccc5)cc4)cc3)c3cccc(-c4ccccc4)c3)cc3ccccc3c21 ; CC1(C)c2ccccc2-c2c(N(c3ccc(-c4cccc(-c5ccccc5)c4)cc3)c3cccc(-c4ccccc4)c3)cc3ccccc3c21 ; CC1(C)c2ccccc2-c2c(N(c3cccc(-c4ccccc4)c3)c3cccc(-c4cccc(-c5ccccc5)c4)c3)cc3ccccc3c21 ; CC1(C)c2ccccc2-c2c(N(c3cccc(-c4ccccc4)c3)c3cccc(-c4ccc(-c5ccccc5)cc4)c3)cc3ccccc3c21]

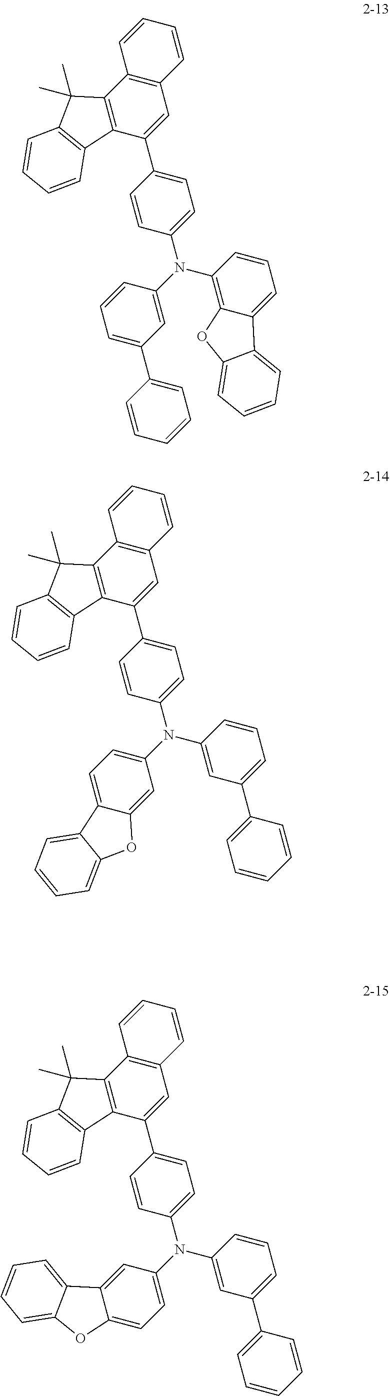 [SMILES: CC1(C)c2ccccc2-c2c(-c3ccc(N(c4cccc(-c5ccccc5)c4)c4cccc5c4oc4ccccc45)cc3)cc3ccccc3c21 ; CC1(C)c2ccccc2-c2c(-c3ccc(N(c4cccc(-c5ccccc5)c4)c4ccc5c(c4)oc4ccccc45)cc3)cc3ccccc3c21 ; CC1(C)c2ccccc2-c2c(-c3ccc(N(c4cccc(-c5ccccc5)c4)c4ccc5oc6ccccc6c5c4)cc3)cc3ccccc3c21]

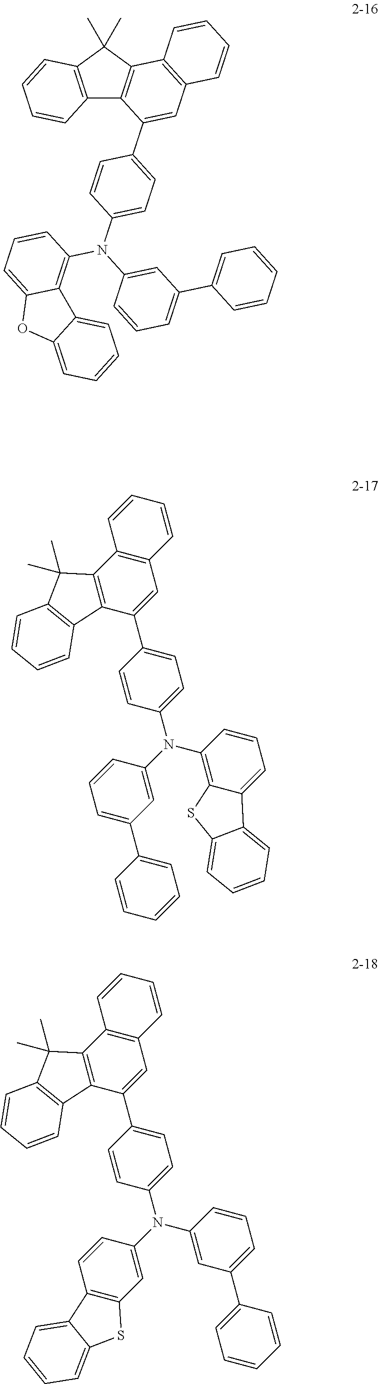 [SMILES: CC1(C)c2ccccc2-c2c(-c3ccc(N(c4cccc(-c5ccccc5)c4)c4cccc5oc6ccccc6c45)cc3)cc3ccccc3c21 ; CC1(C)c2ccccc2-c2c(-c3ccc(N(c4cccc(-c5ccccc5)c4)c4cccc5c4sc4ccccc45)cc3)cc3ccccc3c21 ; CC1(C)c2ccccc2-c2c(-c3ccc(N(c4cccc(-c5ccccc5)c4)c4ccc5c(c4)sc4ccccc45)cc3)cc3ccccc3c21]

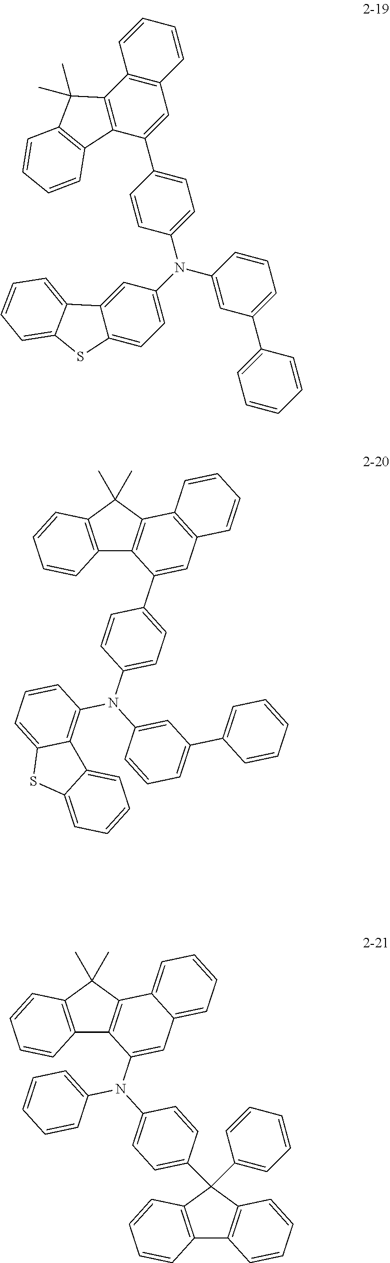 [SMILES: CC1(C)c2ccccc2-c2c(-c3ccc(N(c4cccc(-c5ccccc5)c4)c4ccc5sc6ccccc6c5c4)cc3)cc3ccccc3c21 ; CC1(C)c2ccccc2-c2c(-c3ccc(N(c4cccc(-c5ccccc5)c4)c4cccc5sc6ccccc6c45)cc3)cc3ccccc3c21 ; CC1(C)c2ccccc2-c2c(N(c3ccccc3)c3ccc(C4(c5ccccc5)c5ccccc5-c5ccccc54)cc3)cc3ccccc3c21]

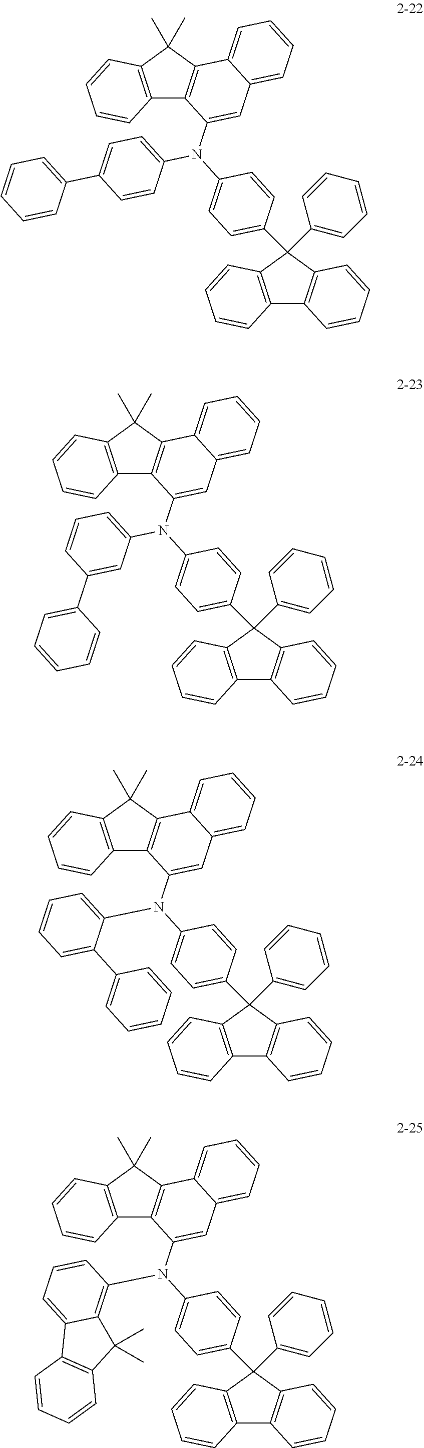 [SMILES: CC1(C)c2ccccc2-c2c(N(c3ccc(-c4ccccc4)cc3)c3ccc(C4(c5ccccc5)c5ccccc5-c5ccccc54)cc3)cc3ccccc3c21 ; CC1(C)c2ccccc2-c2c(N(c3ccc(C4(c5ccccc5)c5ccccc5-c5ccccc54)cc3)c3cccc(-c4ccccc4)c3)cc3ccccc3c21 ; CC1(C)c2ccccc2-c2c(N(c3ccc(C4(c5ccccc5)c5ccccc5-c5ccccc54)cc3)c3ccccc3-c3ccccc3)cc3ccccc3c21 ; CC1(C)c2ccccc2-c2cccc(N(c3ccc(C4(c5ccccc5)c5ccccc5-c5ccccc54)cc3)c3cc4ccccc4c4c3-c3ccccc3C4(C)C)c21]

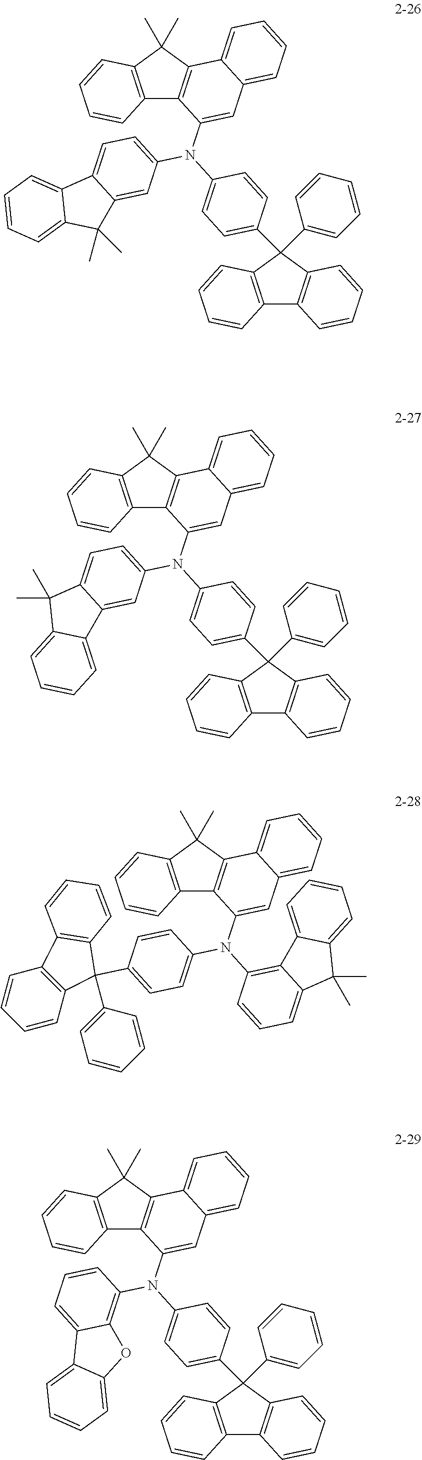 [SMILES: CC1(C)c2ccccc2-c2ccc(N(c3ccc(C4(c5ccccc5)c5ccccc5-c5ccccc54)cc3)c3cc4ccccc4c4c3-c3ccccc3C4(C)C)cc21 ; CC1(C)c2ccccc2-c2cc(N(c3ccc(C4(c5ccccc5)c5ccccc5-c5ccccc54)cc3)c3cc4ccccc4c4c3-c3ccccc3C4(C)C)ccc21 ; CC1(C)c2ccccc2-c2c(N(c3ccc(C4(c5ccccc5)c5ccccc5-c5ccccc54)cc3)c3cc4ccccc4c4c3-c3ccccc3C4(C)C)cccc21 ; CC1(C)c2ccccc2-c2c(N(c3ccc(C4(c5ccccc5)c5ccccc5-c5ccccc54)cc3)c3cccc4c3oc3ccccc34)cc3ccccc3c21]

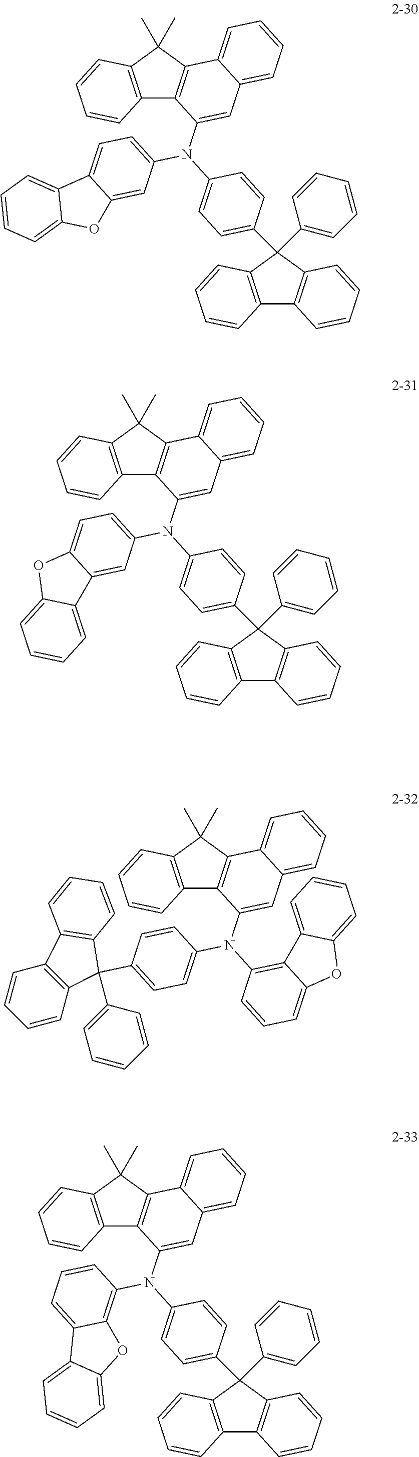 [SMILES: CC1(C)c2ccccc2-c2c(N(c3ccc(C4(c5ccccc5)c5ccccc5-c5ccccc54)cc3)c3ccc4c(c3)oc3ccccc34)cc3ccccc3c21 ; CC1(C)c2ccccc2-c2c(N(c3ccc(C4(c5ccccc5)c5ccccc5-c5ccccc54)cc3)c3ccc4oc5ccccc5c4c3)cc3ccccc3c21 ; CC1(C)c2ccccc2-c2c(N(c3ccc(C4(c5ccccc5)c5ccccc5-c5ccccc54)cc3)c3cccc4oc5ccccc5c34)cc3ccccc3c21 ; CC1(C)c2ccccc2-c2c(N(c3ccc(C4(c5ccccc5)c5ccccc5-c5ccccc54)cc3)c3cccc4c3oc3ccccc34)cc3ccccc3c21]

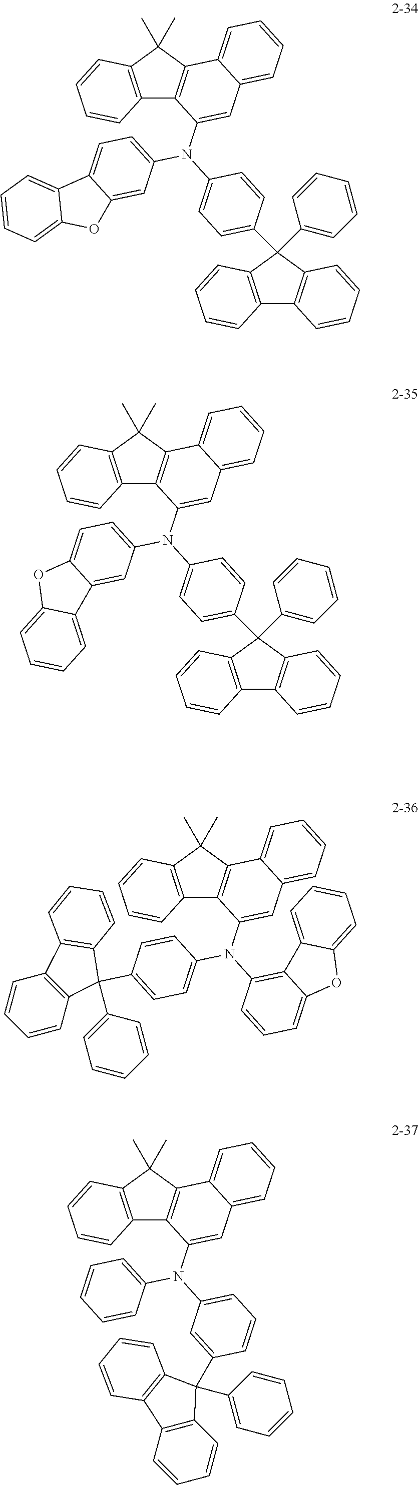 [SMILES: CC1(C)c2ccccc2-c2c(N(c3ccc(C4(c5ccccc5)c5ccccc5-c5ccccc54)cc3)c3ccc4c(c3)oc3ccccc34)cc3ccccc3c21 ; CC1(C)c2ccccc2-c2c(N(c3ccc(C4(c5ccccc5)c5ccccc5-c5ccccc54)cc3)c3ccc4oc5ccccc5c4c3)cc3ccccc3c21 ; CC1(C)c2ccccc2-c2c(N(c3ccc(C4(c5ccccc5)c5ccccc5-c5ccccc54)cc3)c3cccc4oc5ccccc5c34)cc3ccccc3c21 ; CC1(C)c2ccccc2-c2c(N(c3ccccc3)c3cccc(C4(c5ccccc5)c5ccccc5-c5ccccc54)c3)cc3ccccc3c21]

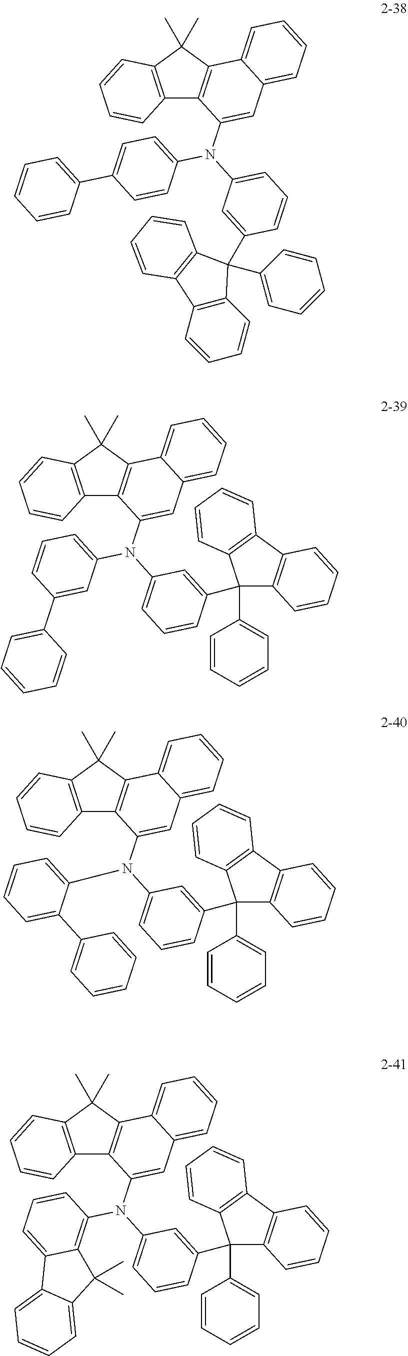 [SMILES: CC1(C)c2ccccc2-c2c(N(c3ccc(-c4ccccc4)cc3)c3cccc(C4(c5ccccc5)c5ccccc5-c5ccccc54)c3)cc3ccccc3c21 ; CC1(C)c2ccccc2-c2c(N(c3cccc(-c4ccccc4)c3)c3cccc(C4(c5ccccc5)c5ccccc5-c5ccccc54)c3)cc3ccccc3c21 ; CC1(C)c2ccccc2-c2c(N(c3cccc(C4(c5ccccc5)c5ccccc5-c5ccccc54)c3)c3ccccc3-c3ccccc3)cc3ccccc3c21 ; CC1(C)c2ccccc2-c2cccc(N(c3cccc(C4(c5ccccc5)c5ccccc5-c5ccccc54)c3)c3cc4ccccc4c4c3-c3ccccc3C4(C)C)c21]

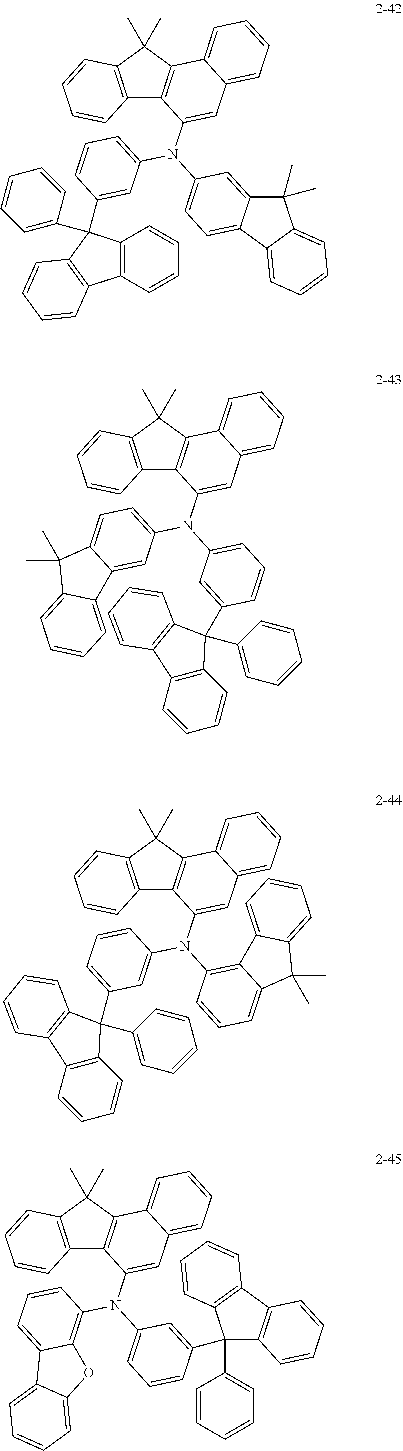

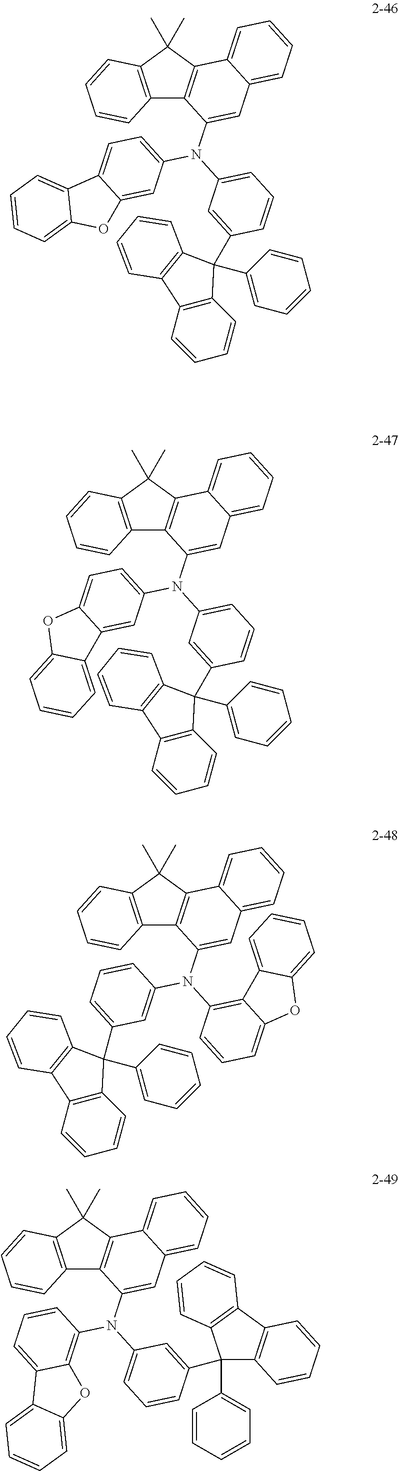

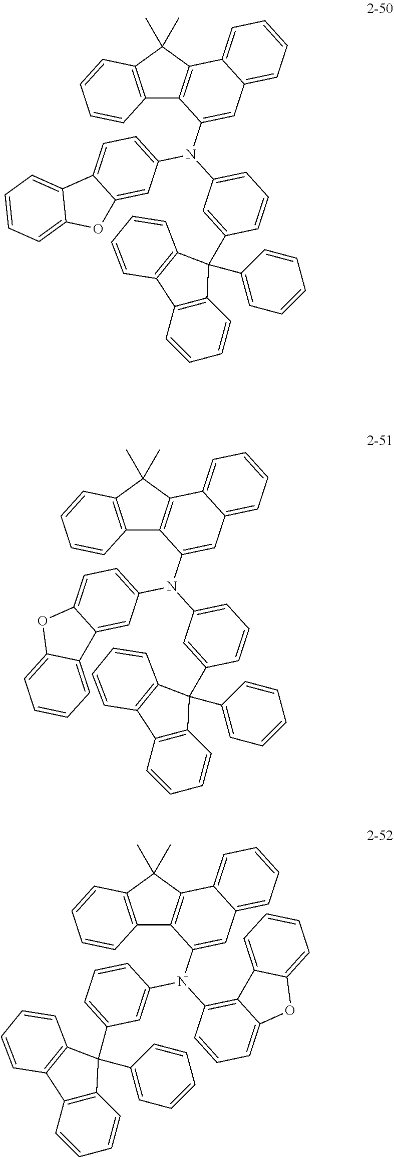 [SMILES: CC1(C)c2ccccc2-c2c(N(c3cccc(C4(c5ccccc5)c5ccccc5-c5ccccc54)c3)c3ccc4c(c3)oc3ccccc34)cc3ccccc3c21 ; CC1(C)c2ccccc2-c2c(N(c3cccc(C4(c5ccccc5)c5ccccc5-c5ccccc54)c3)c3ccc4oc5ccccc5c4c3)cc3ccccc3c21 ; CC1(C)c2ccccc2-c2c(N(c3cccc(C4(c5ccccc5)c5ccccc5-c5ccccc54)c3)c3cccc4oc5ccccc5c34)cc3ccccc3c21]

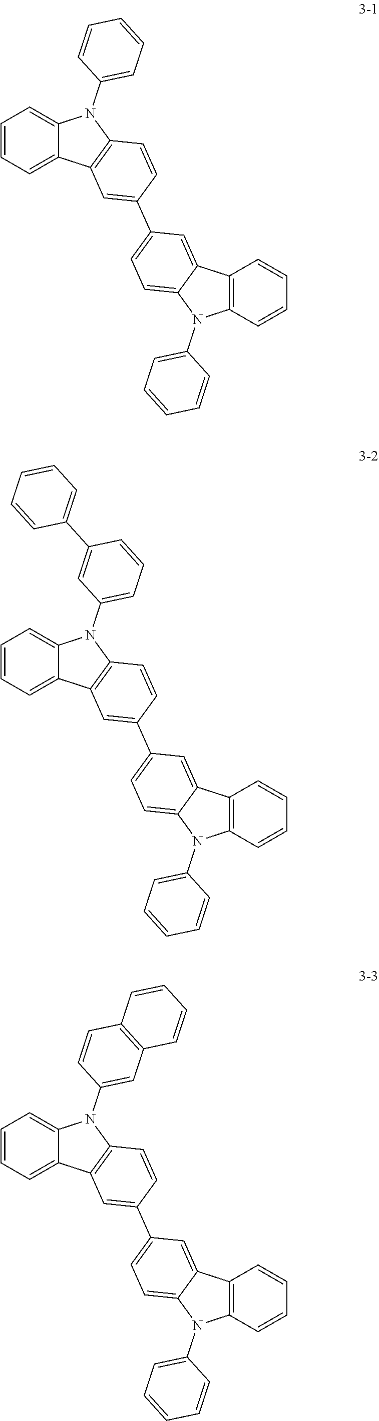 [SMILES: c1ccc(-n2c3ccccc3c3cc(-c4ccc5c(c4)c4ccccc4n5-c4ccccc4)ccc32)cc1 ; c1ccc(-c2cccc(-n3c4ccccc4c4cc(-c5ccc6c(c5)c5ccccc5n6-c5ccccc5)ccc43)c2)cc1 ; c1ccc(-n2c3ccccc3c3cc(-c4ccc5c(c4)c4ccccc4n5-c4ccc5ccccc5c4)ccc32)cc1]

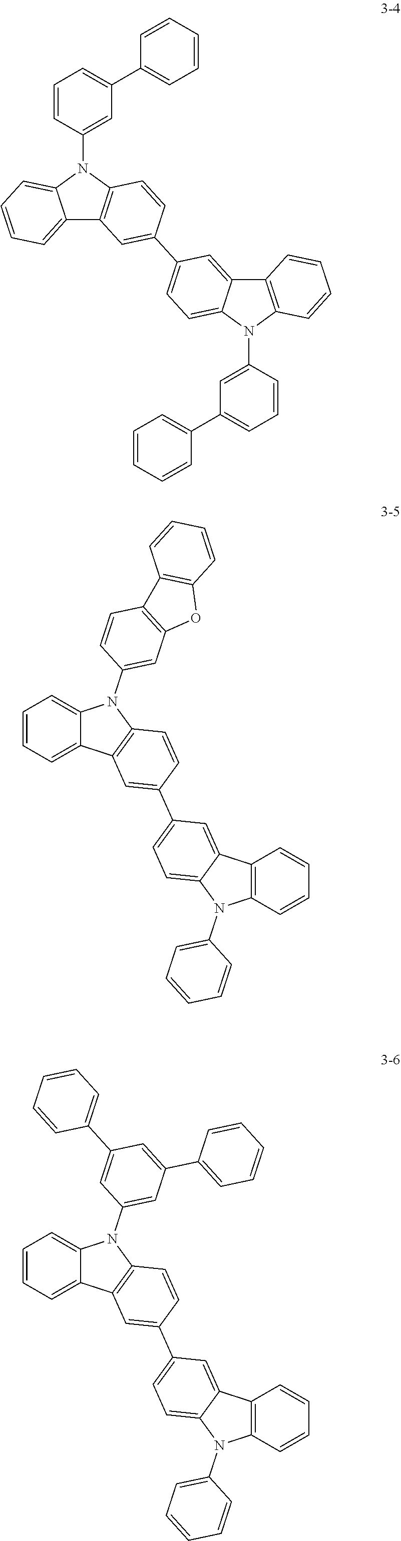

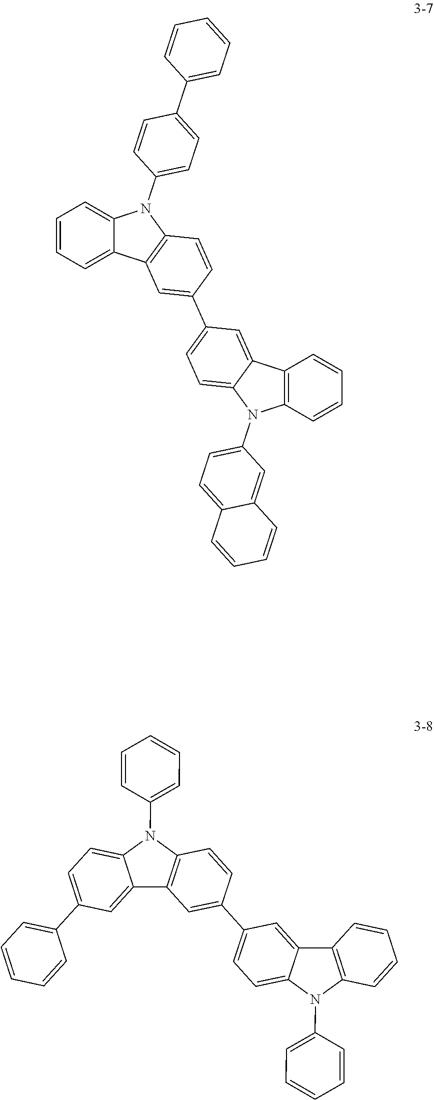 [SMILES: c1ccc(-c2ccc(-n3c4ccccc4c4cc(-c5ccc6c(c5)c5ccccc5n6-c5ccc6ccccc6c5)ccc43)cc2)cc1 ; c1ccc(-c2ccc3c(c2)c2cc(-c4ccc5c(c4)c4ccccc4n5-c4ccccc4)ccc2n3-c2ccccc2)cc1]

As can be seen from the results in Table 5, it could be confirmed that when the heterocyclic compound of the present invention was used as an N-type host and mixed with a P-type host and deposited, the driving voltage, efficiency, and service life of the organic light emitting device were improved. When a donor (P-type host) with good hole transport ability and an acceptor (N-type host) with good electron transport ability are used as hosts of the light emitting layer, the charge balance within the device can be adjusted because holes are injected into the P-type host and electrons are injected into the N-type host due to the exciplex phenomenon of the N+P compound. Therefore, it can be seen that a combination of an N-type host compound with appropriate electron transfer properties and a P-type host compound with appropriate hole transfer properties in an appropriate ratio can help improve driving voltage, efficiency, and service life.

1. A heterocyclic compound represented by the following Chemical Formula 1: 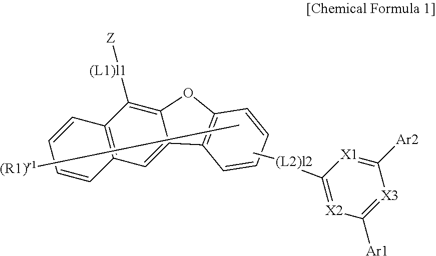
wherein, in Chemical Formula 1,
L1 and L2 are each independently a direct bond; a phenylene group; or a naphthylene group,
l1 and l2 are each independently an integer from 0 to 3, and when l1 and l2 are each 2 or higher, substituents in the parenthesis are the same as or different from each other,
R1 is hydrogen; or deuterium,
r1 is an integer from 0 to 8, and when r1 is 2 or higher, R1's are the same as or different from each other,
Z is —NR′R″; a substituted or unsubstituted alkyl group having 1 to 60 carbon atoms; a substituted or unsubstituted cycloalkyl group having 3 to 60 carbon atoms; a substituted or unsubstituted heterocycloalkyl group having 3 to 60 carbon atoms; an aryl group having 6 to 15 carbon atoms, which is unsubstituted or substituted with deuterium or an aryl group; a triphenylene group which is unsubstituted or substituted with deuterium or an aryl group; an aryl group having 20 to 60 carbon atoms, which is unsubstituted or substituted with deuterium or an aryl group; a substituted or unsubstituted dibenzofuran group; a substituted or unsubstituted dibenzothiophene group; or a substituted or unsubstituted benzocarbazole group,
X1 to X3 are each independently N or CR, and at least one thereof is N,
Ar1 and Ar2 are each independently hydrogen; deuterium; a halogen group; a cyano group; a substituted or unsubstituted alkyl group having 1 to 60 carbon atoms; a substituted or unsubstituted cycloalkyl group having 3 to 60 carbon atoms; a substituted or unsubstituted heterocycloalkyl group having 2 to 60 carbon atoms; an aryl group having 6 to 60 carbon atoms, which is unsubstituted or substituted with deuterium or an aryl group; or a substituted or unsubstituted heteroaryl group having 2 to 60 carbon atoms,
R, R′ and R″ are each independently hydrogen; deuterium; a halogen group; a cyano group; a substituted or unsubstituted alkyl group having 1 to 60 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; or a substituted or unsubstituted heteroaryl group having 2 to 60 carbon atoms, or
R is bonded to Ar1 or Ar2 to form a substituted or unsubstituted hetero ring having 2 to 60 carbon atoms, and
when Z is an aryl group having 6 to 15 carbon atoms, which is unsubstituted or substituted with an aryl group, Ar1 and Ar2 are hydrogen; deuterium; a halogen group; a cyano group; a substituted or unsubstituted alkyl group having 1 to 60 carbon atoms; a substituted or unsubstituted cycloalkyl group having 3 to 60 carbon atoms; a substituted or unsubstituted heterocycloalkyl group having 2 to 60 carbon atoms; an aryl group having 6 to 60 carbon atoms, which is unsubstituted or substituted with deuterium or an aryl group; a substituted or unsubstituted dibenzofuran group; or a substituted or unsubstituted dibenzothiophene group.

2. The heterocyclic compound of claim 1, wherein Chemical Formula 1 is represented by any one of the following Chemical Formulae 1-1 to 1-4: 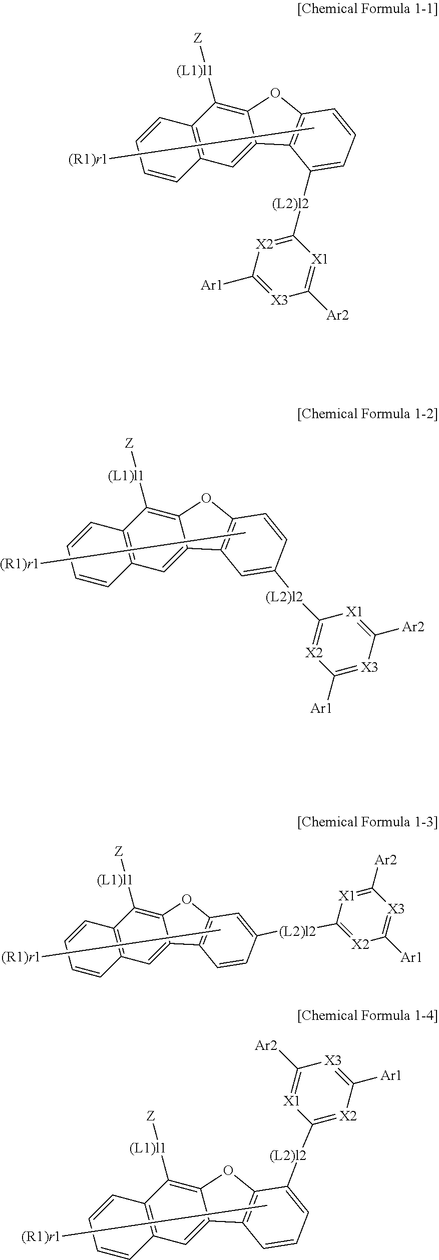
in Chemical Formulae 1-1 to 1-4,
the definition of each substituent is the same as that in Chemical Formula 1.

3. The heterocyclic compound of claim 1, wherein Ar1 and Ar2 are each independently a phenyl group which is unsubstituted or substituted with deuterium or an aryl group; a biphenyl group which is unsubstituted or substituted with deuterium or an aryl group; a naphthyl group which is unsubstituted or substituted with deuterium or an aryl group; a phenanthrene group which is unsubstituted or substituted with deuterium or an aryl group; a triphenylene group which is unsubstituted or substituted with deuterium or an aryl group; a substituted or unsubstituted dibenzofuran group; or a substituted or unsubstituted dibenzothiophene group.

4. The heterocyclic compound of claim 1, wherein Z is —NR′R″; a phenyl group which is unsubstituted or substituted with deuterium or an aryl group; a biphenyl group which is unsubstituted or substituted with deuterium or an aryl group; a naphthyl group which is unsubstituted or substituted with deuterium or an aryl group; a phenanthrene group which is unsubstituted or substituted with deuterium or an aryl group; a triphenylene group which is unsubstituted or substituted with deuterium or an aryl group; or a substituted or unsubstituted benzocarbazole group, and
the definitions of R′ and R″ are the same as those in Chemical Formula 1.

5. The heterocyclic compound of claim 1, wherein a deuterium content of Chemical Formula 1 is 0%, or 10% to 100%.

6. The heterocyclic compound of claim 1, wherein Chemical Formula 1 is represented by any one of the following compounds:

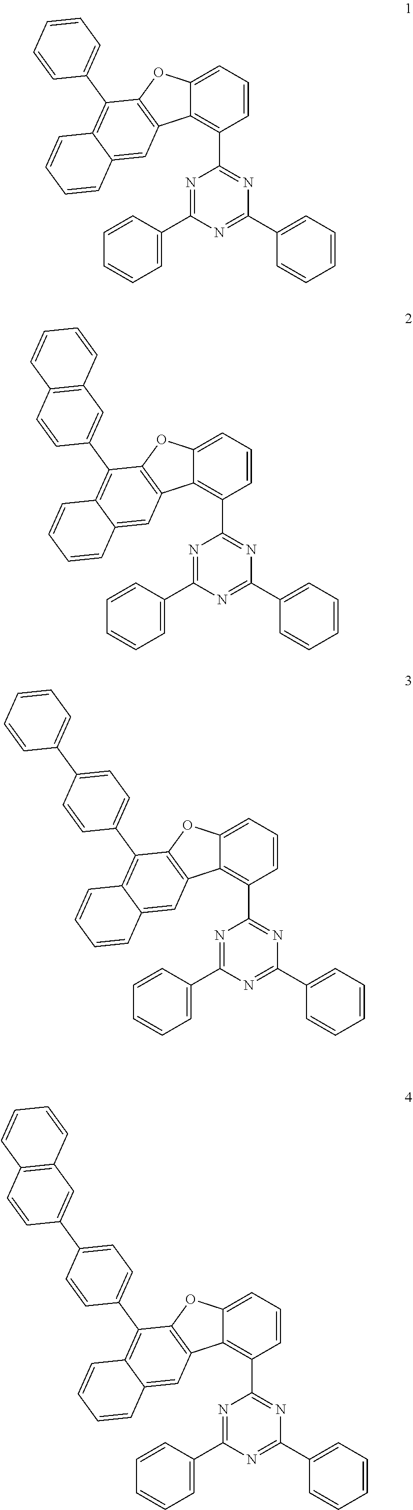 [SMILES: c1ccc(-c2nc(-c3ccccc3)nc(-c3cccc4oc5c(-c6ccccc6)c6ccccc6cc5c34)n2)cc1 ; c1ccc(-c2nc(-c3ccccc3)nc(-c3cccc4oc5c(-c6ccc7ccccc7c6)c6ccccc6cc5c34)n2)cc1 ; c1ccc(-c2ccc(-c3c4ccccc4cc4c3oc3cccc(-c5nc(-c6ccccc6)nc(-c6ccccc6)n5)c34)cc2)cc1 ; c1ccc(-c2nc(-c3ccccc3)nc(-c3cccc4oc5c(-c6ccc(-c7ccc8ccccc8c7)cc6)c6ccccc6cc5c34)n2)cc1]

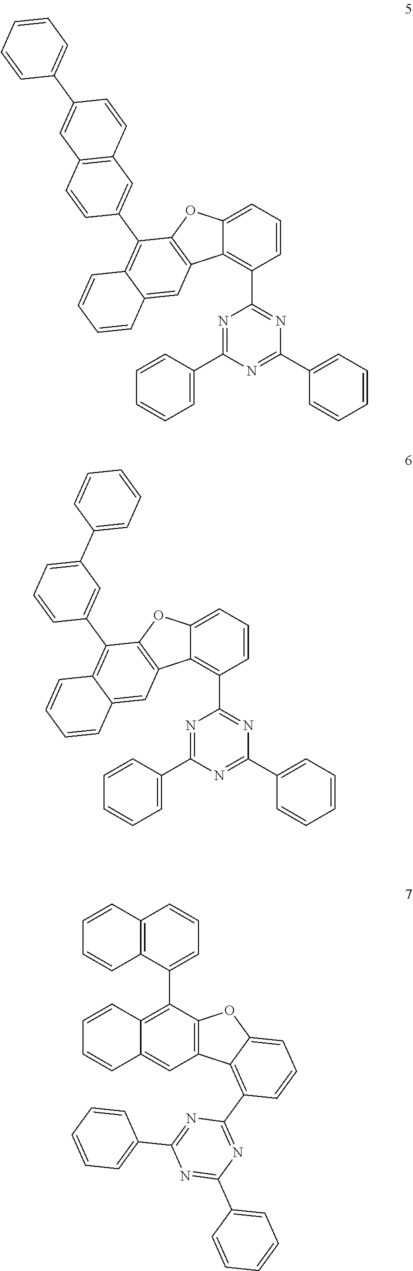 [SMILES: c1ccc(-c2ccc3cc(-c4c5ccccc5cc5c4oc4cccc(-c6nc(-c7ccccc7)nc(-c7ccccc7)n6)c45)ccc3c2)cc1 ; c1ccc(-c2cccc(-c3c4ccccc4cc4c3oc3cccc(-c5nc(-c6ccccc6)nc(-c6ccccc6)n5)c34)c2)cc1 ; c1ccc(-c2nc(-c3ccccc3)nc(-c3cccc4oc5c(-c6cccc7ccccc67)c6ccccc6cc5c34)n2)cc1]

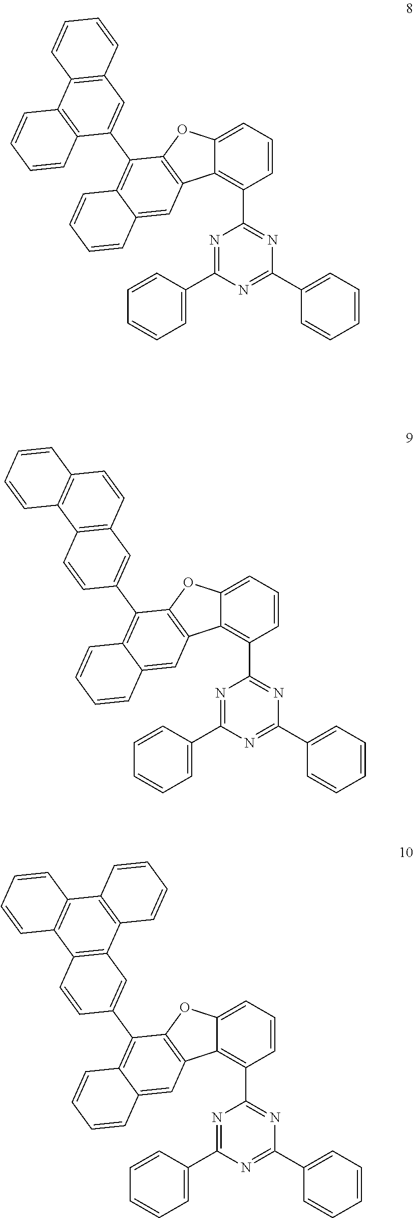 [SMILES: c1ccc(-c2nc(-c3ccccc3)nc(-c3cccc4oc5c(-c6cc7ccccc7c7ccccc67)c6ccccc6cc5c34)n2)cc1 ; c1ccc(-c2nc(-c3ccccc3)nc(-c3cccc4oc5c(-c6ccc7c(ccc8ccccc87)c6)c6ccccc6cc5c34)n2)cc1 ; c1ccc(-c2nc(-c3ccccc3)nc(-c3cccc4oc5c(-c6ccc7c8ccccc8c8ccccc8c7c6)c6ccccc6cc5c34)n2)cc1]

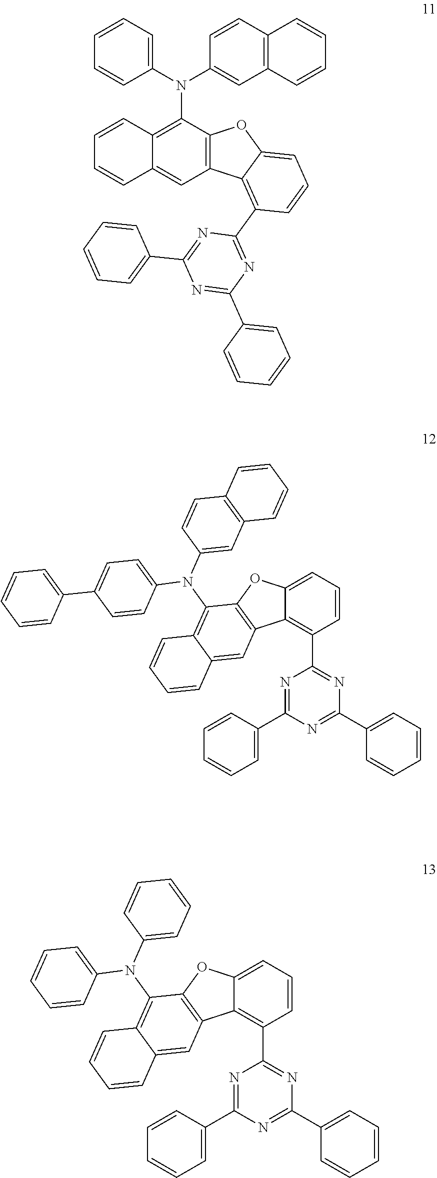 [SMILES: c1ccc(-c2nc(-c3ccccc3)nc(-c3cccc4oc5c(N(c6ccccc6)c6ccc7ccccc7c6)c6ccccc6cc5c34)n2)cc1 ; c1ccc(-c2ccc(N(c3ccc4ccccc4c3)c3c4ccccc4cc4c3oc3cccc(-c5nc(-c6ccccc6)nc(-c6ccccc6)n5)c34)cc2)cc1 ; c1ccc(-c2nc(-c3ccccc3)nc(-c3cccc4oc5c(N(c6ccccc6)c6ccccc6)c6ccccc6cc5c34)n2)cc1]

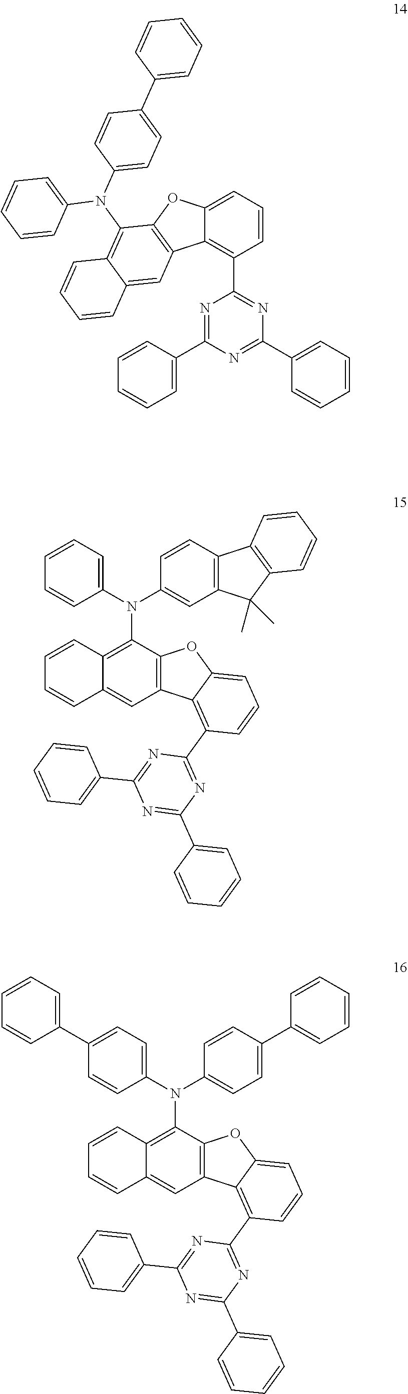 [SMILES: c1ccc(-c2ccc(N(c3ccccc3)c3c4ccccc4cc4c3oc3cccc(-c5nc(-c6ccccc6)nc(-c6ccccc6)n5)c34)cc2)cc1 ; CC1(C)c2ccccc2-c2ccc(N(c3ccccc3)c3c4ccccc4cc4c3oc3cccc(-c5nc(-c6ccccc6)nc(-c6ccccc6)n5)c34)cc21 ; c1ccc(-c2ccc(N(c3ccc(-c4ccccc4)cc3)c3c4ccccc4cc4c3oc3cccc(-c5nc(-c6ccccc6)nc(-c6ccccc6)n5)c34)cc2)cc1]

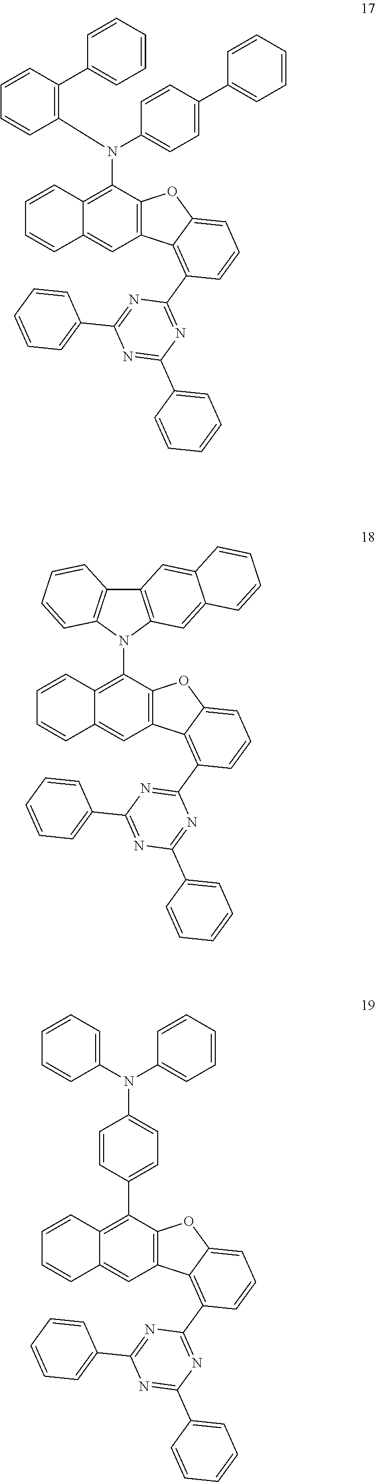 [SMILES: c1ccc(-c2ccc(N(c3ccccc3-c3ccccc3)c3c4ccccc4cc4c3oc3cccc(-c5nc(-c6ccccc6)nc(-c6ccccc6)n5)c34)cc2)cc1 ; c1ccc(-c2nc(-c3ccccc3)nc(-c3cccc4oc5c(-n6c7ccccc7c7cc8ccccc8cc76)c6ccccc6cc5c34)n2)cc1 ; c1ccc(-c2nc(-c3ccccc3)nc(-c3cccc4oc5c(-c6ccc(N(c7ccccc7)c7ccccc7)cc6)c6ccccc6cc5c34)n2)cc1]

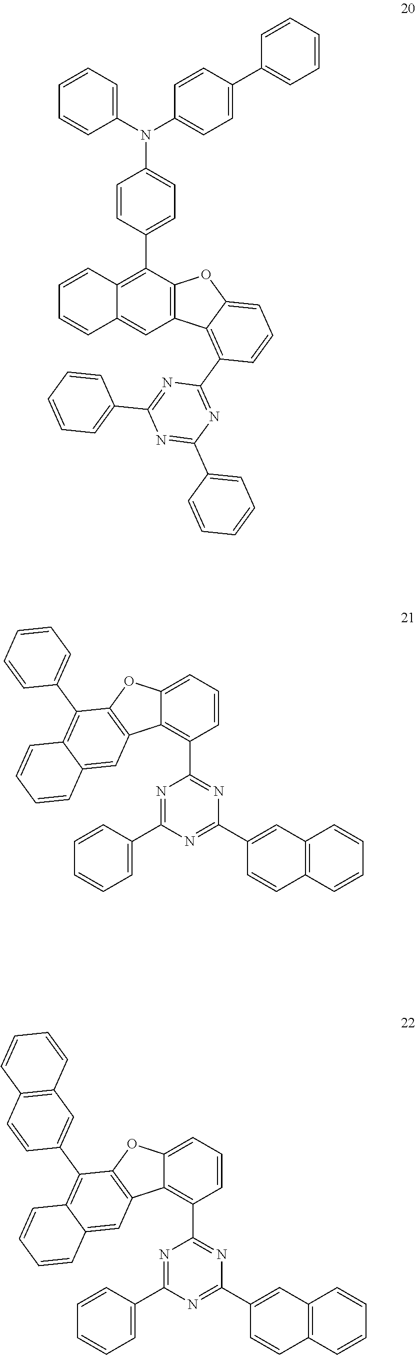 [SMILES: c1ccc(-c2ccc(N(c3ccccc3)c3ccc(-c4c5ccccc5cc5c4oc4cccc(-c6nc(-c7ccccc7)nc(-c7ccccc7)n6)c45)cc3)cc2)cc1 ; c1ccc(-c2nc(-c3ccc4ccccc4c3)nc(-c3cccc4oc5c(-c6ccccc6)c6ccccc6cc5c34)n2)cc1 ; c1ccc(-c2nc(-c3ccc4ccccc4c3)nc(-c3cccc4oc5c(-c6ccc7ccccc7c6)c6ccccc6cc5c34)n2)cc1]

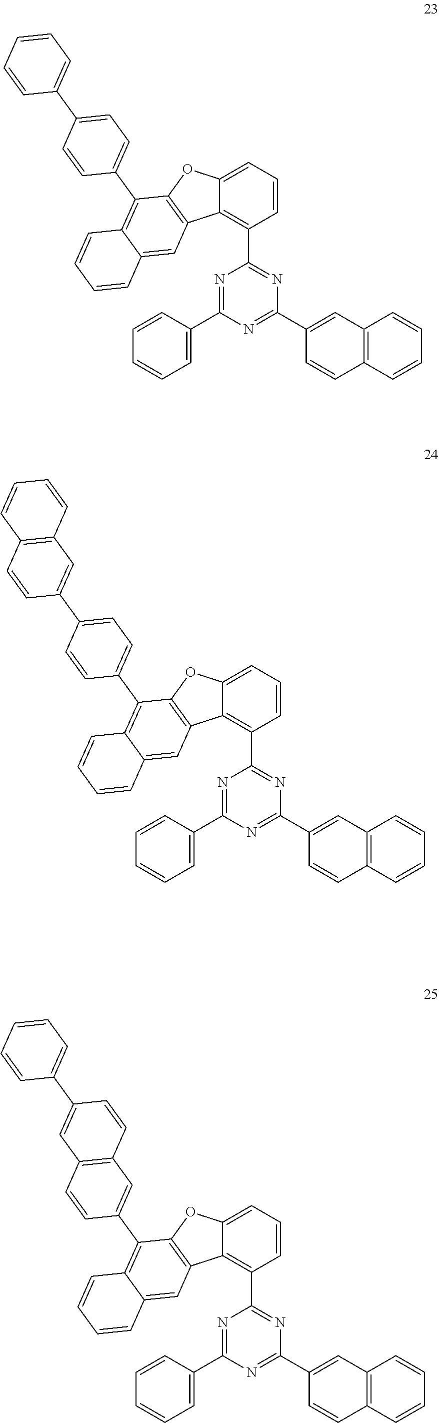 [SMILES: c1ccc(-c2ccc(-c3c4ccccc4cc4c3oc3cccc(-c5nc(-c6ccccc6)nc(-c6ccc7ccccc7c6)n5)c34)cc2)cc1 ; c1ccc(-c2nc(-c3ccc4ccccc4c3)nc(-c3cccc4oc5c(-c6ccc(-c7ccc8ccccc8c7)cc6)c6ccccc6cc5c34)n2)cc1 ; c1ccc(-c2ccc3cc(-c4c5ccccc5cc5c4oc4cccc(-c6nc(-c7ccccc7)nc(-c7ccc8ccccc8c7)n6)c45)ccc3c2)cc1]

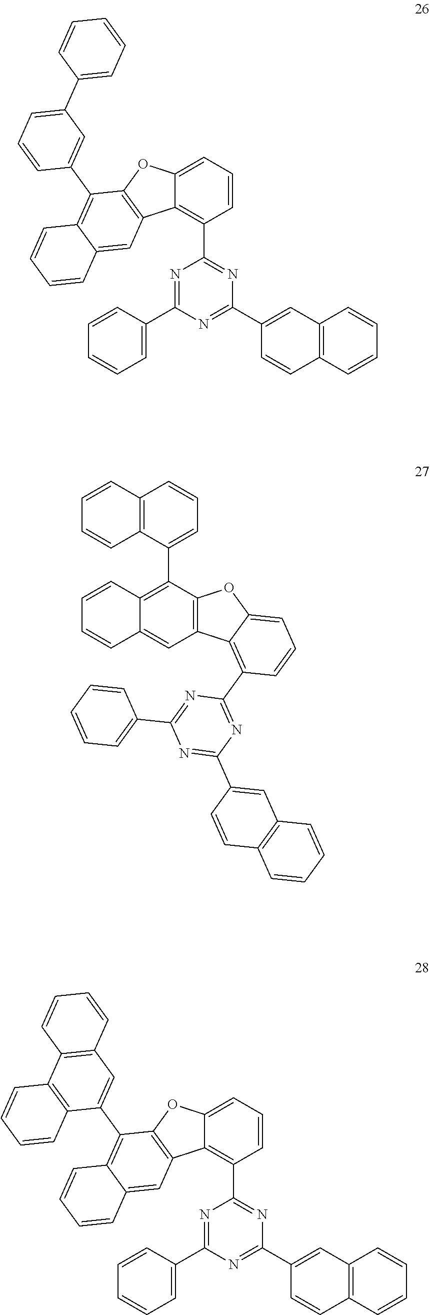 [SMILES: c1ccc(-c2cccc(-c3c4ccccc4cc4c3oc3cccc(-c5nc(-c6ccccc6)nc(-c6ccc7ccccc7c6)n5)c34)c2)cc1 ; c1ccc(-c2nc(-c3ccc4ccccc4c3)nc(-c3cccc4oc5c(-c6cccc7ccccc67)c6ccccc6cc5c34)n2)cc1 ; c1ccc(-c2nc(-c3ccc4ccccc4c3)nc(-c3cccc4oc5c(-c6cc7ccccc7c7ccccc67)c6ccccc6cc5c34)n2)cc1]

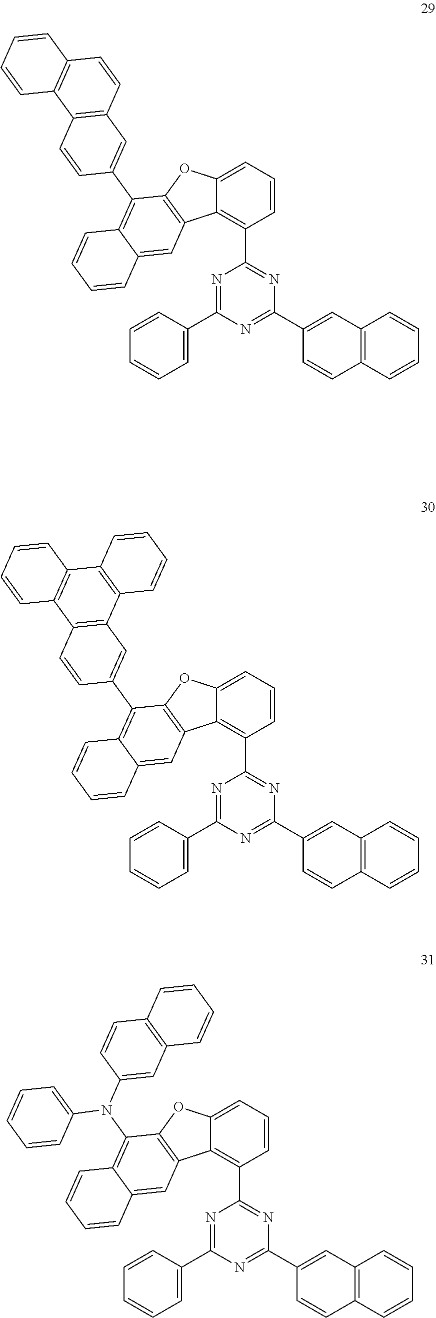 [SMILES: c1ccc(-c2nc(-c3ccc4ccccc4c3)nc(-c3cccc4oc5c(-c6ccc7c(ccc8ccccc87)c6)c6ccccc6cc5c34)n2)cc1 ; c1ccc(-c2nc(-c3ccc4ccccc4c3)nc(-c3cccc4oc5c(-c6ccc7c8ccccc8c8ccccc8c7c6)c6ccccc6cc5c34)n2)cc1 ; c1ccc(-c2nc(-c3ccc4ccccc4c3)nc(-c3cccc4oc5c(N(c6ccccc6)c6ccc7ccccc7c6)c6ccccc6cc5c34)n2)cc1]

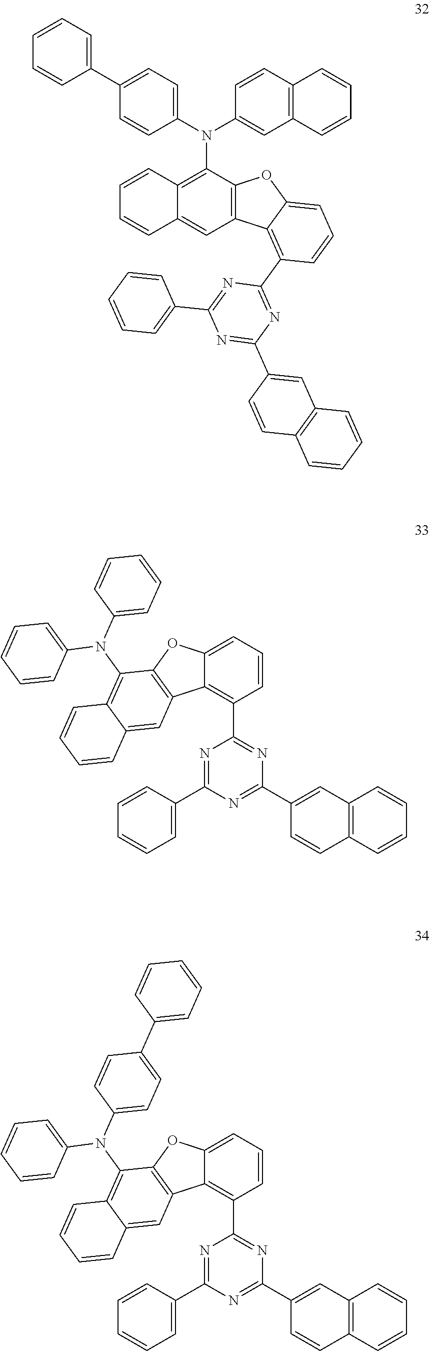 [SMILES: c1ccc(-c2ccc(N(c3ccc4ccccc4c3)c3c4ccccc4cc4c3oc3cccc(-c5nc(-c6ccccc6)nc(-c6ccc7ccccc7c6)n5)c34)cc2)cc1 ; c1ccc(-c2nc(-c3ccc4ccccc4c3)nc(-c3cccc4oc5c(N(c6ccccc6)c6ccccc6)c6ccccc6cc5c34)n2)cc1 ; c1ccc(-c2ccc(N(c3ccccc3)c3c4ccccc4cc4c3oc3cccc(-c5nc(-c6ccccc6)nc(-c6ccc7ccccc7c6)n5)c34)cc2)cc1]

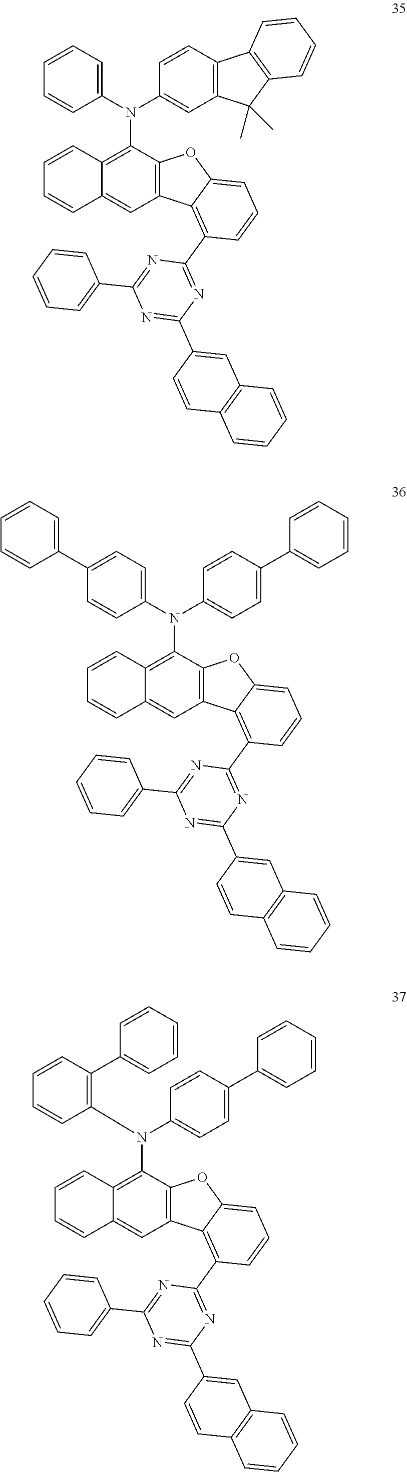 [SMILES: CC1(C)c2ccccc2-c2ccc(N(c3ccccc3)c3c4ccccc4cc4c3oc3cccc(-c5nc(-c6ccccc6)nc(-c6ccc7ccccc7c6)n5)c34)cc21 ; c1ccc(-c2ccc(N(c3ccc(-c4ccccc4)cc3)c3c4ccccc4cc4c3oc3cccc(-c5nc(-c6ccccc6)nc(-c6ccc7ccccc7c6)n5)c34)cc2)cc1 ; c1ccc(-c2ccc(N(c3ccccc3-c3ccccc3)c3c4ccccc4cc4c3oc3cccc(-c5nc(-c6ccccc6)nc(-c6ccc7ccccc7c6)n5)c34)cc2)cc1]

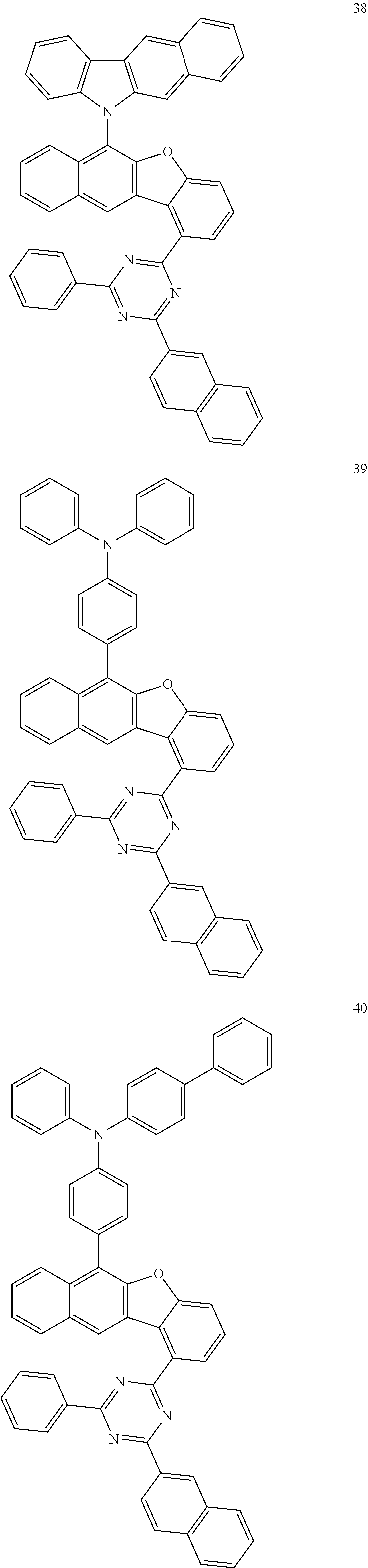

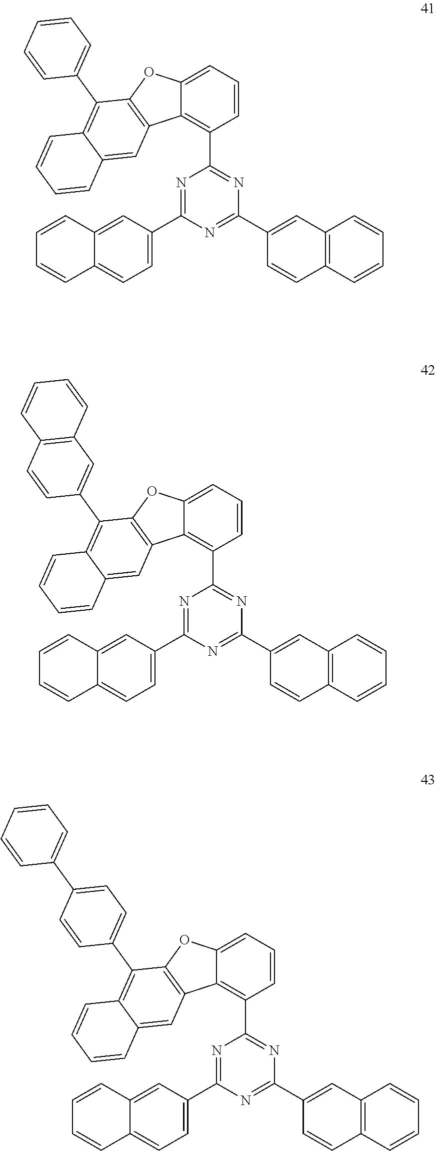 [SMILES: c1ccc(-c2c3ccccc3cc3c2oc2cccc(-c4nc(-c5ccc6ccccc6c5)nc(-c5ccc6ccccc6c5)n4)c23)cc1 ; c1ccc2cc(-c3nc(-c4ccc5ccccc5c4)nc(-c4cccc5oc6c(-c7ccc8ccccc8c7)c7ccccc7cc6c45)n3)ccc2c1 ; c1ccc(-c2ccc(-c3c4ccccc4cc4c3oc3cccc(-c5nc(-c6ccc7ccccc7c6)nc(-c6ccc7ccccc7c6)n5)c34)cc2)cc1]

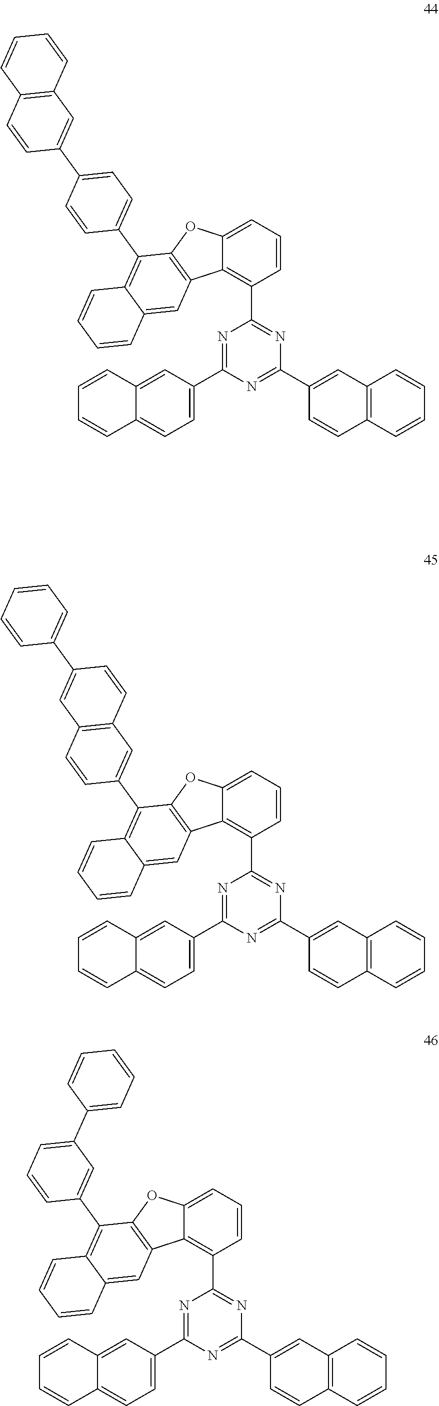 [SMILES: c1ccc2cc(-c3ccc(-c4c5ccccc5cc5c4oc4cccc(-c6nc(-c7ccc8ccccc8c7)nc(-c7ccc8ccccc8c7)n6)c45)cc3)ccc2c1 ; c1ccc(-c2ccc3cc(-c4c5ccccc5cc5c4oc4cccc(-c6nc(-c7ccc8ccccc8c7)nc(-c7ccc8ccccc8c7)n6)c45)ccc3c2)cc1 ; c1ccc(-c2cccc(-c3c4ccccc4cc4c3oc3cccc(-c5nc(-c6ccc7ccccc7c6)nc(-c6ccc7ccccc7c6)n5)c34)c2)cc1]

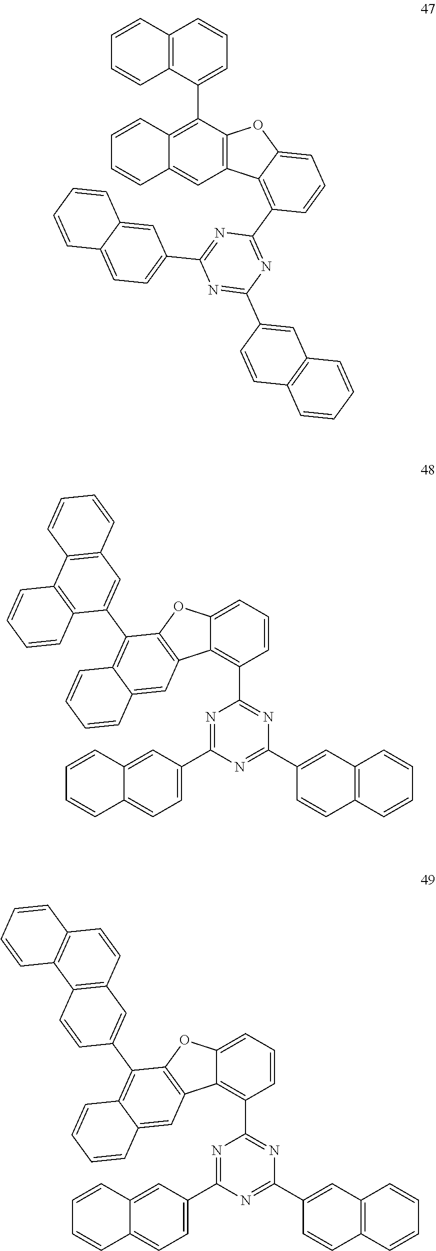 [SMILES: c1ccc2cc(-c3nc(-c4ccc5ccccc5c4)nc(-c4cccc5oc6c(-c7cccc8ccccc78)c7ccccc7cc6c45)n3)ccc2c1 ; c1ccc2cc(-c3nc(-c4ccc5ccccc5c4)nc(-c4cccc5oc6c(-c7cc8ccccc8c8ccccc78)c7ccccc7cc6c45)n3)ccc2c1 ; c1ccc2cc(-c3nc(-c4ccc5ccccc5c4)nc(-c4cccc5oc6c(-c7ccc8c(ccc9ccccc98)c7)c7ccccc7cc6c45)n3)ccc2c1]

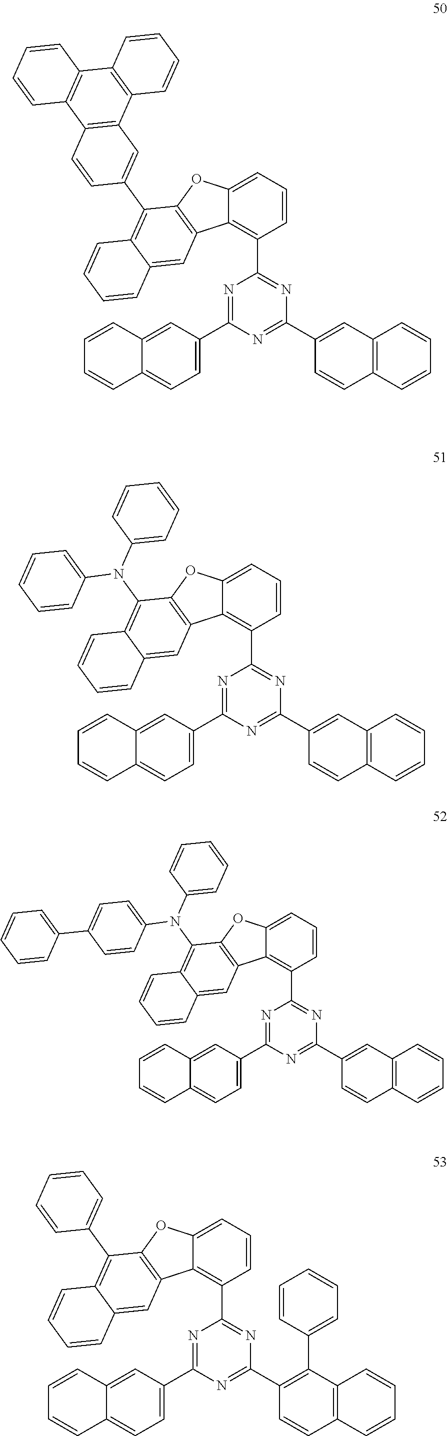 [SMILES: c1ccc2cc(-c3nc(-c4ccc5ccccc5c4)nc(-c4cccc5oc6c(-c7ccc8c9ccccc9c9ccccc9c8c7)c7ccccc7cc6c45)n3)ccc2c1 ; c1ccc(N(c2ccccc2)c2c3ccccc3cc3c2oc2cccc(-c4nc(-c5ccc6ccccc6c5)nc(-c5ccc6ccccc6c5)n4)c23)cc1 ; c1ccc(-c2ccc(N(c3ccccc3)c3c4ccccc4cc4c3oc3cccc(-c5nc(-c6ccc7ccccc7c6)nc(-c6ccc7ccccc7c6)n5)c34)cc2)cc1 ; c1ccc(-c2c(-c3nc(-c4ccc5ccccc5c4)nc(-c4cccc5oc6c(-c7ccccc7)c7ccccc7cc6c45)n3)ccc3ccccc23)cc1]

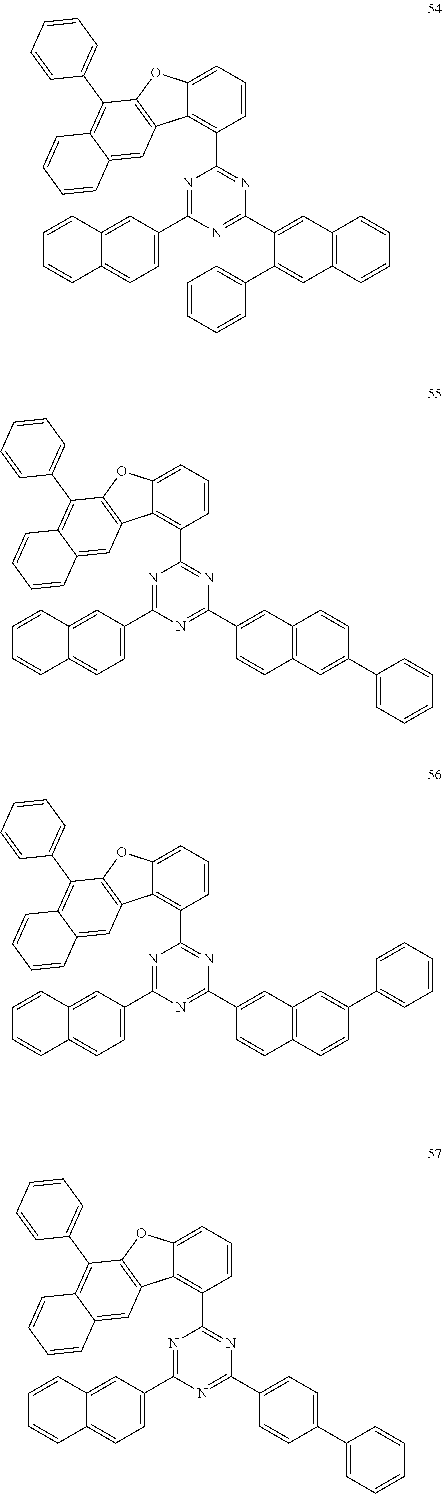 [SMILES: c1ccc(-c2cc3ccccc3cc2-c2nc(-c3ccc4ccccc4c3)nc(-c3cccc4oc5c(-c6ccccc6)c6ccccc6cc5c34)n2)cc1 ; c1ccc(-c2ccc3cc(-c4nc(-c5ccc6ccccc6c5)nc(-c5cccc6oc7c(-c8ccccc8)c8ccccc8cc7c56)n4)ccc3c2)cc1 ; c1ccc(-c2ccc3ccc(-c4nc(-c5ccc6ccccc6c5)nc(-c5cccc6oc7c(-c8ccccc8)c8ccccc8cc7c56)n4)cc3c2)cc1 ; c1ccc(-c2ccc(-c3nc(-c4ccc5ccccc5c4)nc(-c4cccc5oc6c(-c7ccccc7)c7ccccc7cc6c45)n3)cc2)cc1]

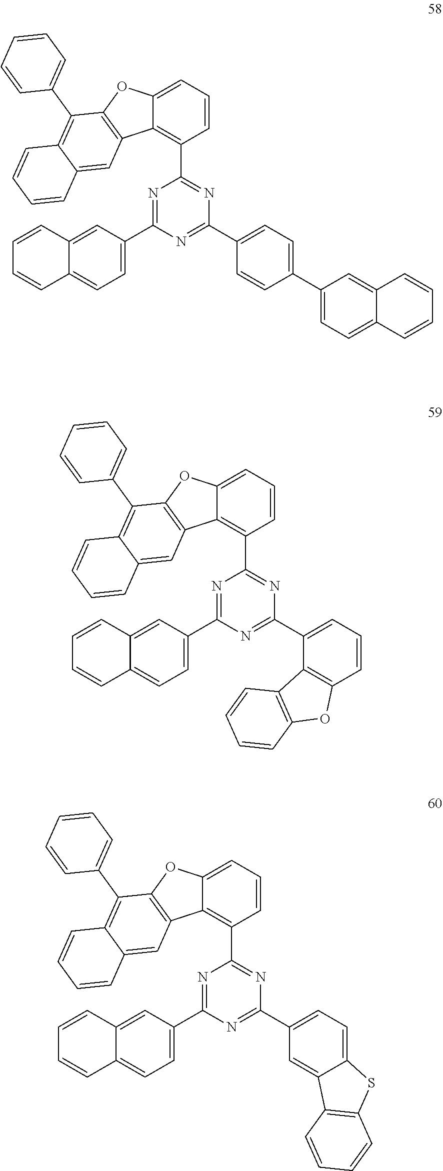 [SMILES: c1ccc(-c2c3ccccc3cc3c2oc2cccc(-c4nc(-c5ccc(-c6ccc7ccccc7c6)cc5)nc(-c5ccc6ccccc6c5)n4)c23)cc1 ; c1ccc(-c2c3ccccc3cc3c2oc2cccc(-c4nc(-c5ccc6ccccc6c5)nc(-c5cccc6oc7ccccc7c56)n4)c23)cc1 ; c1ccc(-c2c3ccccc3cc3c2oc2cccc(-c4nc(-c5ccc6ccccc6c5)nc(-c5ccc6sc7ccccc7c6c5)n4)c23)cc1]

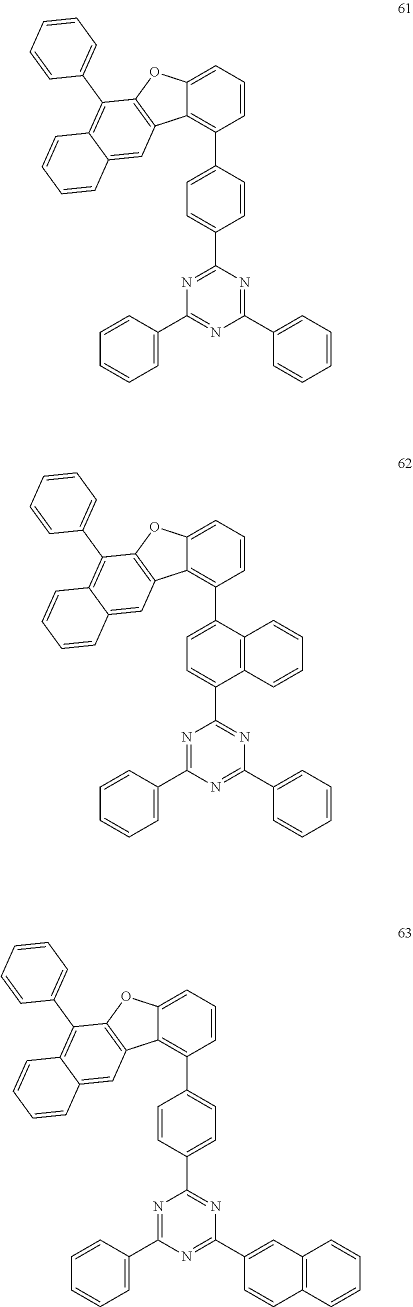 [SMILES: c1ccc(-c2nc(-c3ccccc3)nc(-c3ccc(-c4cccc5oc6c(-c7ccccc7)c7ccccc7cc6c45)cc3)n2)cc1 ; c1ccc(-c2nc(-c3ccccc3)nc(-c3ccc(-c4cccc5oc6c(-c7ccccc7)c7ccccc7cc6c45)c4ccccc34)n2)cc1 ; c1ccc(-c2nc(-c3ccc(-c4cccc5oc6c(-c7ccccc7)c7ccccc7cc6c45)cc3)nc(-c3ccc4ccccc4c3)n2)cc1]

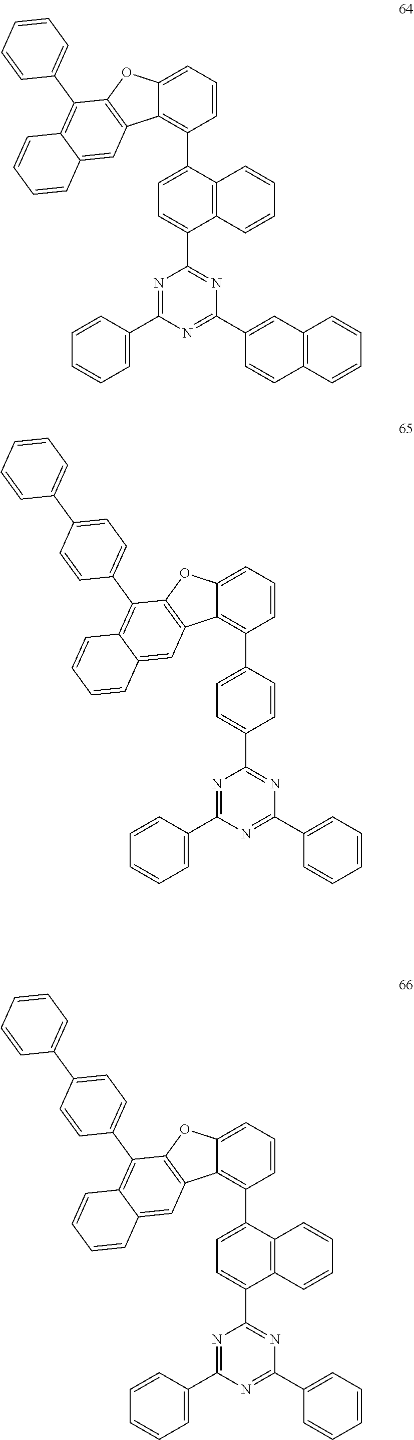 [SMILES: c1ccc(-c2nc(-c3ccc4ccccc4c3)nc(-c3ccc(-c4cccc5oc6c(-c7ccccc7)c7ccccc7cc6c45)c4ccccc34)n2)cc1 ; c1ccc(-c2ccc(-c3c4ccccc4cc4c3oc3cccc(-c5ccc(-c6nc(-c7ccccc7)nc(-c7ccccc7)n6)cc5)c34)cc2)cc1 ; c1ccc(-c2ccc(-c3c4ccccc4cc4c3oc3cccc(-c5ccc(-c6nc(-c7ccccc7)nc(-c7ccccc7)n6)c6ccccc56)c34)cc2)cc1]

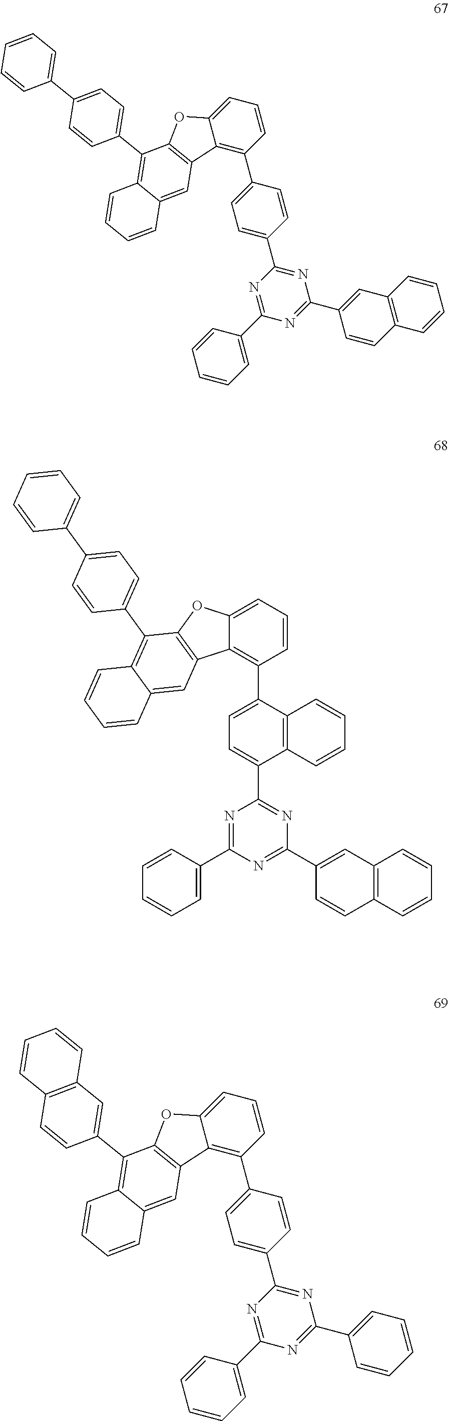 [SMILES: c1ccc(-c2ccc(-c3c4ccccc4cc4c3oc3cccc(-c5ccc(-c6nc(-c7ccccc7)nc(-c7ccc8ccccc8c7)n6)cc5)c34)cc2)cc1 ; c1ccc(-c2ccc(-c3c4ccccc4cc4c3oc3cccc(-c5ccc(-c6nc(-c7ccccc7)nc(-c7ccc8ccccc8c7)n6)c6ccccc56)c34)cc2)cc1 ; c1ccc(-c2nc(-c3ccccc3)nc(-c3ccc(-c4cccc5oc6c(-c7ccc8ccccc8c7)c7ccccc7cc6c45)cc3)n2)cc1]

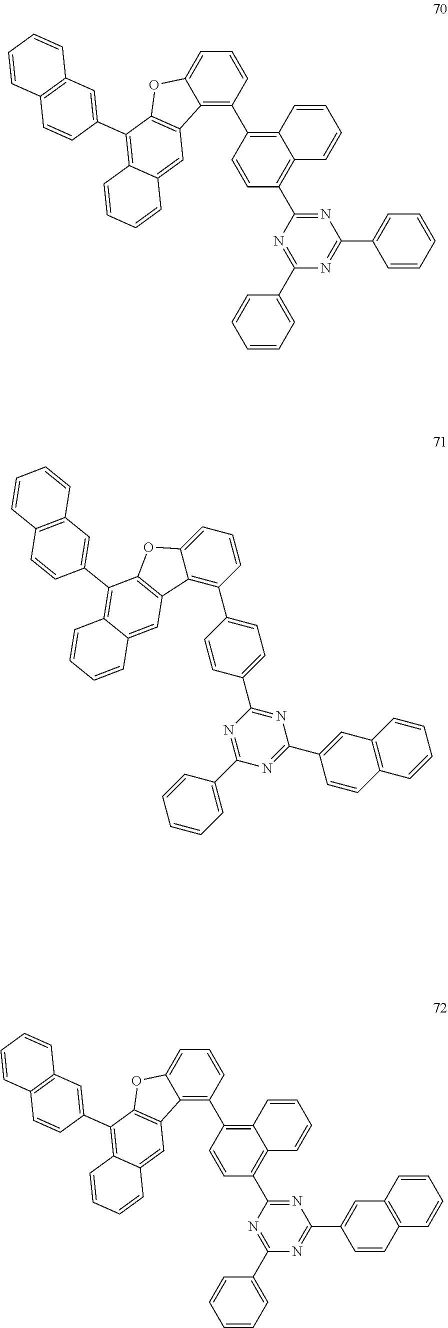 [SMILES: c1ccc(-c2nc(-c3ccccc3)nc(-c3ccc(-c4cccc5oc6c(-c7ccc8ccccc8c7)c7ccccc7cc6c45)c4ccccc34)n2)cc1 ; c1ccc(-c2nc(-c3ccc(-c4cccc5oc6c(-c7ccc8ccccc8c7)c7ccccc7cc6c45)cc3)nc(-c3ccc4ccccc4c3)n2)cc1 ; c1ccc(-c2nc(-c3ccc4ccccc4c3)nc(-c3ccc(-c4cccc5oc6c(-c7ccc8ccccc8c7)c7ccccc7cc6c45)c4ccccc34)n2)cc1]

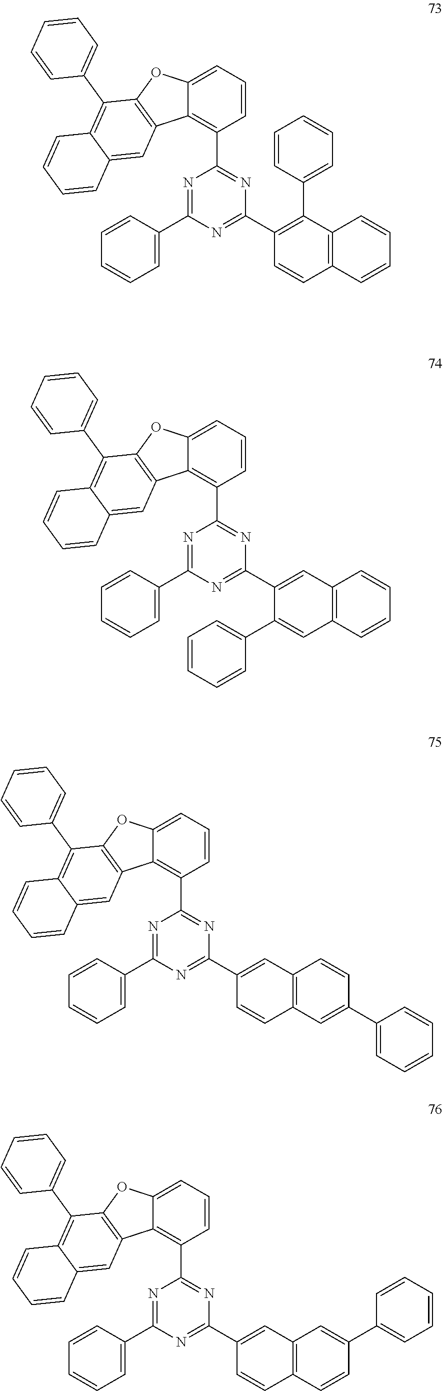 [SMILES: c1ccc(-c2nc(-c3ccc4ccccc4c3-c3ccccc3)nc(-c3cccc4oc5c(-c6ccccc6)c6ccccc6cc5c34)n2)cc1 ; c1ccc(-c2nc(-c3cc4ccccc4cc3-c3ccccc3)nc(-c3cccc4oc5c(-c6ccccc6)c6ccccc6cc5c34)n2)cc1 ; c1ccc(-c2ccc3cc(-c4nc(-c5ccccc5)nc(-c5cccc6oc7c(-c8ccccc8)c8ccccc8cc7c56)n4)ccc3c2)cc1 ; c1ccc(-c2ccc3ccc(-c4nc(-c5ccccc5)nc(-c5cccc6oc7c(-c8ccccc8)c8ccccc8cc7c56)n4)cc3c2)cc1]

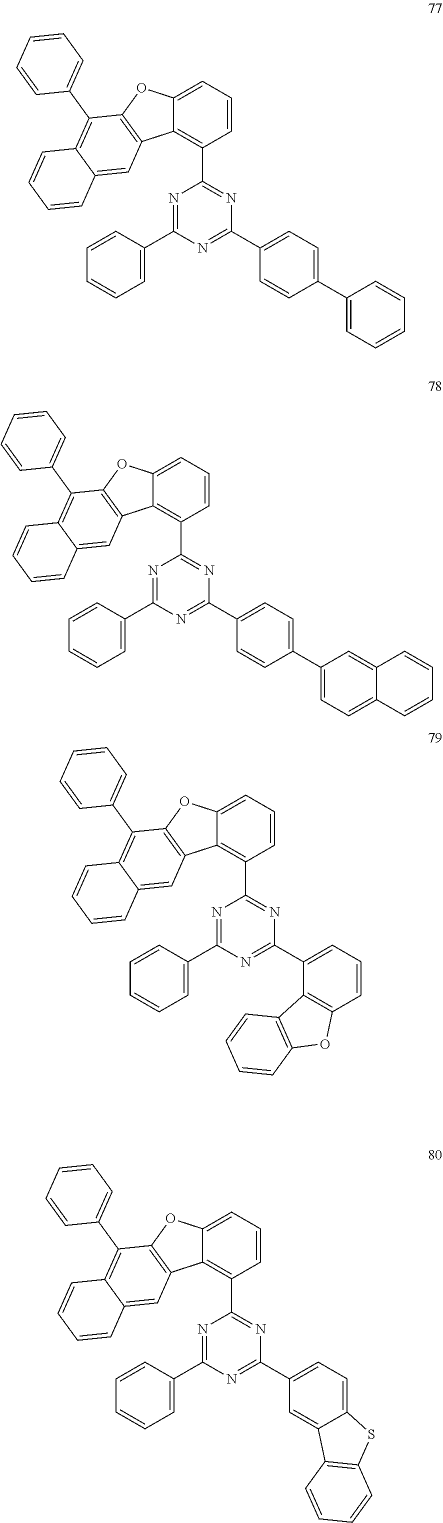 [SMILES: c1ccc(-c2ccc(-c3nc(-c4ccccc4)nc(-c4cccc5oc6c(-c7ccccc7)c7ccccc7cc6c45)n3)cc2)cc1 ; c1ccc(-c2nc(-c3ccc(-c4ccc5ccccc5c4)cc3)nc(-c3cccc4oc5c(-c6ccccc6)c6ccccc6cc5c34)n2)cc1 ; c1ccc(-c2nc(-c3cccc4oc5ccccc5c34)nc(-c3cccc4oc5c(-c6ccccc6)c6ccccc6cc5c34)n2)cc1 ; c1ccc(-c2nc(-c3ccc4sc5ccccc5c4c3)nc(-c3cccc4oc5c(-c6ccccc6)c6ccccc6cc5c34)n2)cc1]

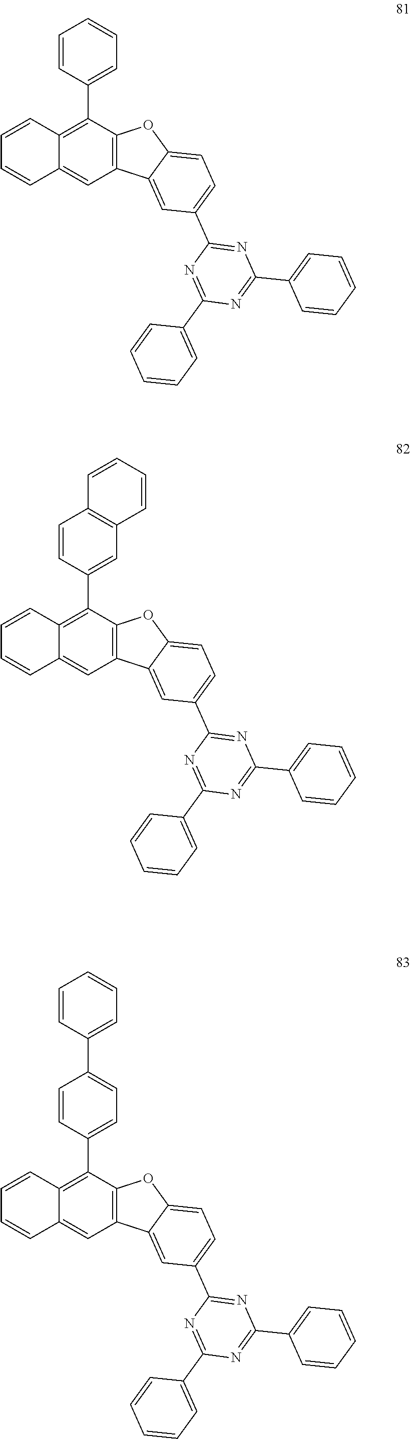 [SMILES: c1ccc(-c2nc(-c3ccccc3)nc(-c3ccc4oc5c(-c6ccccc6)c6ccccc6cc5c4c3)n2)cc1 ; c1ccc(-c2nc(-c3ccccc3)nc(-c3ccc4oc5c(-c6ccc7ccccc7c6)c6ccccc6cc5c4c3)n2)cc1 ; c1ccc(-c2ccc(-c3c4ccccc4cc4c3oc3ccc(-c5nc(-c6ccccc6)nc(-c6ccccc6)n5)cc34)cc2)cc1]

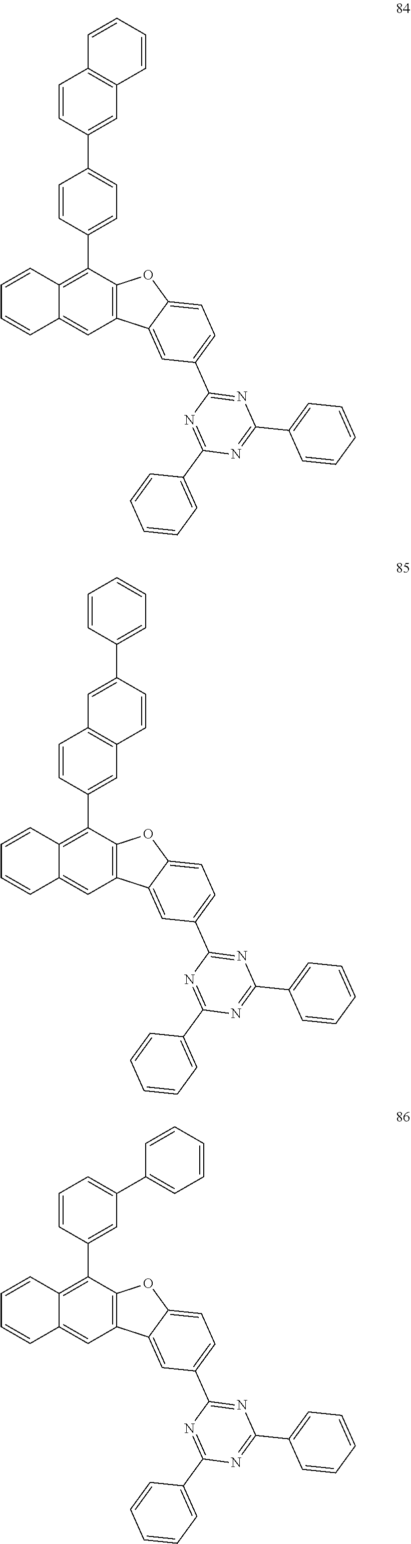

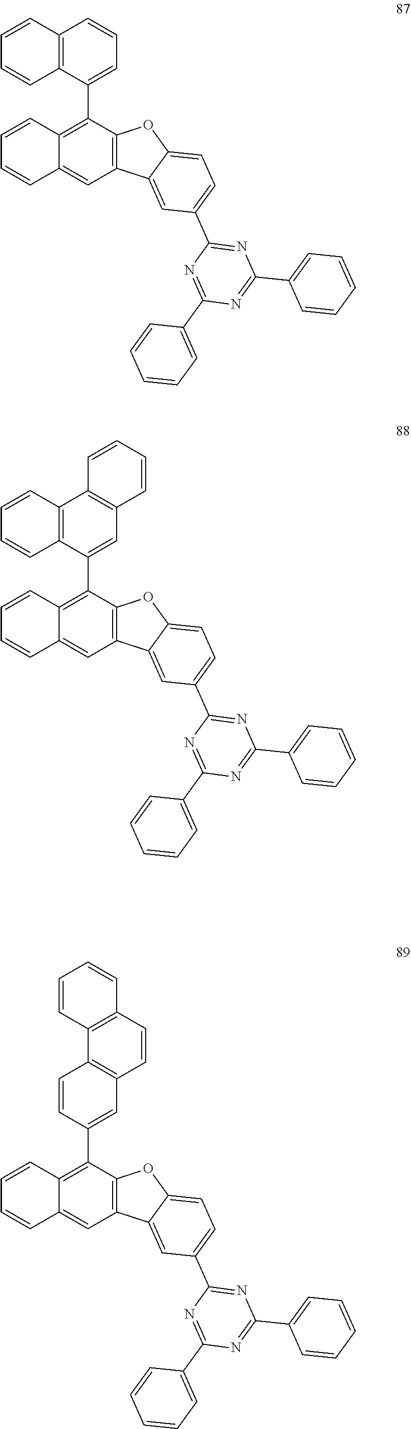 [SMILES: c1ccc(-c2nc(-c3ccccc3)nc(-c3ccc4oc5c(-c6cccc7ccccc67)c6ccccc6cc5c4c3)n2)cc1 ; c1ccc(-c2nc(-c3ccccc3)nc(-c3ccc4oc5c(-c6cc7ccccc7c7ccccc67)c6ccccc6cc5c4c3)n2)cc1 ; c1ccc(-c2nc(-c3ccccc3)nc(-c3ccc4oc5c(-c6ccc7c(ccc8ccccc87)c6)c6ccccc6cc5c4c3)n2)cc1]

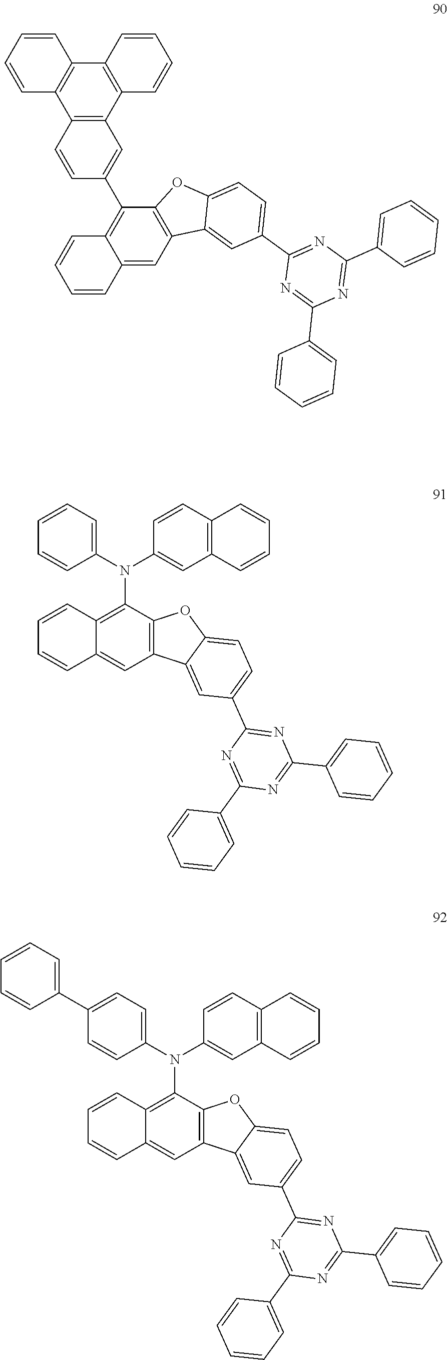 [SMILES: c1ccc(-c2nc(-c3ccccc3)nc(-c3ccc4oc5c(-c6ccc7c8ccccc8c8ccccc8c7c6)c6ccccc6cc5c4c3)n2)cc1 ; c1ccc(-c2nc(-c3ccccc3)nc(-c3ccc4oc5c(N(c6ccccc6)c6ccc7ccccc7c6)c6ccccc6cc5c4c3)n2)cc1 ; c1ccc(-c2ccc(N(c3ccc4ccccc4c3)c3c4ccccc4cc4c3oc3ccc(-c5nc(-c6ccccc6)nc(-c6ccccc6)n5)cc34)cc2)cc1]

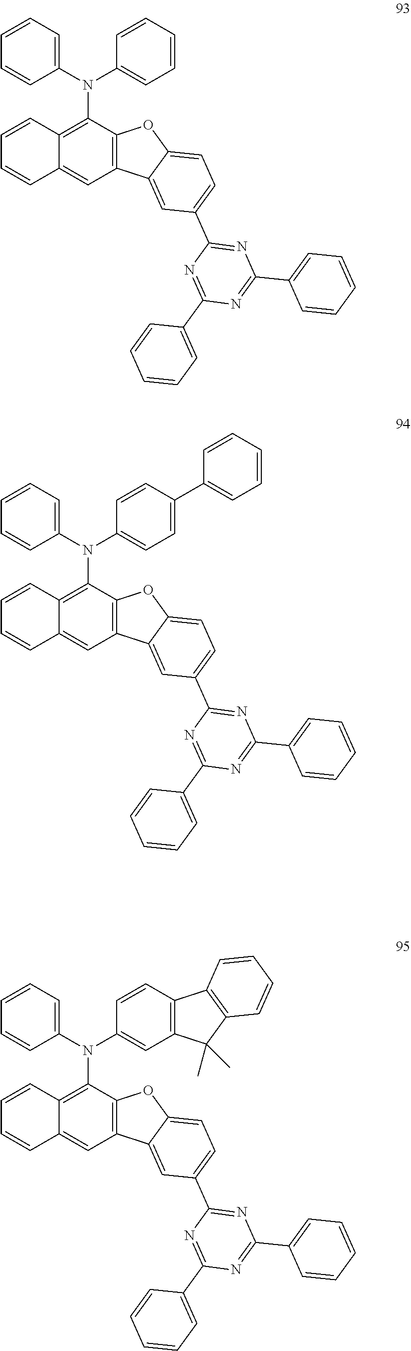 [SMILES: c1ccc(-c2nc(-c3ccccc3)nc(-c3ccc4oc5c(N(c6ccccc6)c6ccccc6)c6ccccc6cc5c4c3)n2)cc1 ; c1ccc(-c2ccc(N(c3ccccc3)c3c4ccccc4cc4c3oc3ccc(-c5nc(-c6ccccc6)nc(-c6ccccc6)n5)cc34)cc2)cc1 ; CC1(C)c2ccccc2-c2ccc(N(c3ccccc3)c3c4ccccc4cc4c3oc3ccc(-c5nc(-c6ccccc6)nc(-c6ccccc6)n5)cc34)cc21]

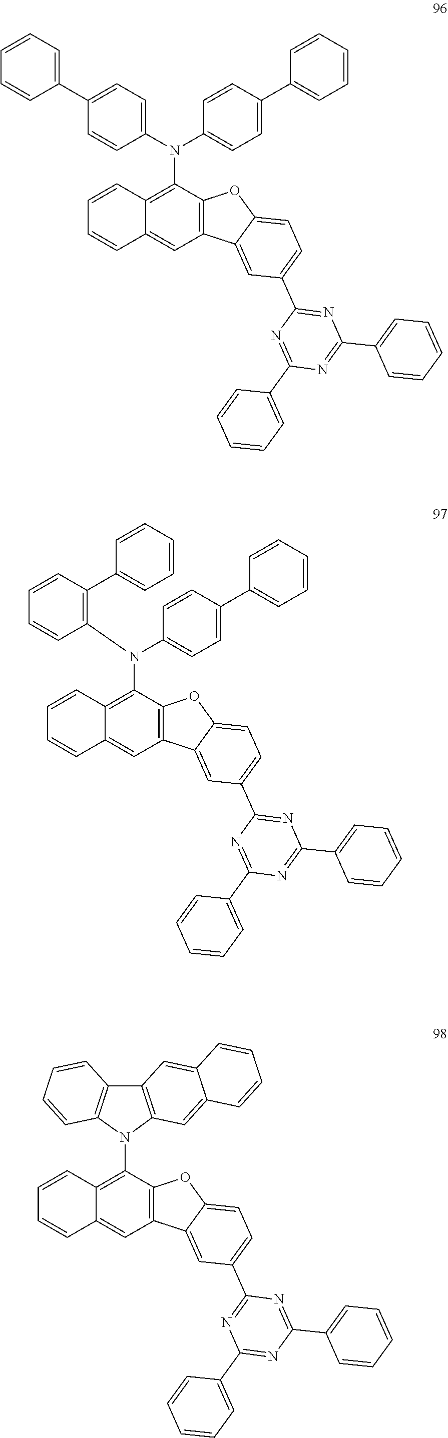 [SMILES: c1ccc(-c2ccc(N(c3ccc(-c4ccccc4)cc3)c3c4ccccc4cc4c3oc3ccc(-c5nc(-c6ccccc6)nc(-c6ccccc6)n5)cc34)cc2)cc1 ; c1ccc(-c2ccc(N(c3ccccc3-c3ccccc3)c3c4ccccc4cc4c3oc3ccc(-c5nc(-c6ccccc6)nc(-c6ccccc6)n5)cc34)cc2)cc1 ; c1ccc(-c2nc(-c3ccccc3)nc(-c3ccc4oc5c(-n6c7ccccc7c7cc8ccccc8cc76)c6ccccc6cc5c4c3)n2)cc1]

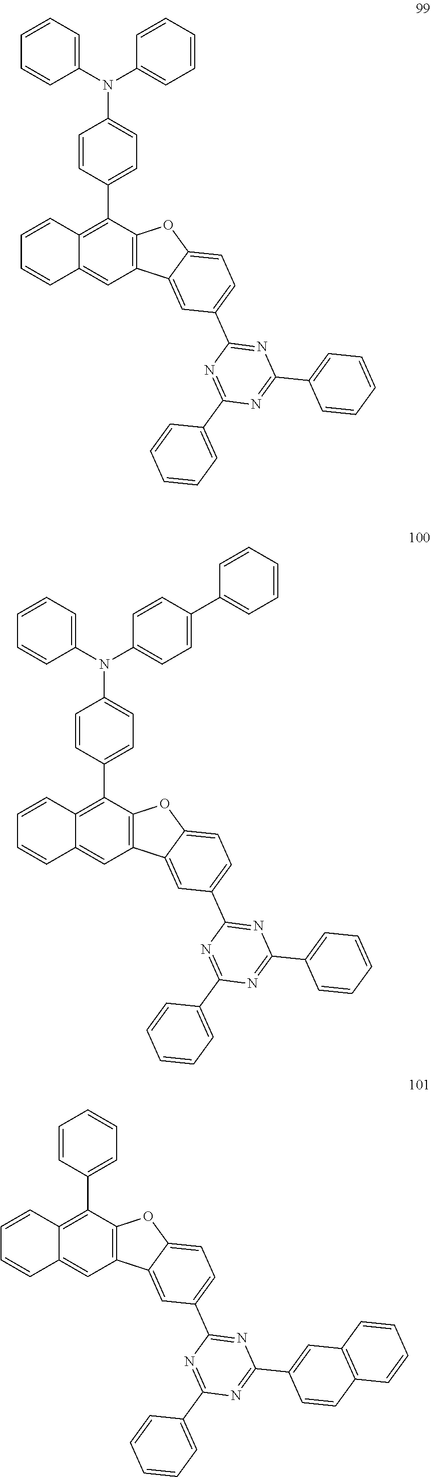 [SMILES: c1ccc(-c2nc(-c3ccccc3)nc(-c3ccc4oc5c(-c6ccc(N(c7ccccc7)c7ccccc7)cc6)c6ccccc6cc5c4c3)n2)cc1 ; c1ccc(-c2ccc(N(c3ccccc3)c3ccc(-c4c5ccccc5cc5c4oc4ccc(-c6nc(-c7ccccc7)nc(-c7ccccc7)n6)cc45)cc3)cc2)cc1 ; c1ccc(-c2nc(-c3ccc4ccccc4c3)nc(-c3ccc4oc5c(-c6ccccc6)c6ccccc6cc5c4c3)n2)cc1]

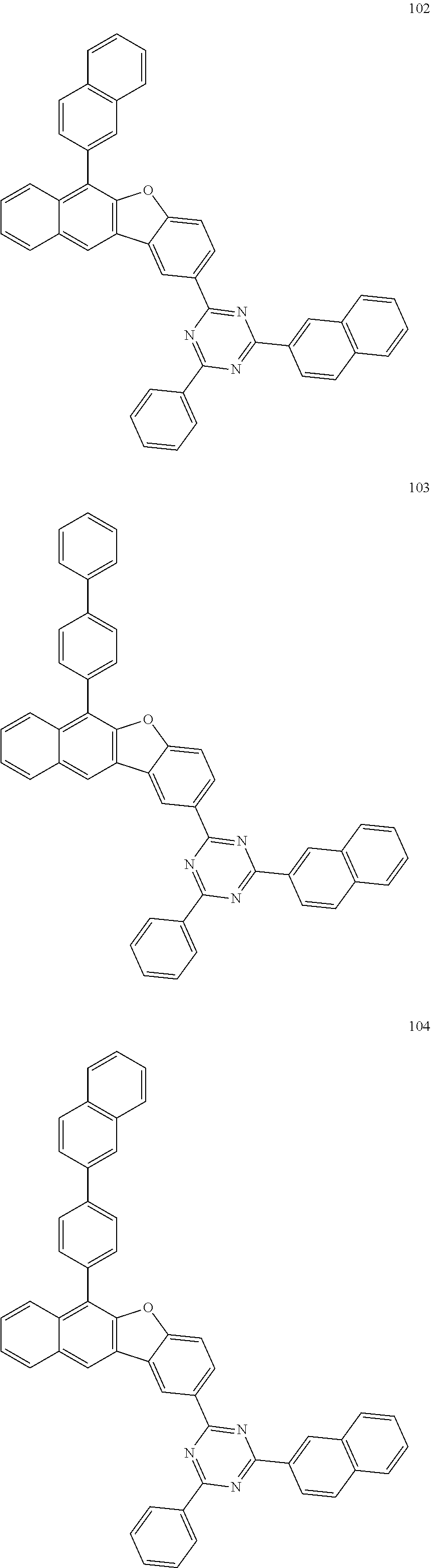 [SMILES: c1ccc(-c2nc(-c3ccc4ccccc4c3)nc(-c3ccc4oc5c(-c6ccc7ccccc7c6)c6ccccc6cc5c4c3)n2)cc1 ; c1ccc(-c2ccc(-c3c4ccccc4cc4c3oc3ccc(-c5nc(-c6ccccc6)nc(-c6ccc7ccccc7c6)n5)cc34)cc2)cc1 ; c1ccc(-c2nc(-c3ccc4ccccc4c3)nc(-c3ccc4oc5c(-c6ccc(-c7ccc8ccccc8c7)cc6)c6ccccc6cc5c4c3)n2)cc1]

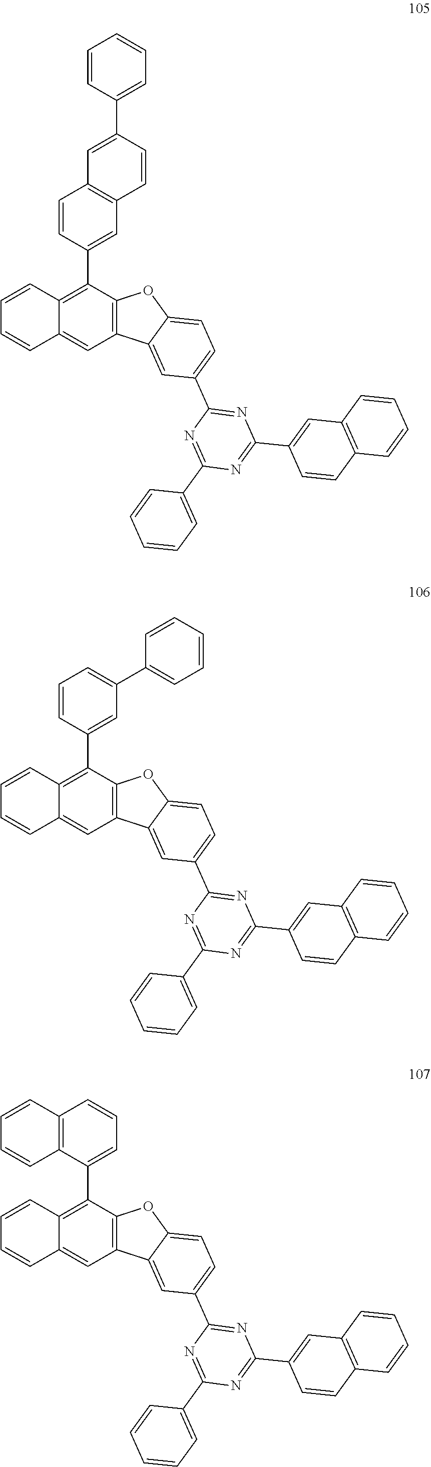 [SMILES: c1ccc(-c2ccc3cc(-c4c5ccccc5cc5c4oc4ccc(-c6nc(-c7ccccc7)nc(-c7ccc8ccccc8c7)n6)cc45)ccc3c2)cc1 ; c1ccc(-c2cccc(-c3c4ccccc4cc4c3oc3ccc(-c5nc(-c6ccccc6)nc(-c6ccc7ccccc7c6)n5)cc34)c2)cc1 ; c1ccc(-c2nc(-c3ccc4ccccc4c3)nc(-c3ccc4oc5c(-c6cccc7ccccc67)c6ccccc6cc5c4c3)n2)cc1]

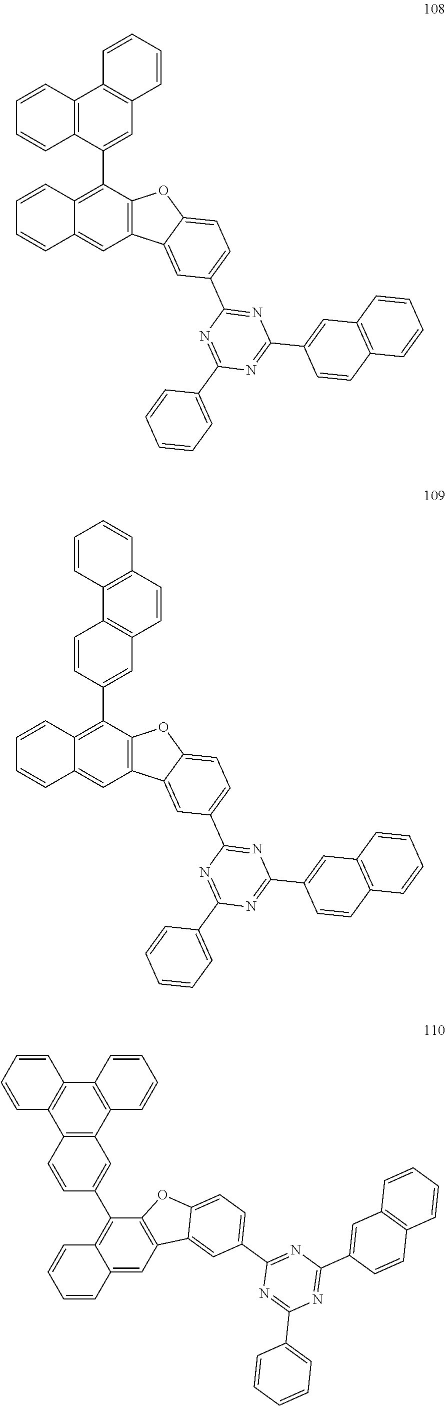 [SMILES: c1ccc(-c2nc(-c3ccc4ccccc4c3)nc(-c3ccc4oc5c(-c6cc7ccccc7c7ccccc67)c6ccccc6cc5c4c3)n2)cc1 ; c1ccc(-c2nc(-c3ccc4ccccc4c3)nc(-c3ccc4oc5c(-c6ccc7c(ccc8ccccc87)c6)c6ccccc6cc5c4c3)n2)cc1 ; c1ccc(-c2nc(-c3ccc4ccccc4c3)nc(-c3ccc4oc5c(-c6ccc7c8ccccc8c8ccccc8c7c6)c6ccccc6cc5c4c3)n2)cc1]

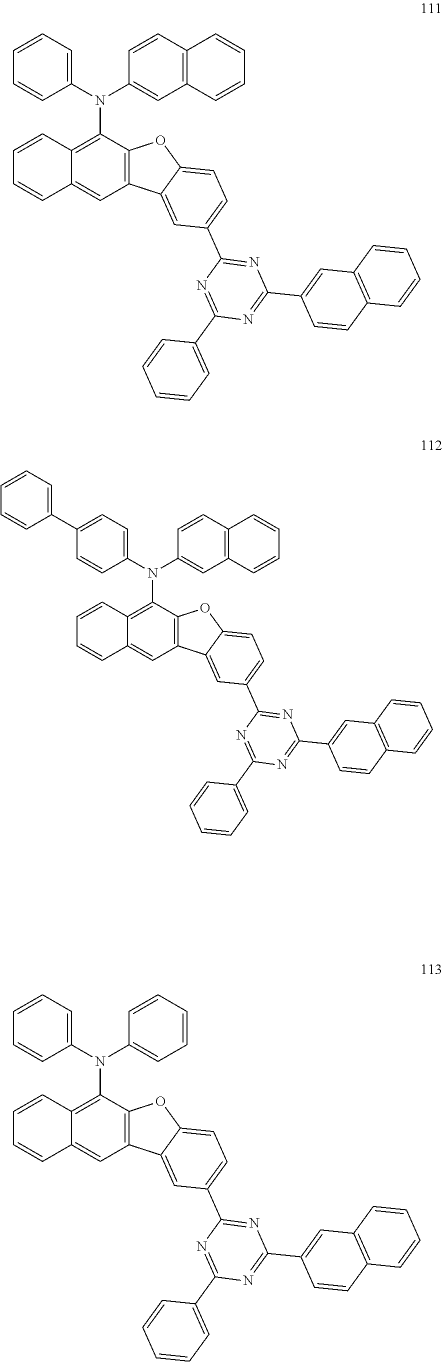 [SMILES: c1ccc(-c2nc(-c3ccc4ccccc4c3)nc(-c3ccc4oc5c(N(c6ccccc6)c6ccc7ccccc7c6)c6ccccc6cc5c4c3)n2)cc1 ; c1ccc(-c2ccc(N(c3ccc4ccccc4c3)c3c4ccccc4cc4c3oc3ccc(-c5nc(-c6ccccc6)nc(-c6ccc7ccccc7c6)n5)cc34)cc2)cc1 ; c1ccc(-c2nc(-c3ccc4ccccc4c3)nc(-c3ccc4oc5c(N(c6ccccc6)c6ccccc6)c6ccccc6cc5c4c3)n2)cc1]

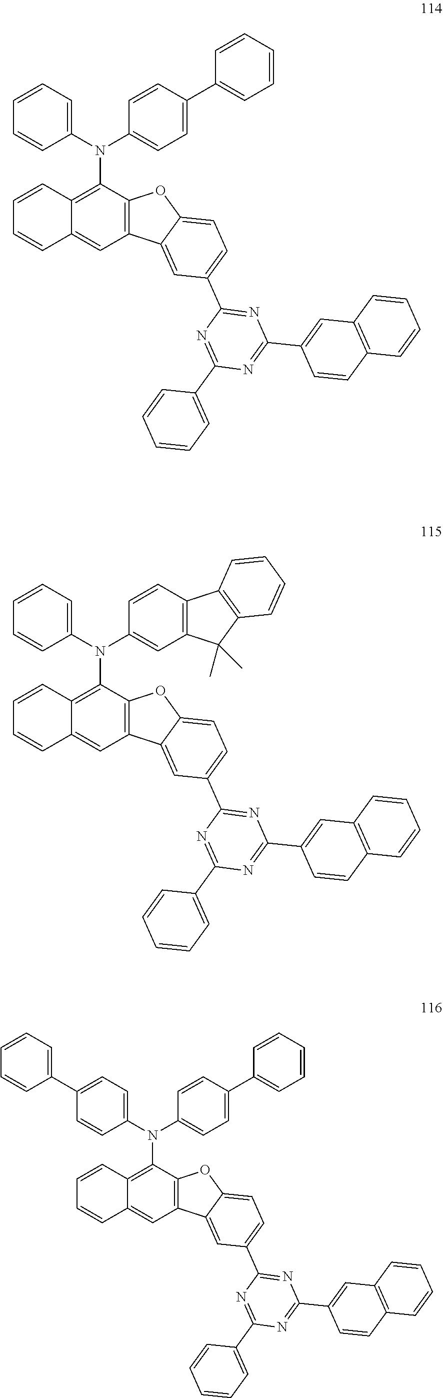 [SMILES: c1ccc(-c2ccc(N(c3ccccc3)c3c4ccccc4cc4c3oc3ccc(-c5nc(-c6ccccc6)nc(-c6ccc7ccccc7c6)n5)cc34)cc2)cc1 ; CC1(C)c2ccccc2-c2ccc(N(c3ccccc3)c3c4ccccc4cc4c3oc3ccc(-c5nc(-c6ccccc6)nc(-c6ccc7ccccc7c6)n5)cc34)cc21 ; c1ccc(-c2ccc(N(c3ccc(-c4ccccc4)cc3)c3c4ccccc4cc4c3oc3ccc(-c5nc(-c6ccccc6)nc(-c6ccc7ccccc7c6)n5)cc34)cc2)cc1]

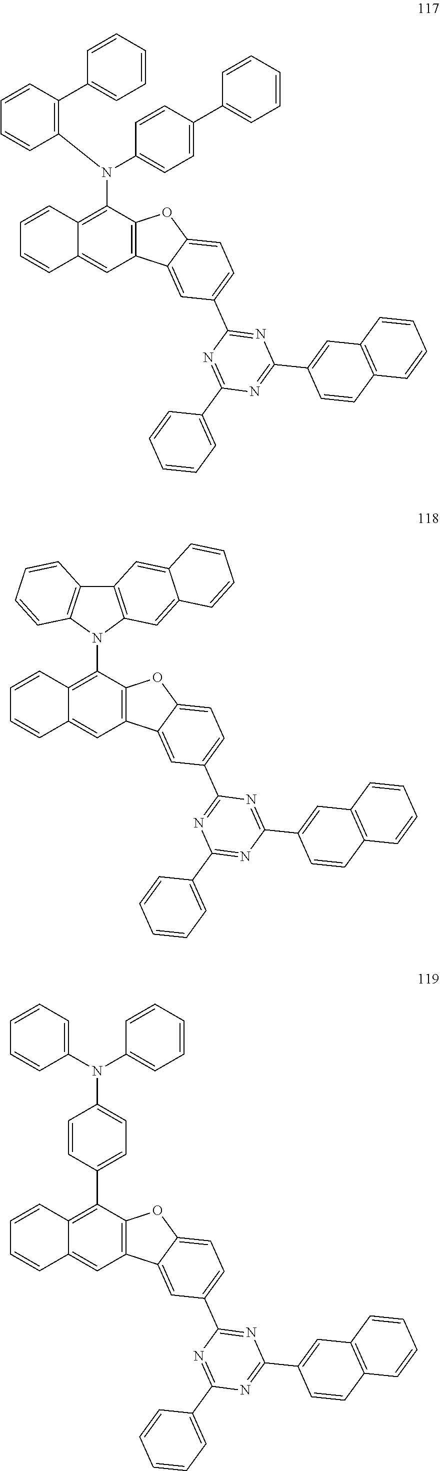 [SMILES: c1ccc(-c2ccc(N(c3ccccc3-c3ccccc3)c3c4ccccc4cc4c3oc3ccc(-c5nc(-c6ccccc6)nc(-c6ccc7ccccc7c6)n5)cc34)cc2)cc1 ; c1ccc(-c2nc(-c3ccc4ccccc4c3)nc(-c3ccc4oc5c(-n6c7ccccc7c7cc8ccccc8cc76)c6ccccc6cc5c4c3)n2)cc1 ; c1ccc(-c2nc(-c3ccc4ccccc4c3)nc(-c3ccc4oc5c(-c6ccc(N(c7ccccc7)c7ccccc7)cc6)c6ccccc6cc5c4c3)n2)cc1]

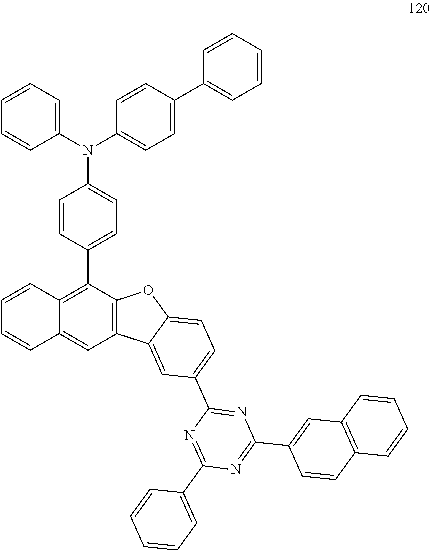 [SMILES: c1ccc(-c2ccc(N(c3ccccc3)c3ccc(-c4c5ccccc5cc5c4oc4ccc(-c6nc(-c7ccccc7)nc(-c7ccc8ccccc8c7)n6)cc45)cc3)cc2)cc1]

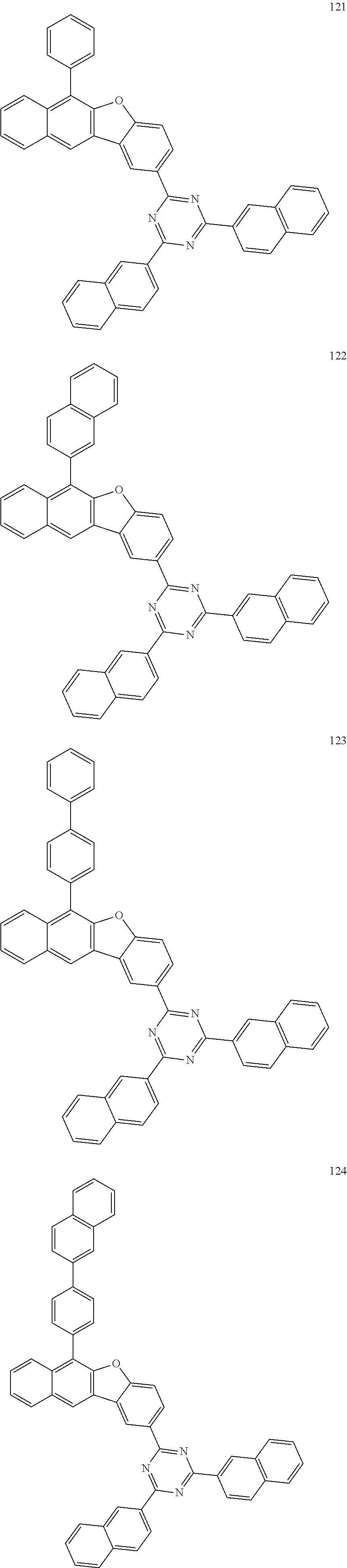

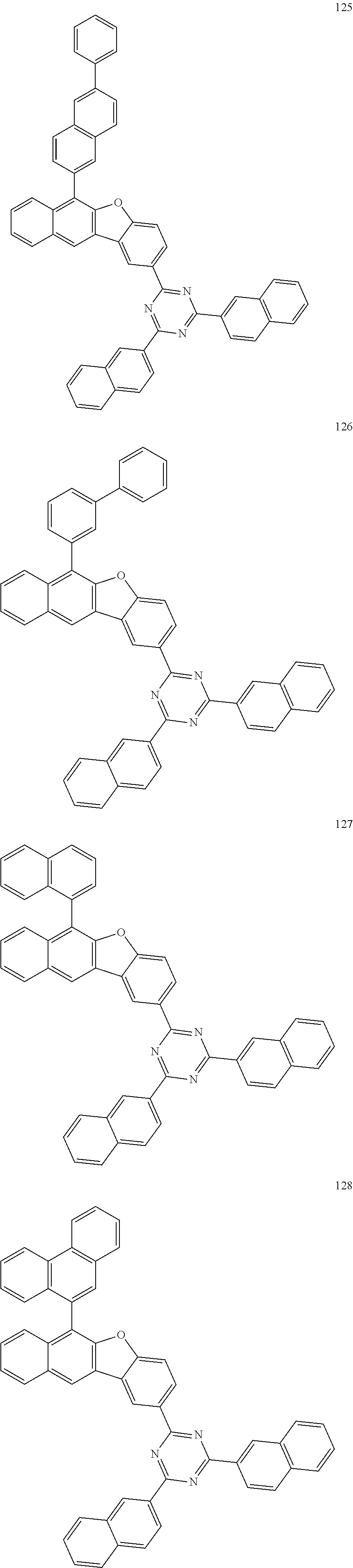 [SMILES: c1ccc(-c2ccc3cc(-c4c5ccccc5cc5c4oc4ccc(-c6nc(-c7ccc8ccccc8c7)nc(-c7ccc8ccccc8c7)n6)cc45)ccc3c2)cc1 ; c1ccc(-c2cccc(-c3c4ccccc4cc4c3oc3ccc(-c5nc(-c6ccc7ccccc7c6)nc(-c6ccc7ccccc7c6)n5)cc34)c2)cc1 ; c1ccc2cc(-c3nc(-c4ccc5ccccc5c4)nc(-c4ccc5oc6c(-c7cccc8ccccc78)c7ccccc7cc6c5c4)n3)ccc2c1 ; c1ccc2cc(-c3nc(-c4ccc5ccccc5c4)nc(-c4ccc5oc6c(-c7cc8ccccc8c8ccccc78)c7ccccc7cc6c5c4)n3)ccc2c1]

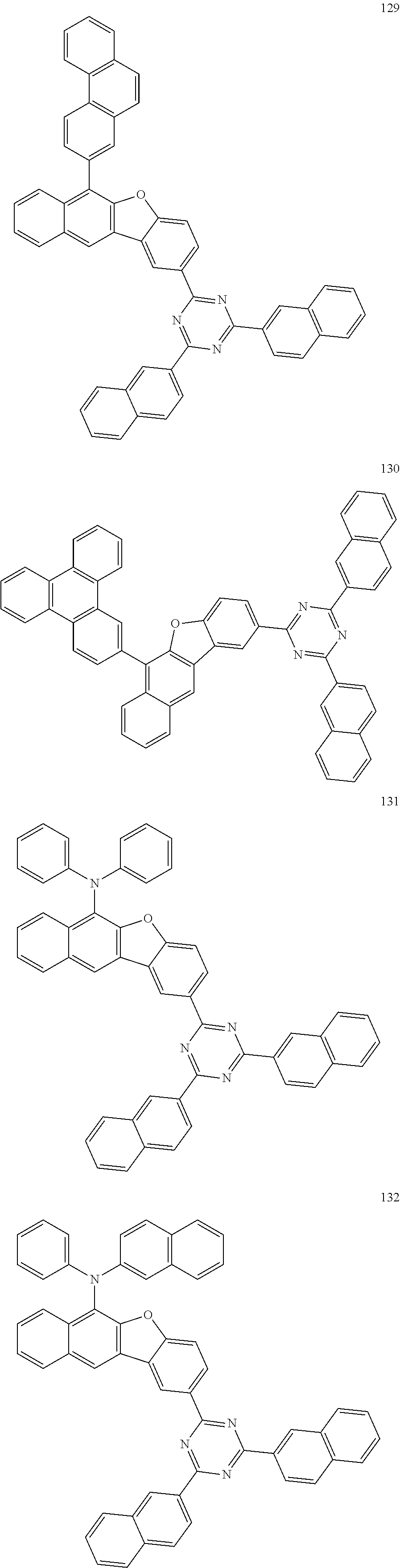 [SMILES: c1ccc2cc(-c3nc(-c4ccc5ccccc5c4)nc(-c4ccc5oc6c(-c7ccc8c(ccc9ccccc98)c7)c7ccccc7cc6c5c4)n3)ccc2c1 ; c1ccc2cc(-c3nc(-c4ccc5ccccc5c4)nc(-c4ccc5oc6c(-c7ccc8c9ccccc9c9ccccc9c8c7)c7ccccc7cc6c5c4)n3)ccc2c1 ; c1ccc(N(c2ccccc2)c2c3ccccc3cc3c2oc2ccc(-c4nc(-c5ccc6ccccc6c5)nc(-c5ccc6ccccc6c5)n4)cc23)cc1 ; c1ccc(N(c2ccc3ccccc3c2)c2c3ccccc3cc3c2oc2ccc(-c4nc(-c5ccc6ccccc6c5)nc(-c5ccc6ccccc6c5)n4)cc23)cc1]

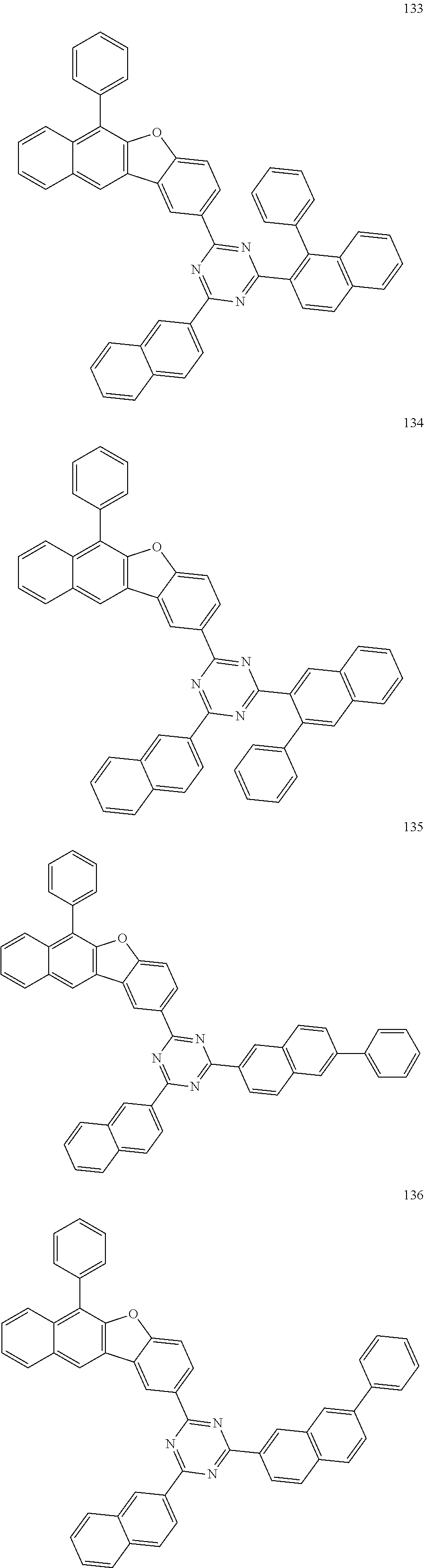 [SMILES: c1ccc(-c2c(-c3nc(-c4ccc5ccccc5c4)nc(-c4ccc5oc6c(-c7ccccc7)c7ccccc7cc6c5c4)n3)ccc3ccccc23)cc1 ; c1ccc(-c2cc3ccccc3cc2-c2nc(-c3ccc4ccccc4c3)nc(-c3ccc4oc5c(-c6ccccc6)c6ccccc6cc5c4c3)n2)cc1 ; c1ccc(-c2ccc3cc(-c4nc(-c5ccc6ccccc6c5)nc(-c5ccc6oc7c(-c8ccccc8)c8ccccc8cc7c6c5)n4)ccc3c2)cc1 ; c1ccc(-c2ccc3ccc(-c4nc(-c5ccc6ccccc6c5)nc(-c5ccc6oc7c(-c8ccccc8)c8ccccc8cc7c6c5)n4)cc3c2)cc1]

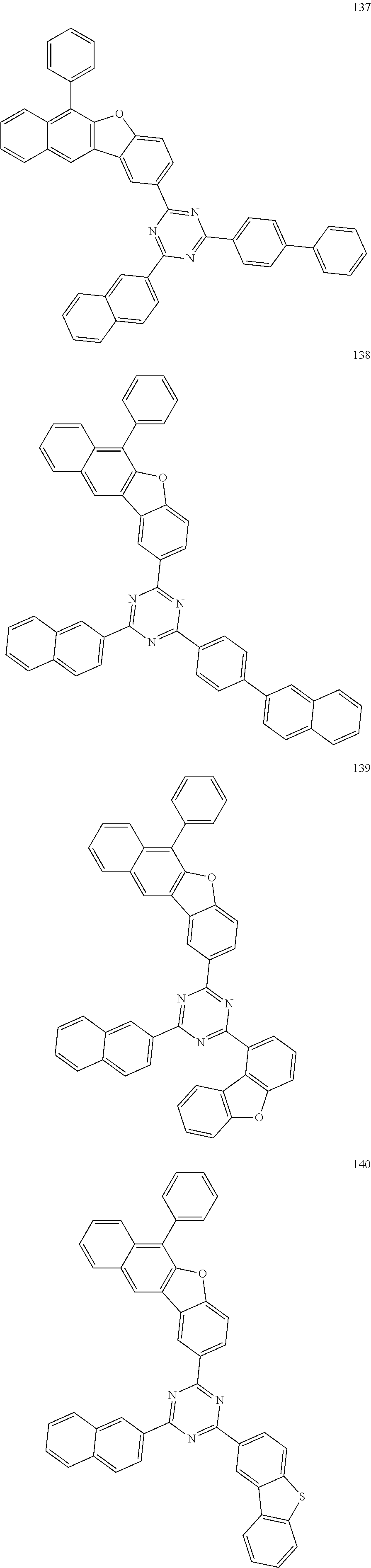

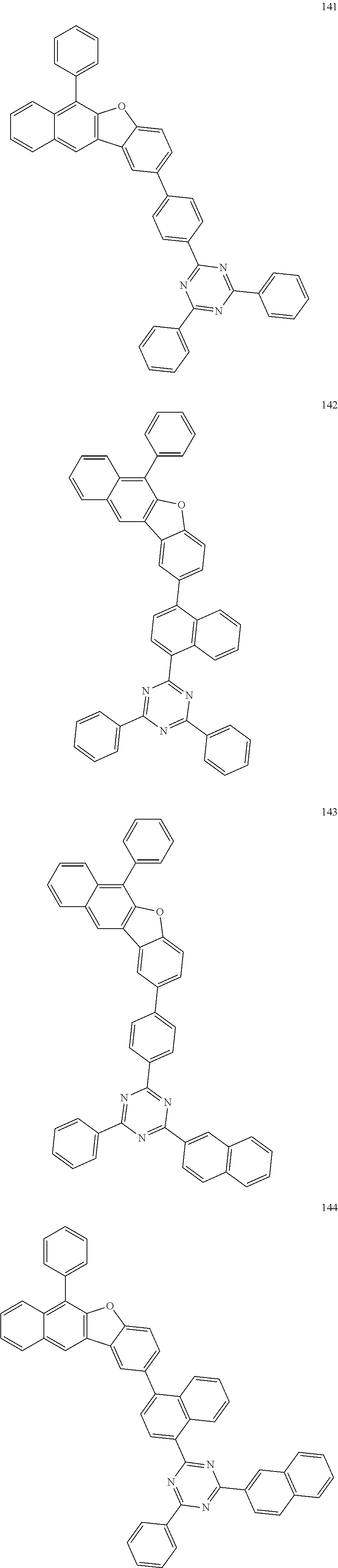 [SMILES: c1ccc(-c2nc(-c3ccccc3)nc(-c3ccc(-c4ccc5oc6c(-c7ccccc7)c7ccccc7cc6c5c4)cc3)n2)cc1 ; c1ccc(-c2nc(-c3ccccc3)nc(-c3ccc(-c4ccc5oc6c(-c7ccccc7)c7ccccc7cc6c5c4)c4ccccc34)n2)cc1 ; c1ccc(-c2nc(-c3ccc(-c4ccc5oc6c(-c7ccccc7)c7ccccc7cc6c5c4)cc3)nc(-c3ccc4ccccc4c3)n2)cc1 ; c1ccc(-c2nc(-c3ccc4ccccc4c3)nc(-c3ccc(-c4ccc5oc6c(-c7ccccc7)c7ccccc7cc6c5c4)c4ccccc34)n2)cc1]

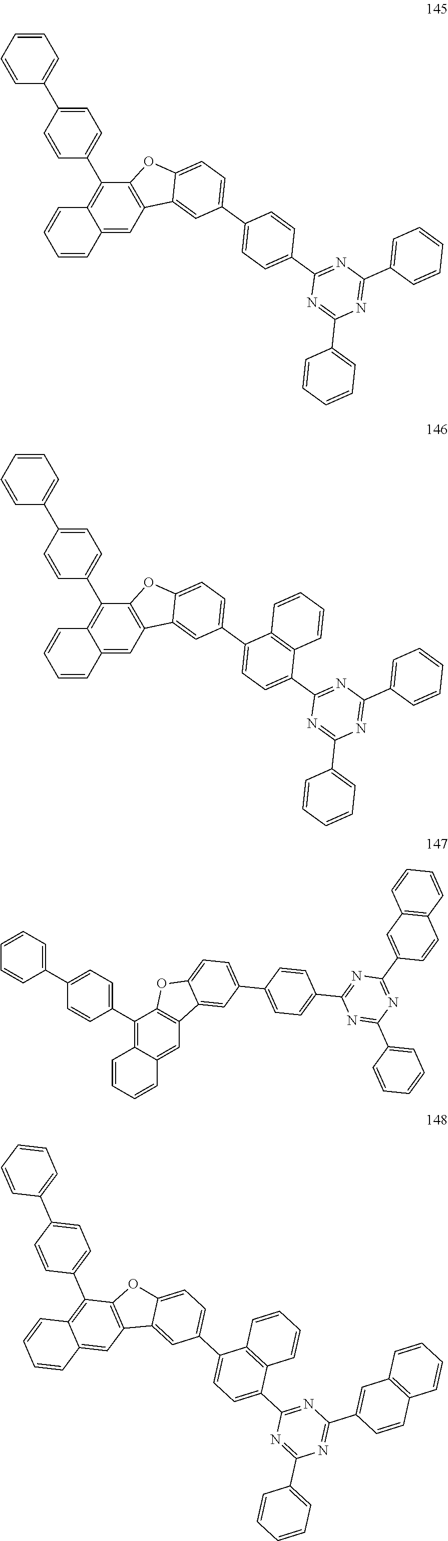 [SMILES: c1ccc(-c2ccc(-c3c4ccccc4cc4c3oc3ccc(-c5ccc(-c6nc(-c7ccccc7)nc(-c7ccccc7)n6)cc5)cc34)cc2)cc1 ; c1ccc(-c2ccc(-c3c4ccccc4cc4c3oc3ccc(-c5ccc(-c6nc(-c7ccccc7)nc(-c7ccccc7)n6)c6ccccc56)cc34)cc2)cc1 ; c1ccc(-c2ccc(-c3c4ccccc4cc4c3oc3ccc(-c5ccc(-c6nc(-c7ccccc7)nc(-c7ccc8ccccc8c7)n6)cc5)cc34)cc2)cc1 ; c1ccc(-c2ccc(-c3c4ccccc4cc4c3oc3ccc(-c5ccc(-c6nc(-c7ccccc7)nc(-c7ccc8ccccc8c7)n6)c6ccccc56)cc34)cc2)cc1]

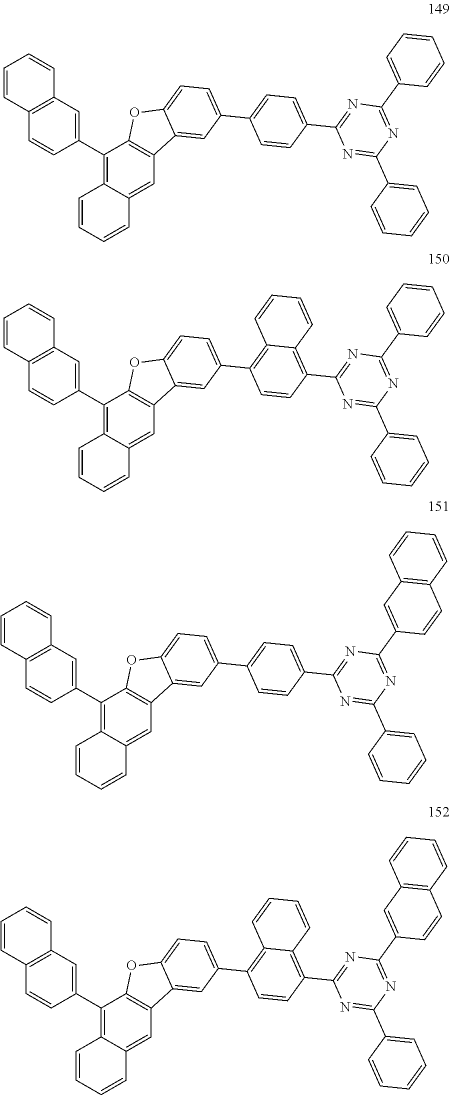 [SMILES: c1ccc(-c2nc(-c3ccccc3)nc(-c3ccc(-c4ccc5oc6c(-c7ccc8ccccc8c7)c7ccccc7cc6c5c4)cc3)n2)cc1 ; c1ccc(-c2nc(-c3ccccc3)nc(-c3ccc(-c4ccc5oc6c(-c7ccc8ccccc8c7)c7ccccc7cc6c5c4)c4ccccc34)n2)cc1 ; c1ccc(-c2nc(-c3ccc(-c4ccc5oc6c(-c7ccc8ccccc8c7)c7ccccc7cc6c5c4)cc3)nc(-c3ccc4ccccc4c3)n2)cc1 ; c1ccc(-c2nc(-c3ccc4ccccc4c3)nc(-c3ccc(-c4ccc5oc6c(-c7ccc8ccccc8c7)c7ccccc7cc6c5c4)c4ccccc34)n2)cc1]

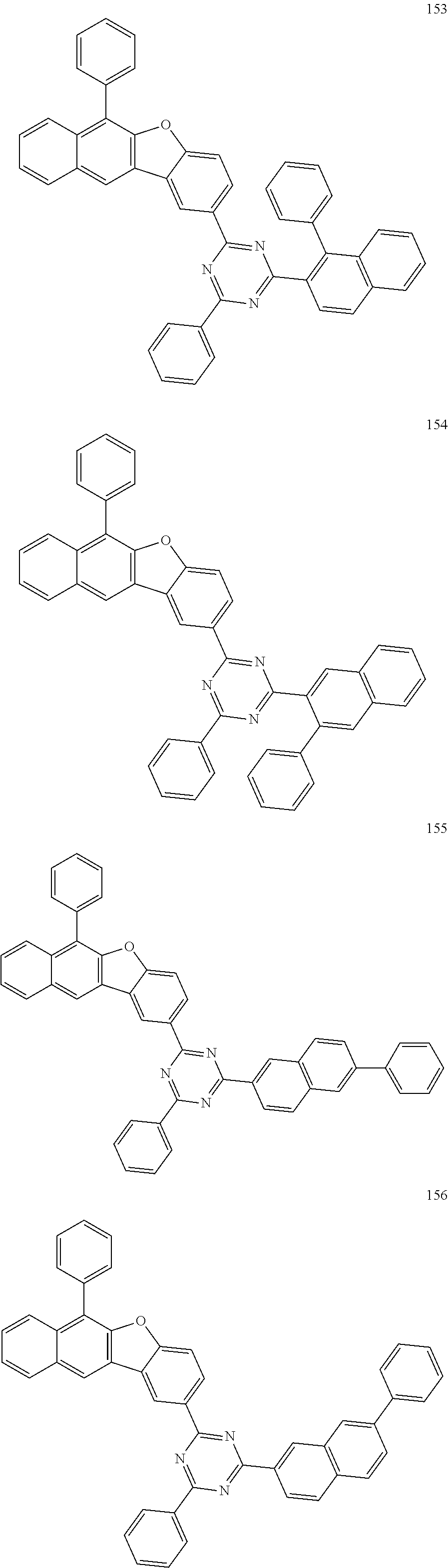 [SMILES: c1ccc(-c2nc(-c3ccc4oc5c(-c6ccccc6)c6ccccc6cc5c4c3)nc(-c3ccc4ccccc4c3-c3ccccc3)n2)cc1 ; c1ccc(-c2nc(-c3ccc4oc5c(-c6ccccc6)c6ccccc6cc5c4c3)nc(-c3cc4ccccc4cc3-c3ccccc3)n2)cc1 ; c1ccc(-c2ccc3cc(-c4nc(-c5ccccc5)nc(-c5ccc6oc7c(-c8ccccc8)c8ccccc8cc7c6c5)n4)ccc3c2)cc1 ; c1ccc(-c2ccc3ccc(-c4nc(-c5ccccc5)nc(-c5ccc6oc7c(-c8ccccc8)c8ccccc8cc7c6c5)n4)cc3c2)cc1]

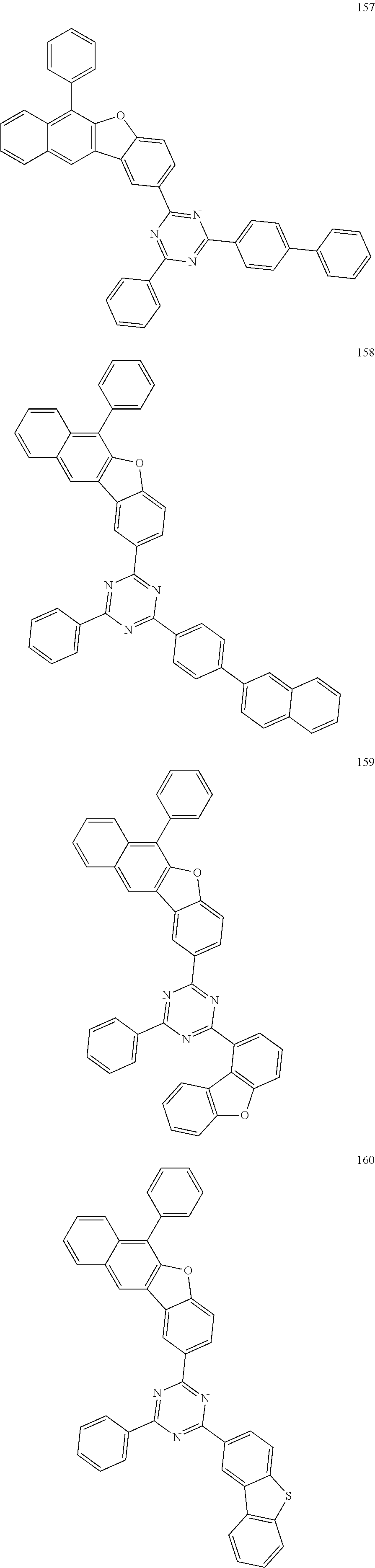

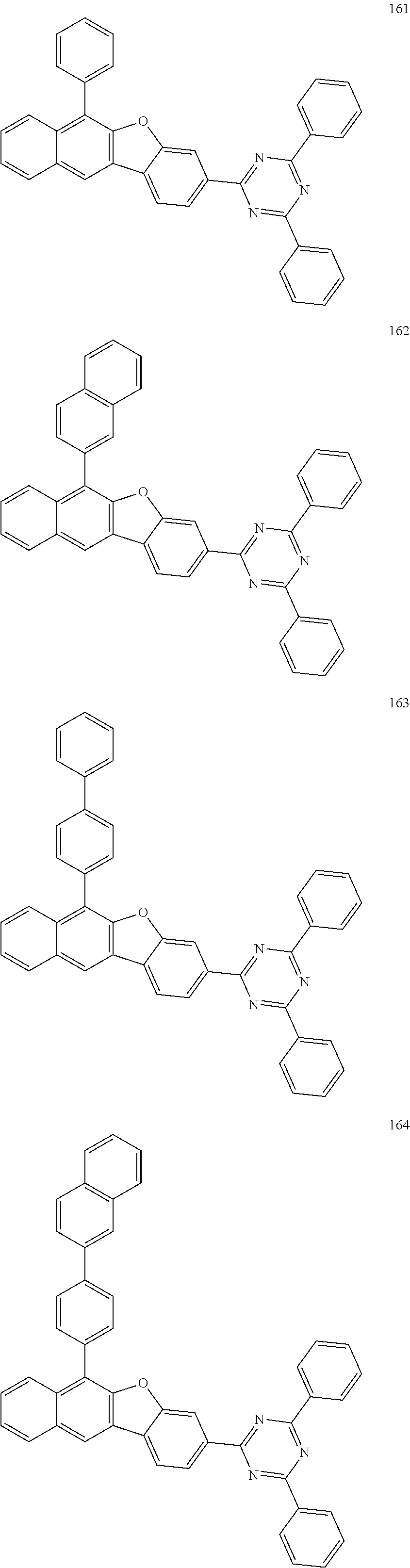 [SMILES: c1ccc(-c2nc(-c3ccccc3)nc(-c3ccc4c(c3)oc3c(-c5ccccc5)c5ccccc5cc34)n2)cc1 ; c1ccc(-c2nc(-c3ccccc3)nc(-c3ccc4c(c3)oc3c(-c5ccc6ccccc6c5)c5ccccc5cc34)n2)cc1 ; c1ccc(-c2ccc(-c3c4ccccc4cc4c3oc3cc(-c5nc(-c6ccccc6)nc(-c6ccccc6)n5)ccc34)cc2)cc1 ; c1ccc(-c2nc(-c3ccccc3)nc(-c3ccc4c(c3)oc3c(-c5ccc(-c6ccc7ccccc7c6)cc5)c5ccccc5cc34)n2)cc1]

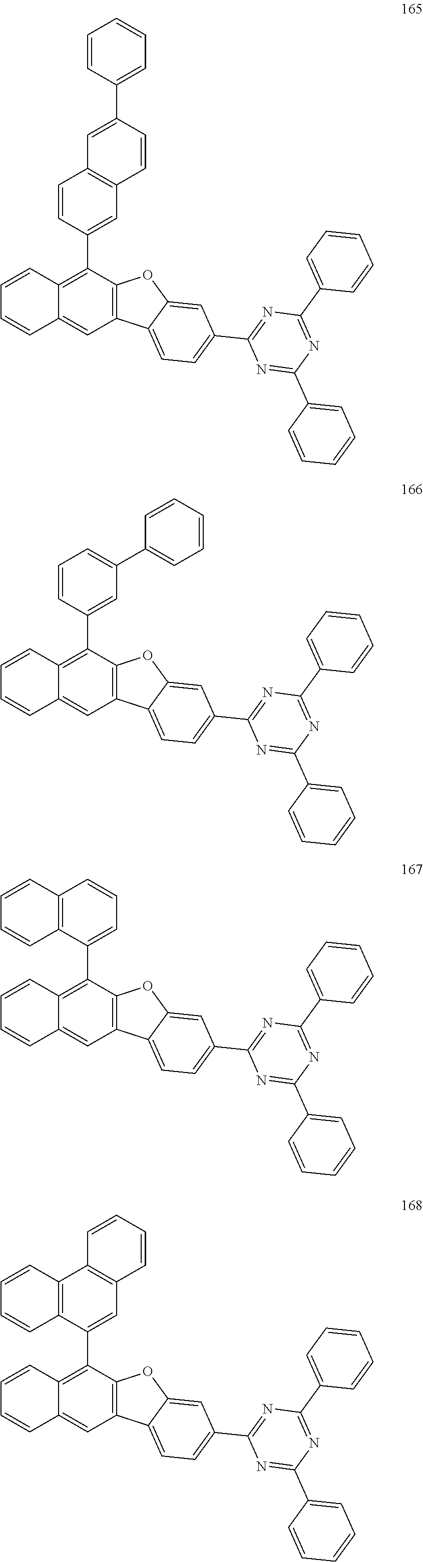 [SMILES: c1ccc(-c2ccc3cc(-c4c5ccccc5cc5c4oc4cc(-c6nc(-c7ccccc7)nc(-c7ccccc7)n6)ccc45)ccc3c2)cc1 ; c1ccc(-c2cccc(-c3c4ccccc4cc4c3oc3cc(-c5nc(-c6ccccc6)nc(-c6ccccc6)n5)ccc34)c2)cc1 ; c1ccc(-c2nc(-c3ccccc3)nc(-c3ccc4c(c3)oc3c(-c5cccc6ccccc56)c5ccccc5cc34)n2)cc1 ; c1ccc(-c2nc(-c3ccccc3)nc(-c3ccc4c(c3)oc3c(-c5cc6ccccc6c6ccccc56)c5ccccc5cc34)n2)cc1]

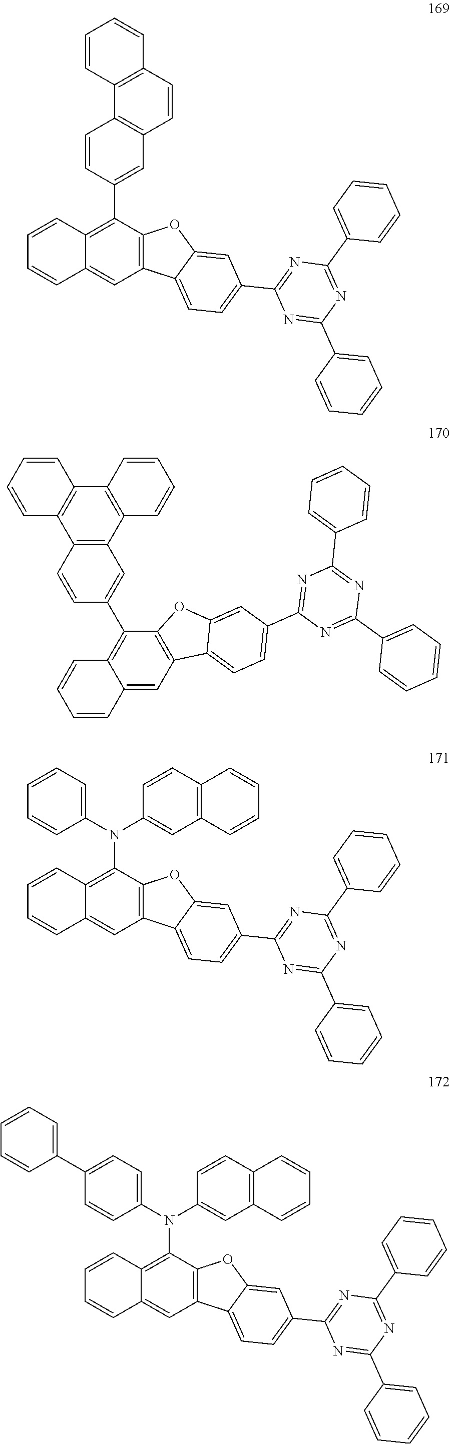 [SMILES: c1ccc(-c2nc(-c3ccccc3)nc(-c3ccc4c(c3)oc3c(-c5ccc6c(ccc7ccccc76)c5)c5ccccc5cc34)n2)cc1 ; c1ccc(-c2nc(-c3ccccc3)nc(-c3ccc4c(c3)oc3c(-c5ccc6c7ccccc7c7ccccc7c6c5)c5ccccc5cc34)n2)cc1 ; c1ccc(-c2nc(-c3ccccc3)nc(-c3ccc4c(c3)oc3c(N(c5ccccc5)c5ccc6ccccc6c5)c5ccccc5cc34)n2)cc1 ; c1ccc(-c2ccc(N(c3ccc4ccccc4c3)c3c4ccccc4cc4c3oc3cc(-c5nc(-c6ccccc6)nc(-c6ccccc6)n5)ccc34)cc2)cc1]

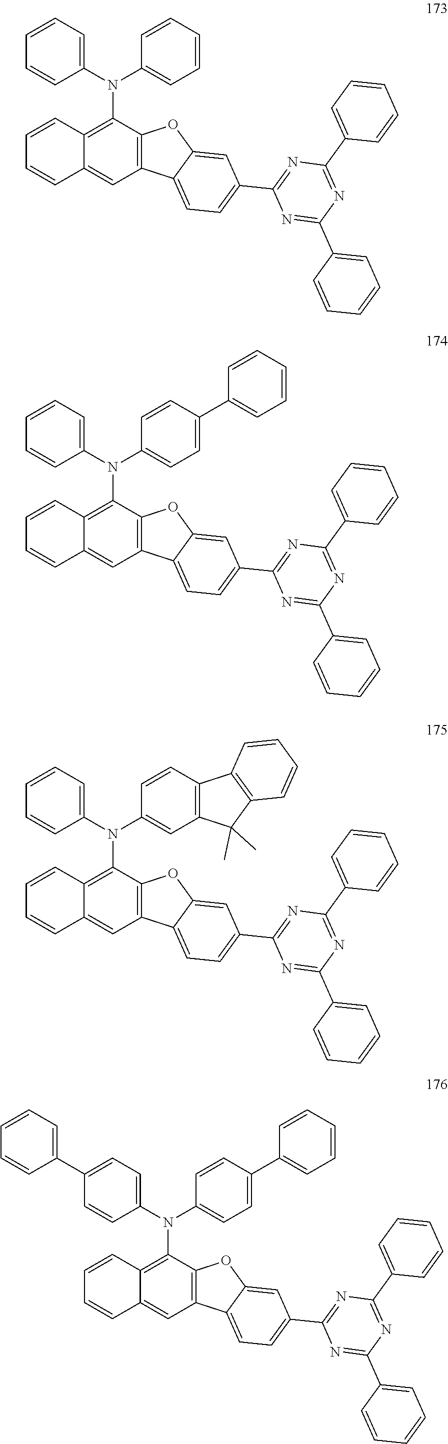 [SMILES: c1ccc(-c2nc(-c3ccccc3)nc(-c3ccc4c(c3)oc3c(N(c5ccccc5)c5ccccc5)c5ccccc5cc34)n2)cc1 ; c1ccc(-c2ccc(N(c3ccccc3)c3c4ccccc4cc4c3oc3cc(-c5nc(-c6ccccc6)nc(-c6ccccc6)n5)ccc34)cc2)cc1 ; CC1(C)c2ccccc2-c2ccc(N(c3ccccc3)c3c4ccccc4cc4c3oc3cc(-c5nc(-c6ccccc6)nc(-c6ccccc6)n5)ccc34)cc21 ; c1ccc(-c2ccc(N(c3ccc(-c4ccccc4)cc3)c3c4ccccc4cc4c3oc3cc(-c5nc(-c6ccccc6)nc(-c6ccccc6)n5)ccc34)cc2)cc1]

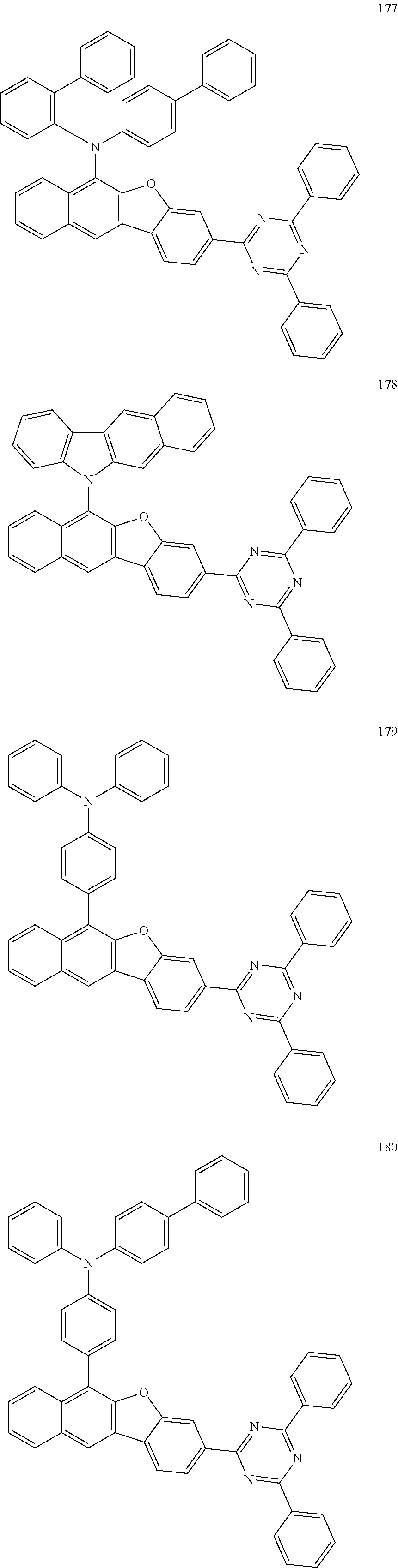 [SMILES: c1ccc(-c2ccc(N(c3ccccc3-c3ccccc3)c3c4ccccc4cc4c3oc3cc(-c5nc(-c6ccccc6)nc(-c6ccccc6)n5)ccc34)cc2)cc1 ; c1ccc(-c2nc(-c3ccccc3)nc(-c3ccc4c(c3)oc3c(-n5c6ccccc6c6cc7ccccc7cc65)c5ccccc5cc34)n2)cc1 ; c1ccc(-c2nc(-c3ccccc3)nc(-c3ccc4c(c3)oc3c(-c5ccc(N(c6ccccc6)c6ccccc6)cc5)c5ccccc5cc34)n2)cc1 ; c1ccc(-c2ccc(N(c3ccccc3)c3ccc(-c4c5ccccc5cc5c4oc4cc(-c6nc(-c7ccccc7)nc(-c7ccccc7)n6)ccc45)cc3)cc2)cc1]

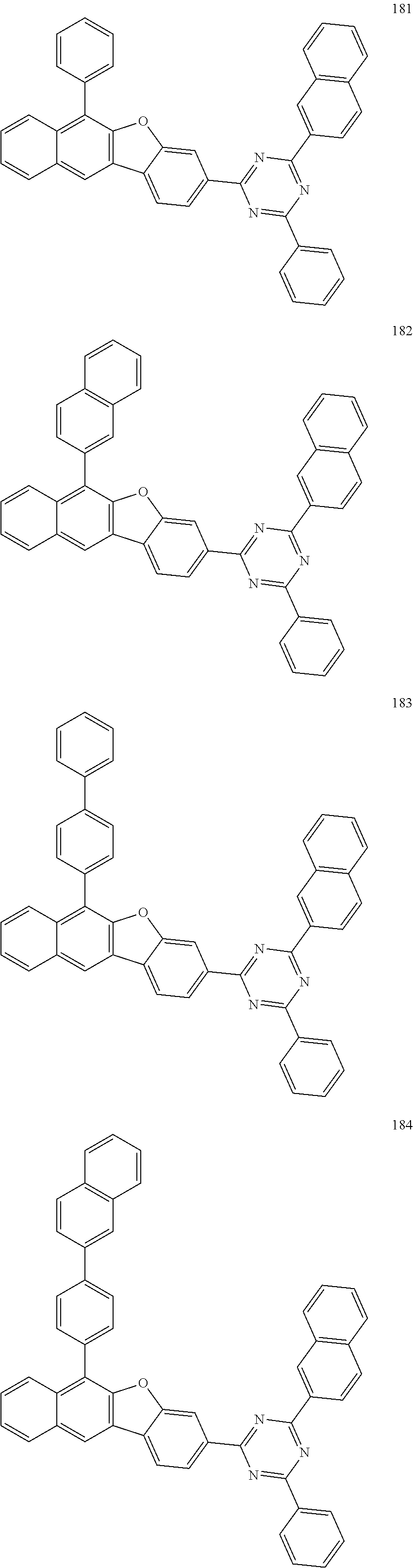 [SMILES: c1ccc(-c2nc(-c3ccc4ccccc4c3)nc(-c3ccc4c(c3)oc3c(-c5ccccc5)c5ccccc5cc34)n2)cc1 ; c1ccc(-c2nc(-c3ccc4ccccc4c3)nc(-c3ccc4c(c3)oc3c(-c5ccc6ccccc6c5)c5ccccc5cc34)n2)cc1 ; c1ccc(-c2ccc(-c3c4ccccc4cc4c3oc3cc(-c5nc(-c6ccccc6)nc(-c6ccc7ccccc7c6)n5)ccc34)cc2)cc1 ; c1ccc(-c2nc(-c3ccc4ccccc4c3)nc(-c3ccc4c(c3)oc3c(-c5ccc(-c6ccc7ccccc7c6)cc5)c5ccccc5cc34)n2)cc1]

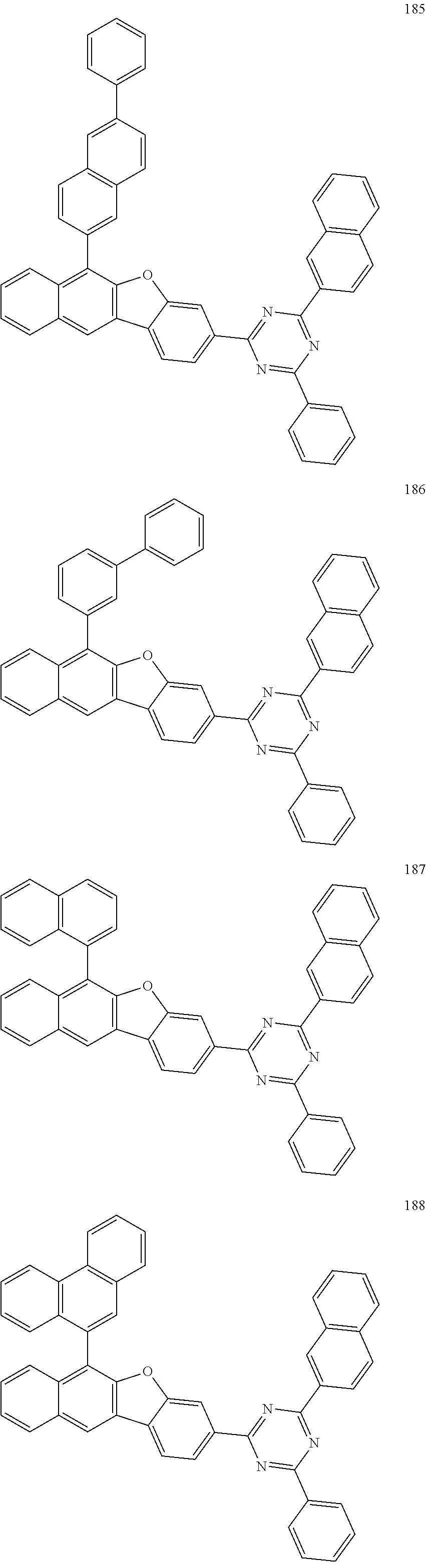

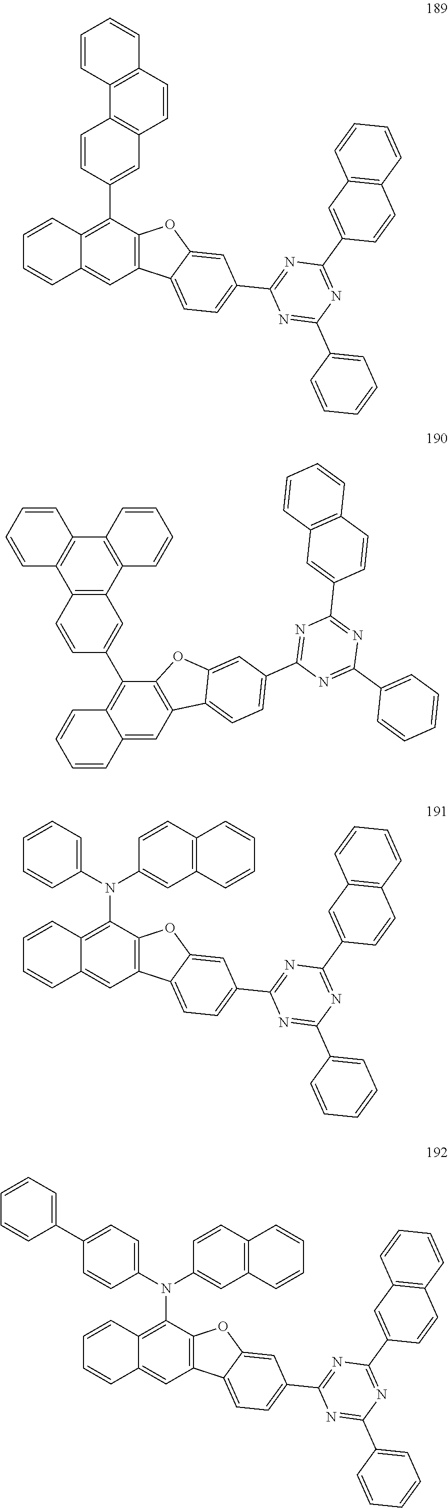 [SMILES: c1ccc(-c2nc(-c3ccc4ccccc4c3)nc(-c3ccc4c(c3)oc3c(-c5ccc6c(ccc7ccccc76)c5)c5ccccc5cc34)n2)cc1 ; c1ccc(-c2nc(-c3ccc4ccccc4c3)nc(-c3ccc4c(c3)oc3c(-c5ccc6c7ccccc7c7ccccc7c6c5)c5ccccc5cc34)n2)cc1 ; c1ccc(-c2nc(-c3ccc4ccccc4c3)nc(-c3ccc4c(c3)oc3c(N(c5ccccc5)c5ccc6ccccc6c5)c5ccccc5cc34)n2)cc1 ; c1ccc(-c2ccc(N(c3ccc4ccccc4c3)c3c4ccccc4cc4c3oc3cc(-c5nc(-c6ccccc6)nc(-c6ccc7ccccc7c6)n5)ccc34)cc2)cc1]

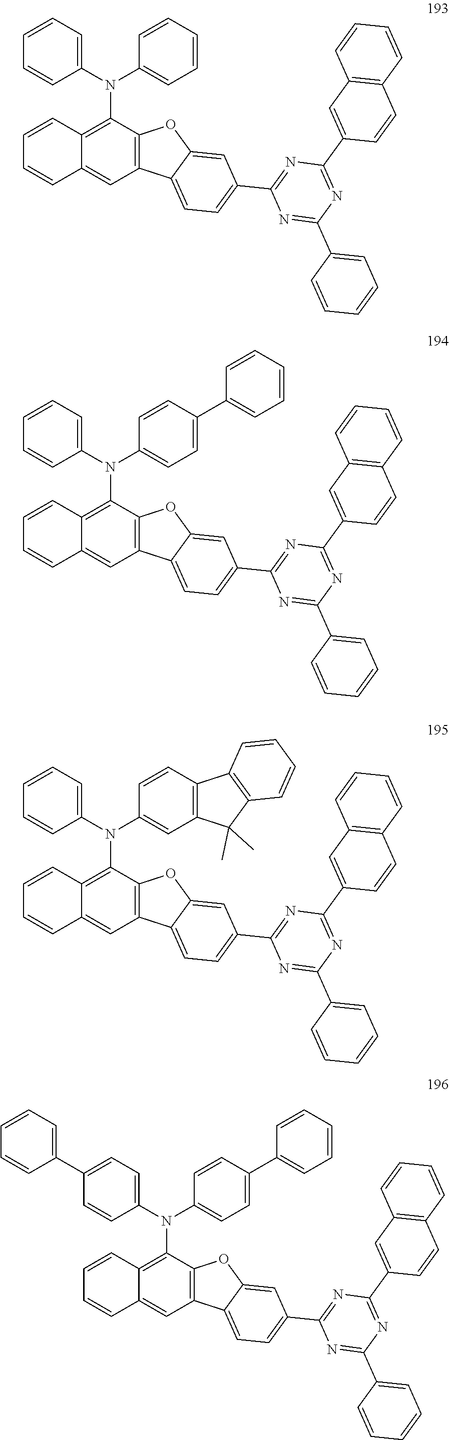 [SMILES: c1ccc(-c2nc(-c3ccc4ccccc4c3)nc(-c3ccc4c(c3)oc3c(N(c5ccccc5)c5ccccc5)c5ccccc5cc34)n2)cc1 ; c1ccc(-c2ccc(N(c3ccccc3)c3c4ccccc4cc4c3oc3cc(-c5nc(-c6ccccc6)nc(-c6ccc7ccccc7c6)n5)ccc34)cc2)cc1 ; CC1(C)c2ccccc2-c2ccc(N(c3ccccc3)c3c4ccccc4cc4c3oc3cc(-c5nc(-c6ccccc6)nc(-c6ccc7ccccc7c6)n5)ccc34)cc21 ; c1ccc(-c2ccc(N(c3ccc(-c4ccccc4)cc3)c3c4ccccc4cc4c3oc3cc(-c5nc(-c6ccccc6)nc(-c6ccc7ccccc7c6)n5)ccc34)cc2)cc1]

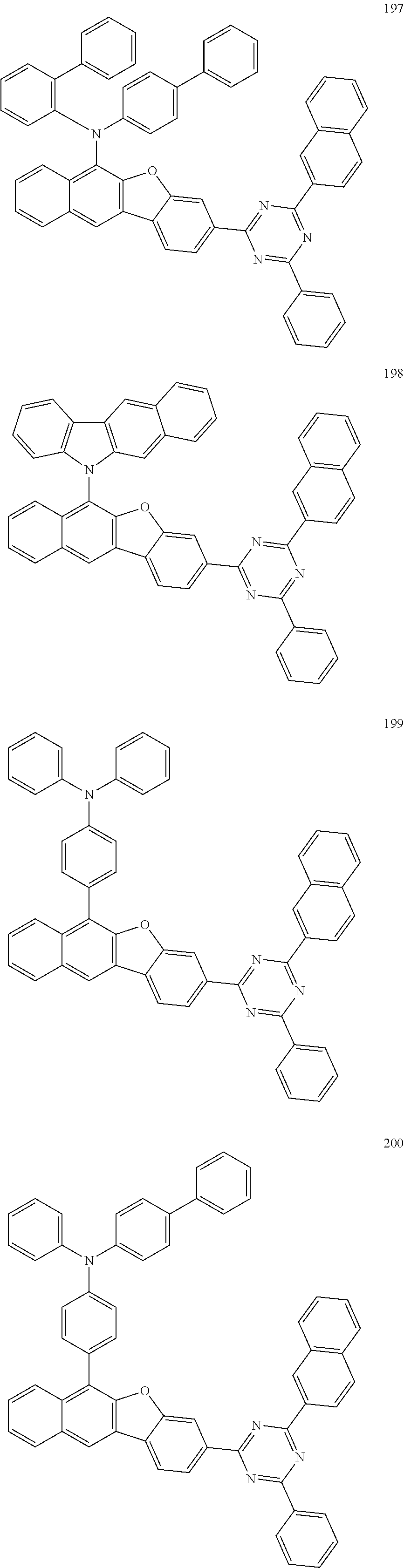 [SMILES: c1ccc(-c2ccc(N(c3ccccc3-c3ccccc3)c3c4ccccc4cc4c3oc3cc(-c5nc(-c6ccccc6)nc(-c6ccc7ccccc7c6)n5)ccc34)cc2)cc1 ; c1ccc(-c2nc(-c3ccc4ccccc4c3)nc(-c3ccc4c(c3)oc3c(-n5c6ccccc6c6cc7ccccc7cc65)c5ccccc5cc34)n2)cc1 ; c1ccc(-c2nc(-c3ccc4ccccc4c3)nc(-c3ccc4c(c3)oc3c(-c5ccc(N(c6ccccc6)c6ccccc6)cc5)c5ccccc5cc34)n2)cc1 ; c1ccc(-c2ccc(N(c3ccccc3)c3ccc(-c4c5ccccc5cc5c4oc4cc(-c6nc(-c7ccccc7)nc(-c7ccc8ccccc8c7)n6)ccc45)cc3)cc2)cc1]

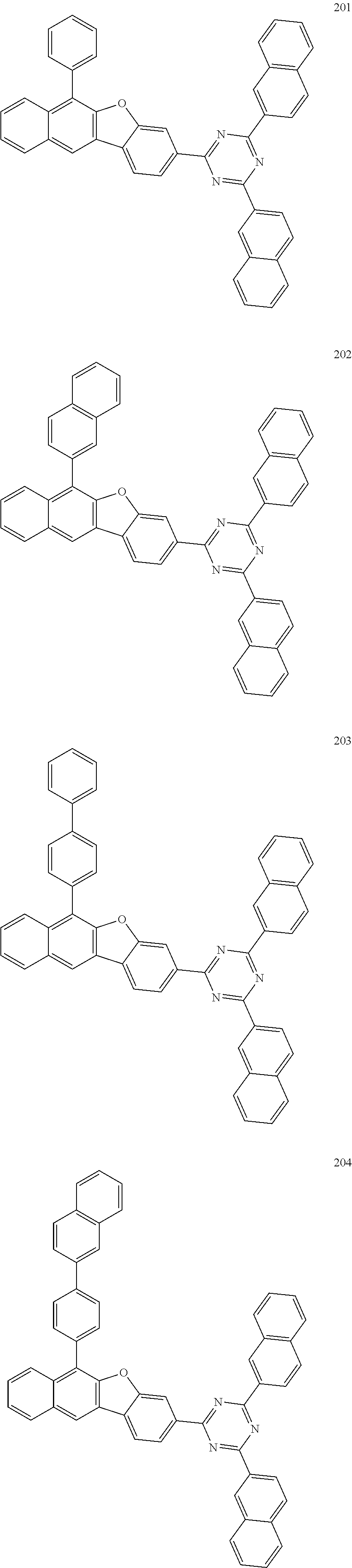 [SMILES: c1ccc(-c2c3ccccc3cc3c2oc2cc(-c4nc(-c5ccc6ccccc6c5)nc(-c5ccc6ccccc6c5)n4)ccc23)cc1 ; c1ccc2cc(-c3nc(-c4ccc5ccccc5c4)nc(-c4ccc5c(c4)oc4c(-c6ccc7ccccc7c6)c6ccccc6cc45)n3)ccc2c1 ; c1ccc(-c2ccc(-c3c4ccccc4cc4c3oc3cc(-c5nc(-c6ccc7ccccc7c6)nc(-c6ccc7ccccc7c6)n5)ccc34)cc2)cc1 ; c1ccc2cc(-c3ccc(-c4c5ccccc5cc5c4oc4cc(-c6nc(-c7ccc8ccccc8c7)nc(-c7ccc8ccccc8c7)n6)ccc45)cc3)ccc2c1]

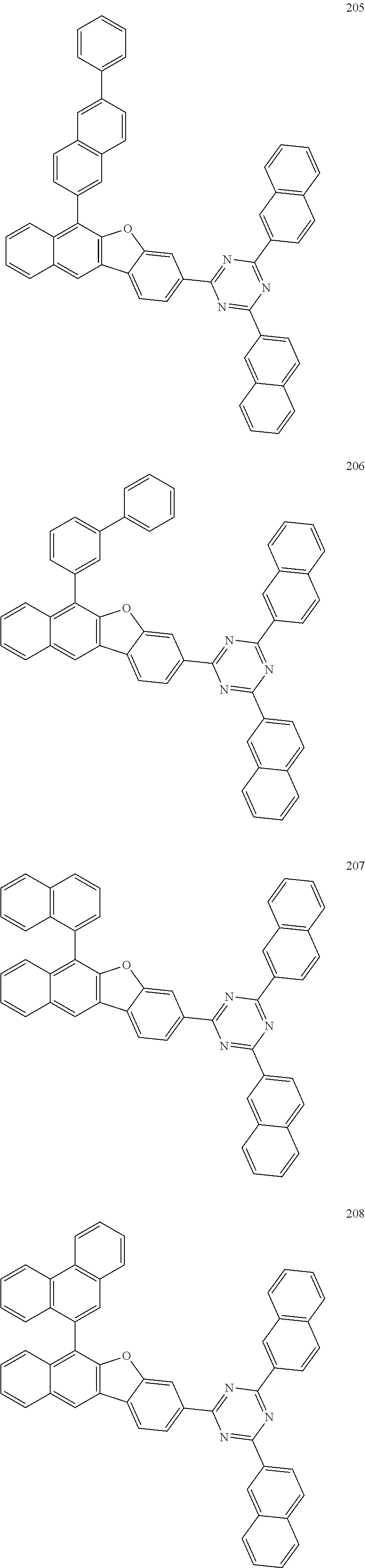 [SMILES: c1ccc(-c2ccc3cc(-c4c5ccccc5cc5c4oc4cc(-c6nc(-c7ccc8ccccc8c7)nc(-c7ccc8ccccc8c7)n6)ccc45)ccc3c2)cc1 ; c1ccc(-c2cccc(-c3c4ccccc4cc4c3oc3cc(-c5nc(-c6ccc7ccccc7c6)nc(-c6ccc7ccccc7c6)n5)ccc34)c2)cc1 ; c1ccc2cc(-c3nc(-c4ccc5ccccc5c4)nc(-c4ccc5c(c4)oc4c(-c6cccc7ccccc67)c6ccccc6cc45)n3)ccc2c1 ; c1ccc2cc(-c3nc(-c4ccc5ccccc5c4)nc(-c4ccc5c(c4)oc4c(-c6cc7ccccc7c7ccccc67)c6ccccc6cc45)n3)ccc2c1]

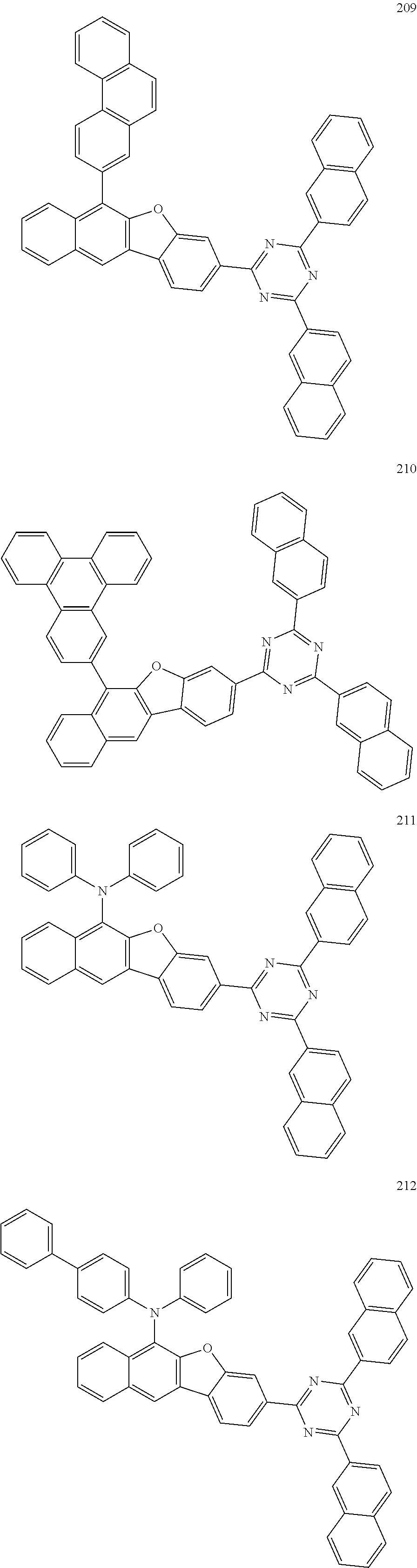 [SMILES: c1ccc2cc(-c3nc(-c4ccc5ccccc5c4)nc(-c4ccc5c(c4)oc4c(-c6ccc7c(ccc8ccccc87)c6)c6ccccc6cc45)n3)ccc2c1 ; c1ccc2cc(-c3nc(-c4ccc5ccccc5c4)nc(-c4ccc5c(c4)oc4c(-c6ccc7c8ccccc8c8ccccc8c7c6)c6ccccc6cc45)n3)ccc2c1 ; c1ccc(N(c2ccccc2)c2c3ccccc3cc3c2oc2cc(-c4nc(-c5ccc6ccccc6c5)nc(-c5ccc6ccccc6c5)n4)ccc23)cc1 ; c1ccc(-c2ccc(N(c3ccccc3)c3c4ccccc4cc4c3oc3cc(-c5nc(-c6ccc7ccccc7c6)nc(-c6ccc7ccccc7c6)n5)ccc34)cc2)cc1]

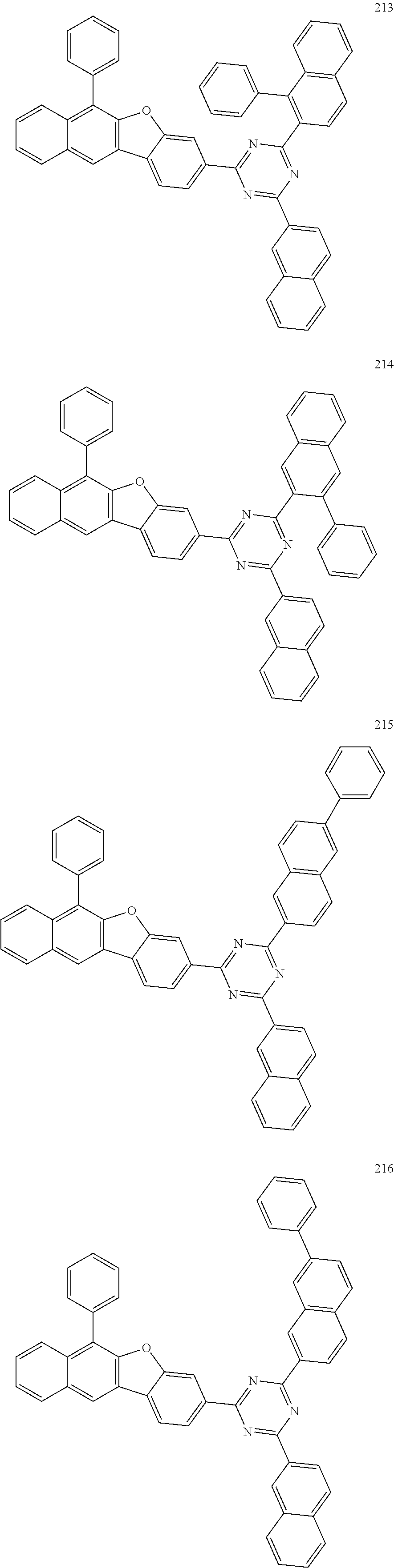 [SMILES: c1ccc(-c2c(-c3nc(-c4ccc5ccccc5c4)nc(-c4ccc5c(c4)oc4c(-c6ccccc6)c6ccccc6cc45)n3)ccc3ccccc23)cc1 ; c1ccc(-c2cc3ccccc3cc2-c2nc(-c3ccc4ccccc4c3)nc(-c3ccc4c(c3)oc3c(-c5ccccc5)c5ccccc5cc34)n2)cc1 ; c1ccc(-c2ccc3cc(-c4nc(-c5ccc6ccccc6c5)nc(-c5ccc6c(c5)oc5c(-c7ccccc7)c7ccccc7cc56)n4)ccc3c2)cc1 ; c1ccc(-c2ccc3ccc(-c4nc(-c5ccc6ccccc6c5)nc(-c5ccc6c(c5)oc5c(-c7ccccc7)c7ccccc7cc56)n4)cc3c2)cc1]

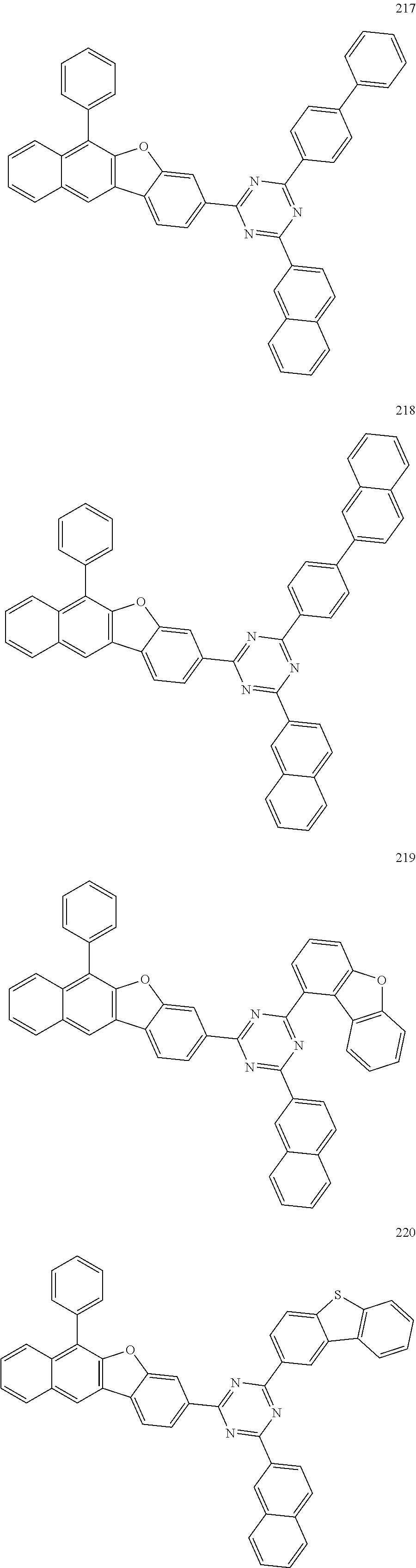 [SMILES: c1ccc(-c2ccc(-c3nc(-c4ccc5ccccc5c4)nc(-c4ccc5c(c4)oc4c(-c6ccccc6)c6ccccc6cc45)n3)cc2)cc1 ; c1ccc(-c2c3ccccc3cc3c2oc2cc(-c4nc(-c5ccc(-c6ccc7ccccc7c6)cc5)nc(-c5ccc6ccccc6c5)n4)ccc23)cc1 ; c1ccc(-c2c3ccccc3cc3c2oc2cc(-c4nc(-c5ccc6ccccc6c5)nc(-c5cccc6oc7ccccc7c56)n4)ccc23)cc1 ; c1ccc(-c2c3ccccc3cc3c2oc2cc(-c4nc(-c5ccc6ccccc6c5)nc(-c5ccc6sc7ccccc7c6c5)n4)ccc23)cc1]

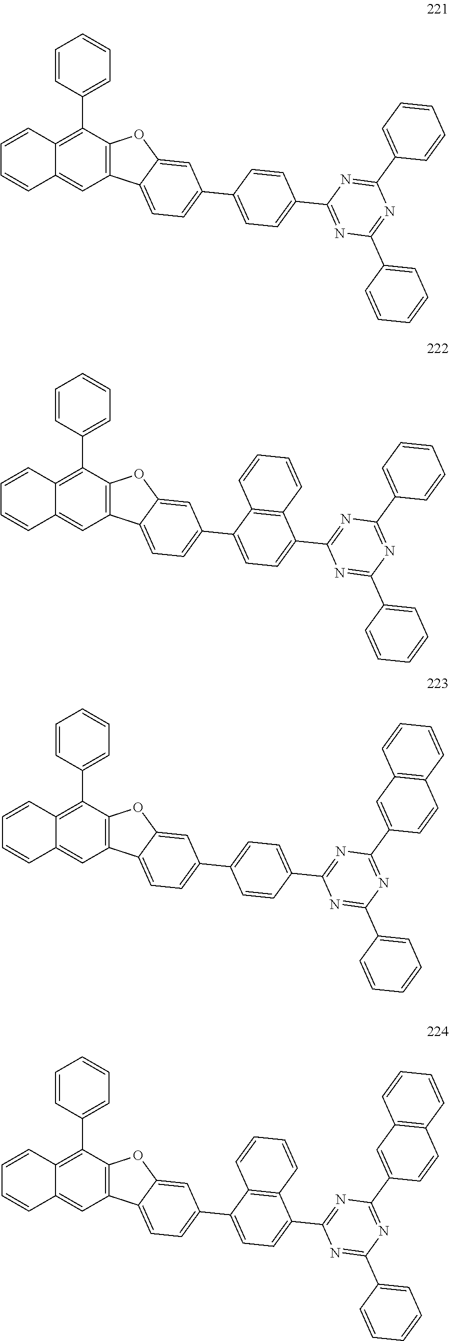 [SMILES: c1ccc(-c2nc(-c3ccccc3)nc(-c3ccc(-c4ccc5c(c4)oc4c(-c6ccccc6)c6ccccc6cc45)cc3)n2)cc1 ; c1ccc(-c2nc(-c3ccccc3)nc(-c3ccc(-c4ccc5c(c4)oc4c(-c6ccccc6)c6ccccc6cc45)c4ccccc34)n2)cc1 ; c1ccc(-c2nc(-c3ccc(-c4ccc5c(c4)oc4c(-c6ccccc6)c6ccccc6cc45)cc3)nc(-c3ccc4ccccc4c3)n2)cc1 ; c1ccc(-c2nc(-c3ccc4ccccc4c3)nc(-c3ccc(-c4ccc5c(c4)oc4c(-c6ccccc6)c6ccccc6cc45)c4ccccc34)n2)cc1]

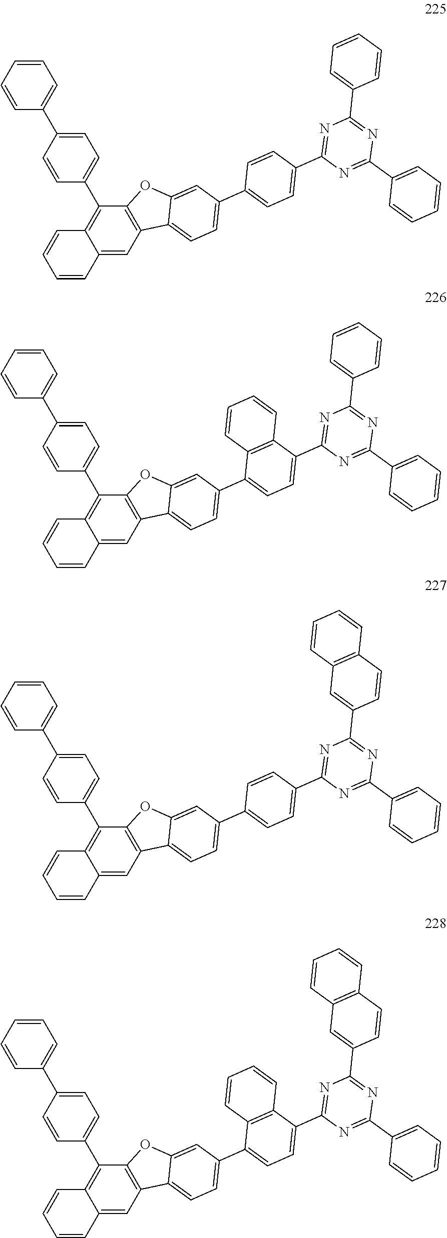 [SMILES: c1ccc(-c2ccc(-c3c4ccccc4cc4c3oc3cc(-c5ccc(-c6nc(-c7ccccc7)nc(-c7ccccc7)n6)cc5)ccc34)cc2)cc1 ; c1ccc(-c2ccc(-c3c4ccccc4cc4c3oc3cc(-c5ccc(-c6nc(-c7ccccc7)nc(-c7ccccc7)n6)c6ccccc56)ccc34)cc2)cc1 ; c1ccc(-c2ccc(-c3c4ccccc4cc4c3oc3cc(-c5ccc(-c6nc(-c7ccccc7)nc(-c7ccc8ccccc8c7)n6)cc5)ccc34)cc2)cc1 ; c1ccc(-c2ccc(-c3c4ccccc4cc4c3oc3cc(-c5ccc(-c6nc(-c7ccccc7)nc(-c7ccc8ccccc8c7)n6)c6ccccc56)ccc34)cc2)cc1]

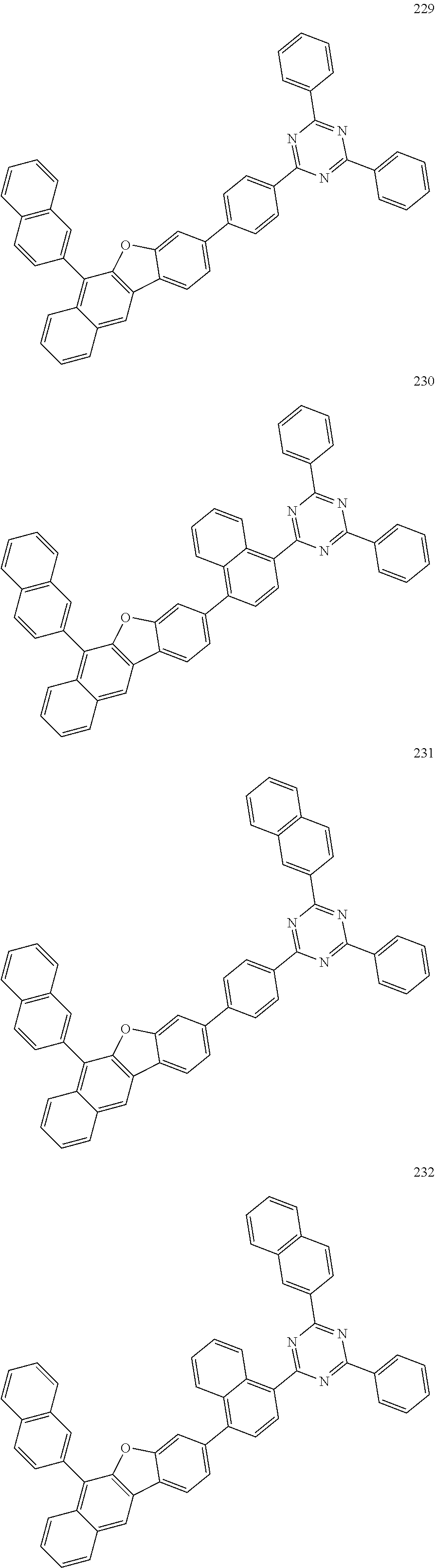 [SMILES: c1ccc(-c2nc(-c3ccccc3)nc(-c3ccc(-c4ccc5c(c4)oc4c(-c6ccc7ccccc7c6)c6ccccc6cc45)cc3)n2)cc1 ; c1ccc(-c2nc(-c3ccccc3)nc(-c3ccc(-c4ccc5c(c4)oc4c(-c6ccc7ccccc7c6)c6ccccc6cc45)c4ccccc34)n2)cc1 ; c1ccc(-c2nc(-c3ccc(-c4ccc5c(c4)oc4c(-c6ccc7ccccc7c6)c6ccccc6cc45)cc3)nc(-c3ccc4ccccc4c3)n2)cc1 ; c1ccc(-c2nc(-c3ccc4ccccc4c3)nc(-c3ccc(-c4ccc5c(c4)oc4c(-c6ccc7ccccc7c6)c6ccccc6cc45)c4ccccc34)n2)cc1]

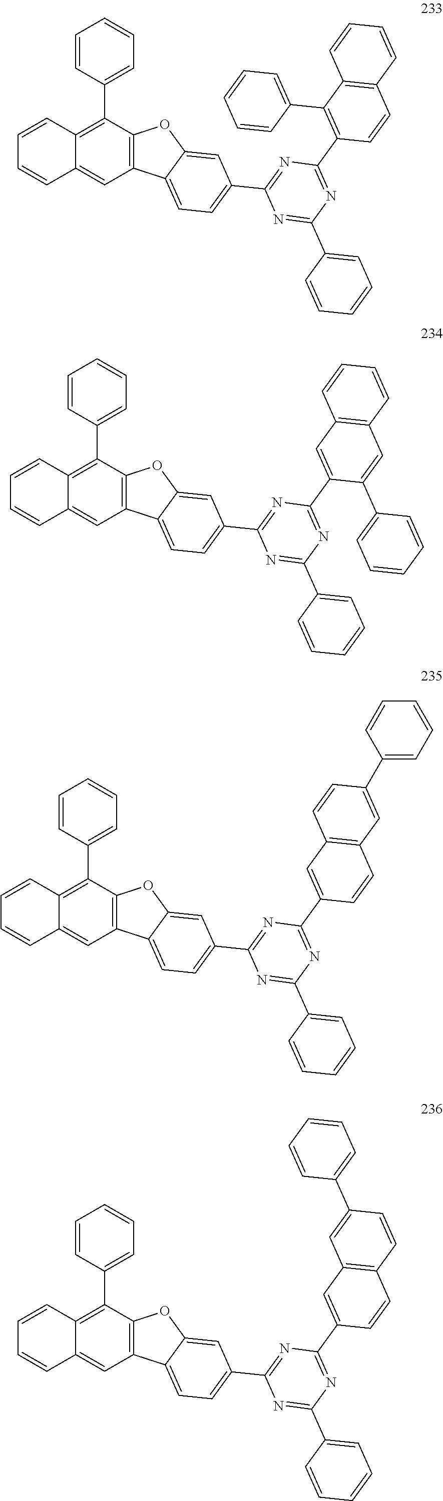 [SMILES: c1ccc(-c2nc(-c3ccc4c(c3)oc3c(-c5ccccc5)c5ccccc5cc34)nc(-c3ccc4ccccc4c3-c3ccccc3)n2)cc1 ; c1ccc(-c2nc(-c3ccc4c(c3)oc3c(-c5ccccc5)c5ccccc5cc34)nc(-c3cc4ccccc4cc3-c3ccccc3)n2)cc1 ; c1ccc(-c2ccc3cc(-c4nc(-c5ccccc5)nc(-c5ccc6c(c5)oc5c(-c7ccccc7)c7ccccc7cc56)n4)ccc3c2)cc1 ; c1ccc(-c2ccc3ccc(-c4nc(-c5ccccc5)nc(-c5ccc6c(c5)oc5c(-c7ccccc7)c7ccccc7cc56)n4)cc3c2)cc1]

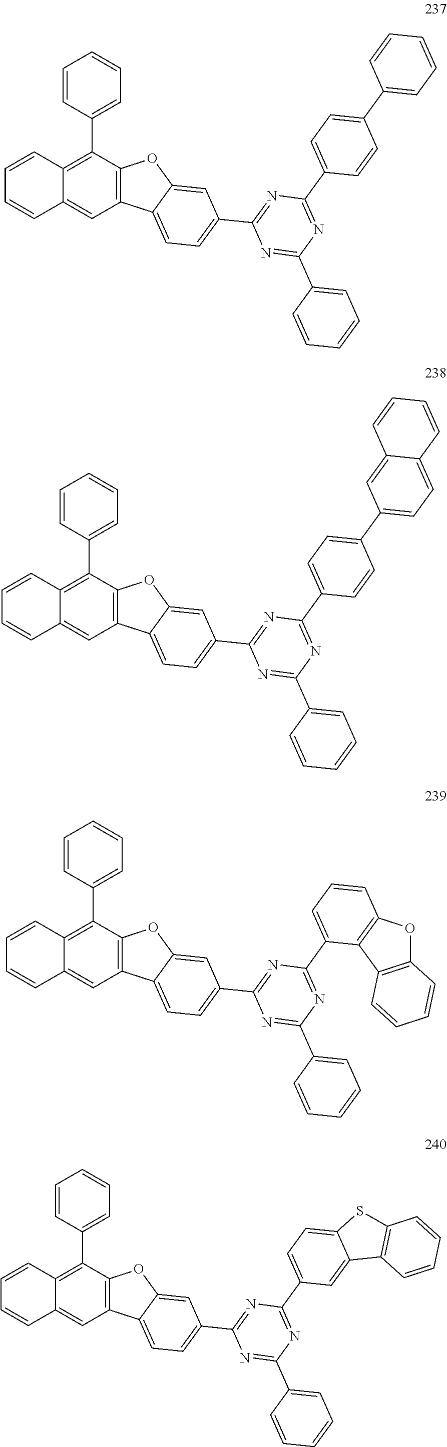 [SMILES: c1ccc(-c2ccc(-c3nc(-c4ccccc4)nc(-c4ccc5c(c4)oc4c(-c6ccccc6)c6ccccc6cc45)n3)cc2)cc1 ; c1ccc(-c2nc(-c3ccc(-c4ccc5ccccc5c4)cc3)nc(-c3ccc4c(c3)oc3c(-c5ccccc5)c5ccccc5cc34)n2)cc1 ; c1ccc(-c2nc(-c3ccc4c(c3)oc3c(-c5ccccc5)c5ccccc5cc34)nc(-c3cccc4oc5ccccc5c34)n2)cc1 ; c1ccc(-c2nc(-c3ccc4c(c3)oc3c(-c5ccccc5)c5ccccc5cc34)nc(-c3ccc4sc5ccccc5c4c3)n2)cc1]

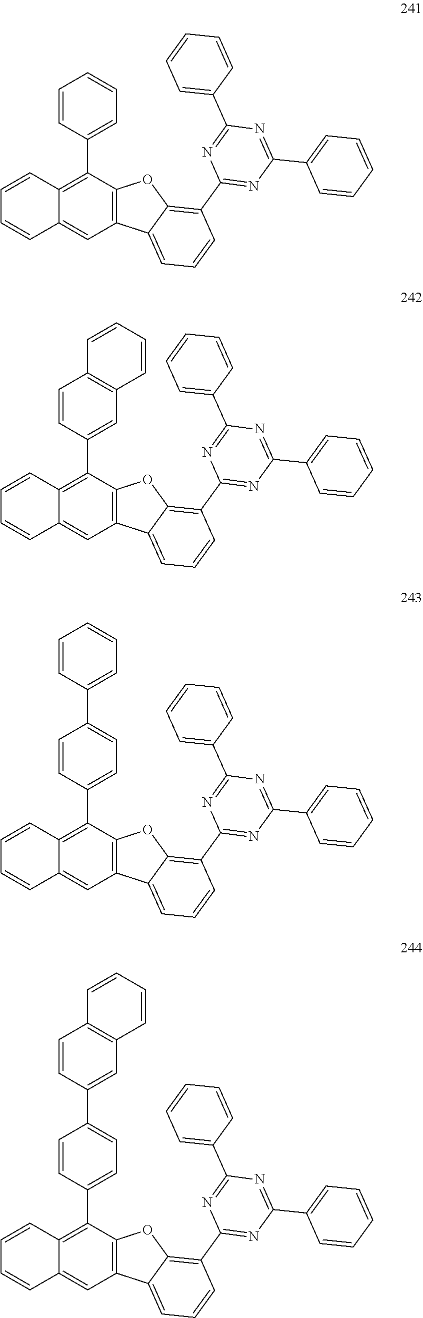 [SMILES: c1ccc(-c2nc(-c3ccccc3)nc(-c3cccc4c3oc3c(-c5ccccc5)c5ccccc5cc34)n2)cc1 ; c1ccc(-c2nc(-c3ccccc3)nc(-c3cccc4c3oc3c(-c5ccc6ccccc6c5)c5ccccc5cc34)n2)cc1 ; c1ccc(-c2ccc(-c3c4ccccc4cc4c3oc3c(-c5nc(-c6ccccc6)nc(-c6ccccc6)n5)cccc34)cc2)cc1 ; c1ccc(-c2nc(-c3ccccc3)nc(-c3cccc4c3oc3c(-c5ccc(-c6ccc7ccccc7c6)cc5)c5ccccc5cc34)n2)cc1]

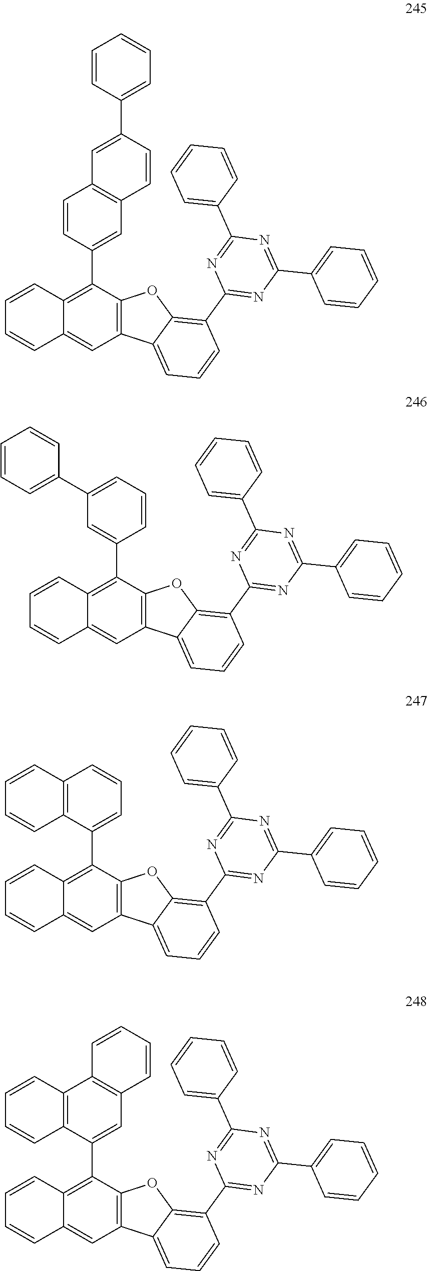 [SMILES: c1ccc(-c2ccc3cc(-c4c5ccccc5cc5c4oc4c(-c6nc(-c7ccccc7)nc(-c7ccccc7)n6)cccc45)ccc3c2)cc1 ; c1ccc(-c2cccc(-c3c4ccccc4cc4c3oc3c(-c5nc(-c6ccccc6)nc(-c6ccccc6)n5)cccc34)c2)cc1 ; c1ccc(-c2nc(-c3ccccc3)nc(-c3cccc4c3oc3c(-c5cccc6ccccc56)c5ccccc5cc34)n2)cc1 ; c1ccc(-c2nc(-c3ccccc3)nc(-c3cccc4c3oc3c(-c5cc6ccccc6c6ccccc56)c5ccccc5cc34)n2)cc1]

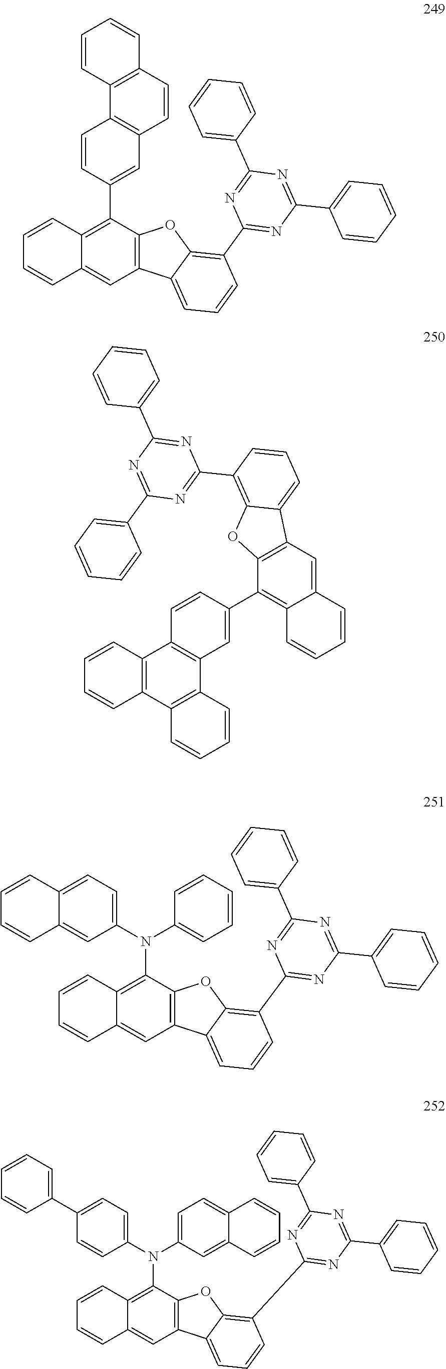 [SMILES: c1ccc(-c2nc(-c3ccccc3)nc(-c3cccc4c3oc3c(-c5ccc6c(ccc7ccccc76)c5)c5ccccc5cc34)n2)cc1 ; c1ccc(-c2nc(-c3ccccc3)nc(-c3cccc4c3oc3c(-c5ccc6c7ccccc7c7ccccc7c6c5)c5ccccc5cc34)n2)cc1 ; c1ccc(-c2nc(-c3ccccc3)nc(-c3cccc4c3oc3c(N(c5ccccc5)c5ccc6ccccc6c5)c5ccccc5cc34)n2)cc1 ; c1ccc(-c2ccc(N(c3ccc4ccccc4c3)c3c4ccccc4cc4c3oc3c(-c5nc(-c6ccccc6)nc(-c6ccccc6)n5)cccc34)cc2)cc1]

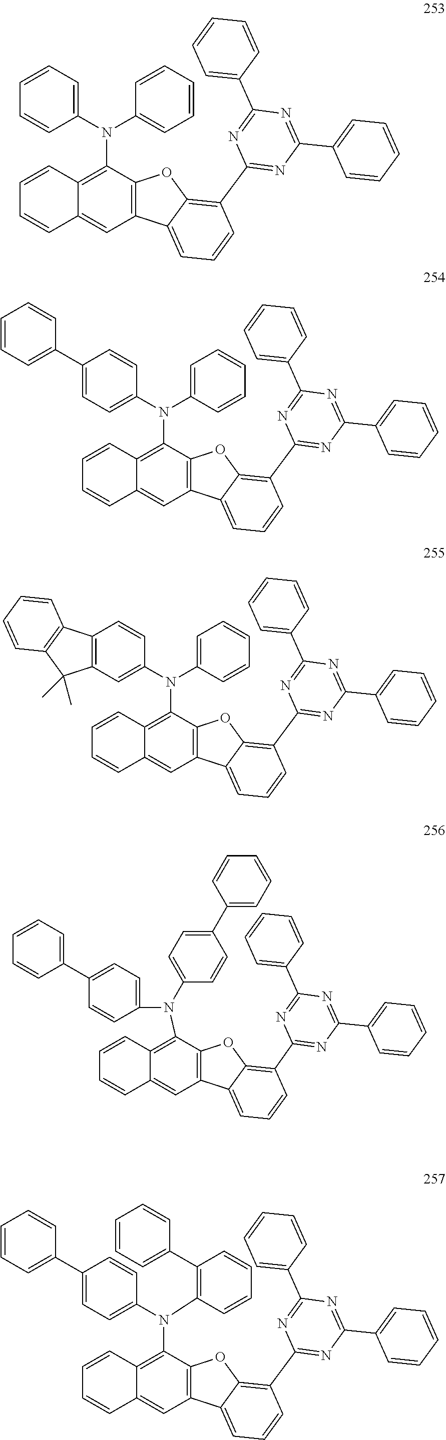 [SMILES: c1ccc(-c2nc(-c3ccccc3)nc(-c3cccc4c3oc3c(N(c5ccccc5)c5ccccc5)c5ccccc5cc34)n2)cc1 ; c1ccc(-c2ccc(N(c3ccccc3)c3c4ccccc4cc4c3oc3c(-c5nc(-c6ccccc6)nc(-c6ccccc6)n5)cccc34)cc2)cc1 ; CC1(C)c2ccccc2-c2ccc(N(c3ccccc3)c3c4ccccc4cc4c3oc3c(-c5nc(-c6ccccc6)nc(-c6ccccc6)n5)cccc34)cc21 ; c1ccc(-c2ccc(N(c3ccc(-c4ccccc4)cc3)c3c4ccccc4cc4c3oc3c(-c5nc(-c6ccccc6)nc(-c6ccccc6)n5)cccc34)cc2)cc1 ; c1ccc(-c2ccc(N(c3ccccc3-c3ccccc3)c3c4ccccc4cc4c3oc3c(-c5nc(-c6ccccc6)nc(-c6ccccc6)n5)cccc34)cc2)cc1]

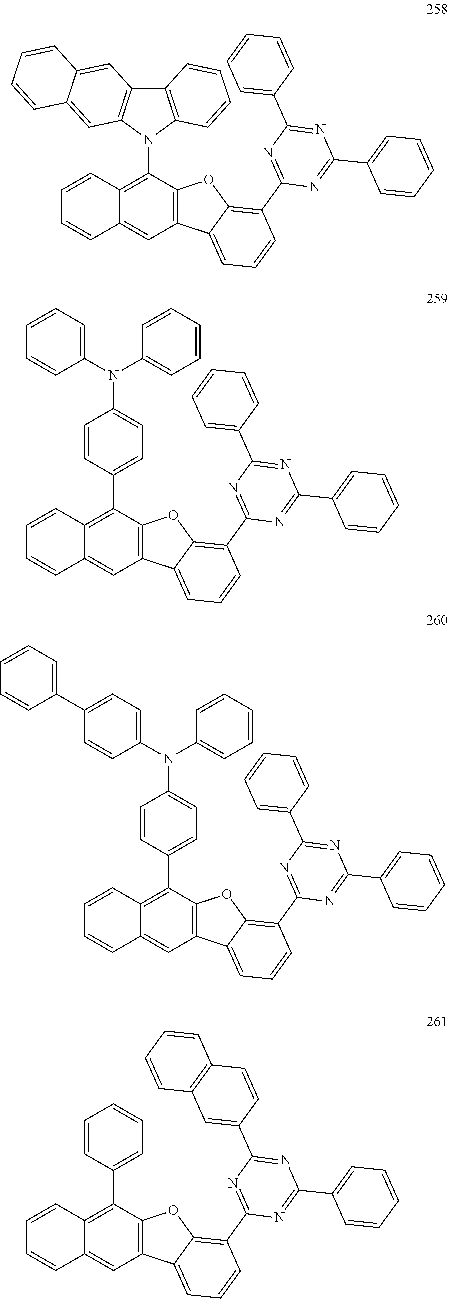 [SMILES: c1ccc(-c2nc(-c3ccccc3)nc(-c3cccc4c3oc3c(-n5c6ccccc6c6cc7ccccc7cc65)c5ccccc5cc34)n2)cc1 ; c1ccc(-c2nc(-c3ccccc3)nc(-c3cccc4c3oc3c(-c5ccc(N(c6ccccc6)c6ccccc6)cc5)c5ccccc5cc34)n2)cc1 ; c1ccc(-c2ccc(N(c3ccccc3)c3ccc(-c4c5ccccc5cc5c4oc4c(-c6nc(-c7ccccc7)nc(-c7ccccc7)n6)cccc45)cc3)cc2)cc1 ; c1ccc(-c2nc(-c3ccc4ccccc4c3)nc(-c3cccc4c3oc3c(-c5ccccc5)c5ccccc5cc34)n2)cc1]

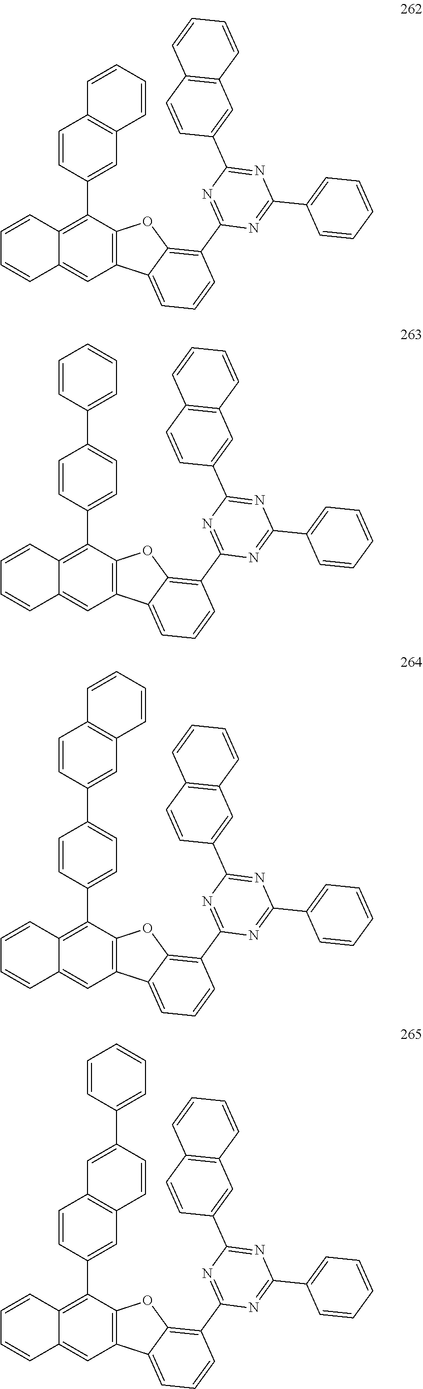

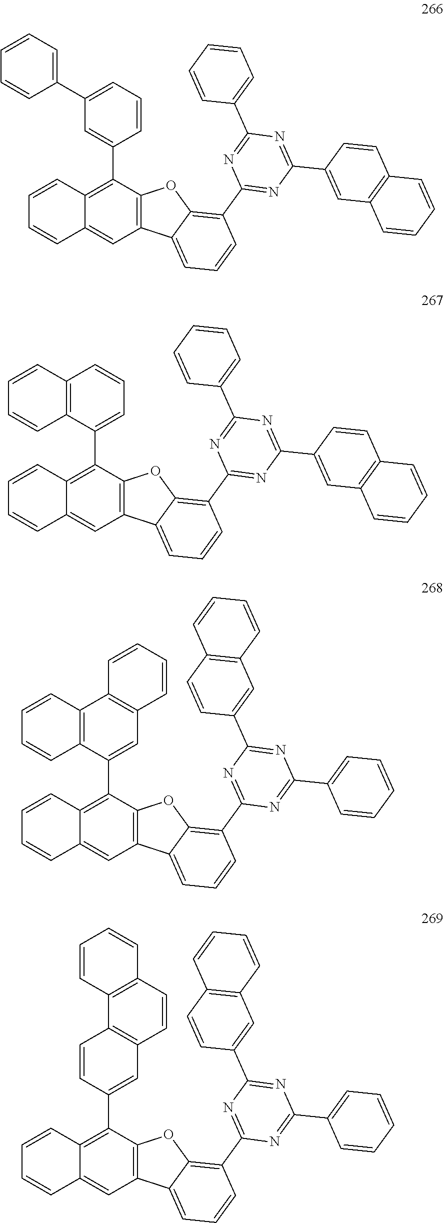

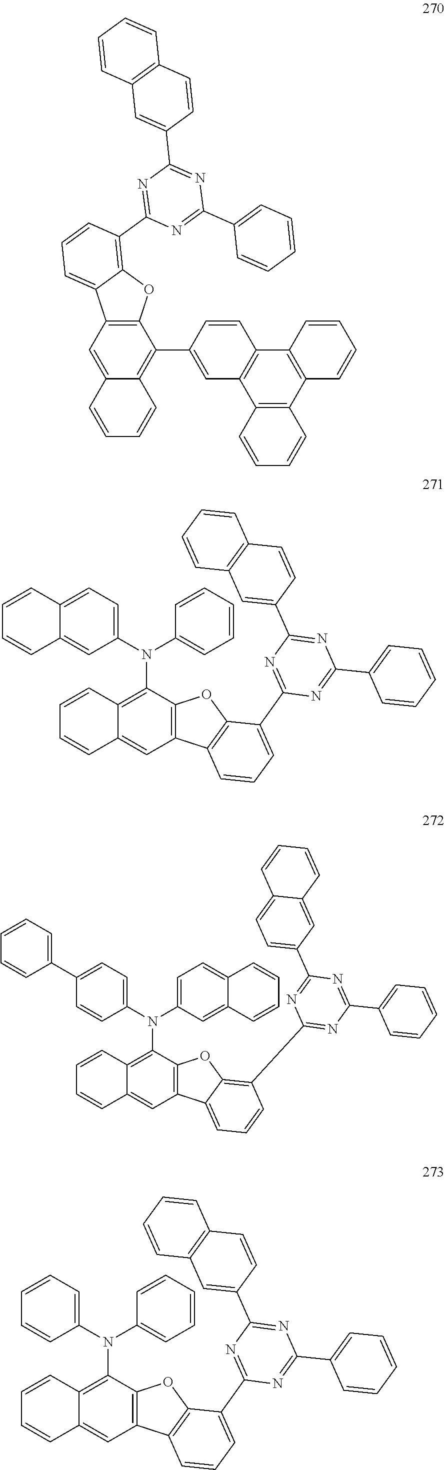 [SMILES: c1ccc(-c2nc(-c3ccc4ccccc4c3)nc(-c3cccc4c3oc3c(-c5ccc6c7ccccc7c7ccccc7c6c5)c5ccccc5cc34)n2)cc1 ; c1ccc(-c2nc(-c3ccc4ccccc4c3)nc(-c3cccc4c3oc3c(N(c5ccccc5)c5ccc6ccccc6c5)c5ccccc5cc34)n2)cc1 ; c1ccc(-c2ccc(N(c3ccc4ccccc4c3)c3c4ccccc4cc4c3oc3c(-c5nc(-c6ccccc6)nc(-c6ccc7ccccc7c6)n5)cccc34)cc2)cc1 ; c1ccc(-c2nc(-c3ccc4ccccc4c3)nc(-c3cccc4c3oc3c(N(c5ccccc5)c5ccccc5)c5ccccc5cc34)n2)cc1]

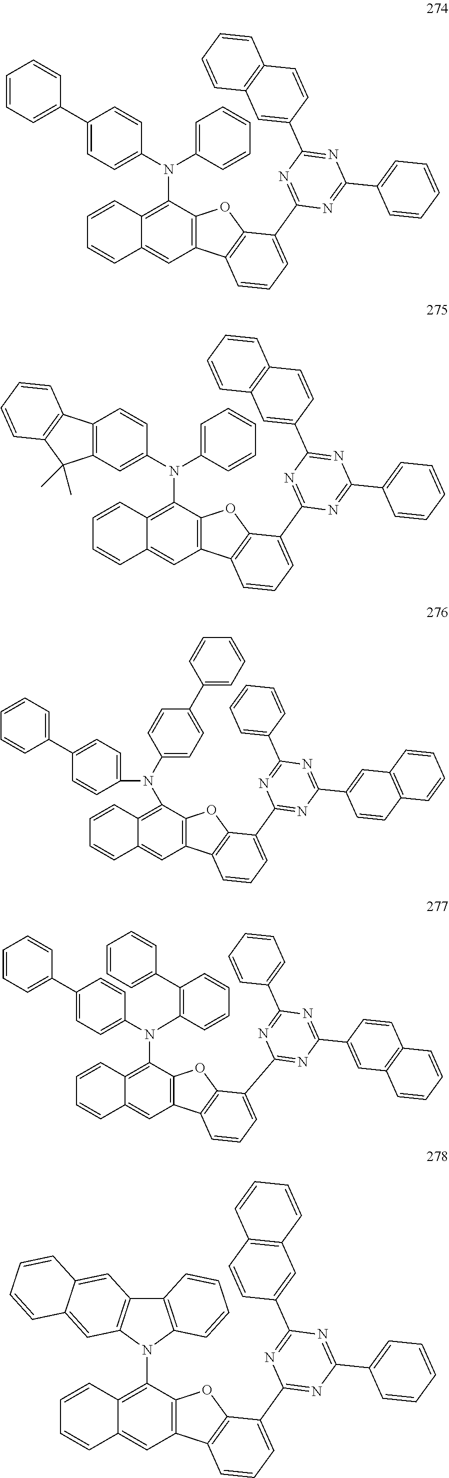

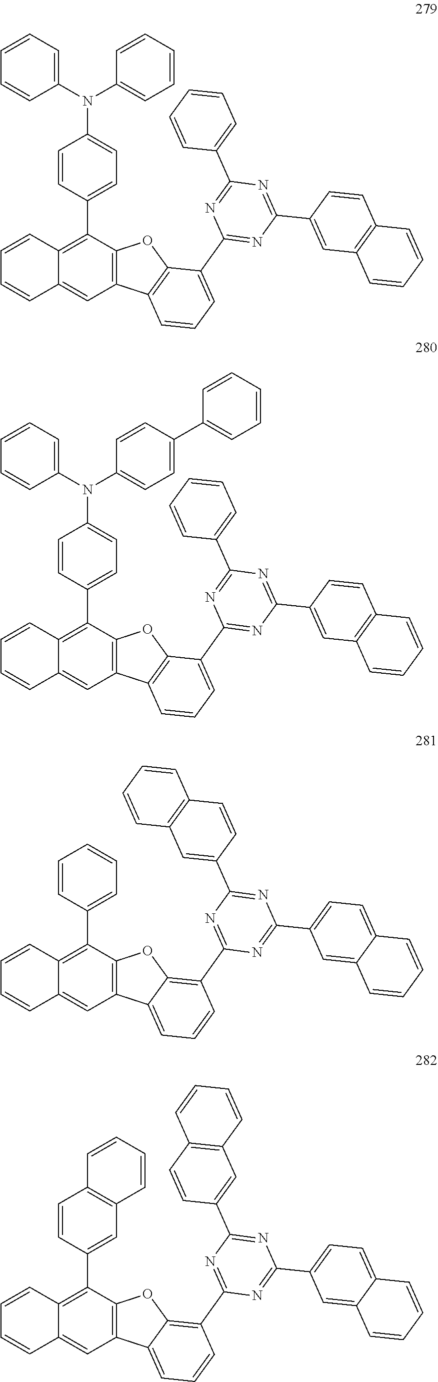 [SMILES: c1ccc(-c2nc(-c3ccc4ccccc4c3)nc(-c3cccc4c3oc3c(-c5ccc(N(c6ccccc6)c6ccccc6)cc5)c5ccccc5cc34)n2)cc1 ; c1ccc(-c2ccc(N(c3ccccc3)c3ccc(-c4c5ccccc5cc5c4oc4c(-c6nc(-c7ccccc7)nc(-c7ccc8ccccc8c7)n6)cccc45)cc3)cc2)cc1 ; c1ccc(-c2c3ccccc3cc3c2oc2c(-c4nc(-c5ccc6ccccc6c5)nc(-c5ccc6ccccc6c5)n4)cccc23)cc1 ; c1ccc2cc(-c3nc(-c4ccc5ccccc5c4)nc(-c4cccc5c4oc4c(-c6ccc7ccccc7c6)c6ccccc6cc45)n3)ccc2c1]

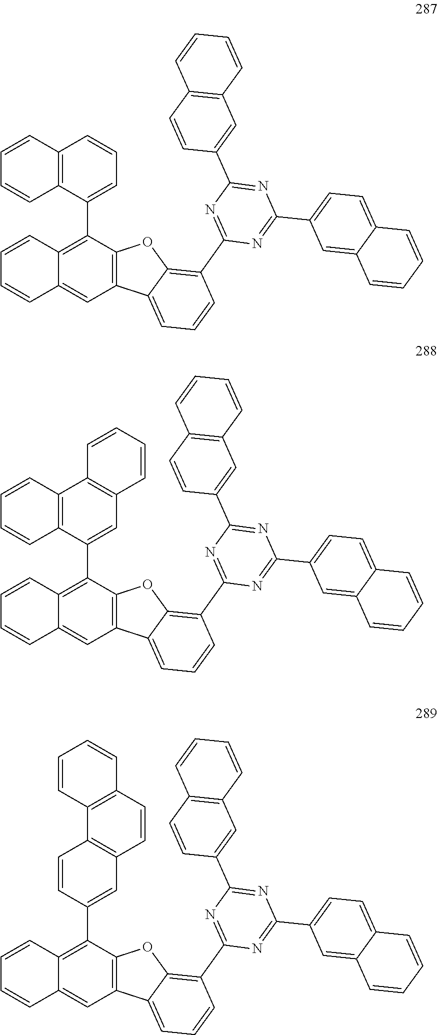

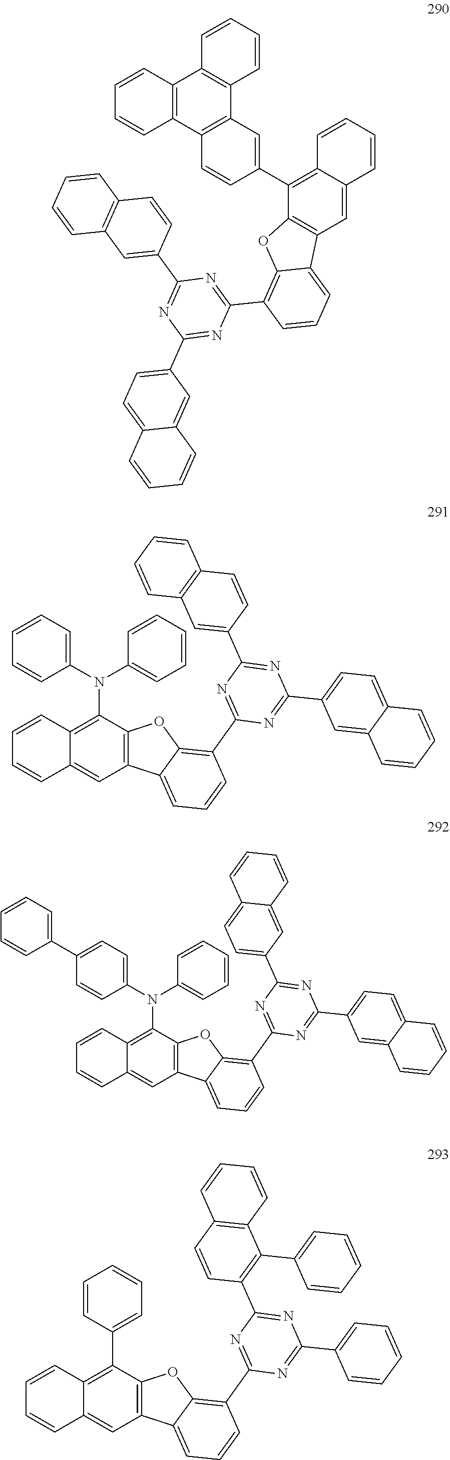 [SMILES: c1ccc2cc(-c3nc(-c4ccc5ccccc5c4)nc(-c4cccc5c4oc4c(-c6ccc7c8ccccc8c8ccccc8c7c6)c6ccccc6cc45)n3)ccc2c1 ; c1ccc(N(c2ccccc2)c2c3ccccc3cc3c2oc2c(-c4nc(-c5ccc6ccccc6c5)nc(-c5ccc6ccccc6c5)n4)cccc23)cc1 ; c1ccc(-c2ccc(N(c3ccccc3)c3c4ccccc4cc4c3oc3c(-c5nc(-c6ccc7ccccc7c6)nc(-c6ccc7ccccc7c6)n5)cccc34)cc2)cc1 ; c1ccc(-c2nc(-c3ccc4ccccc4c3-c3ccccc3)nc(-c3cccc4c3oc3c(-c5ccccc5)c5ccccc5cc34)n2)cc1]

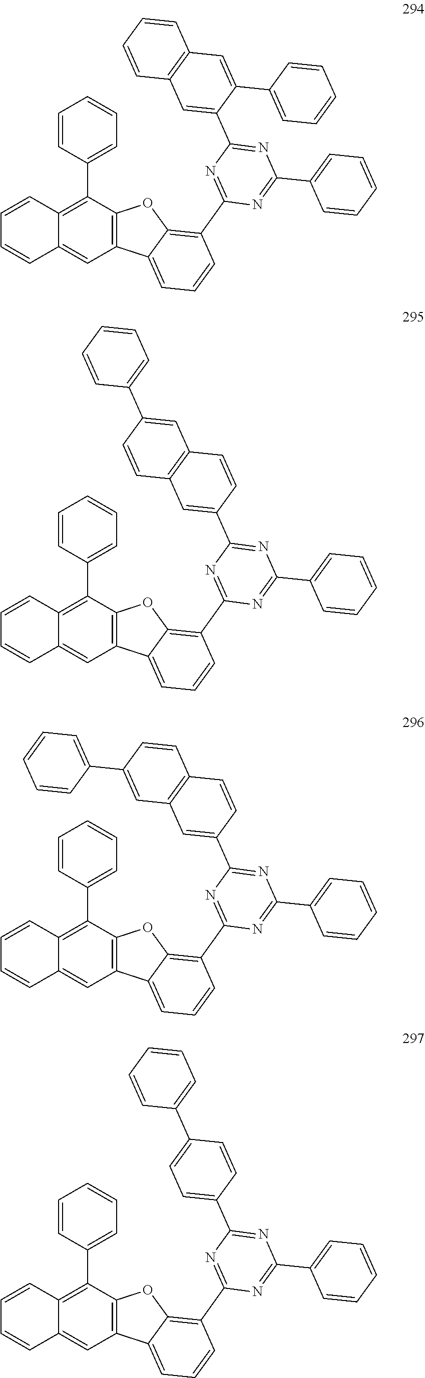

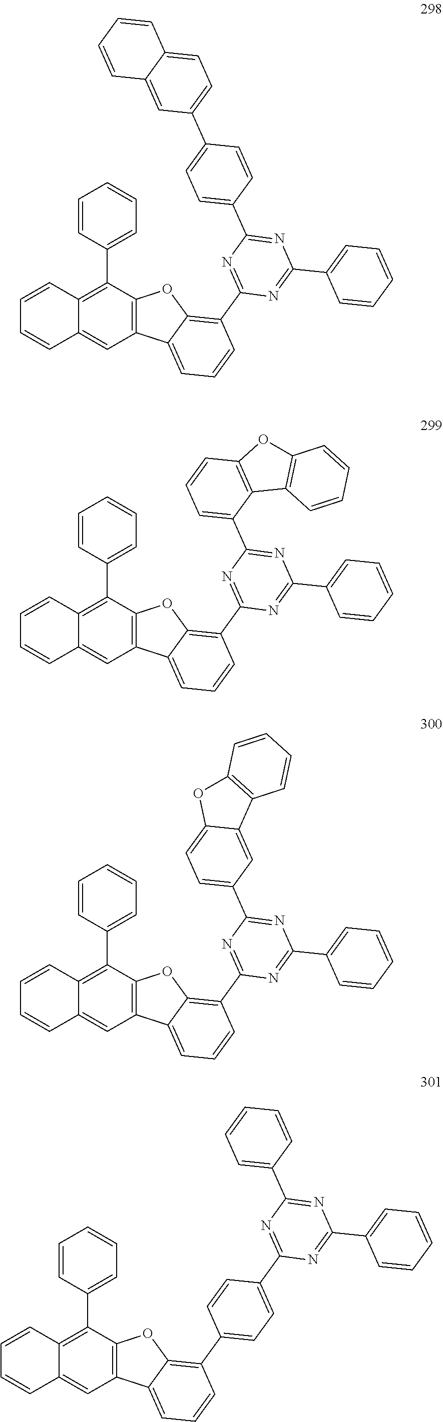 [SMILES: c1ccc(-c2nc(-c3ccc(-c4ccc5ccccc5c4)cc3)nc(-c3cccc4c3oc3c(-c5ccccc5)c5ccccc5cc34)n2)cc1 ; c1ccc(-c2nc(-c3cccc4c3oc3c(-c5ccccc5)c5ccccc5cc34)nc(-c3cccc4oc5ccccc5c34)n2)cc1 ; c1ccc(-c2nc(-c3ccc4oc5ccccc5c4c3)nc(-c3cccc4c3oc3c(-c5ccccc5)c5ccccc5cc34)n2)cc1 ; c1ccc(-c2nc(-c3ccccc3)nc(-c3ccc(-c4cccc5c4oc4c(-c6ccccc6)c6ccccc6cc45)cc3)n2)cc1]

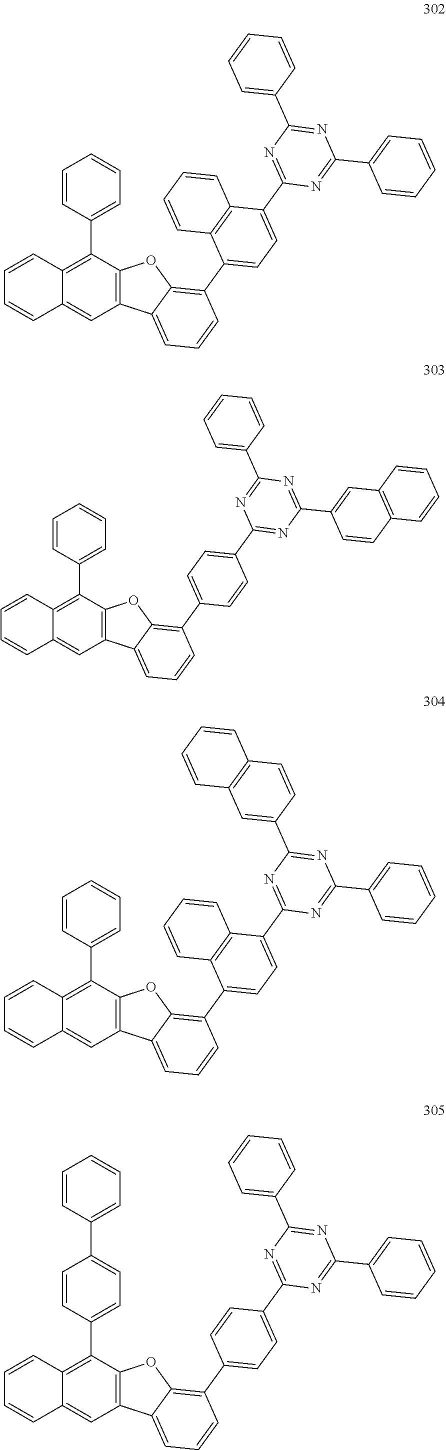 [SMILES: c1ccc(-c2nc(-c3ccccc3)nc(-c3ccc(-c4cccc5c4oc4c(-c6ccccc6)c6ccccc6cc45)c4ccccc34)n2)cc1 ; c1ccc(-c2nc(-c3ccc(-c4cccc5c4oc4c(-c6ccccc6)c6ccccc6cc45)cc3)nc(-c3ccc4ccccc4c3)n2)cc1 ; c1ccc(-c2nc(-c3ccc4ccccc4c3)nc(-c3ccc(-c4cccc5c4oc4c(-c6ccccc6)c6ccccc6cc45)c4ccccc34)n2)cc1 ; c1ccc(-c2ccc(-c3c4ccccc4cc4c3oc3c(-c5ccc(-c6nc(-c7ccccc7)nc(-c7ccccc7)n6)cc5)cccc34)cc2)cc1]

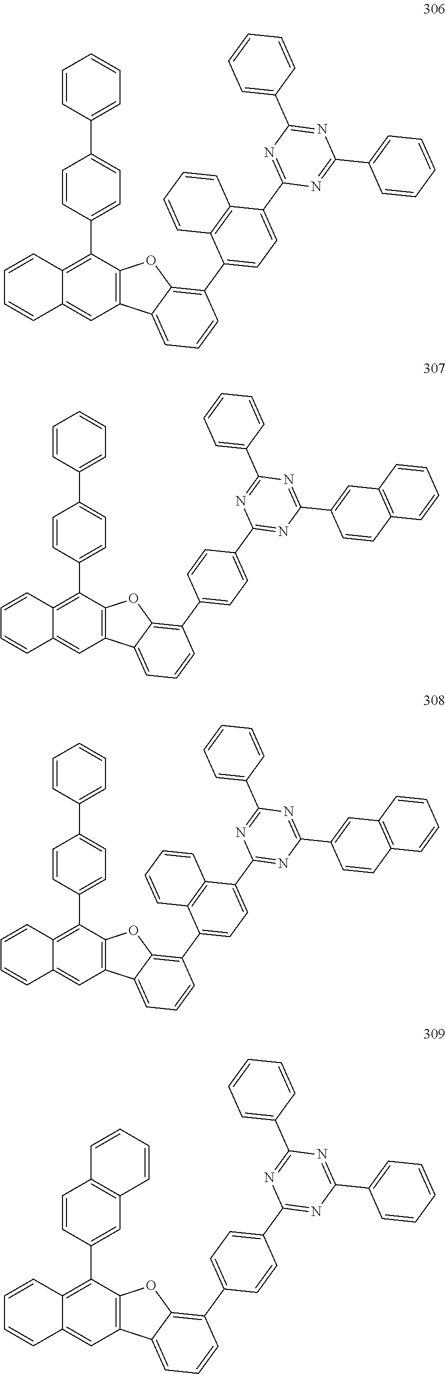 [SMILES: c1ccc(-c2ccc(-c3c4ccccc4cc4c3oc3c(-c5ccc(-c6nc(-c7ccccc7)nc(-c7ccccc7)n6)c6ccccc56)cccc34)cc2)cc1 ; c1ccc(-c2ccc(-c3c4ccccc4cc4c3oc3c(-c5ccc(-c6nc(-c7ccccc7)nc(-c7ccc8ccccc8c7)n6)cc5)cccc34)cc2)cc1 ; c1ccc(-c2ccc(-c3c4ccccc4cc4c3oc3c(-c5ccc(-c6nc(-c7ccccc7)nc(-c7ccc8ccccc8c7)n6)c6ccccc56)cccc34)cc2)cc1 ; c1ccc(-c2nc(-c3ccccc3)nc(-c3ccc(-c4cccc5c4oc4c(-c6ccc7ccccc7c6)c6ccccc6cc45)cc3)n2)cc1]

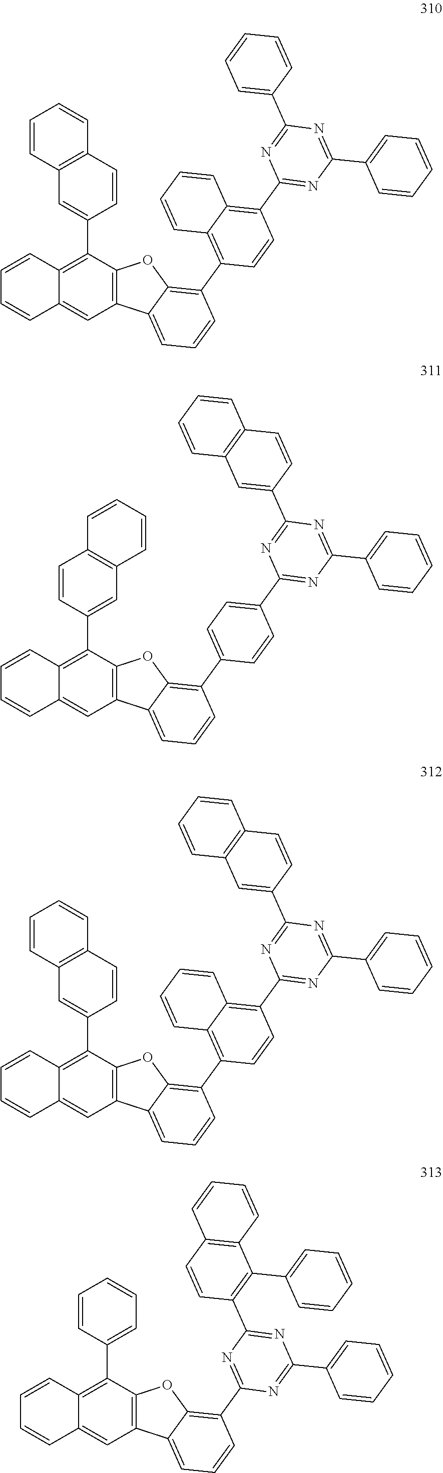

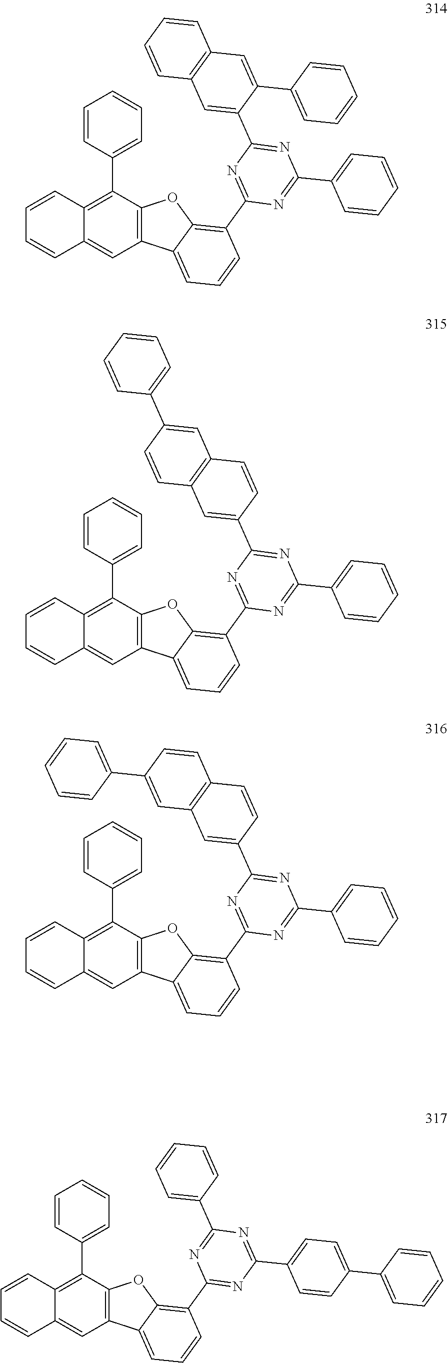 [SMILES: c1ccc(-c2nc(-c3cc4ccccc4cc3-c3ccccc3)nc(-c3cccc4c3oc3c(-c5ccccc5)c5ccccc5cc34)n2)cc1 ; c1ccc(-c2ccc3cc(-c4nc(-c5ccccc5)nc(-c5cccc6c5oc5c(-c7ccccc7)c7ccccc7cc56)n4)ccc3c2)cc1 ; c1ccc(-c2ccc3ccc(-c4nc(-c5ccccc5)nc(-c5cccc6c5oc5c(-c7ccccc7)c7ccccc7cc56)n4)cc3c2)cc1 ; c1ccc(-c2ccc(-c3nc(-c4ccccc4)nc(-c4cccc5c4oc4c(-c6ccccc6)c6ccccc6cc45)n3)cc2)cc1]

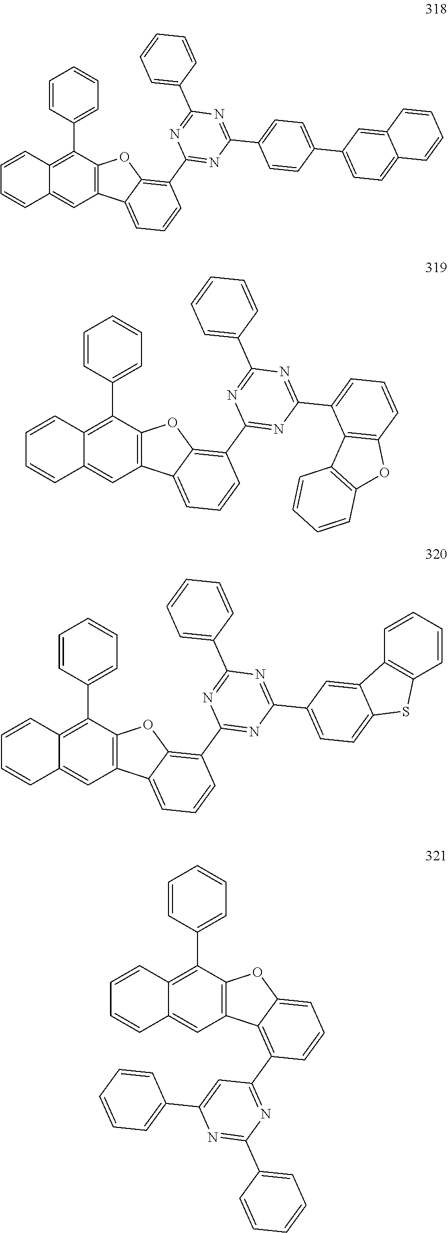 [SMILES: c1ccc(-c2nc(-c3ccc(-c4ccc5ccccc5c4)cc3)nc(-c3cccc4c3oc3c(-c5ccccc5)c5ccccc5cc34)n2)cc1 ; c1ccc(-c2nc(-c3cccc4c3oc3c(-c5ccccc5)c5ccccc5cc34)nc(-c3cccc4oc5ccccc5c34)n2)cc1 ; c1ccc(-c2nc(-c3ccc4sc5ccccc5c4c3)nc(-c3cccc4c3oc3c(-c5ccccc5)c5ccccc5cc34)n2)cc1 ; c1ccc(-c2cc(-c3cccc4oc5c(-c6ccccc6)c6ccccc6cc5c34)nc(-c3ccccc3)n2)cc1]

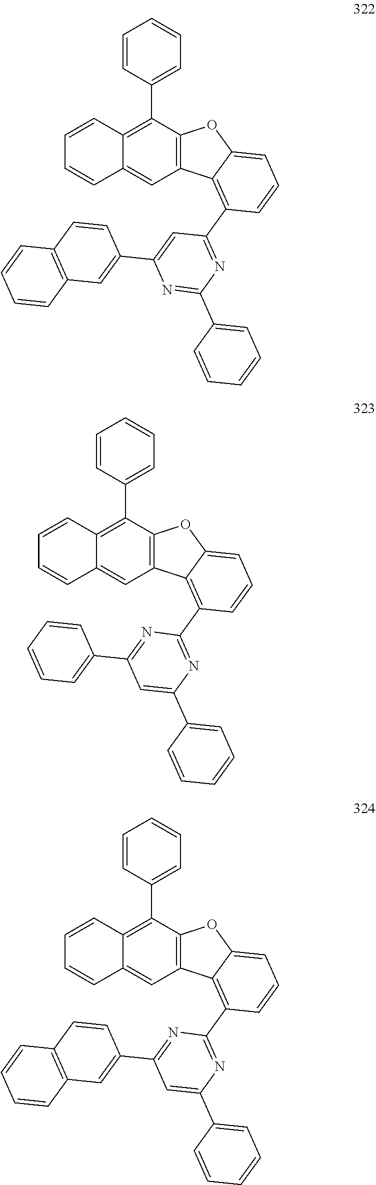 [SMILES: c1ccc(-c2nc(-c3ccc4ccccc4c3)cc(-c3cccc4oc5c(-c6ccccc6)c6ccccc6cc5c34)n2)cc1 ; c1ccc(-c2cc(-c3ccccc3)nc(-c3cccc4oc5c(-c6ccccc6)c6ccccc6cc5c34)n2)cc1 ; c1ccc(-c2cc(-c3ccc4ccccc4c3)nc(-c3cccc4oc5c(-c6ccccc6)c6ccccc6cc5c34)n2)cc1]

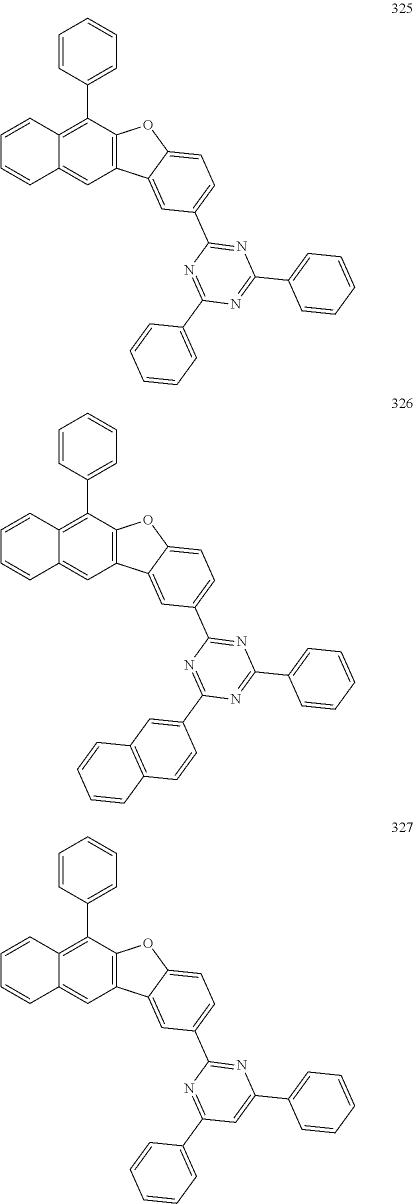

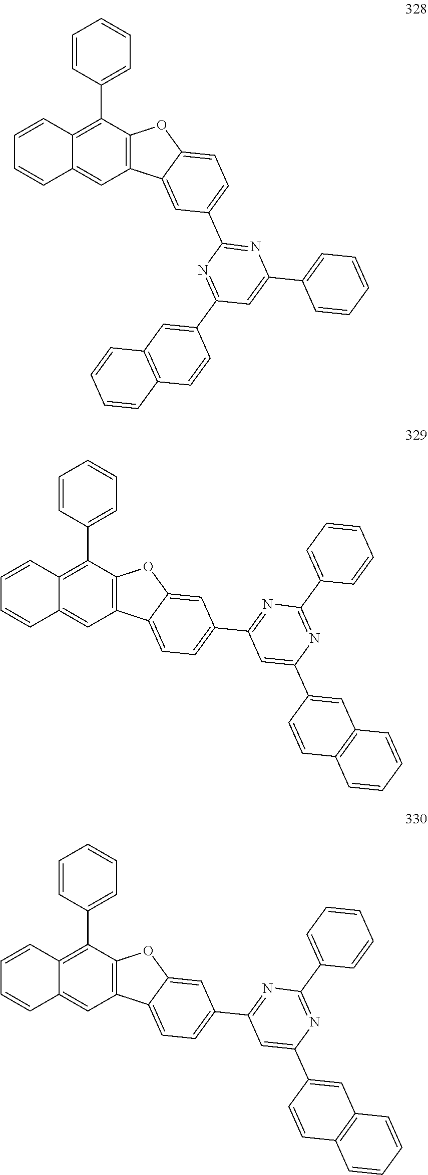 [SMILES: c1ccc(-c2cc(-c3ccc4ccccc4c3)nc(-c3ccc4oc5c(-c6ccccc6)c6ccccc6cc5c4c3)n2)cc1 ; c1ccc(-c2nc(-c3ccc4ccccc4c3)cc(-c3ccc4c(c3)oc3c(-c5ccccc5)c5ccccc5cc34)n2)cc1 ; c1ccc(-c2nc(-c3ccc4ccccc4c3)cc(-c3ccc4c(c3)oc3c(-c5ccccc5)c5ccccc5cc34)n2)cc1]

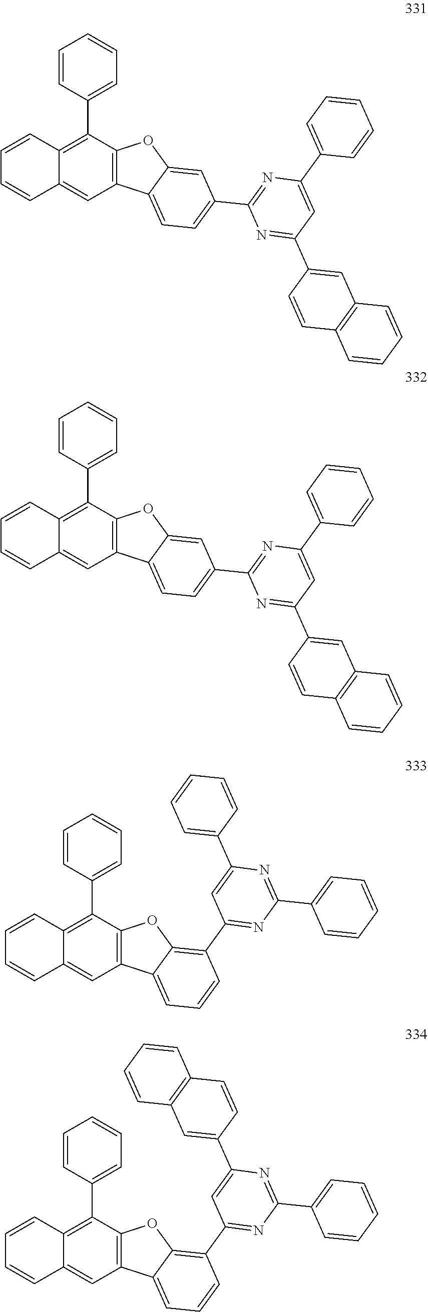

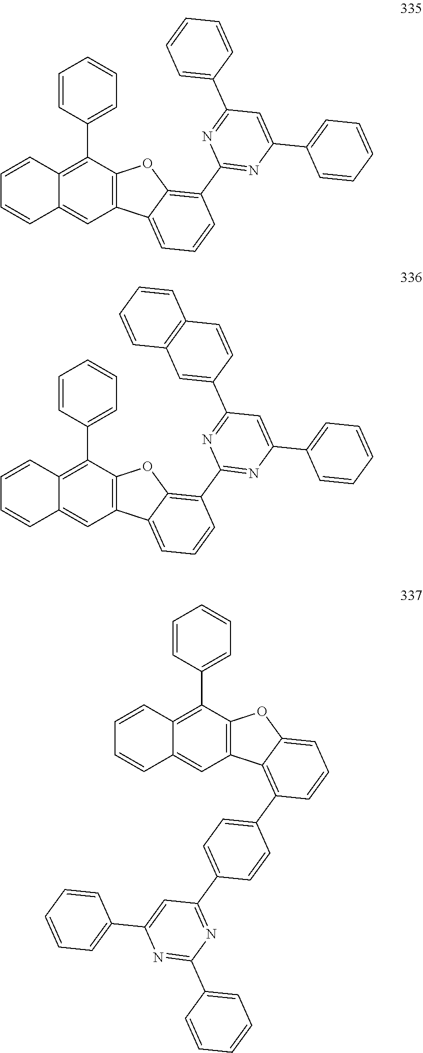

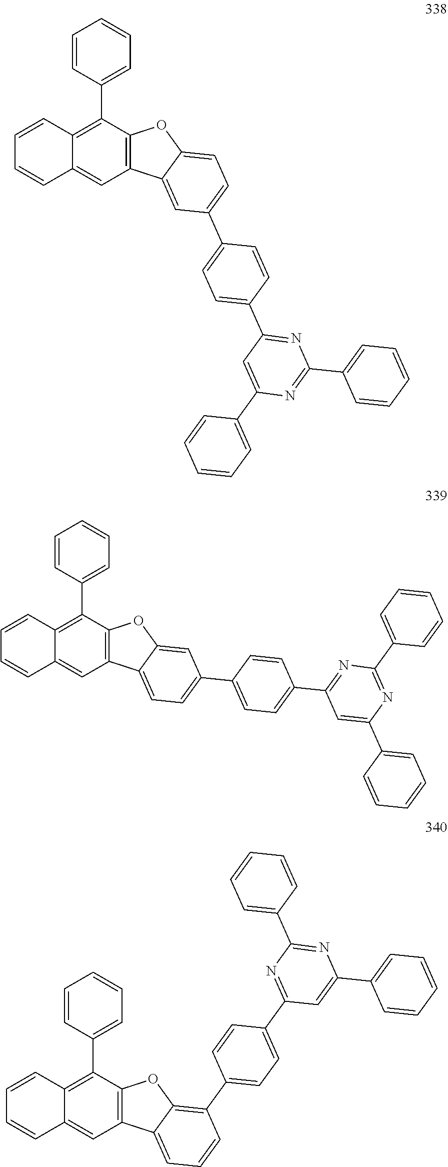 [SMILES: c1ccc(-c2cc(-c3ccc(-c4ccc5oc6c(-c7ccccc7)c7ccccc7cc6c5c4)cc3)nc(-c3ccccc3)n2)cc1 ; c1ccc(-c2cc(-c3ccc(-c4ccc5c(c4)oc4c(-c6ccccc6)c6ccccc6cc45)cc3)nc(-c3ccccc3)n2)cc1 ; c1ccc(-c2cc(-c3ccc(-c4cccc5c4oc4c(-c6ccccc6)c6ccccc6cc45)cc3)nc(-c3ccccc3)n2)cc1]

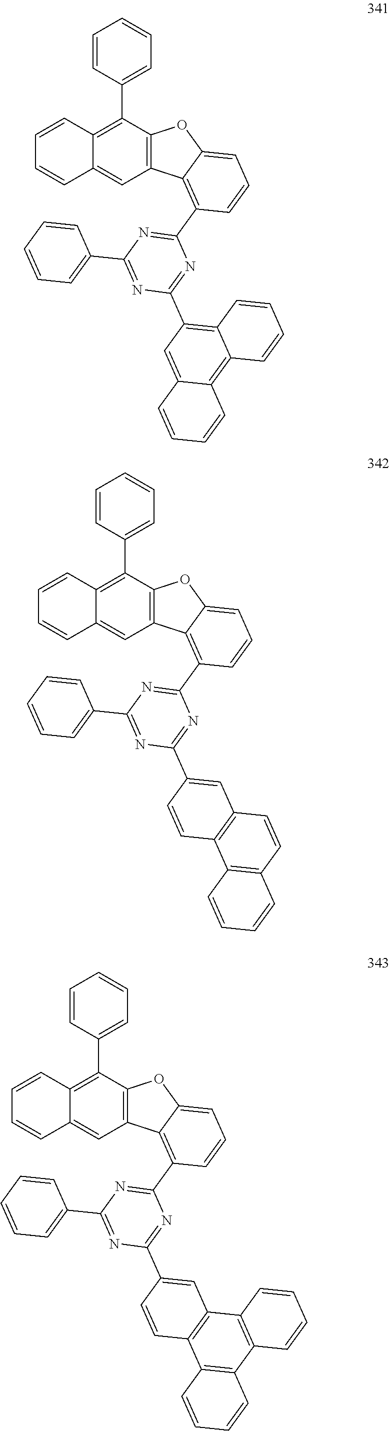 [SMILES: c1ccc(-c2nc(-c3cc4ccccc4c4ccccc34)nc(-c3cccc4oc5c(-c6ccccc6)c6ccccc6cc5c34)n2)cc1 ; c1ccc(-c2nc(-c3ccc4c(ccc5ccccc54)c3)nc(-c3cccc4oc5c(-c6ccccc6)c6ccccc6cc5c34)n2)cc1 ; c1ccc(-c2nc(-c3ccc4c5ccccc5c5ccccc5c4c3)nc(-c3cccc4oc5c(-c6ccccc6)c6ccccc6cc5c34)n2)cc1]

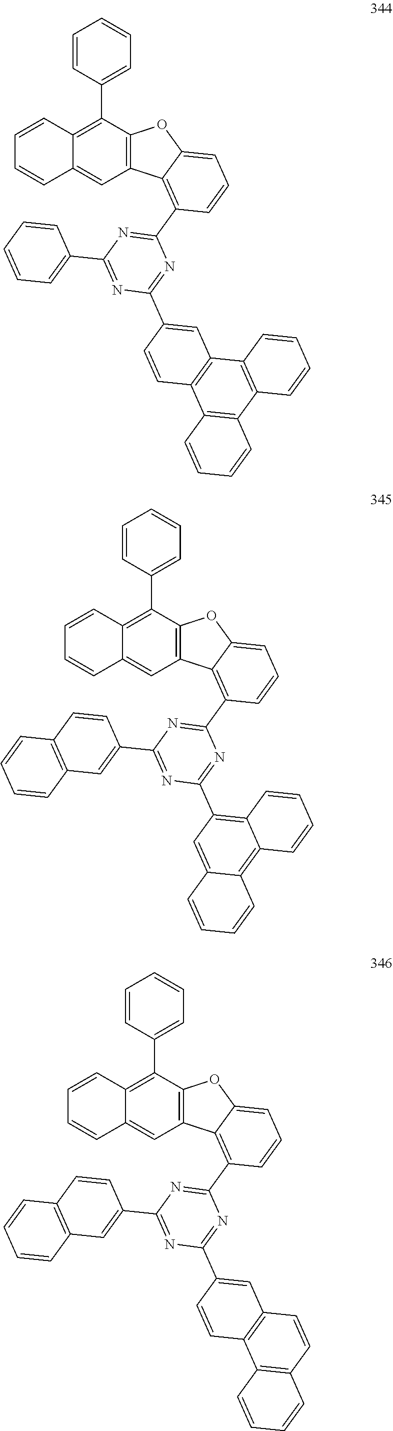 [SMILES: c1ccc(-c2nc(-c3ccc4c5ccccc5c5ccccc5c4c3)nc(-c3cccc4oc5c(-c6ccccc6)c6ccccc6cc5c34)n2)cc1 ; c1ccc(-c2c3ccccc3cc3c2oc2cccc(-c4nc(-c5ccc6ccccc6c5)nc(-c5cc6ccccc6c6ccccc56)n4)c23)cc1 ; c1ccc(-c2c3ccccc3cc3c2oc2cccc(-c4nc(-c5ccc6ccccc6c5)nc(-c5ccc6c(ccc7ccccc76)c5)n4)c23)cc1]

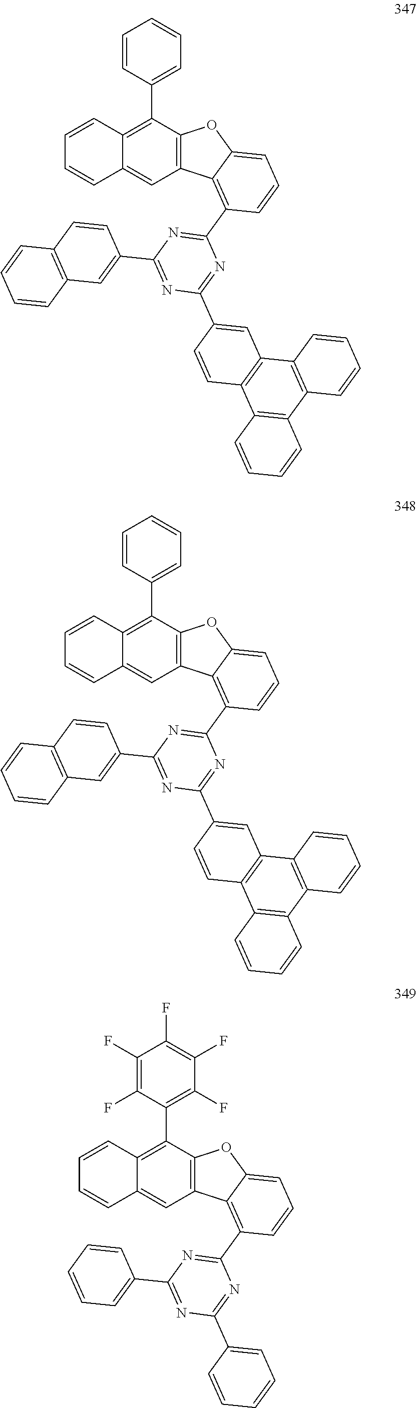 [SMILES: c1ccc(-c2c3ccccc3cc3c2oc2cccc(-c4nc(-c5ccc6ccccc6c5)nc(-c5ccc6c7ccccc7c7ccccc7c6c5)n4)c23)cc1 ; c1ccc(-c2c3ccccc3cc3c2oc2cccc(-c4nc(-c5ccc6ccccc6c5)nc(-c5ccc6c7ccccc7c7ccccc7c6c5)n4)c23)cc1 ; Fc1c(F)c(F)c(-c2c3ccccc3cc3c2oc2cccc(-c4nc(-c5ccccc5)nc(-c5ccccc5)n4)c23)c(F)c1F]

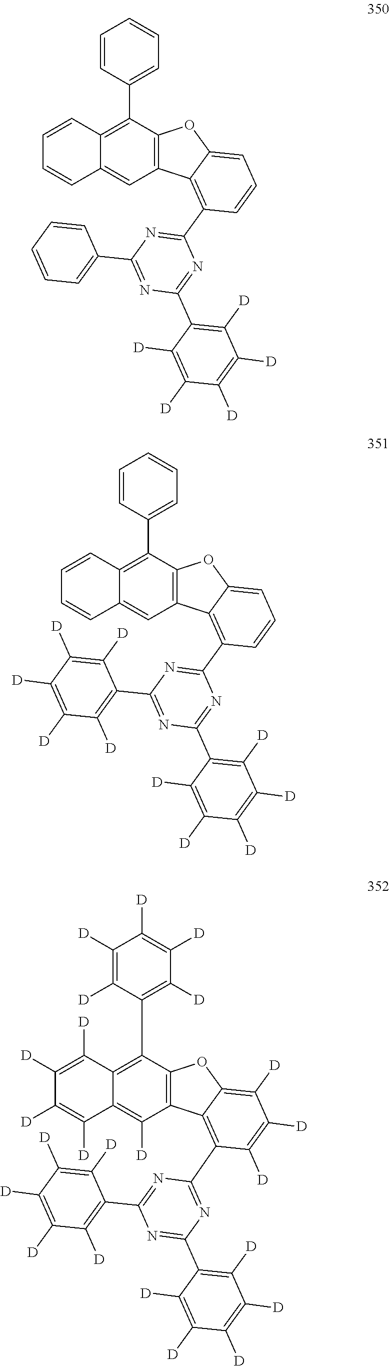 [SMILES: [2H]c1c([2H])c([2H])c(-c2nc(-c3ccccc3)nc(-c3cccc4oc5c(-c6ccccc6)c6ccccc6cc5c34)n2)c([2H])c1[2H] ; [2H]c1c([2H])c([2H])c(-c2nc(-c3c([2H])c([2H])c([2H])c([2H])c3[2H])nc(-c3cccc4oc5c(-c6ccccc6)c6ccccc6cc5c34)n2)c([2H])c1[2H] ; [2H]c1c([2H])c([2H])c(-c2nc(-c3c([2H])c([2H])c([2H])c([2H])c3[2H])nc(-c3c([2H])c([2H])c([2H])c4oc5c(-c6c([2H])c([2H])c([2H])c([2H])c6[2H])c6c([2H])c([2H])c([2H])c([2H])c6c([2H])c5c34)n2)c([2H])c1[2H]]

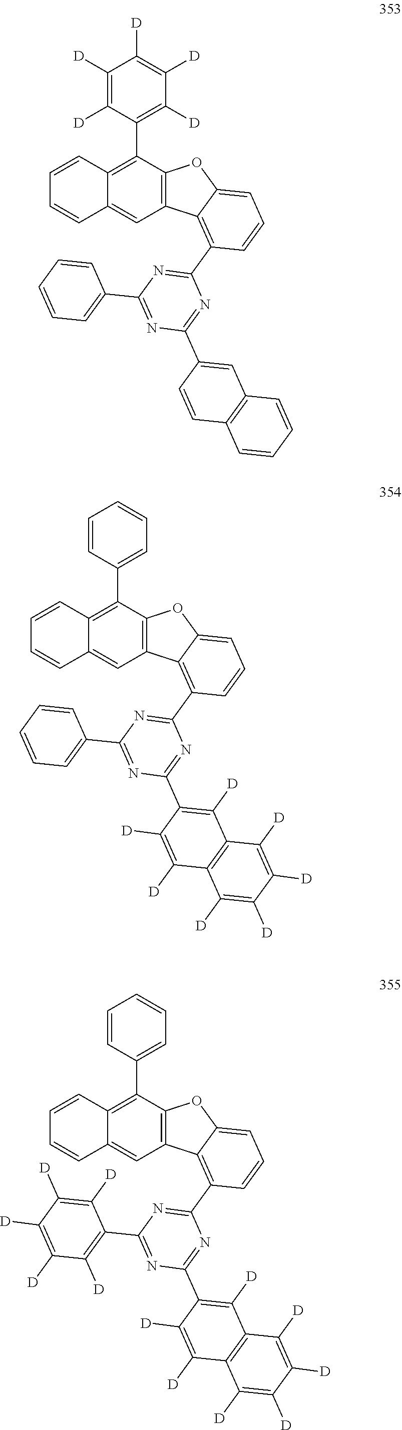 [SMILES: [2H]c1c([2H])c([2H])c(-c2c3ccccc3cc3c2oc2cccc(-c4nc(-c5ccccc5)nc(-c5ccc6ccccc6c5)n4)c23)c([2H])c1[2H] ; [2H]c1c([2H])c([2H])c2c([2H])c(-c3nc(-c4ccccc4)nc(-c4cccc5oc6c(-c7ccccc7)c7ccccc7cc6c45)n3)c([2H])c([2H])c2c1[2H] ; [2H]c1c([2H])c([2H])c(-c2nc(-c3c([2H])c([2H])c4c([2H])c([2H])c([2H])c([2H])c4c3[2H])nc(-c3cccc4oc5c(-c6ccccc6)c6ccccc6cc5c34)n2)c([2H])c1[2H]]

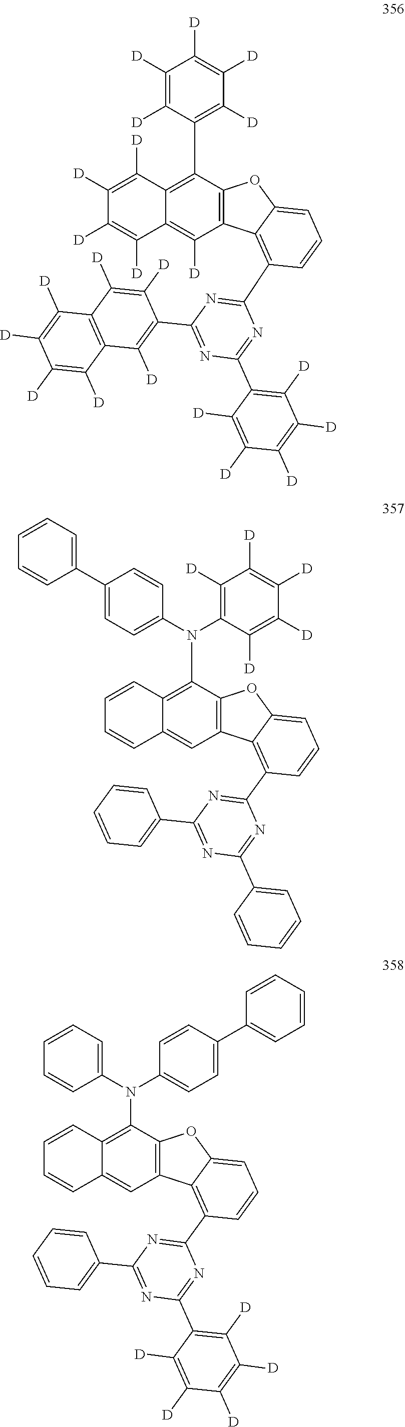

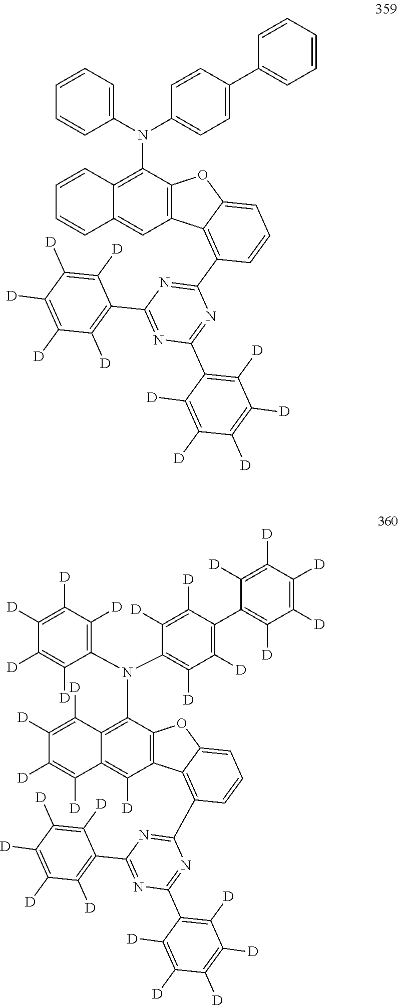 [SMILES: [2H]c1c([2H])c([2H])c(-c2nc(-c3c([2H])c([2H])c([2H])c([2H])c3[2H])nc(-c3cccc4oc5c(N(c6ccccc6)c6ccc(-c7ccccc7)cc6)c6ccccc6cc5c34)n2)c([2H])c1[2H] ; [2H]c1c([2H])c([2H])c(-c2nc(-c3c([2H])c([2H])c([2H])c([2H])c3[2H])nc(-c3cccc4oc5c(N(c6c([2H])c([2H])c([2H])c([2H])c6[2H])c6c([2H])c([2H])c(-c7c([2H])c([2H])c([2H])c([2H])c7[2H])c([2H])c6[2H])c6c([2H])c([2H])c([2H])c([2H])c6c([2H])c5c34)n2)c([2H])c1[2H]]

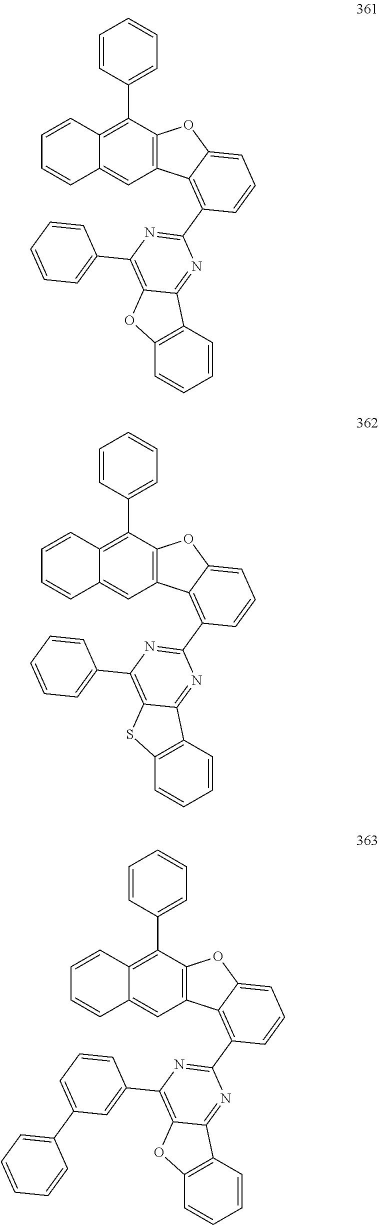 [SMILES: c1ccc(-c2c3ccccc3cc3c2oc2cccc(-c4nc(-c5ccccc5)c5oc6ccccc6c5n4)c23)cc1 ; c1ccc(-c2c3ccccc3cc3c2oc2cccc(-c4nc(-c5ccccc5)c5sc6ccccc6c5n4)c23)cc1 ; c1ccc(-c2cccc(-c3nc(-c4cccc5oc6c(-c7ccccc7)c7ccccc7cc6c45)nc4c3oc3ccccc34)c2)cc1]

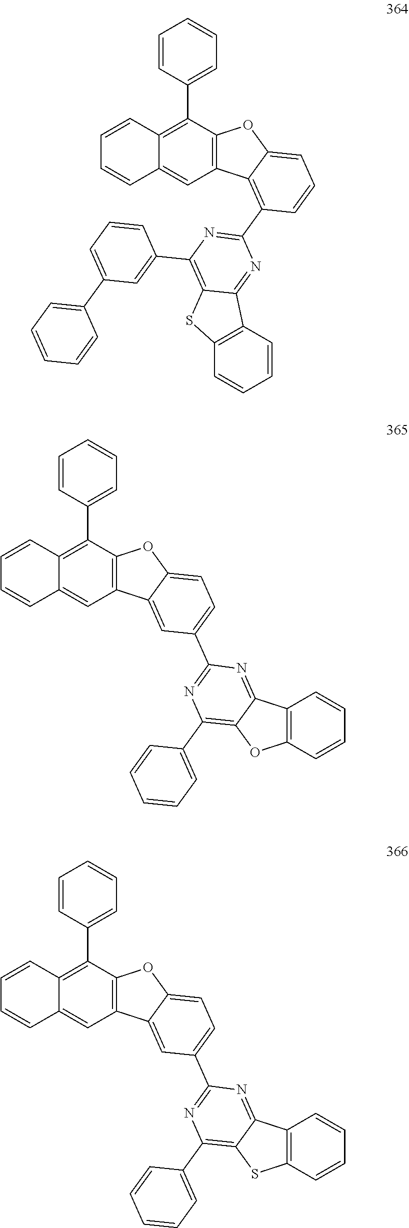 [SMILES: c1ccc(-c2cccc(-c3nc(-c4cccc5oc6c(-c7ccccc7)c7ccccc7cc6c45)nc4c3sc3ccccc34)c2)cc1 ; c1ccc(-c2c3ccccc3cc3c2oc2ccc(-c4nc(-c5ccccc5)c5oc6ccccc6c5n4)cc23)cc1 ; c1ccc(-c2c3ccccc3cc3c2oc2ccc(-c4nc(-c5ccccc5)c5sc6ccccc6c5n4)cc23)cc1]

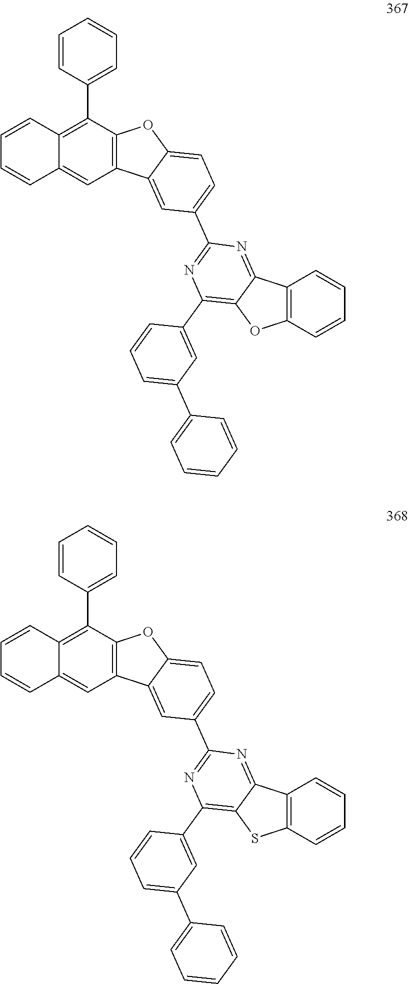 [SMILES: c1ccc(-c2cccc(-c3nc(-c4ccc5oc6c(-c7ccccc7)c7ccccc7cc6c5c4)nc4c3oc3ccccc34)c2)cc1 ; c1ccc(-c2cccc(-c3nc(-c4ccc5oc6c(-c7ccccc7)c7ccccc7cc6c5c4)nc4c3sc3ccccc34)c2)cc1]

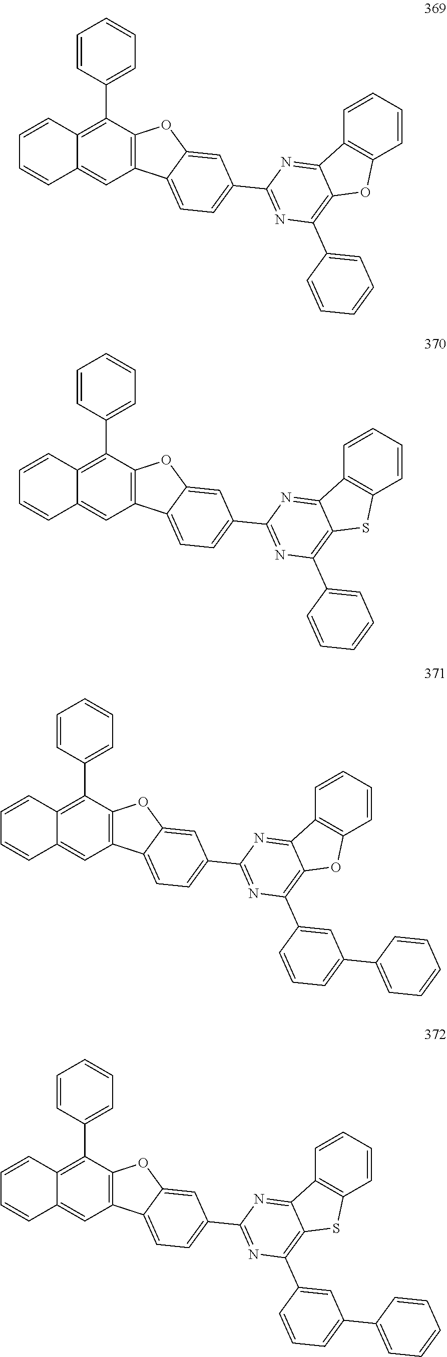 [SMILES: c1ccc(-c2c3ccccc3cc3c2oc2cc(-c4nc(-c5ccccc5)c5oc6ccccc6c5n4)ccc23)cc1 ; c1ccc(-c2c3ccccc3cc3c2oc2cc(-c4nc(-c5ccccc5)c5sc6ccccc6c5n4)ccc23)cc1 ; c1ccc(-c2cccc(-c3nc(-c4ccc5c(c4)oc4c(-c6ccccc6)c6ccccc6cc45)nc4c3oc3ccccc34)c2)cc1 ; c1ccc(-c2cccc(-c3nc(-c4ccc5c(c4)oc4c(-c6ccccc6)c6ccccc6cc45)nc4c3sc3ccccc34)c2)cc1]

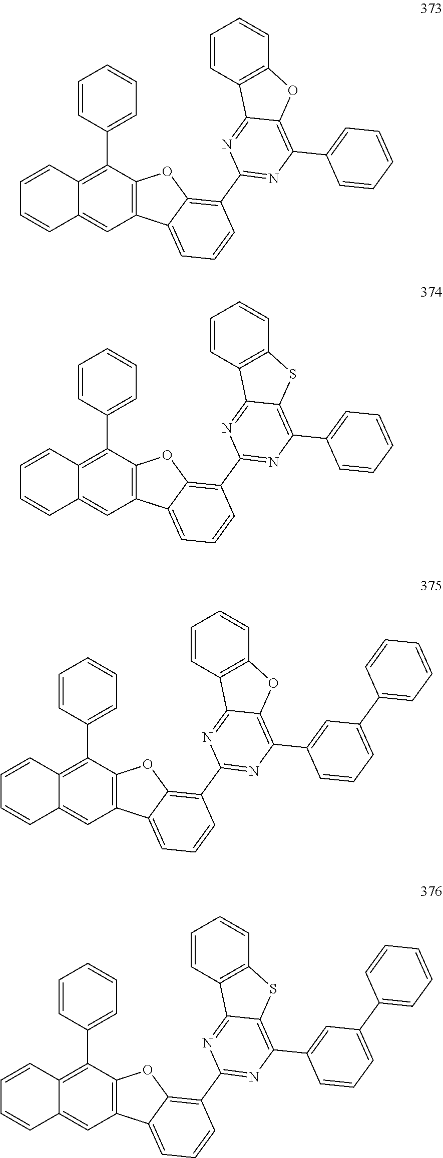 [SMILES: c1ccc(-c2c3ccccc3cc3c2oc2c(-c4nc(-c5ccccc5)c5oc6ccccc6c5n4)cccc23)cc1 ; c1ccc(-c2c3ccccc3cc3c2oc2c(-c4nc(-c5ccccc5)c5sc6ccccc6c5n4)cccc23)cc1 ; c1ccc(-c2cccc(-c3nc(-c4cccc5c4oc4c(-c6ccccc6)c6ccccc6cc45)nc4c3oc3ccccc34)c2)cc1 ; c1ccc(-c2cccc(-c3nc(-c4cccc5c4oc4c(-c6ccccc6)c6ccccc6cc45)nc4c3sc3ccccc34)c2)cc1]

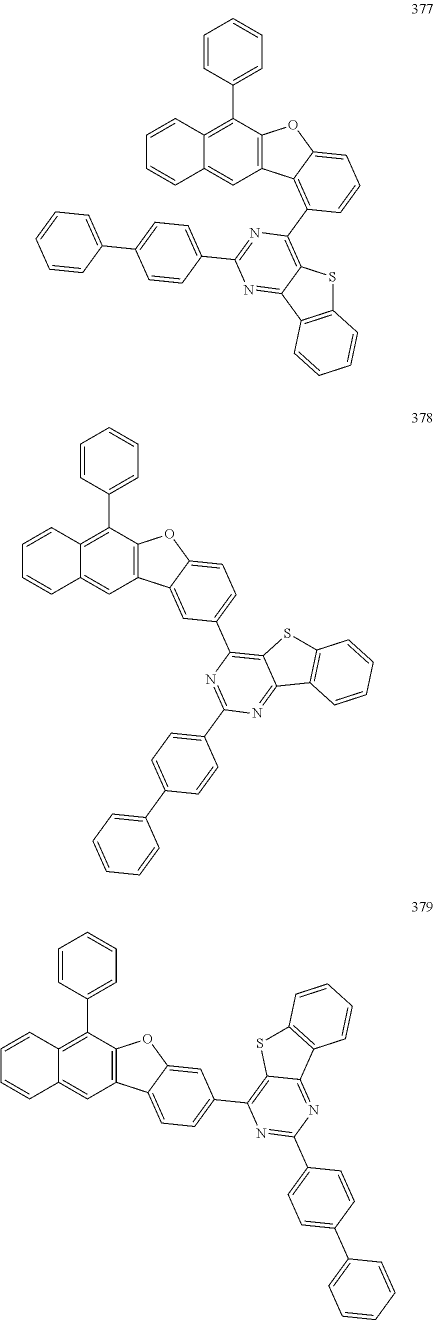 [SMILES: c1ccc(-c2ccc(-c3nc(-c4cccc5oc6c(-c7ccccc7)c7ccccc7cc6c45)c4sc5ccccc5c4n3)cc2)cc1 ; c1ccc(-c2ccc(-c3nc(-c4ccc5oc6c(-c7ccccc7)c7ccccc7cc6c5c4)c4sc5ccccc5c4n3)cc2)cc1 ; c1ccc(-c2ccc(-c3nc(-c4ccc5c(c4)oc4c(-c6ccccc6)c6ccccc6cc45)c4sc5ccccc5c4n3)cc2)cc1]

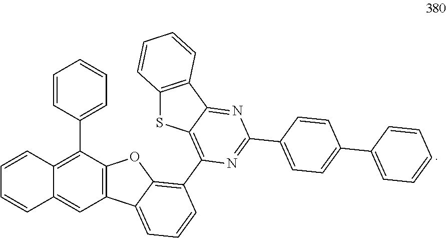 [SMILES: c1ccc(-c2ccc(-c3nc(-c4cccc5c4oc4c(-c6ccccc6)c6ccccc6cc45)c4sc5ccccc5c4n3)cc2)cc1]

7. An organic light emitting device comprising:
a first electrode;
a second electrode; and
an organic material layer having one or more layers provided between the first electrode and the second electrode,
wherein the one or more layers of the organic material layer comprise one or more heterocyclic compounds of claim 1.

8. The organic light emitting device of claim 7, wherein the organic material layer comprises a light emitting layer, and the light emitting layer comprises one or more of the heterocyclic compounds.

9. The organic light emitting device of claim 7, wherein the organic material layer comprises a light emitting layer, the light emitting layer comprises a host, and the host comprises one or more of the heterocyclic compounds.

10. The organic light emitting device of claim 7, wherein the organic material layer comprising the heterocyclic compound further comprises a compound of the following Chemical Formula 2 or 3: 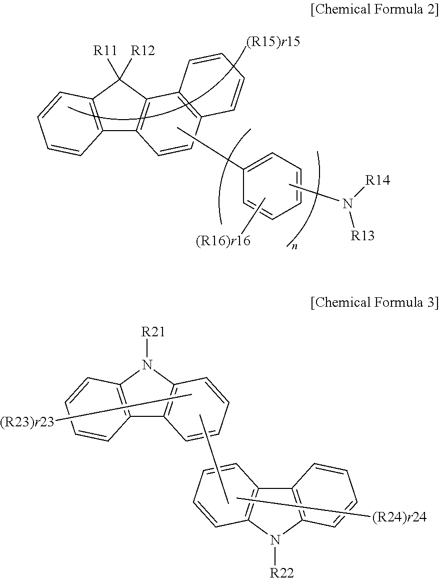
in Chemical Formulae 2 and 3,
R11 to R14 and R21 to R24 are each independently hydrogen; deuterium; a substituted or unsubstituted alkyl group having 1 to 60 carbon atoms; a substituted or unsubstituted aryl group having 6 to 60 carbon atoms; or a substituted or unsubstituted heteroaryl group having 2 to 60 carbon atoms,
R15 and R16 are each independently hydrogen; or deuterium,
n is 0 or 1,
r15 is an integer from 0 to 9,
r16 is an integer from 0 to 4,
r23 and r24 are each an integer from 0 to 7, and
when r15, r16, r23 and r24 are each 2 or higher, substituents in the parenthesis are the same as or different from each other.

11. The organic light emitting device of claim 10, wherein Chemical Formula 2 is represented by any one of the following compounds:

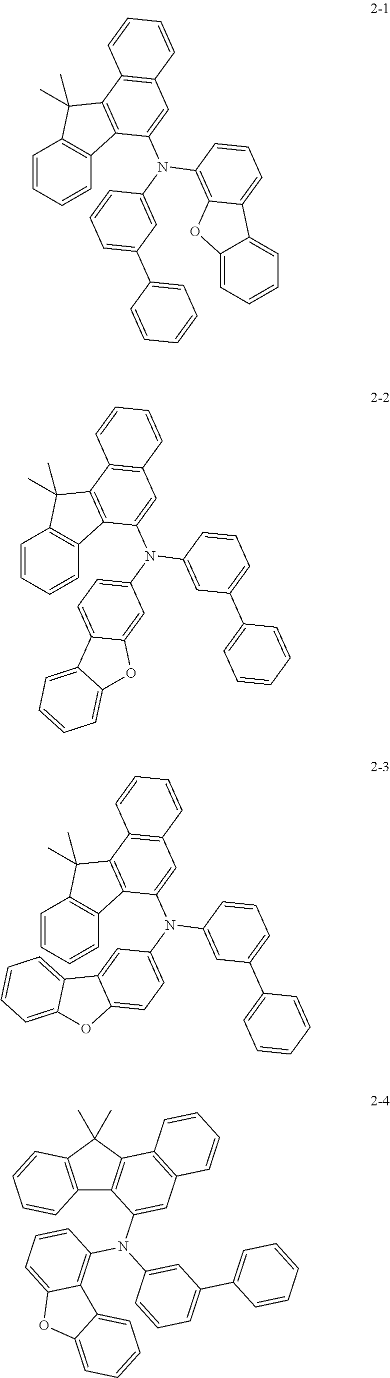 [SMILES: CC1(C)c2ccccc2-c2c(N(c3cccc(-c4ccccc4)c3)c3cccc4c3oc3ccccc34)cc3ccccc3c21 ; CC1(C)c2ccccc2-c2c(N(c3cccc(-c4ccccc4)c3)c3ccc4c(c3)oc3ccccc34)cc3ccccc3c21 ; CC1(C)c2ccccc2-c2c(N(c3cccc(-c4ccccc4)c3)c3ccc4oc5ccccc5c4c3)cc3ccccc3c21 ; CC1(C)c2ccccc2-c2c(N(c3cccc(-c4ccccc4)c3)c3cccc4oc5ccccc5c34)cc3ccccc3c21]

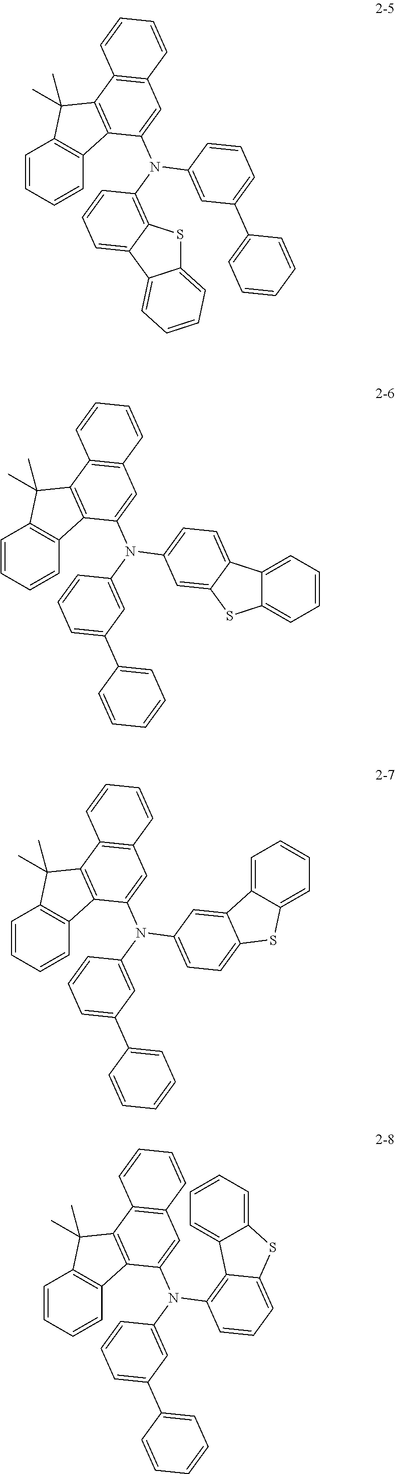

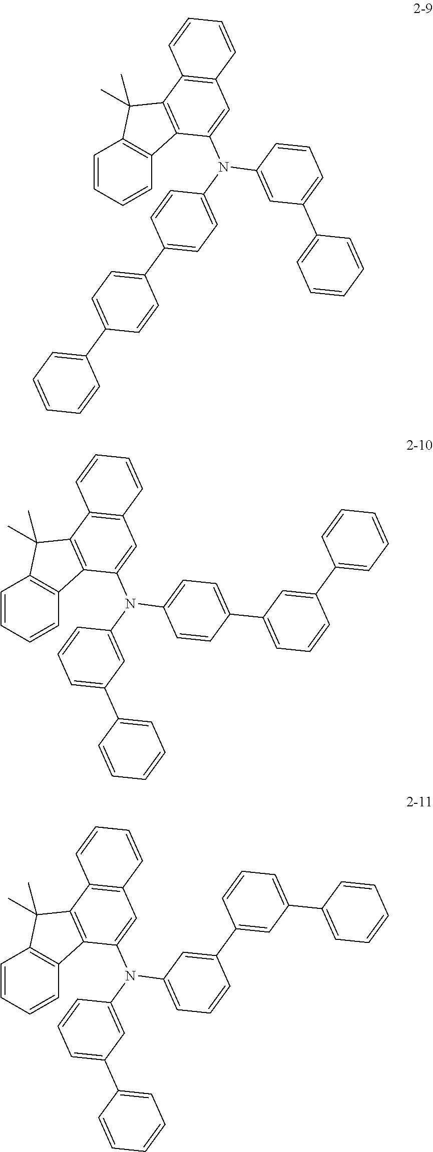

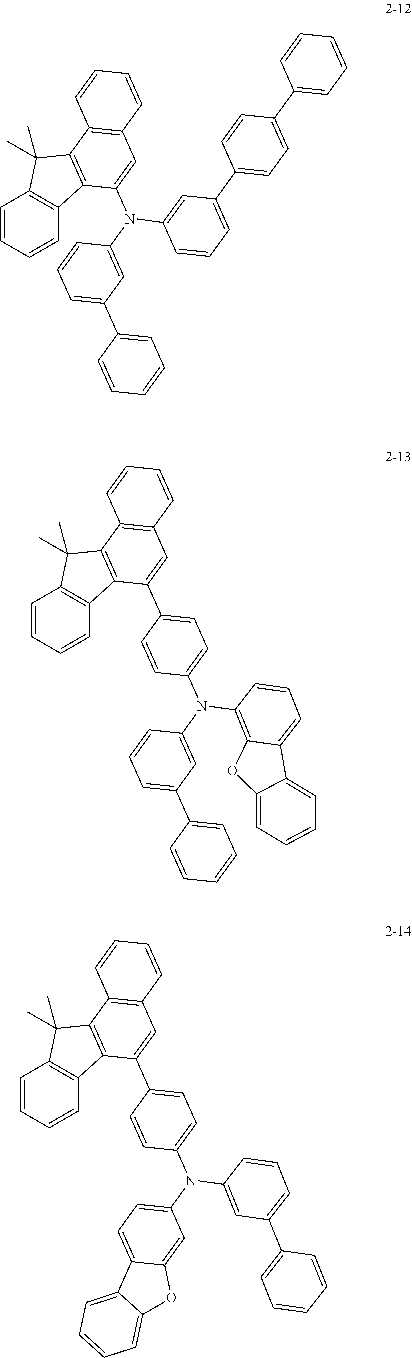 [SMILES: CC1(C)c2ccccc2-c2c(N(c3cccc(-c4ccccc4)c3)c3cccc(-c4ccc(-c5ccccc5)cc4)c3)cc3ccccc3c21 ; CC1(C)c2ccccc2-c2c(-c3ccc(N(c4cccc(-c5ccccc5)c4)c4cccc5c4oc4ccccc45)cc3)cc3ccccc3c21 ; CC1(C)c2ccccc2-c2c(-c3ccc(N(c4cccc(-c5ccccc5)c4)c4ccc5c(c4)oc4ccccc45)cc3)cc3ccccc3c21]

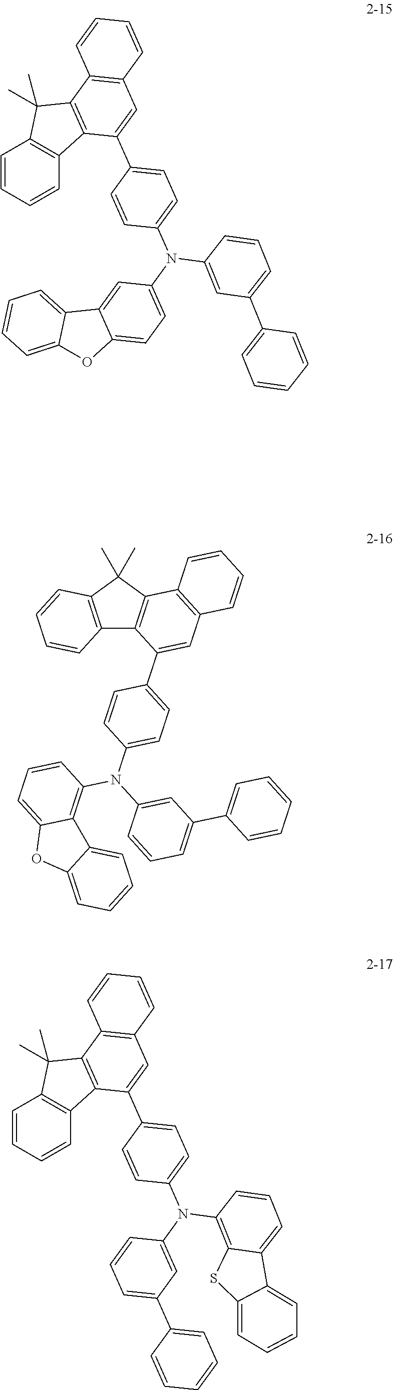 [SMILES: CC1(C)c2ccccc2-c2c(-c3ccc(N(c4cccc(-c5ccccc5)c4)c4ccc5oc6ccccc6c5c4)cc3)cc3ccccc3c21 ; CC1(C)c2ccccc2-c2c(-c3ccc(N(c4cccc(-c5ccccc5)c4)c4cccc5oc6ccccc6c45)cc3)cc3ccccc3c21 ; CC1(C)c2ccccc2-c2c(-c3ccc(N(c4cccc(-c5ccccc5)c4)c4cccc5c4sc4ccccc45)cc3)cc3ccccc3c21]

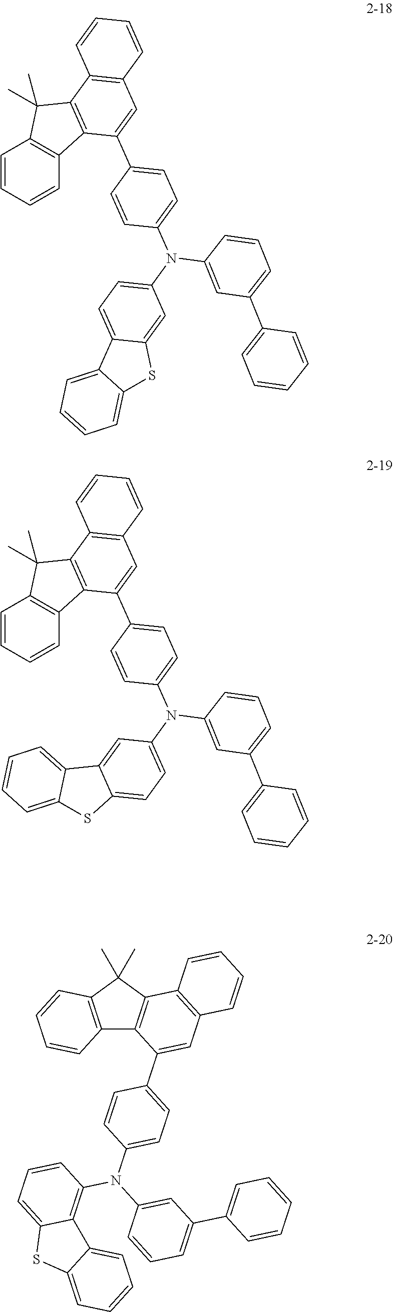

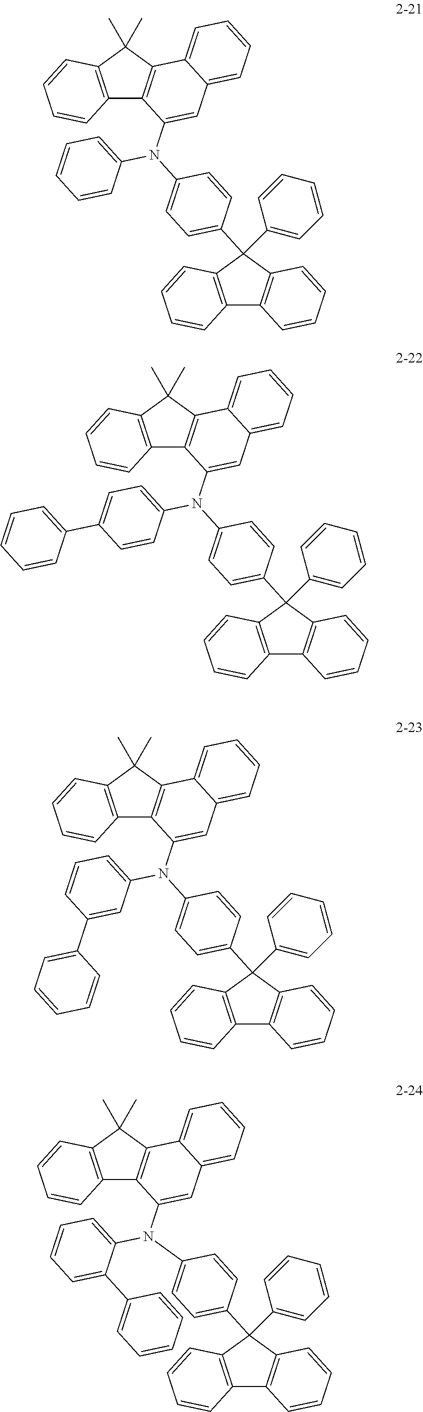

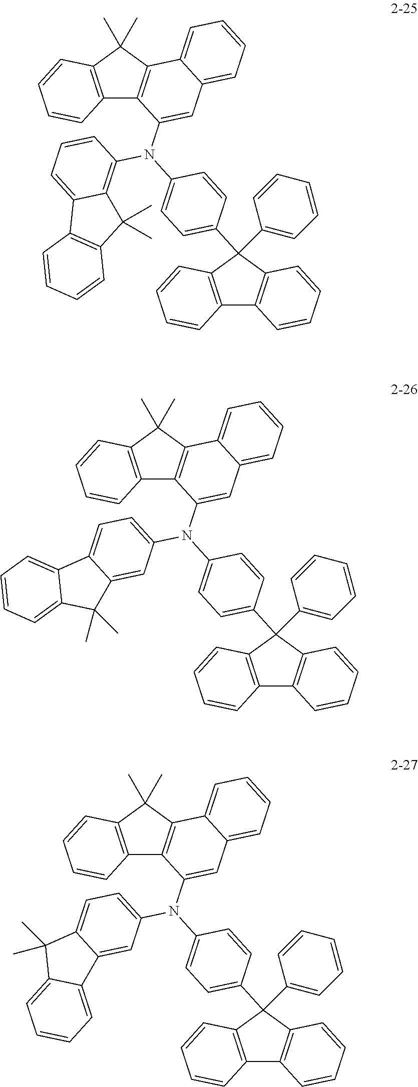 [SMILES: CC1(C)c2ccccc2-c2cccc(N(c3ccc(C4(c5ccccc5)c5ccccc5-c5ccccc54)cc3)c3cc4ccccc4c4c3-c3ccccc3C4(C)C)c21 ; CC1(C)c2ccccc2-c2ccc(N(c3ccc(C4(c5ccccc5)c5ccccc5-c5ccccc54)cc3)c3cc4ccccc4c4c3-c3ccccc3C4(C)C)cc21 ; CC1(C)c2ccccc2-c2cc(N(c3ccc(C4(c5ccccc5)c5ccccc5-c5ccccc54)cc3)c3cc4ccccc4c4c3-c3ccccc3C4(C)C)ccc21]

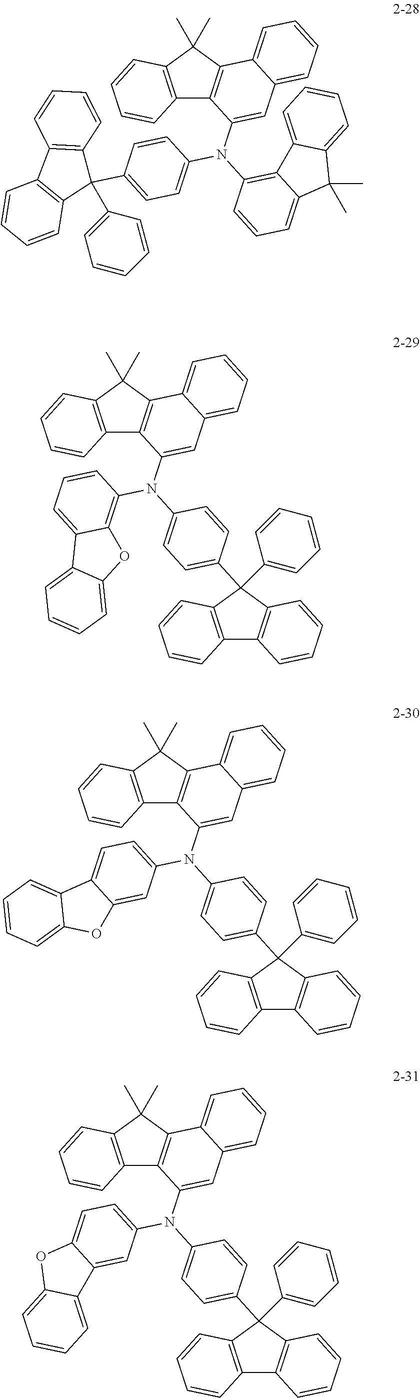 [SMILES: CC1(C)c2ccccc2-c2c(N(c3ccc(C4(c5ccccc5)c5ccccc5-c5ccccc54)cc3)c3cc4ccccc4c4c3-c3ccccc3C4(C)C)cccc21 ; CC1(C)c2ccccc2-c2c(N(c3ccc(C4(c5ccccc5)c5ccccc5-c5ccccc54)cc3)c3cccc4c3oc3ccccc34)cc3ccccc3c21 ; CC1(C)c2ccccc2-c2c(N(c3ccc(C4(c5ccccc5)c5ccccc5-c5ccccc54)cc3)c3ccc4c(c3)oc3ccccc34)cc3ccccc3c21 ; CC1(C)c2ccccc2-c2c(N(c3ccc(C4(c5ccccc5)c5ccccc5-c5ccccc54)cc3)c3ccc4oc5ccccc5c4c3)cc3ccccc3c21]

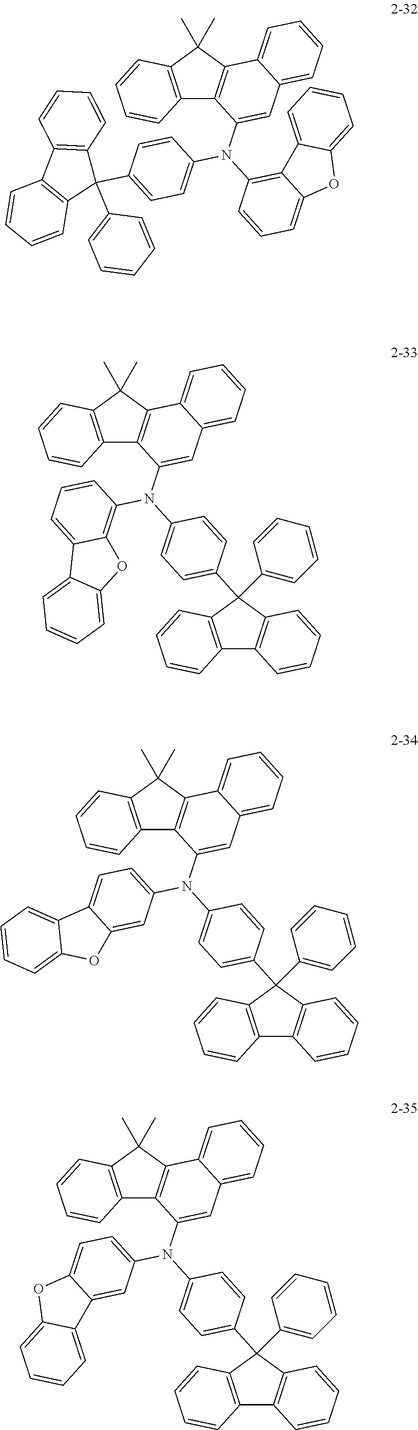 [SMILES: CC1(C)c2ccccc2-c2c(N(c3ccc(C4(c5ccccc5)c5ccccc5-c5ccccc54)cc3)c3cccc4oc5ccccc5c34)cc3ccccc3c21 ; CC1(C)c2ccccc2-c2c(N(c3ccc(C4(c5ccccc5)c5ccccc5-c5ccccc54)cc3)c3cccc4c3oc3ccccc34)cc3ccccc3c21 ; CC1(C)c2ccccc2-c2c(N(c3ccc(C4(c5ccccc5)c5ccccc5-c5ccccc54)cc3)c3ccc4c(c3)oc3ccccc34)cc3ccccc3c21 ; CC1(C)c2ccccc2-c2c(N(c3ccc(C4(c5ccccc5)c5ccccc5-c5ccccc54)cc3)c3ccc4oc5ccccc5c4c3)cc3ccccc3c21]

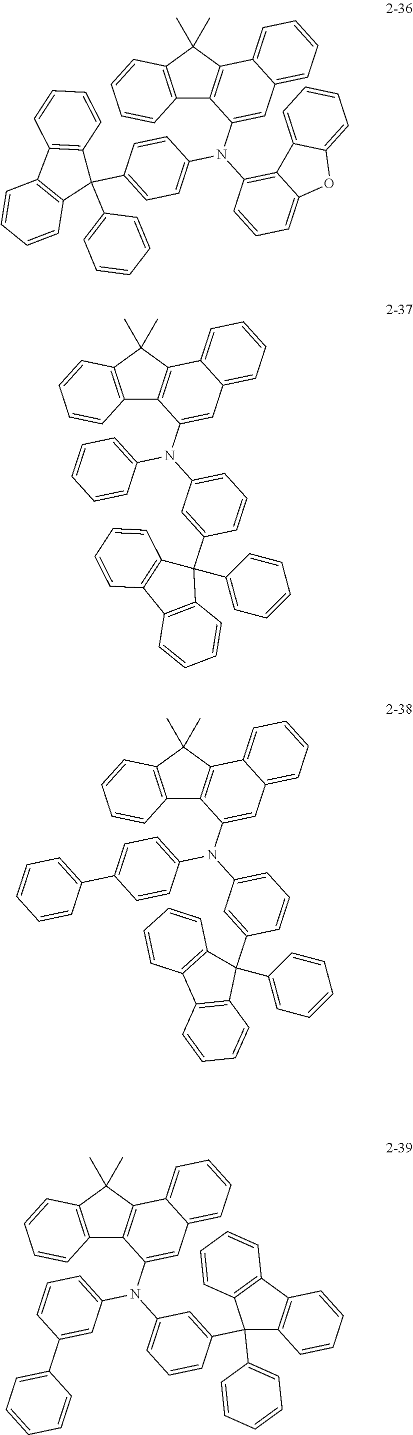

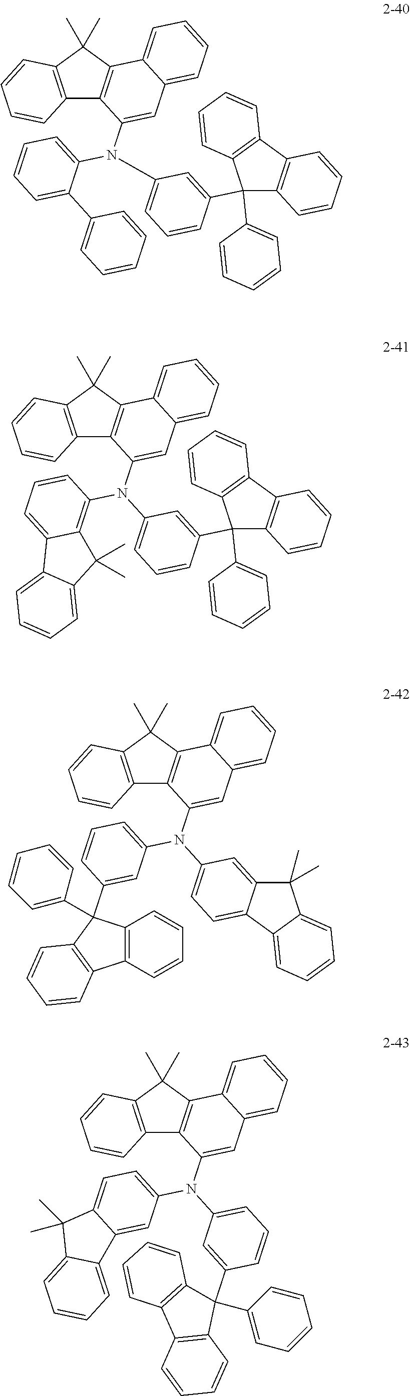 [SMILES: CC1(C)c2ccccc2-c2c(N(c3cccc(C4(c5ccccc5)c5ccccc5-c5ccccc54)c3)c3ccccc3-c3ccccc3)cc3ccccc3c21 ; CC1(C)c2ccccc2-c2cccc(N(c3cccc(C4(c5ccccc5)c5ccccc5-c5ccccc54)c3)c3cc4ccccc4c4c3-c3ccccc3C4(C)C)c21 ; CC1(C)c2ccccc2-c2ccc(N(c3cccc(C4(c5ccccc5)c5ccccc5-c5ccccc54)c3)c3cc4ccccc4c4c3-c3ccccc3C4(C)C)cc21 ; CC1(C)c2ccccc2-c2cc(N(c3cccc(C4(c5ccccc5)c5ccccc5-c5ccccc54)c3)c3cc4ccccc4c4c3-c3ccccc3C4(C)C)ccc21]

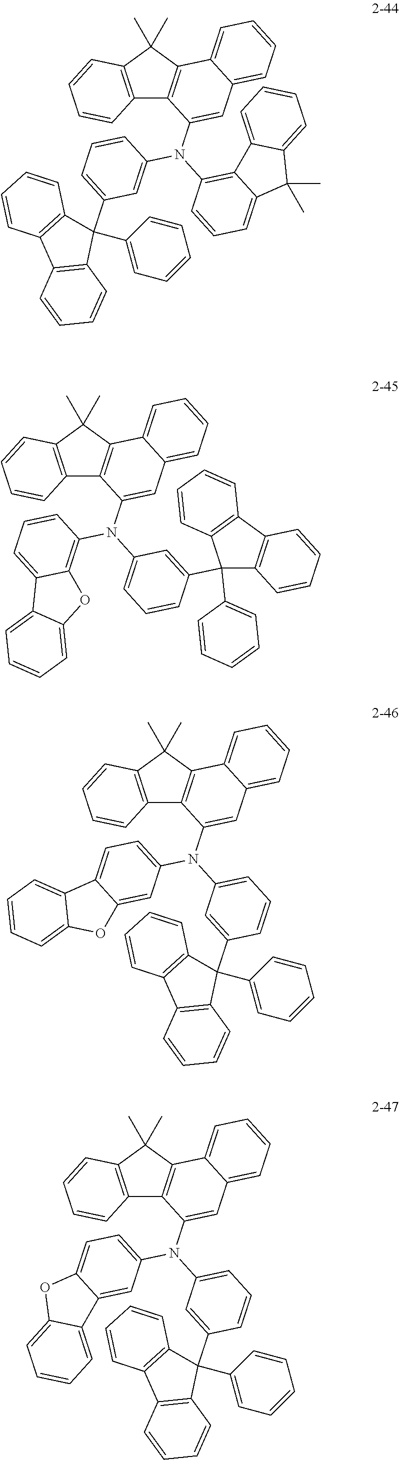 [SMILES: CC1(C)c2ccccc2-c2c(N(c3cccc(C4(c5ccccc5)c5ccccc5-c5ccccc54)c3)c3cc4ccccc4c4c3-c3ccccc3C4(C)C)cccc21 ; CC1(C)c2ccccc2-c2c(N(c3cccc(C4(c5ccccc5)c5ccccc5-c5ccccc54)c3)c3cccc4c3oc3ccccc34)cc3ccccc3c21 ; CC1(C)c2ccccc2-c2c(N(c3cccc(C4(c5ccccc5)c5ccccc5-c5ccccc54)c3)c3ccc4c(c3)oc3ccccc34)cc3ccccc3c21 ; CC1(C)c2ccccc2-c2c(N(c3cccc(C4(c5ccccc5)c5ccccc5-c5ccccc54)c3)c3ccc4oc5ccccc5c4c3)cc3ccccc3c21]

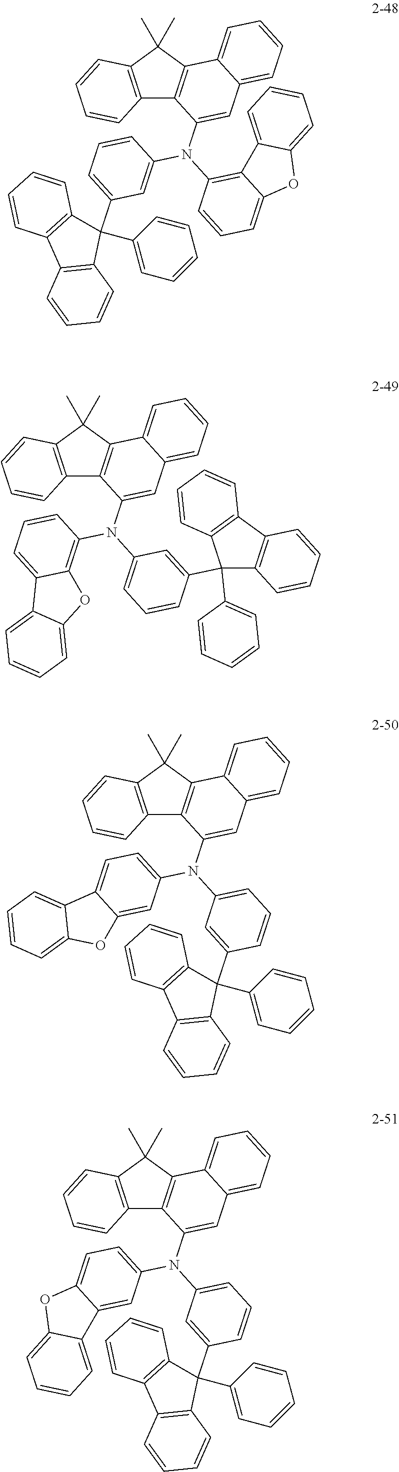 [SMILES: CC1(C)c2ccccc2-c2c(N(c3cccc(C4(c5ccccc5)c5ccccc5-c5ccccc54)c3)c3cccc4oc5ccccc5c34)cc3ccccc3c21 ; CC1(C)c2ccccc2-c2c(N(c3cccc(C4(c5ccccc5)c5ccccc5-c5ccccc54)c3)c3cccc4c3oc3ccccc34)cc3ccccc3c21 ; CC1(C)c2ccccc2-c2c(N(c3cccc(C4(c5ccccc5)c5ccccc5-c5ccccc54)c3)c3ccc4c(c3)oc3ccccc34)cc3ccccc3c21 ; CC1(C)c2ccccc2-c2c(N(c3cccc(C4(c5ccccc5)c5ccccc5-c5ccccc54)c3)c3ccc4oc5ccccc5c4c3)cc3ccccc3c21]

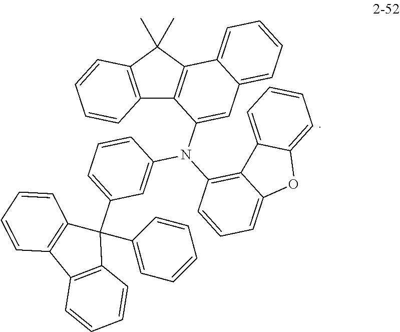 [SMILES: CC1(C)c2ccccc2-c2c(N(c3cccc(C4(c5ccccc5)c5ccccc5-c5ccccc54)c3)c3cccc4oc5ccccc5c34)cc3ccccc3c21]

12. The organic light emitting device of claim 10, wherein Chemical Formula 3 is represented by any one of the following compounds:

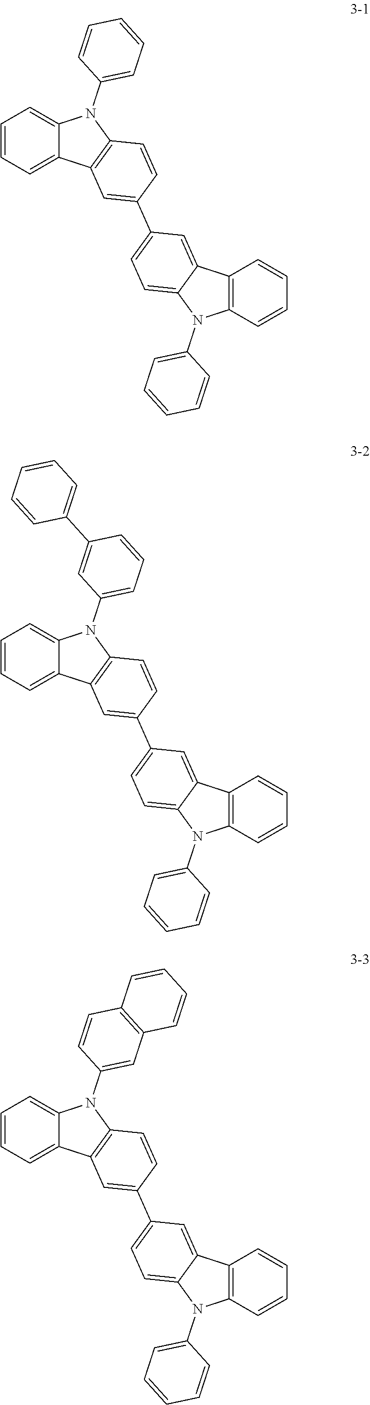

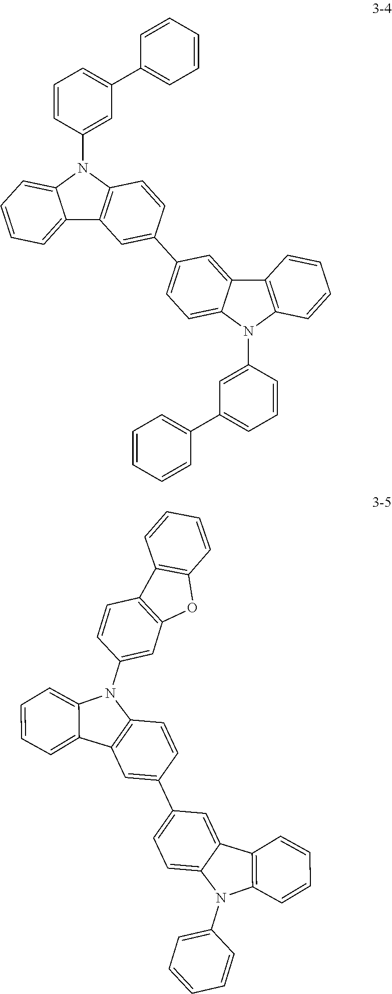

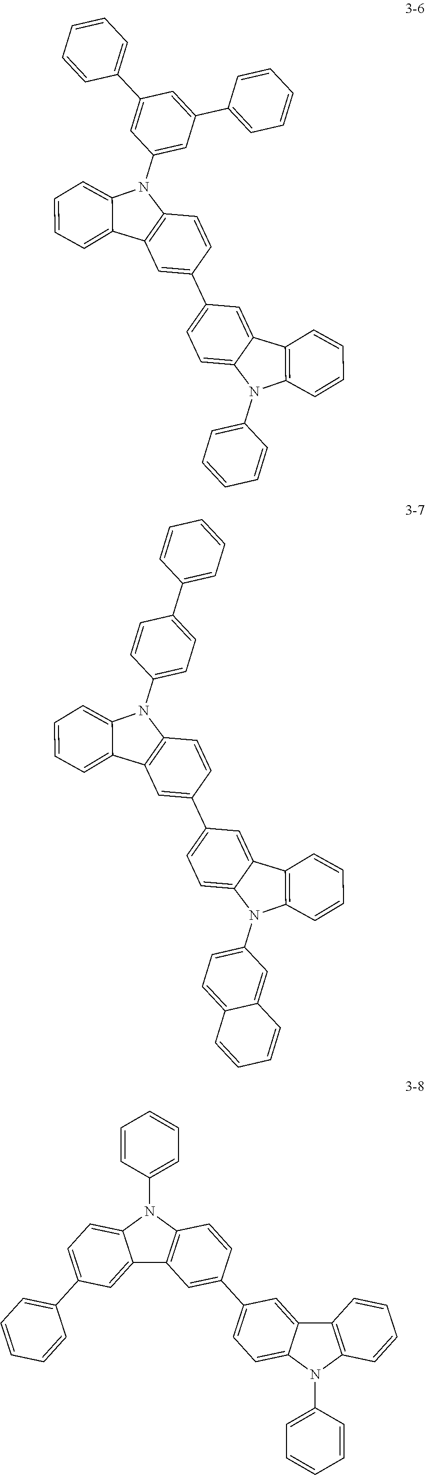

13. A composition for forming an organic material layer, comprising the heterocyclic compound of claim 1.

14. The composition of claim 13, further comprising a compound of the following Chemical Formula 2 or 3: 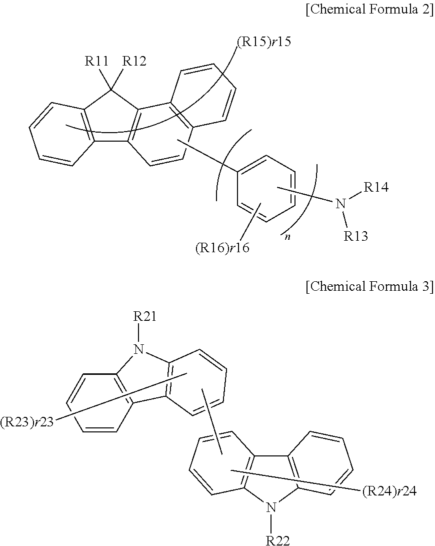
in Chemical Formulae 2 and 3,
R11 to R14 and R21 to R24 are each independently hydrogen; deuterium; a substituted or unsubstituted alkyl group having 1 to 60 carbon atoms; a substituted or unsubstituted aryl group having 6 to 60 carbon atoms; or a substituted or unsubstituted heteroaryl group having 2 to 60 carbon atoms,
R15 and R16 are each independently hydrogen; or deuterium,
n is 0 or 1,
r15 is an integer from 0 to 9,
r16 is an integer from 0 to 4,
r23 and r24 are each an integer from 0 to 7, and
when r15, r16, r23 and r24 are each 2 or higher, substituents in the parenthesis are the same as or different from each other.

15. The composition of claim 14, wherein a weight ratio of the heterocyclic compound and the compound of Chemical Formula 2 or 3 is 1:10 to 10:1.
